(19) United States
(12) Patent Application Publication
(10) Pub. No.: US 2025/0059260 A1
(43) Pub. Date: Feb. 20, 2025

(54) METHODS FOR DETECTING OR TREATING INFLUENZA INFECTIONS

(71) Applicant: THE UNIVERSITY OF CHICAGO, Chicago, IL (US)

(72) Inventors: Jenna GUTHMILLER, Chicago, IL (US); Patrick WILSON, Chicago, IL (US)

(73) Assignee: THE UNIVERSITY OF CHICAGO, Chicago, IL (US)

(21) Appl. No.: 18/723,099

(22) PCT Filed: Dec. 21, 2022
(86) PCT No.: PCT/US2022/082115

§ 371 Date: Jun. 21, 2024

Related U.S. Application Data
(60) Provisional application No. 63/292,804, filed on Dec. 22, 2021.

(51) Int. Cl. C07K 16/10; A61K 39/00; A61K 45/06; A61P 31/16; G01N 33/569

(52) U.S. Cl. CPC C07K 16/1018; A61K 45/06; A61P 31/16; G01N 33/56983; A61K 2039/505; C07K 2317/14; C07K 2317/24; C07K 2317/31; C07K 2317/565; C07K 2317/567; C07K 2317/76; G01N 2333/11

(57) ABSTRACT

To address the need in the art, the inventors have comprehensively characterized Aspects of the disclosure relate to novel antibody and antigen binding fragments. Further aspects relate to polypeptides comprising the antigen binding fragment(s) of the disclosure, and compositions comprising the polypeptides, antibodies, and/or antigen binding fragments of the disclosure. Also described are nucleic acids encoding an antibody or antigen binding fragment of the disclosure.

### CROSS-REFERENCE TO RELATED APPLICATIONS

This application claims the benefit of priority of U.S. Provisional Patent Application No. 63/292,804 filed Dec. 22, 2021, which is hereby incorporated by reference in its entirety.

### STATEMENT OF GOVERNMENT SUPPORT

This invention was made with government support under AI159136, 75N93019C00051, A1109946, A1057266, A1082724, HHSN272201400005C, and 75N93019R00028 awarded by the National Institutes of Health. The government has certain rights in the invention.

### SEQUENCE LISTING

The application contains a Sequence Listing prepared in compliance with ST.26 format and is hereby incorporated by reference in its entirety. Said Sequence Listing, created on Dec. 21, 2022 is named ARCDP0759WO.xml and is 18,165,760 bytes in size.

### FIELD OF THE INVENTION

Aspects of the invention relate to at least the fields of virology and molecular biology.

### BACKGROUND

Antibodies against the major surface glycoprotein hemagglutinin (HA) are critical for providing protection against influenza virus infection^{6,7}. However, most HA-binding antibodies target variable epitopes of the HA head domain, which provide limited protection against antigenically similar influenza virus strains³. Vaccine formulations that preferentially induce antibodies against conserved epitopes of the HA head and stalk domains could provide broad and potent protectionagainst a wide array of influenza viruses. There is a need in the art for the development of better antibodies to combat the flu virus.

### SUMMARY

The disclosure describes novel antibody and antigen binding fragments. Also described are polypeptides comprising the antigen binding fragment(s) of the disclosure, and compositions comprising the polypeptides, antibodies, and/or antigen binding fragments of the disclosure. Also described are nucleic acids encoding an antibody or antigen binding fragment of the disclosure. The disclosure also relates to nucleic acids encoding an antibody heavy chain, wherein the nucleic acid has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of the nucleic acid sequences of a heavy chain of Table 2. Also described are nucleic acids encoding an antibody light chain of the disclosure, wherein the nucleic acid has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100/o (or any derivable range therein) sequence identity to one of the nucleic acid sequences of a light chain of Table 2. Also provided are vectors or expression vectors comprising nucleic acids of the disclosure and host cells comprising polypeptides, nucleic acids, vectors, antibodies, or antigen binding fragments of the disclosure. The nucleic acids of the disclosure may be DNA or RNA.

Also described is a method of a making a cell comprising transferring one or more nucleic acid(s) of the disclosure into a cell. The method may further comprise culturing the cell under conditions that allow for expression of a polypeptide from the nucleic acid. The method may further comprise isolating the expressed polypeptide. Also described is a method for producing a polypeptide comprising transferring one or more nucleic acid(s) or vector(s) of the disclosure into a cell and isolating polypeptides expressed from the nucleic acid. Methods also include a method for producing a polypeptide comprising culturing cells comprising nucleic acid(s) or vectors of the disclosure and isolating polypeptides expressed from the nucleic acid. The cell may be further defined as a human cell, B cell, T cell, Chinese hamster ovary, NS0 murine myeloma cell, PER.C6 cell, or a cell described herein.

Methods include a method for treating, preventing, vaccinating against, and/or inducing an immune response against a coronavirus infection in a subject, the method comprising administering to the subject an antibody, antigen binding fragment, polypeptide, nucleic acid, or host cell of the disclosure. Also provided is a method for evaluating a sample from a subject, the method comprising contacting a biological sample from the subject, or extract thereof, with at least one antibody, antigen binding fragment, or polypeptide of the disclosure. Also disclosed is a method for diagnosing an influenze infection in a subject, the method comprising contacting a biological sample from the subject, or extract thereof, with at least one antibody, antigen binding fragment, or polypeptide of any one of the disclosure. The compositions of the disclosure may be formulated as a vaccine for the treatment or prevention of an influenza infection. The antibodies, antigen binding fragments, or compositions of the disclosure may be used in a vaccine for preventing coronaviral infections in a subject that does not have a influenza infection. The antibodies, antigen binding fragments, or compositions of the disclosure may be used to treat a subject having a influenza infection.

The disclosure describes an antibody or antigen binding fragment comprising a heavy chain variable region and a light chain variable region, wherein the heavy chain variable region comprises a HCDR1, HCDR2, and HCDR3 from a heavy chain variable region of an antibody clone of Table 1 and wherein the light chain variable region comprises a LCDR1, LCDR2, and LCDR3 from the light chain variable region of the same clone of Table 1. Also described is an antibody or antigen binding fragment comprising a heavy chain variable region and a light chain variable region, wherein the heavy chain variable region comprises a HCDR1, HCDR2, and HCDR3 having or having at least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a HCDR1, HCDR2, and HCDR3 from a heavy chain variable region of an antibody clone of Table 1 and wherein the light chain variable region comprises a LCDR1, LCDR2, and LCDR3 having or having at least least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a LCDR1, LCDR2, and LCDR3 from the light chain variable region of the same clone of Table 1. The HCDR1, HCDR2, HCDR3, LCDR1, LCDR2, and/or LCDR3 may be determined from the variable region sequences by methods known in the art. The CDR may be a HCDR1, HCDR2, HCDR3, LCDR1, LCDR2, and/or LCDR3 determined by the Chothia method. The CDR may be a HCDR1, HCDR2, HCDR3, LCDR1, LCDR2, and/or LCDR3 determined by the Kabat method. The CDR may be a HCDR1, HCDR2, HCDR3, LCDR1, LCDR2, and/or LCDR3 determined by the IMGT method.

Also described is an antibody or antigen binding fragment in which the HCDR1, HCDR2, HCDR2, LCDR1, LCDR2, and LCDR3 each comprise an amino acid sequence that has at least 80% sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone. The HCDR1, HCDR2, HCDR2, LCDR1, LCDR2, and LCDR3 may each comprise an amino acid sequence that has or has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone. The HCDR1, HCDR2, HCDR2, LCDR1, LCDR2, and LCDR3 may each comprise the amino acid sequence of an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone.

Also described is an antibody or antigen binding fragment comprising a heavy chain variable region and a light chain variable region, wherein the heavy chain variable region comprises a HCDR1, HCDR2, and HCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the HCDR1, HCR2, HCR3 from a heavy chain variable region of a antibody clone of Table 1 and wherein the light chain variable region comprises a LCDR1, LCDR2, and LCDR3 having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LCDR1, LCDR2, and LCDR3 from the light chain variable region of the same antibody clone of Table 1. The antibody or antigen binding fragment may comprise a heavy chain variable region and a light chain variable region, wherein the heavy chain variable region comprises a HCDR1, HCDR2, and HCDR3 having or having at least 80% sequence identity to the HCDR1, HCR2, HCR3 from a heavy chain variable region of a antibody clone of Table 1 and wherein the light chain variable region comprises a LCDR1, LCDR2, and LCDR3 having at least 80% sequence identity to the LCDR1, LCDR2, and LCDR3 from the light chain variable region of the same antibody clone of Table 1. In some aspsects, the heavy chain variable region comprises a HCDR1, HCDR2, and HCDR3 having the amino acid sequence of an of a HCDR1, HCDR2, and HCDR3 of a clone of Table 1 and the light chain variable region comprises a LCDR1, LCDR2, and LCDR3 comprising the amino acid sequence of the LCDR1, LCDR2, and LCDR3 from the light chain variable region of the same clone of Table 1.

The polypeptides of the disclosure may comprise at least two antigen binding fragments or antibodies, wherein each antigen binding fragment or antibody is independently selected from an antigen binding fragment or antibody of the disclosure, such as those disclosed in Table 1. The polypeptide may be multivalent. The polypeptide may be multispecific. The polypeptide may be bispecific. The polypeptide may comprise, comprise at least, or comprise at most 1, 2, 3, 4, 5, 6, 7, 8, 9, or 10 antigen binding regions or antibodies. Each antigen binding region or antibody may be independently selected from an antigen binding region or antibody of the disclosure, such as those in Table 1. The polypeptide may have repeated units of the same antigen binding region, such as at least, at most, or exactly 1, 2, 3, 4, 5, 6, 7, 8, 9, or 10 repeated units.

The heavy chain variable region may comprise an amino acid sequence with at least 80% sequence identity to a heavy chain variable region of an antibody clone of Table 1 and/or the light chain variable region may comprise an amino acid sequence with at least 80% sequence identity to the light chain variable region of the same antibody clone of Table 1. The heavy chain variable region may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain variable region of an antibody clone of Table 1 and/or the light chain variable region may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain variable region of the same antibody clone of Table 1. The heavy chain variable region may comprise the amino acid sequence of a heavy chain variable region of an antibody clone of Table 1 and/or the light chain variable region may comprise the amino acid sequence of the same antibody clone of Table 1.

The framework regions are the regions between the CDR sequences. For example, HFR1 is the heavy chain framework region immediately before (amino proximal) to the HCDR1. HFR2 is between HCDR1 and HCDR2. HFR3 is between HCDR2 and HCDR3. HFR4 is after (carboxy proximal) to HCDR3. LFR1 is the light chain framework region immediately before (amino proximal) to the LCDR1. LFR2 is between LCDR1 and LCDR2. LFR3 is between LCDR2 and LCDR3. LFR4 is after (carboxy proximal) LCDR3. The antibody or antigen binding fragment may comprise a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 comprises an amino acid sequence with at least 80% sequence identity to an HFR1, HFR2, HFR3, and HFR4, respectively, of an antibody clone of Table 1, and the LFR1, LFR2, LFR3, and LFR4 may comprise an amino acid sequence with at least 80% sequence identity to the LFR1, LFR2, LFR3, and LFR4, respectively, of the same antibody clone of Table 1. The antibody or antigen binding fragment may comprise a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to an HFR1, HFR2, HFR3, and HFR4, respectively, of an antibody clone of Table 1, and the LFR1, LFR2, LFR3, and LFR4 may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LFR1, LFR2, LFR3, and LFR4, respectively, of the same antibody clone of Table 1. The HFR1, HFR2, HFR3, and HFR4 may comprise the amino acid sequence of an HFR1, HFR2, HFR3, and HFR4, respectively, of an antibody clone of Table 1, and the LFR1, LFR2, LFR3, and LFR4 may comprise the amino acid sequence of the LFR1, LFR2, LFR3, and LFR4, respectively, of the same antibody clone of Table 1. The antibody or antigen binding fragment may comprise a heavy chain and a light chain and wherein the heavy chain may comprise an amino acid sequence with at least 70% sequence identity to a heavy chain of an antibody clone of Table 1 and the light chain may comprise an amino acid sequence with at least 70% sequence identity to the light chain of the same antibody clone of Table 1. The antibody or antigen binding fragment may comprise a heavy chain and a light chain and wherein the heavy chain may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain of an antibody clone of Table 1 and the light chain may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain of the same antibody clone of Table 1. The antibody or antigen binding fragment may comprise a heavy chain and a light chain and wherein the heavy chain may comprise the amino acid sequence of an antibody clone of Table 1 and the light chain may comprise the amino acid sequence of the same antibody clone of Table 1.

Aspects of the disclosure relate to an antibody or antigen binding fragment comprising a heavy chain variable region and a light chain variable region, wherein the heavy chain variable region comprises a HCDR1, HCDR2, and HCDR3 from a heavy chain variable region of an antibody clone encoded by a heavy chain nucleic acid of Table 2 and wherein the light chain variable region comprises a LCDR1, LCDR2, and LCDR3 from the light chain variable region of the same clone encoded by the light chain nucleic acid of Table 2. Further aspects relate to an antibody or antigen binding fragment comprising a heavy chain variable region and a light chain variable region, wherein the heavy chain variable region comprises a HCDR1, HCDR2, and HCDR3 having or having at least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a HCDR1, HCDR2, and HCDR3 from a heavy chain variable region of an antibody clone encoded by a heavy chain nucleic acid of Table 2 and wherein the light chain variable region comprises a LCDR1, LCDR2, and LCDR3 having or having at least least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a LCDR1, LCDR2, and LCDR3 from the light chain variable region of the same clone encoded by a light chain nucleic acid of Table 2. The HCDR1, HCDR2, HCDR3, LCDR1, LCDR2, and/or LCDR3 may be determined from the variable region sequences by methods known in the art. In some aspects, the CDR is HCDR1, HCDR2, HCDR3, LCDR1, LCDR2, and/or LCDR3 determined by the Chothia method. In some aspects, the CDR is HCDR1, HCDR2, HCDR3, LCDR1, LCDR2, and/or LCDR3 determined by the Kabat method. In some aspects, the CDR is HCDR1, HCDR2, HCDR3, LCDR1, LCDR2, and/or LCDR3 determined by the IMGT method.

Aspects of the disclosure relate to an antibody or antigen binding fragment in which the HCDR1, HCDR2, HCDR2, LCDR1, LCDR2, and LCDR3 each comprise an amino acid sequence that has at least 80% sequence identity to an HCDR1, HCDR2, HCDR2, LCDR1, LCDR2, and LCDR3 of Table 2, wherein the HCDR1, HCDR2, HCDR2, LCDR1, LCDR2, and LCDR3 are from the same antibody clone encoded by a heavy and light chain nucleic acid of Table 2.

Aspects of the disclosure relate to an antibody or antigen binding fragment comprising a heavy chain variable region and a light chain variable region, wherein the heavy chain variable region comprises a HCDR1, HCDR2, and HCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 10000 (or any derivable range therein) sequence identity to the HCDR1, HCR2, HCR3 from a heavy chain variable region of a antibody clone encoded by a heavy chain nucleic acid of Table 2 and wherein the light chain variable region comprises a LCDR1, LCDR2, and LCDR3 having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LCDR1, LCDR2, and LCDR3 from the light chain variable region of the same antibody clone encoded by a light chain nucleic acid of Table 2. In some aspects, the antibody or antigen binding fragment comprises a heavy chain variable region and a light chain variable region, wherein the heavy chain variable region comprises a HCDR1, HCDR2, and HCDR3 having or having at least 80% sequence identity to the HCDR1, HCR2, HCR3 from a heavy chain variable region of a antibody clone encoded by a heavy chain nucleic acid of Table 2 and wherein the light chain variable region comprises a LCDR1, LCDR2, and LCDR3 having at least 80% sequence identity to the LCDR1, LCDR2, and LCDR3 from the light chain variable region of the same antibody clone encoded by a light chain nucleic acid of Table 2. In some aspsects, the heavy chain variable region comprises a HCDR1, HCDR2, and HCDR3 having the amino acid sequence of an of a HCDR1, HCDR2, and HCDR3 of a clone encoded by a heavy chain nucleic acid of Table 2 and the light chain variable region comprises a LCDR1, LCDR2, and LCDR3 comprising the amino acid sequence of the LCDR1, LCDR2, and LCDR3 from the light chain variable region of the same clone encoded by a light chain nucleic acid of Table 2.

The polypeptides of the disclosure may comprise at least two antigen binding fragments, wherein each antigen binding fragment is independently selected from an antigen binding fragment of the disclosure. In some aspects, the polypeptide is multivalent. In some aspects, the polypeptide is multispecific. In some aspects, the polypeptide is bispecific. In some aspects, the polypeptide comprises, comprises at least, or comprises at most 1, 2, 3, 4, 5, 6, 7, 8, 9, or 10 antigen binding regions. Each antigen binding region may be independently selected from an antigen binding region of the disclosure. In some aspects, the polypeptide may have repeated units of the same antigen binding region, such as at least, at most, or exactly 1, 2, 3, 4, 5, 6, 7, 8, 9, or 10 repeated units.

In some aspects, the heavy chain variable region comprises an amino acid sequence with at least 80% sequence identity to a heavy chain variable region of an antibody clone encoded by a heavy chain nucleic acid of Table 2 and/or the light chain variable region comprises an amino acid sequence with at least 80% sequence identity to the light chain variable region of the same antibody clone encoded by a light chain nucleic acid of Table 2. In some aspects, the heavy chain variable region comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain variable region of an antibody clone encoded by a heavy chain nucleic acid of Table 2 and/or the light chain variable region comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain variable region of the same antibody clone encoded by a light chain nucleic acid of Table 2. In some aspects, the heavy chain variable region comprises the amino acid sequence of a heavy chain variable region of an antibody clone encoded by a heavy chain nucleic acid of Table 2 and/or the light chain variable region comprises the amino acid sequence of the same antibody clone encoded by a light chain nucleic acid of Table 2. In some aspects, the antibody or antigen binding fragment comprises a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 comprises an amino acid sequence with at least 80% sequence identity to an HFR1, HFR2, HFR3, and HFR4, respectively, of an antibody clone encoded by a heavy chain nucleic acid of Table 2, and the LFR1, LFR2, LFR3, and LFR4 comprises an amino acid sequence with at least 80% sequence identity to the LFR1, LFR2, LFR3, and LFR4, respectively, of the same antibody clone encoded by a light chain nucleic acid of Table 2. In some aspects, the antibody or antigen binding fragment comprises a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to an HFR1, HFR2, HFR3, and HFR4, respectively, of an antibody clone encoded by a heavy chain nucleic acid of Table 2, and the LFR1, LFR2, LFR3, and LFR4 comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LFR1, LFR2, LFR3, and LFR4, respectively, of the same antibody clone encoded by a light chain nucleic acid of Table 2. In some aspects, the HFR1, HFR2, HFR3, and HFR4 comprises the amino acid sequence of an HFR1, HFR2, HFR3, and HFR4, respectively, of an antibody clone encoded by a heavy chain nucleic acid of Table 2, and the LFR1, LFR2, LFR3, and LFR4 comprises the amino acid sequence of the LFR1, LFR2, LFR3, and LFR4, respectively, of the same antibody clone encoded by a light chain nucleic acid of Table 2. In some aspects, the antibody or antigen binding fragment comprises a heavy chain and a light chain and wherein the heavy chain comprises an amino acid sequence with at least 70% sequence identity to a heavy chain of an antibody clone encoded by a heavy chain nucleic acid of Table 2 and the light chain comprises an amino acid sequence with at least 70% sequence identity to the light chain of the same antibody clone encoded by a light chain nucleic acid of Table 2. In some aspects, the antibody or antigen binding fragment comprises a heavy chain and a light chain and wherein the heavy chain comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 10000 (or any derivable range therein) sequence identity to a heavy chain of an antibody clone encoded by a heavy chain nucleic acid of Table 2 and the light chain comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain of the same antibody clone encoded by a light chain nucleic acid of Table 2. In some aspects, the antibody or antigen binding fragment comprises a heavy chain and a light chain and wherein the heavy chain comprises the amino acid sequence of an antibody clone encoded by a heavy chain nucleic acid of Table 2 and the light chain comprises the amino acid sequence of the same antibody clone encoded by a light chain nucleic acid of Table 2.

In some aspects, the heavy chain variable region comprises a heavy chain framework region that has or has at least 80% sequence identity to a heavy chain framework region of an antibody clone encoded by a heavy chain nucleic acid of Table 2 and the light chain variable region comprises a light chain framework region that has or has at least 80% sequence identity to a light chain framework region of the same antibody clone encoded by a light chain nucleic acid of Table 2. In some aspects, the heavy chain variable region comprises a heavy chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain framework region of an antibody clone encoded by a heavy chain nucleic acid of Table 2 and the light chain variable region comprises a light chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a light chain framework region of the same antibody clone encoded by a light chain nucleic acid of Table 2.

In some aspects, the heavy chain variable region comprises at least 70% sequence identity to the heavy chain variable region of an antibody clone encoded by a heavy chain nucleic acid of Table 2 and the light chain variable region comprises at least 70% sequence identity to the light chain variable region of the same antibody clone encoded by a light chain nucleic acid of Table 2, and wherein the heavy chain and light chain comprise 100% sequence identity to each of the three heavy chain CDRs and three light chain CDRs from the same antibody clone of Table 2. In some aspects, the heavy chain variable region comprises at least 75% sequence identity to the heavy chain variable region of an antibody clone encoded by a heavy chain nucleic acid of Table 2 and the light chain variable region comprises at least 75% sequence identity to the light chain variable region of the same antibody clone encoded by a light chain nucleic acid of Table 2, and wherein the heavy chain and light chain comprise 100% sequence identity to each of the three heavy chain CDRs and three light chain CDRs from the same antibody clone of Table 2. In some aspects, the heavy chain variable region comprises at least 80% sequence identity to the heavy chain variable region of an antibody clone encoded by a heavy chain nucleic acid of Table 2 and the light chain variable region comprises at least 80% sequence identity to the light chain variable region of the same antibody clone encoded by a light chain nucleic acid of Table 2, and wherein the heavy chain and light chain comprise 100% sequence identity to each of the three heavy chain CDRs and three light chain CDRs from the same antibody clone of Table 2. In some aspects, the heavy chain variable region comprises at least 85% sequence identity to the heavy chain variable region of an antibody clone encoded by a heavy chain nucleic acid of Table 2 and the light chain variable region comprises at least 85% sequence identity to the light chain variable region of the same antibody clone encoded by a light chain nucleic acid of Table 2, and wherein the heavy chain and light chain comprise 100% sequence identity to each of the three heavy chain CDRs and three light chain CDRs from the same antibody clone of Table 2. In some aspects, the heavy chain variable region comprises at least 90% sequence identity to the heavy chain variable region of an antibody clone encoded by a heavy chain nucleic acid of Table 2 and the light chain variable region comprises at least 90% sequence identity to the light chain variable region of the same antibody clone encoded by a light chain nucleic acid of Table 2, and wherein the heavy chain and light chain comprise 100% sequence identity to each of the three heavy chain CDRs and three light chain CDRs from the same antibody clone of Table 2. In some aspects, the heavy chain variable region comprises at least 95% sequence identity to the heavy chain variable region of an antibody clone encoded by a heavy chain nucleic acid of Table 2 and the light chain variable region comprises at least 95% sequence identity to the light chain variable region of the same antibody clone encoded by a light chain nucleic acid of Table 2, and wherein the heavy chain and light chain comprise 100% sequence identity to each of the three heavy chain CDRs and three light chain CDRs from the same antibody clone of Table 2.

The antibody or antigen binding fragment of the disclosure may be human, chimeric, or humanized. The antibody, or antigen binding fragment may bind an influenza antigen with a kD of about 10⁻⁶ nM to about 10⁻¹² pM. The antibody, or antigen binding fragment may bind an influenza antigen with a kD of about, a kD of at least, or a kD of at most 10⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶, 10⁻¹⁷, or 10⁻¹⁸ (or any derivable range therein) μM, nM, or pM. The antibody may be further defined as a neutralizing antibody. The antibody or antigen binding fragment may be further defined as a human antibody or antigen binding fragment, humanized antibody or antigen binding fragment, recombinant antibody or antigen binding fragment, chimeric antibody or antigen binding fragment, an antibody or antigen binding fragment derivative, a veneered antibody or antigen binding fragment, a diabody, a monoclonal antibody or antigen binding fragment, a single domain antibody, or a single chain antibody. The antigen binding fragment may be further defined as a single chain variable fragment (scFv), F(ab′)2, Fab′, Fab, Fv, or rIgG. The antibody, antigen binding fragment, or polypeptide may be operatively linked to a detectable label. Detectable labels are described herein.

Also provided are multi-specific and/or multivalent antibodies and polypeptides. The disclosure provides for bivalent or bispecific antibodies that comprise two antigen binding fragments, wherein the antigen binding fragment is two of the same antigen binding fragments or two different antigen binding fragments described herein. The disclosure also provides for multi-specific polypeptides. The polypeptides may comprise at least 2, 3, 4, 5, or 6 antigen binding fragments.

The antigen binding fragment may be at least 2, 3, 4, 5, or 6 scFv, F(ab′)₂, Fab′, Fab, Fv, or rIgG, or combinations thereof. The polypeptide and/or antigen binding fragments of the disclosure may comprise a linker between a heavy chain and light chain variable region or between antigen binding fragments. The linker may be a flexible linker. Exemplary flexible linkers include glycine polymers (G)n, glycine-serine polymers (including, for example, (GS)n, (GSGGS-SEQ ID NO: 18642)n, (G₄S)n and (GGGS-SEQ ID NO: 18643)n, where n is an integer of at least one. n may be at least, at most, or exactly 1, 2, 3, 4, 5, 6, 7, 8, 9, or 10 (or any derivable range therein). Glycine-alanine polymers, alanine-serine polymers, and other flexible linkers known in the art and may be used as a linker in the polypeptides of the disclosure. Exemplary linkers can comprise or consist of GGSG (SEQ ID NO: 18644), GGSGG (SEQ ID NO:18645), GSGSG (SEQ ID NO:18646), GSGGG (SEQ ID NO:18647), GGGSG (SEQ ID NO:18648), GSSSG (SEQ ID NO: 18649), and the like.

Compositions of the disclosure, such as pharmaceutical compositions may comprise a pharmaceutical excipient, carrier, or molecule described herein. The composition may further comprises an adjuvant or an immunostimulator. Such adjuvants or immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherichia coli, Salmonella minnesota, Salmonella typhimurium, or Shigella flexneri or specifically with MPL (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as ASO1, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments. Compositions may comprise more than one antibody and/or antigen binding fragment of the disclosure. Accordingly, compositions of the disclosure may comprise, may comprise at least, or may comprise at most 1, 2, 3, 4, 5, 6, 7, 8, 9, or 10 antibodies and/or antigen binding fragments of the disclosure, wherein each antibody or antigen binding fragment is independently selected from an antibody or antigen binding fragment of the disclosure, such as those shown in Table 1. The compositions of the disclosure may be formulated for a route of administration described herein. The composition, antibody, antigen binding fragment, or polypeptide may be formulated for parenteral, intravenous, subcutaneous, intramuscular, or intranasal administration. The compositions may be formulated for intranasal administration.

The polypeptides, compositions, antibodies, antigen binding fragments, nucleic acids, or host cells, when administered to a subject, may be provided or may be provided at least, or may be provided at most 1, 2, 3, 4, 5, 6, 7, 8, 9, 10, 11, 12, 13, 14, 15, 16, 17, 18, 19, or 20 times (or any derivable range therein) over the course of, over the course of at least, or over the course of at most 1, 2, 3, 4, 5, 6, 7, 8, 9, 10, 11, 12, 13, or 14 days or 1, 2, 3, 4, 5, 6, 7, 8 9, 10, 11, 12, 13, 14, 15, 16, 17, 18, 19, or 20 weeks, or 1, 2, 3, 4, 5, 6, 7, 8, 9, 10, 11, or 12 months, or 1, 2, 3, 4, 5, 6, 7, 8, 9, or 10 years (or any range derivable therein).

The host cell may be a human cell, B cell, T cell, Chinese hamster ovary, NS0 murine myeloma cell, or PER.C6 cell. The host cell may be a cell type or cell population described herein.

The subject or patient may be a human subject or a human patient. The subject or patient may be a non-human animal. The non-human animal may be a bat, monkey, camel, rat, mouse, rabbit, goat, chicken, bird, cat, or dog. The subject may further be defined as an at-risk subject. At-risk subjects include health care workers, immunocompromised subjects, people over the age of 65, or those with at least one or at least two underlying conditions. Example of underlying conditions include obesity, high blood pressure, autoimmunity, cancer, and asthma. The subject may be one has one or more symptoms of an influenza infection. Symptoms of an influenza infection include, but are not limited to elevated temperature or a fever of 100.0° F. or more, difficulty breathing, shortness of breath, fatigue, headache, chills, sore throat, congestion or runny nose, shaking or exaggerated shivering, significant muscle pain or ache, diarrhea, and/or nausea or vomiting. The subject may be one that does not have any symptoms of an influenza infection. The subject may be one that has been diagnosed with an influenza infection. The subject may be one that has not been diagnosed with an influenza infection. The subject may be one that has been previously treated for an influenza infection. The subject may be one that has been previously vaccinated for influenza. The subject may be one that has not been previously vaccinated for influenza. The influenza infection may comprise an H1N1 influenza infection. The influenza infection may comprise an H1H2 infection. The previous treatment may comprise a pain reliever, such as acetaminophen or ibuprofen, a steroid such as dexamethasone, prednisolone, beclomethasone, fluticasone, or methylprednisone or an antiviral such as remdesivir. The subject may be administered an additional therapeutic. The additional therapeutic may comprise one or more of a pain reliever, such as acetaminophen or ibuprofen, a steroid such as dexamethasone, prednisolone, beclomethasone, fluticasone, or methylprednisone or an antiviral such as oseltamivir, zanamivir, peramivir, or baloxavir.

The method may further comprise incubating the antibody, antigen binding fragment, or polypeptide under conditions that allow for the binding of the antibody, antigen binding fragment, or polypeptide to antigens in the biological sample or extract thereof. The method may further comprise detecting the binding of an antigen to the antibody, antigen binding fragment, or polypeptide. The method may further comprise contacting the biological sample with at least one capture antibody, antigen, or polypeptide. The biological sample may comprise a blood sample, urine sample, fecal sample, or nasopharyngeal sample.

Aspects of the disclosure are also described in “A public broadly neutralizing antibody class targets a membrane-proximal anchor epitope of influenza virus hemagglutinin Jenna J. Guthmiller, Julianna Han, Henry A. Utset, Lei Li, Linda Yu-Ling Lan, Carole Henry, Christopher T. Stamper, Olivia Stovicek, Lauren Gentles, Haley L. Dugan, Nai-Ying Zheng, Sara T. Richey, Micah E. Tepora, Dalia J. Bitar, Siriruk Changrob, Shimn Strohmeier, Min Huang, Adolfo Garcia-Sastre, Raffael Nachbagauer, Peter Palese, Jesse D. Bloom, Florian Krammer, Lynda Coughlan, Andrew B. Ward, Patrick C. Wilson; bioRxiv 2021.02.25.432905; and the supplemental materials related thereto: Li, Lei; Guthmiller, Jenna; Lan, Linda; Wilson, Patrick (2021), “Broadly neutralizing antibodies target a hemagglutinin anchor epitope, Guthmiller et al. 2021”, Mendeley Data, V1, doi: 10.17632/jzsx489pmk.1, both of which are herein incorporated by reference for all purposes.

Also described are multi-specific and/or multivalent antibodies and polypeptides. Accordingly, described are bivalent or bispecific antibodies that comprise two antigen binding fragments, wherein the antigen binding fragment is two of the same antigen binding fragments or two different antigen binding fragments described herein. The disclosure also provides for multi-specific polypeptides. Also disclosed are polypeptides comprising or comprising at least 2, 3, 4, 5, or 6 antigen binding fragments.

Methods may comprise or further comprise incubating the antibody, antigen binding fragment, or polypeptide under conditions that allow for the binding of the antibody, antigen binding fragment, or polypeptide to antigens in the biological sample or extract thereof. The method may comprise detecting the binding of an antigen to the antibody, antigen binding fragment, or polypeptide. The method may further comprise contacting the biological sample with at least one capture antibody, antigen, or polypeptide. The at least one capture antibody, antigen binding fragment, or polypeptide may be an antibody, polypeptide, or antigen binding fragment of the disclosure. The capture antibody may be linked or operatively linked to a solid support. The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. The biological sample may comprise a blood sample, urine sample, fecal sample, or nasopharyngeal sample. The at least one antibody, antigen binding fragment, or polypeptide may be operatively linked to a detectable label.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17275, 18029, and 3941, respectively and the LCDR1, LCDR2, and LCDR3 comprises an amino acid sequence of SEQ ID NOS: 16056, 9503, and 12778, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4138, and 5911, respectively and the LCDR1, LCDR2, and LCDR3 comprises an amino acid sequence of SEQ ID NOS: 16037, 4425, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17273, 4149, and 7845, respectively and the LCDR1, LCDR2, and LCDR3 comprises an amino acid sequence of SEQ ID NOS: 15964, 4425, and 10711, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11668, 4139, and 6334, respectively and the LCDR1, LCDR2, and LCDR3 comprises an amino acid sequence of SEQ ID NOS: 16037, 4425, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17628, 18542, and 9462, respectively and the LCDR1, LCDR2, and LCDR3 comprises an amino acid sequence of SEQ ID NOS: 16045, 9503, and 12947, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16519, 4138, and 5907, respectively and the LCDR1, LCDR2, and LCDR3 comprises an amino acid sequence of SEQ ID NOS:15887, 10225, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17634, 18542, and 10208, respectively and the LCDR1, LCDR2, and LCDR3 comprises an amino acid sequence of SEQ ID NOS:16109, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10339, 16888, and 6142, respectively and the LCDR1, LCDR2, and LCDR3 comprises an amino acid sequence of SEQ ID NOS: 16076, 4432, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 18251, 16888, and 6142, respectively and the LCDR1, LCDR2, and LCDR3 comprises an amino acid sequence of SEQ ID NOS: 16076, 4425, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17274, 4138, and 6550, respectively and the LCDR1, LCDR2, and LCDR3 comprises an amino acid sequence of SEQ ID NOS: 16015, 4400, and 12433, respectively. having a LCDR1, LCDR2, and LCDR3, wherein the HCDR1, HCDR2, and HCDR3 comprises an amino acid sequence of SEQ ID NOS:4303, 16921, and 6142, respectively and the LCDR1, LCDR2, and LCDR3 comprises an amino acid sequence of SEQ ID NOS: 16076, 4425, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11622, 18491, and 4403, respectively and the LCDR1, LCDR2, and LCDR3 comprises an amino acid sequence of SEQ ID NOS: 15867, 9502, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10937, 10406, and 4405, respectively and the LCDR1, LCDR2, and LCDR3 comprises an amino acid sequence of SEQ ID NOS: 16045, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 10404, and 4598, respectively and the LCDR1, LCDR2, and LCDR3 comprises an amino acid sequence of SEQ ID NOS: 15958, 9503, and 12724, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17446, 4138, and 10174, respectively and the LCDR1, LCDR2, and LCDR3 comprises an amino acid sequence of SEQ ID NOS: 16045, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4302, 9894, and 10124, respectively and the LCDR1, LCDR2, and LCDR3 comprises an amino acid sequence of SEQ ID NOS: 15968, 6211, and 12666, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17445, 11019, and 7810, respectively and the LCDR1, LCDR2, and LCDR3 comprises an amino acid sequence of SEQ ID NOS: 16746, 17025, and 4263, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10934, 9864, and 4608, respectively and the LCDR1, LCDR2, and LCDR3 comprises an amino acid sequence of SEQ ID NOS: 15727, 9494, and 12687, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 9384, and 9544, respectively and the LCDR1, LCDR2, and LCDR3 comprises an amino acid sequence of SEQ ID NOS:17905, 16330, and 16565, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16990, 10052, and 4205, respectively and the LCDR1, LCDR2, and LCDR3 comprises an amino acid sequence of SEQ ID NOS: 10830, 7825, and 11302, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 11512, and 6669, respectively and the LCDR1, LCDR2, and LCDR3 comprises an amino acid sequence of SEQ ID NOS:15803, 11791, and 12839, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6560, 4156, and 6710, respectively and the LCDR1, LCDR2, and LCDR3 comprises an amino acid sequence of SEQ ID NOS:18044, 11072, and 13612, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11562, 16250, and 10357, respectively and the LCDR1, LCDR2, and LCDR3 comprises an amino acid sequence of SEQ ID NOS:10223, 6213, and 11054, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10100, 18318, and 10167, respectively and the LCDR1, LCDR2, and LCDR3 comprises an amino acid sequence of SEQ ID NOS: 15701, 9496, and 12922, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17275, 9826, and 4035, respectively and the LCDR1, LCDR2, and LCDR3 comprises an amino acid sequence of SEQ ID NOS: 16021, 9494, and 12690, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6156, 11022, and 10173, respectively and the LCDR1, LCDR2, and LCDR3 comprises an amino acid sequence of SEQ ID NOS:16454, 18279, and 12895, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17855, 18573, and 9340, respectively and the LCDR1, LCDR2, and LCDR3 comprises an amino acid sequence of SEQ ID NOS: 16108, 3969, and 12490, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9659, 11531, and 10034, respectively and the LCDR1, LCDR2, and LCDR3 comprises an amino acid sequence of SEQ ID NOS: 15644, 6322, and 12380, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 15582, 18398, and 9468, respectively and the LCDR1, LCDR2, and LCDR3 comprises an amino acid sequence of SEQ ID NOS: 15965, 9503, and 12564, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 15590, 18356, and 9465, respectively and the LCDR1, LCDR2, and LCDR3 comprises an amino acid sequence of SEQ ID NOS: 15986, 9503, and 12787, respectively. having a LCDR1, LCDR2, and LCDR3, wherein the HCDR1, HCDR2, and HCDR3 comprises an amino acid sequence of SEQ ID NOS: 10352, 18315, and 6016, respectively and the LCDR1, LCDR2, and LCDR3 comprises an amino acid sequence of SEQ ID NOS: 15878, 4413, and 12431, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 16478, and 4198, respectively and the LCDR1, LCDR2, and LCDR3 comprises an amino acid sequence of SEQ ID NOS:16693, 16156, and 13624, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9971, and 4487, respectively and the LCDR1, LCDR2, and LCDR3 comprises an amino acid sequence of SEQ ID NOS: 17966, 6717, and 17206, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16994, 10050, and 4205, respectively and the LCDR1, LCDR2, and LCDR3 comprises an amino acid sequence of SEQ ID NOS: 10830, 9273, and 11297, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6558, 9696, and 9548, respectively and the LCDR1, LCDR2, and LCDR3 comprises an amino acid sequence of SEQ ID NOS: 15773, 4415, and 12411, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11679, 11543, and 10252, respectively and the LCDR1, LCDR2, and LCDR3 comprises an amino acid sequence of SEQ ID NOS:10782, 18277, and 12901, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10534, 16275, and 4247, respectively and the LCDR1, LCDR2, and LCDR3 comprises an amino acid sequence of SEQ ID NOS:16388, 18272, and 12007, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6546, 18556, and 6131, respectively and the LCDR1, LCDR2, and LCDR3 comprises an amino acid sequence of SEQ ID NOS: 15804, 4425, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17842, 11502, and 11737, respectively and the LCDR1, LCDR2, and LCDR3 comprises an amino acid sequence of SEQ ID NOS:15739, 11613, and 12880, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17534, 17815, and 16369, respectively and the LCDR1, LCDR2, and LCDR3 comprises an amino acid sequence of SEQ ID NOS:16004, 9494, and 12652, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11632, 4154, and 6035, respectively and the LCDR1, LCDR2, and LCDR3 comprises an amino acid sequence of SEQ ID NOS:16642, 10747, and 13326, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17988, 18293, and 3959, respectively and the LCDR1, LCDR2, and LCDR3 comprises an amino acid sequence of SEQ ID NOS: 15663, 4259, and 12812, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9678, 17782, and 9661, respectively and the LCDR1, LCDR2, and LCDR3 comprises an amino acid sequence of SEQ ID NOS: 15730, 9469, and 11926, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17649, 17996, and 11542, respectively and the LCDR1, LCDR2, and LCDR3 comprises an amino acid sequence of SEQ ID NOS: 16392, 9272, and 11242, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11539, 18037, and 10487, respectively and the LCDR1, LCDR2, and LCDR3 comprises an amino acid sequence of SEQ ID NOS:9584, 5976, and 15576, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6563, 16941, and 16292, respectively and the LCDR1, LCDR2, and LCDR3 comprises an amino acid sequence of SEQ ID NOS: 15849, 3953, and 10456, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17112, 10413, and 9644, respectively and the LCDR1, LCDR2, and LCDR3 comprises an amino acid sequence of SEQ ID NOS: 15850, 6612, and 12824, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17870, 18098, and 18225, respectively and the LCDR1, LCDR2, and LCDR3 comprises an amino acid sequence of SEQ ID NOS:10812, 18275, and 12943, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16611, 4218, and 6159, respectively and the LCDR1, LCDR2, and LCDR3 comprises an amino acid sequence of SEQ ID NOS: 15889, 9494, and 12616, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 16869, and 6128, respectively and the LCDR1, LCDR2, and LCDR3 comprises an amino acid sequence of SEQ ID NOS: 15958, 9503, and 12725, respectively. having a LCDR1, LCDR2, and LCDR3, wherein the HCDR1, HCDR2, and HCDR3 comprises an amino acid sequence of SEQ ID NOS:6566, 11467, and 10415, respectively and the LCDR1, LCDR2, and LCDR3 comprises an amino acid sequence of SEQ ID NOS: 10818, 7977, and 11242, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9904, and 10125, respectively and the LCDR1, LCDR2, and LCDR3 comprises an amino acid sequence of SEQ ID NOS: 15783, 3974, and 12595, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11156, 16875, and 11709, respectively and the LCDR1, LCDR2, and LCDR3 comprises an amino acid sequence of SEQ ID NOS:16129, 4019, and 12553, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10953, 10538, and 17212, respectively and the LCDR1, LCDR2, and LCDR3 comprises an amino acid sequence of SEQ ID NOS:17959, 9261, and 17573, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17144, 10387, and 11138, respectively and the LCDR1, LCDR2, and LCDR3 comprises an amino acid sequence of SEQ ID NOS:15640, 6215, and 12646, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4208, 18095, and 10486, respectively and the LCDR1, LCDR2, and LCDR3 comprises an amino acid sequence of SEQ ID NOS:6704, 4467, and 15574, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10340, 18486, and 6077, respectively and the LCDR1, LCDR2, and LCDR3 comprises an amino acid sequence of SEQ ID NOS:16730, 7918, and 4265, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18100, 10047, and 4202, respectively and the LCDR1, LCDR2, and LCDR3 comprises an amino acid sequence of SEQ ID NOS: 10830, 9272, and 11255, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16596, 18567, and 5967, respectively and the LCDR1, LCDR2, and LCDR3 comprises an amino acid sequence of SEQ ID NOS:16485, 16558, and 12506, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17558, 11036, and 17675, respectively and the LCDR1, LCDR2, and LCDR3 comprises an amino acid sequence of SEQ ID NOS:16708, 16159, and 3993, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11409, 10399, and 9683, respectively and the LCDR1, LCDR2, and LCDR3 comprises an amino acid sequence of SEQ ID NOS: 15634, 4414, and 11930, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17269, 10067, and 9350, respectively and the LCDR1, LCDR2, and LCDR3 comprises an amino acid sequence of SEQ ID NOS:17922, 10088, and 13573, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17609, 9816, and 6023, respectively and the LCDR1, LCDR2, and LCDR3 comprises an amino acid sequence of SEQ ID NOS:16975, 10589, and 13611, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16541, 16242, and 4572, respectively and the LCDR1, LCDR2, and LCDR3 comprises an amino acid sequence of SEQ ID NOS: 17984, 9261, and 17153, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17028, 17823, and 16315, respectively and the LCDR1, LCDR2, and LCDR3 comprises an amino acid sequence of SEQ ID NOS: 17923, 10094, and 13580, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17649, 10395, and 6058, respectively and the LCDR1, LCDR2, and LCDR3 comprises an amino acid sequence of SEQ ID NOS:10820, 11190, and 11308, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17136, 16936, and 10382, respectively and the LCDR1, LCDR2, and LCDR3 comprises an amino acid sequence of SEQ ID NOS:16013, 9503, and 12781, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4305, 9893, and 10123, respectively and the LCDR1, LCDR2, and LCDR3 comprises an amino acid sequence of SEQ ID NOS: 15969, 6326, and 12721, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4631, 11451, and 11105, respectively and the LCDR1, LCDR2, and LCDR3 comprises an amino acid sequence of SEQ ID NOS:16448, 18275, and 12926, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17640, 16907, and 5947, respectively and the LCDR1, LCDR2, and LCDR3 comprises an amino acid sequence of SEQ ID NOS: 15949, 4413, and 12404, respectively. having a LCDR1, LCDR2, and LCDR3, wherein the HCDR1, HCDR2, and HCDR3 comprises an amino acid sequence of SEQ ID NOS:17535, 17736, and 10426, respectively and the LCDR1, LCDR2, and LCDR3 comprises an amino acid sequence of SEQ ID NOS:10809, 18271, and 13008, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18103, 18040, and 11160, respectively and the LCDR1, LCDR2, and LCDR3 comprises an amino acid sequence of SEQ ID NOS:16664, 10749, and 18616, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16990, 4072, and 10078, respectively and the LCDR1, LCDR2, and LCDR3 comprises an amino acid sequence of SEQ ID NOS: 10849, 7824, and 11248, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6566, 11482, and 17716, respectively and the LCDR1, LCDR2, and LCDR3 comprises an amino acid sequence of SEQ ID NOS:10867, 18275, and 12940, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10363, 11440, and 9439, respectively and the LCDR1, LCDR2, and LCDR3 comprises an amino acid sequence of SEQ ID NOS: 16430, 9259, and 11305, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11618, 11024, and 18604, respectively and the LCDR1, LCDR2, and LCDR3 comprises an amino acid sequence of SEQ ID NOS: 16429, 10764, and 11286, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16990, 9755, and 7908, respectively and the LCDR1, LCDR2, and LCDR3 comprises an amino acid sequence of SEQ ID NOS:16051, 4442, and 12617, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11624, 10040, and 18255, respectively and the LCDR1, LCDR2, and LCDR3 comprises an amino acid sequence of SEQ ID NOS:15781, 17254, and 12909, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17849, 11494, and 18196, respectively and the LCDR1, LCDR2, and LCDR3 comprises an amino acid sequence of SEQ ID NOS:10899, 18279, and 12906, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6577, 4330, and 6135, respectively and the LCDR1, LCDR2, and LCDR3 comprises an amino acid sequence of SEQ ID NOS:16037, 4425, and 10721, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10686, 17775, and 4562, respectively and the LCDR1, LCDR2, and LCDR3 comprises an amino acid sequence of SEQ ID NOS: 16045, 9503, and 12998, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16200, 9401, and 4033, respectively and the LCDR1, LCDR2, and LCDR3 comprises an amino acid sequence of SEQ ID NOS:16698, 10093, and 17255, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6576, 18326, and 9668, respectively and the LCDR1, LCDR2, and LCDR3 comprises an amino acid sequence of SEQ ID NOS:15890, 10708, and 12570, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11595, 10391, and 10530, respectively and the LCDR1, LCDR2, and LCDR3 comprises an amino acid sequence of SEQ ID NOS:9448, 4467, and 15568, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11679, 11448, and 17831, respectively and the LCDR1, LCDR2, and LCDR3 comprises an amino acid sequence of SEQ ID NOS:7914, 18275, and 12961, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17570, 16916, and 4582, respectively and the LCDR1, LCDR2, and LCDR3 comprises an amino acid sequence of SEQ ID NOS:10862, 18275, and 12346, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16545, 11440, and 9439, respectively and the LCDR1, LCDR2, and LCDR3 comprises an amino acid sequence of SEQ ID NOS: 16419, 9259, and 11304, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17881, 9997, and 4583, respectively and the LCDR1, LCDR2, and LCDR3 comprises an amino acid sequence of SEQ ID NOS: 15667, 4256, and 11091, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17613, 18411, and 11938, respectively and the LCDR1, LCDR2, and LCDR3 comprises an amino acid sequence of SEQ ID NOS: 16484, 6317, and 12362, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6546, 18526, and 6134, respectively and the LCDR1, LCDR2, and LCDR3 comprises an amino acid sequence of SEQ ID NOS: 16067, 4424, and 12433, respectively. having a LCDR1, LCDR2, and LCDR3, wherein the HCDR1, HCDR2, and HCDR3 comprises an amino acid sequence of SEQ ID NOS:11551, 9906, and 10121, respectively and the LCDR1, LCDR2, and LCDR3 comprises an amino acid sequence of SEQ ID NOS: 16004, 9479, and 12619, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 16919, and 9601, respectively and the LCDR1, LCDR2, and LCDR3 comprises an amino acid sequence of SEQ ID NOS: 15704, 3969, and 12828, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11615, 16248, and 7911, respectively and the LCDR1, LCDR2, and LCDR3 comprises an amino acid sequence of SEQ ID NOS:16660, 11903, and 11891, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10356, 16838, and 10688, respectively and the LCDR1, LCDR2, and LCDR3 comprises an amino acid sequence of SEQ ID NOS:13333, 18275, and 12955, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17534, 11524, and 16206, respectively and the LCDR1, LCDR2, and LCDR3 comprises an amino acid sequence of SEQ ID NOS: 15755, 4425, and 12422, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10934, 9866, and 4607, respectively and the LCDR1, LCDR2, and LCDR3 comprises an amino acid sequence of SEQ ID NOS: 15723, 9480, and 12598, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6535, 9383, and 6019, respectively and the LCDR1, LCDR2, and LCDR3 comprises an amino acid sequence of SEQ ID NOS:17909, 16325, and 17236, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17649, 11475, and 18205, respectively and the LCDR1, LCDR2, and LCDR3 comprises an amino acid sequence of SEQ ID NOS:16447, 18275, and 12400, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16689, 18458, and 9558, respectively and the LCDR1, LCDR2, and LCDR3 comprises an amino acid sequence of SEQ ID NOS: 16066, 6607, and 12822, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16992, 18002, and 10072, respectively and the LCDR1, LCDR2, and LCDR3 comprises an amino acid sequence of SEQ ID NOS:10838, 9267, and 11249, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9969, and 4042, respectively and the LCDR1, LCDR2, and LCDR3 comprises an amino acid sequence of SEQ ID NOS: 16489, 9478, and 12666, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11648, 18357, and 9467, respectively and the LCDR1, LCDR2, and LCDR3 comprises an amino acid sequence of SEQ ID NOS:15975, 10316, and 12786, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16990, 10050, and 4201, respectively and the LCDR1, LCDR2, and LCDR3 comprises an amino acid sequence of SEQ ID NOS: 10828, 6793, and 11296, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11651, 16900, and 7904, respectively and the LCDR1, LCDR2, and LCDR3 comprises an amino acid sequence of SEQ ID NOS:18082, 18135, and 13621, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11538, 16874, and 10488, respectively and the LCDR1, LCDR2, and LCDR3 comprises an amino acid sequence of SEQ ID NOS:9583, 6537, and 15575, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17028, 10578, and 10186, respectively and the LCDR1, LCDR2, and LCDR3 comprises an amino acid sequence of SEQ ID NOS: 16045, 9500, and 12787, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11387, 18487, and 6075, respectively and the LCDR1, LCDR2, and LCDR3 comprises an amino acid sequence of SEQ ID NOS:16770, 16574, and 17670, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11679, 16843, and 11162, respectively and the LCDR1, LCDR2, and LCDR3 comprises an amino acid sequence of SEQ ID NOS:16451, 18273, and 10471, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6546, 18528, and 6133, respectively and the LCDR1, LCDR2, and LCDR3 comprises an amino acid sequence of SEQ ID NOS: 15794, 4425, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16990, 9699, and 10501, respectively and the LCDR1, LCDR2, and LCDR3 comprises an amino acid sequence of SEQ ID NOS: 16072, 9494, and 12717, respectively. having a LCDR1, LCDR2, and LCDR3, wherein the HCDR1, HCDR2, and HCDR3 comprises an amino acid sequence of SEQ ID NOS:4302, 9896, and 10125, respectively and the LCDR1, LCDR2, and LCDR3 comprises an amino acid sequence of SEQ ID NOS: 16087, 9480, and 12636, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17576, 8078, and 4486, respectively and the LCDR1, LCDR2, and LCDR3 comprises an amino acid sequence of SEQ ID NOS: 16747, 10089, and 3997, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11558, 10572, and 11121, respectively and the LCDR1, LCDR2, and LCDR3 comprises an amino acid sequence of SEQ ID NOS:16667, 11895, and 11867, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17293, 18416, and 15587, respectively and the LCDR1, LCDR2, and LCDR3 comprises an amino acid sequence of SEQ ID NOS: 15641, 6319, and 12358, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16990, 10250, and 11693, respectively and the LCDR1, LCDR2, and LCDR3 comprises an amino acid sequence of SEQ ID NOS: 16097, 10737, and 12020, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11639, 16918, and 9663, respectively and the LCDR1, LCDR2, and LCDR3 comprises an amino acid sequence of SEQ ID NOS:16114, 10727, and 12847, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17474, 16231, and 4596, respectively and the LCDR1, LCDR2, and LCDR3 comprises an amino acid sequence of SEQ ID NOS:16408, 10987, and 11213, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17843, 10396, and 6057, respectively and the LCDR1, LCDR2, and LCDR3 comprises an amino acid sequence of SEQ ID NOS:10831, 11161, and 11308, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17464, 18542, and 4591, respectively and the LCDR1, LCDR2, and LCDR3 comprises an amino acid sequence of SEQ ID NOS: 15808, 6310, and 10711, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17648, 5434, and 6727, respectively and the LCDR1, LCDR2, and LCDR3 comprises an amino acid sequence of SEQ ID NOS:17959, 9275, and 17150, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9901, and 10118, respectively and the LCDR1, LCDR2, and LCDR3 comprises an amino acid sequence of SEQ ID NOS: 15814, 9493, and 12666, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16581, 18505, and 9556, respectively and the LCDR1, LCDR2, and LCDR3 comprises an amino acid sequence of SEQ ID NOS:15890, 10726, and 12808, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16990, 9719, and 10500, respectively and the LCDR1, LCDR2, and LCDR3 comprises an amino acid sequence of SEQ ID NOS: 15923, 9503, and 9654, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11672, 11521, and 16205, respectively and the LCDR1, LCDR2, and LCDR3 comprises an amino acid sequence of SEQ ID NOS: 15999, 4425, and 12434, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16999, 18402, and 6705, respectively and the LCDR1, LCDR2, and LCDR3 comprises an amino acid sequence of SEQ ID NOS: 16045, 9503, and 10776, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17561, 10766, and 9537, respectively and the LCDR1, LCDR2, and LCDR3 comprises an amino acid sequence of SEQ ID NOS:15861, 10702, and 12854, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11386, 9700, and 10431, respectively and the LCDR1, LCDR2, and LCDR3 comprises an amino acid sequence of SEQ ID NOS: 10819, 9267, and 11333, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4634, 4328, and 17145, respectively and the LCDR1, LCDR2, and LCDR3 comprises an amino acid sequence of SEQ ID NOS:15683, 17864, and 12728, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16594, 16232, and 10038, respectively and the LCDR1, LCDR2, and LCDR3 comprises an amino acid sequence of SEQ ID NOS: 15883, 9503, and 12892, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10516, 11452, and 10528, respectively and the LCDR1, LCDR2, and LCDR3 comprises an amino acid sequence of SEQ ID NOS: 15752, 10705, and 12734, respectively. having a LCDR1, LCDR2, and LCDR3, wherein the HCDR1, HCDR2, and HCDR3 comprises an amino acid sequence of SEQ ID NOS:4034, 9691, and 18058, respectively and the LCDR1, LCDR2, and LCDR3 comprises an amino acid sequence of SEQ ID NOS: 15602, 4425, and 12414, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16707, 11529, and 10034, respectively and the LCDR1, LCDR2, and LCDR3 comprises an amino acid sequence of SEQ ID NOS:15714, 6321, and 12379, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16701, 11530, and 10034, respectively and the LCDR1, LCDR2, and LCDR3 comprises an amino acid sequence of SEQ ID NOS: 15709, 6323, and 12380, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16572, 17894, and 9536, respectively and the LCDR1, LCDR2, and LCDR3 comprises an amino acid sequence of SEQ ID NOS:15890, 10705, and 12808, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4031, 9749, and 17119, respectively and the LCDR1, LCDR2, and LCDR3 comprises an amino acid sequence of SEQ ID NOS: 10778, 9255, and 11250, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16596, 18566, and 5946, respectively and the LCDR1, LCDR2, and LCDR3 comprises an amino acid sequence of SEQ ID NOS:16486, 16558, and 12505, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11652, 16928, and 6118, respectively and the LCDR1, LCDR2, and LCDR3 comprises an amino acid sequence of SEQ ID NOS: 15838, 9503, and 12725, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17274, 9949, and 4592, respectively and the LCDR1, LCDR2, and LCDR3 comprises an amino acid sequence of SEQ ID NOS:10858, 18268, and 16346, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16618, 10003, and 17119, respectively and the LCDR1, LCDR2, and LCDR3 comprises an amino acid sequence of SEQ ID NOS:10823, 9272, and 11234, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16990, 4073, and 10079, respectively and the LCDR1, LCDR2, and LCDR3 comprises an amino acid sequence of SEQ ID NOS:16411, 7824, and 11248, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10934, 9865, and 4608, respectively and the LCDR1, LCDR2, and LCDR3 comprises an amino acid sequence of SEQ ID NOS: 15726, 9494, and 12716, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9920, and 17713, respectively and the LCDR1, LCDR2, and LCDR3 comprises an amino acid sequence of SEQ ID NOS:15891, 10705, and 12601, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16990, 10051, and 4200, respectively and the LCDR1, LCDR2, and LCDR3 comprises an amino acid sequence of SEQ ID NOS: 10830, 9269, and 11246, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17855, 18574, and 4036, respectively and the LCDR1, LCDR2, and LCDR3 comprises an amino acid sequence of SEQ ID NOS: 17959, 6311, and 4363, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16686, 18586, and 9539, respectively and the LCDR1, LCDR2, and LCDR3 comprises an amino acid sequence of SEQ ID NOS: 15895, 6610, and 12752, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9896, and 10125, respectively and the LCDR1, LCDR2, and LCDR3 comprises an amino acid sequence of SEQ ID NOS: 15837, 9494, and 12666, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9677, 18485, and 6076, respectively and the LCDR1, LCDR2, and LCDR3 comprises an amino acid sequence of SEQ ID NOS: 16739, 10205, and 4261, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6576, 18331, and 5979, respectively and the LCDR1, LCDR2, and LCDR3 comprises an amino acid sequence of SEQ ID NOS: 17959, 8080, and 17165, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11669, 16870, and 4438, respectively and the LCDR1, LCDR2, and LCDR3 comprises an amino acid sequence of SEQ ID NOS:16085, 16563, and 12725, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11552, 9907, and 10126, respectively and the LCDR1, LCDR2, and LCDR3 comprises an amino acid sequence of SEQ ID NOS: 15915, 9503, and 12607, respectively. having a LCDR1, LCDR2, and LCDR3, wherein the HCDR1, HCDR2, and HCDR3 comprises an amino acid sequence of SEQ ID NOS:16610, 9890, and 4050, respectively and the LCDR1, LCDR2, and LCDR3 comprises an amino acid sequence of SEQ ID NOS: 10830, 9272, and 11236, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9929, and 9528, respectively and the LCDR1, LCDR2, and LCDR3 comprises an amino acid sequence of SEQ ID NOS: 10841, 9274, and 11238, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6539, 17893, and 9517, respectively and the LCDR1, LCDR2, and LCDR3 comprises an amino acid sequence of SEQ ID NOS:10914, 18272, and 12893, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17837, 11460, and 10394, respectively and the LCDR1, LCDR2, and LCDR3 comprises an amino acid sequence of SEQ ID NOS:10818, 7978, and 11244, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17872, 10048, and 4202, respectively and the LCDR1, LCDR2, and LCDR3 comprises an amino acid sequence of SEQ ID NOS: 10830, 9272, and 11255, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11648, 18358, and 9466, respectively and the LCDR1, LCDR2, and LCDR3 comprises an amino acid sequence of SEQ ID NOS: 16030, 9503, and 12754, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18623, 18602, and 16968, respectively and the LCDR1, LCDR2, and LCDR3 comprises an amino acid sequence of SEQ ID NOS:16098, 10521, and 12537, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17638, 16504, and 4482, respectively and the LCDR1, LCDR2, and LCDR3 comprises an amino acid sequence of SEQ ID NOS:16466, 15521, and 11272, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 9389, and 4624, respectively and the LCDR1, LCDR2, and LCDR3 comprises an amino acid sequence of SEQ ID NOS:15811, 10705, and 12888, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16990, 10046, and 4202, respectively and the LCDR1, LCDR2, and LCDR3 comprises an amino acid sequence of SEQ ID NOS:10819, 9272, and 11301, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6573, 17743, and 6666, respectively and the LCDR1, LCDR2, and LCDR3 comprises an amino acid sequence of SEQ ID NOS: 17934, 4283, and 17170, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17284, 18415, and 10133, respectively and the LCDR1, LCDR2, and LCDR3 comprises an amino acid sequence of SEQ ID NOS:10890, 18275, and 12976, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6549, 18552, and 6092, respectively and the LCDR1, LCDR2, and LCDR3 comprises an amino acid sequence of SEQ ID NOS: 15863, 4425, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 11433, and 11134, respectively and the LCDR1, LCDR2, and LCDR3 comprises an amino acid sequence of SEQ ID NOS:18091, 11073, and 13615, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17441, 9735, and 17123, respectively and the LCDR1, LCDR2, and LCDR3 comprises an amino acid sequence of SEQ ID NOS: 10839, 9255, and 11247, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16954, and 10482, respectively and the LCDR1, LCDR2, and LCDR3 comprises an amino acid sequence of SEQ ID NOS: 17919, 9278, and 4346, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10510, 18354, and 9467, respectively and the LCDR1, LCDR2, and LCDR3 comprises an amino acid sequence of SEQ ID NOS: 15626, 9503, and 12756, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17293, 18414, and 15586, respectively and the LCDR1, LCDR2, and LCDR3 comprises an amino acid sequence of SEQ ID NOS: 16483, 6325, and 12358, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17264, 9891, and 16776, respectively and the LCDR1, LCDR2, and LCDR3 comprises an amino acid sequence of SEQ ID NOS:9482, 4425, and 12685, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10947, 16929, and 6068, respectively and the LCDR1, LCDR2, and LCDR3 comprises an amino acid sequence of SEQ ID NOS: 16045, 9503, and 12868, respectively. having a LCDR1, LCDR2, and LCDR3, wherein the HCDR1, HCDR2, and HCDR3 comprises an amino acid sequence of SEQ ID NOS: 10944, 9981, and 9523, respectively and the LCDR1, LCDR2, and LCDR3 comprises an amino acid sequence of SEQ ID NOS: 10837, 9274, and 11085, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16545, 11441, and 9439, respectively and the LCDR1, LCDR2, and LCDR3 comprises an amino acid sequence of SEQ ID NOS: 16393, 9259, and 11306, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17278, 9745, and 6159, respectively and the LCDR1, LCDR2, and LCDR3 comprises an amino acid sequence of SEQ ID NOS: 16028, 9494, and 12872, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16990, 9903, and 7922, respectively and the LCDR1, LCDR2, and LCDR3 comprises an amino acid sequence of SEQ ID NOS: 16010, 9480, and 11996, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11552, 9724, and 10200, respectively and the LCDR1, LCDR2, and LCDR3 comprises an amino acid sequence of SEQ ID NOS:15654, 16561, and 12093, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9832, and 10745, respectively and the LCDR1, LCDR2, and LCDR3 comprises an amino acid sequence of SEQ ID NOS: 16061, 9494, and 12693, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17857, 9367, and 10499, respectively and the LCDR1, LCDR2, and LCDR3 comprises an amino acid sequence of SEQ ID NOS: 18083, 17678, and 9447, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17028, 17825, and 17567, respectively and the LCDR1, LCDR2, and LCDR3 comprises an amino acid sequence of SEQ ID NOS:15805, 18442, and 10449, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10071, 9887, and 10073, respectively and the LCDR1, LCDR2, and LCDR3 comprises an amino acid sequence of SEQ ID NOS: 10838, 9272, and 11248, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17014, 16501, and 4046, respectively and the LCDR1, LCDR2, and LCDR3 comprises an amino acid sequence of SEQ ID NOS: 16061, 9494, and 12870, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16990, 10049, and 4201, respectively and the LCDR1, LCDR2, and LCDR3 comprises an amino acid sequence of SEQ ID NOS: 10828, 9273, and 11298, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11561, 18233, and 6724, respectively and the LCDR1, LCDR2, and LCDR3 comprises an amino acid sequence of SEQ ID NOS: 16116, 6609, and 12821, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9980, and 9524, respectively and the LCDR1, LCDR2, and LCDR3 comprises an amino acid sequence of SEQ ID NOS: 10841, 9274, and 11238, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4320, 16210, and 4315, respectively and the LCDR1, LCDR2, and LCDR3 comprises an amino acid sequence of SEQ ID NOS: 17937, 9276, and 17125, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11632, 4133, and 16185, respectively and the LCDR1, LCDR2, and LCDR3 comprises an amino acid sequence of SEQ ID NOS: 15703, 3982, and 12092, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6145, 11023, and 10215, respectively and the LCDR1, LCDR2, and LCDR3 comprises an amino acid sequence of SEQ ID NOS:16454, 18279, and 10472, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6524, 18317, and 10216, respectively and the LCDR1, LCDR2, and LCDR3 comprises an amino acid sequence of SEQ ID NOS: 15892, 9480, and 11999, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11623, 9841, and 10108, respectively and the LCDR1, LCDR2, and LCDR3 comprises an amino acid sequence of SEQ ID NOS:15968, 10222, and 12664, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11553, 9906, and 10128, respectively and the LCDR1, LCDR2, and LCDR3 comprises an amino acid sequence of SEQ ID NOS: 15920, 9494, and 12568, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16542, 11459, and 11159, respectively and the LCDR1, LCDR2, and LCDR3 comprises an amino acid sequence of SEQ ID NOS:10909, 18275, and 12905, respectively. having a LCDR1, LCDR2, and LCDR3, wherein the HCDR1, HCDR2, and HCDR3 comprises an amino acid sequence of SEQ ID NOS:17449, 9736, and 17118, respectively and the LCDR1, LCDR2, and LCDR3 comprises an amino acid sequence of SEQ ID NOS: 10830, 9272, and 11234, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17147, 10571, and 16314, respectively and the LCDR1, LCDR2, and LCDR3 comprises an amino acid sequence of SEQ ID NOS:15803, 6611, and 12869, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11392, 9382, and 9599, respectively and the LCDR1, LCDR2, and LCDR3 comprises an amino acid sequence of SEQ ID NOS: 17955, 8077, and 17183, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6231, 11504, and 9351, respectively and the LCDR1, LCDR2, and LCDR3 comprises an amino acid sequence of SEQ ID NOS: 16385, 9266, and 11279, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17272, 18015, and 4610, respectively and the LCDR1, LCDR2, and LCDR3 comprises an amino acid sequence of SEQ ID NOS: 16045, 9503, and 12767, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11689, 18573, and 9342, respectively and the LCDR1, LCDR2, and LCDR3 comprises an amino acid sequence of SEQ ID NOS: 16110, 3953, and 12499, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9892, and 10127, respectively and the LCDR1, LCDR2, and LCDR3 comprises an amino acid sequence of SEQ ID NOS: 16001, 9494, and 12666, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11623, 9813, and 16776, respectively and the LCDR1, LCDR2, and LCDR3 comprises an amino acid sequence of SEQ ID NOS:9485, 4425, and 12685, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9820, and 7921, respectively and the LCDR1, LCDR2, and LCDR3 comprises an amino acid sequence of SEQ ID NOS: 16049, 9480, and 12712, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6549, 18511, and 6132, respectively and the LCDR1, LCDR2, and LCDR3 comprises an amino acid sequence of SEQ ID NOS: 15857, 4425, and 12406, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16990, 9886, and 10073, respectively and the LCDR1, LCDR2, and LCDR3 comprises an amino acid sequence of SEQ ID NOS: 10840, 9272, and 11248, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 10571, and 16181, respectively and the LCDR1, LCDR2, and LCDR3 comprises an amino acid sequence of SEQ ID NOS:16674, 6637, and 11887, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17534, 11525, and 16206, respectively and the LCDR1, LCDR2, and LCDR3 comprises an amino acid sequence of SEQ ID NOS: 15994, 4425, and 12422, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17261, 9906, and 10119, respectively and the LCDR1, LCDR2, and LCDR3 comprises an amino acid sequence of SEQ ID NOS: 16004, 9494, and 12666, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17072, 16854, and 10425, respectively and the LCDR1, LCDR2, and LCDR3 comprises an amino acid sequence of SEQ ID NOS:17917, 9277, and 17203, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 10010, and 7923, respectively and the LCDR1, LCDR2, and LCDR3 comprises an amino acid sequence of SEQ ID NOS: 15886, 9494, and 12279, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10952, 11514, and 11153, respectively and the LCDR1, LCDR2, and LCDR3 comprises an amino acid sequence of SEQ ID NOS: 16375, 9266, and 17542, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17855, 18573, and 9341, respectively and the LCDR1, LCDR2, and LCDR3 comprises an amino acid sequence of SEQ ID NOS: 16108, 3969, and 12490, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10384, 11465, and 11541, respectively and the LCDR1, LCDR2, and LCDR3 comprises an amino acid sequence of SEQ ID NOS: 10821, 9272, and 11243, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17448, 9371, and 6567, respectively and the LCDR1, LCDR2, and LCDR3 comprises an amino acid sequence of SEQ ID NOS: 15701, 6605, and 12990, respectively. having a LCDR1, LCDR2, and LCDR3, wherein the HCDR1, HCDR2, and HCDR3 comprises an amino acid sequence of SEQ ID NOS:4305, 9895, and 10120, respectively and the LCDR1, LCDR2, and LCDR3 comprises an amino acid sequence of SEQ ID NOS: 15774, 9503, and 12721, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10919, 11445, and 9440, respectively and the LCDR1, LCDR2, and LCDR3 comprises an amino acid sequence of SEQ ID NOS: 16401, 9259, and 11305, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16200, 9400, and 4032, respectively and the LCDR1, LCDR2, and LCDR3 comprises an amino acid sequence of SEQ ID NOS: 15692, 3953, and 11097, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4316, 9908, and 10122, respectively and the LCDR1, LCDR2, and LCDR3 comprises an amino acid sequence of SEQ ID NOS:16120, 10225, and 12630, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4619, 11510, and 9552, respectively and the LCDR1, LCDR2, and LCDR3 comprises an amino acid sequence of SEQ ID NOS: 17943, 9270, and 17155, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4319, 11524, and 16204, respectively and the LCDR1, LCDR2, and LCDR3 comprises an amino acid sequence of SEQ ID NOS: 15994, 4422, and 12422, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6531, 18022, and 16977, respectively and the LCDR1, LCDR2, and LCDR3 comprises an amino acid sequence of SEQ ID NOS:16387, 10926, and 11230, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17534, 17815, and 16369, respectively and the LCDR1, LCDR2, and LCDR3 comprises an amino acid sequence of SEQ ID NOS:10714, 4424, and 12435, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11552, 9905, and 10125, respectively and the LCDR1, LCDR2, and LCDR3 comprises an amino acid sequence of SEQ ID NOS: 15921, 9494, and 12565, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6559, 16866, and 5952, respectively and the LCDR1, LCDR2, and LCDR3 comprises an amino acid sequence of SEQ ID NOS:15821, 4417, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17293, 18418, and 15588, respectively and the LCDR1, LCDR2, and LCDR3 comprises an amino acid sequence of SEQ ID NOS:15718, 6325, and 12358, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9896, and 10125, respectively and the LCDR1, LCDR2, and LCDR3 comprises an amino acid sequence of SEQ ID NOS: 15831, 9503, and 12666, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10936, 9928, and 9527, respectively and the LCDR1, LCDR2, and LCDR3 comprises an amino acid sequence of SEQ ID NOS: 16461, 9274, and 11264, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17277, 9902, and 10121, respectively and the LCDR1, LCDR2, and LCDR3 comprises an amino acid sequence of SEQ ID NOS: 16088, 9494, and 12711, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11551, 9897, and 10121, respectively and the LCDR1, LCDR2, and LCDR3 comprises an amino acid sequence of SEQ ID NOS: 16004, 9480, and 12666, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11645, 18353, and 17031, respectively and the LCDR1, LCDR2, and LCDR3 comprises an amino acid sequence of SEQ ID NOS: 15656, 3969, and 12339, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11425, 18601, and 10301, respectively and the LCDR1, LCDR2, and LCDR3 comprises an amino acid sequence of SEQ ID NOS:16115, 9490, and 12511, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17042, 9993, and 10379, respectively and the LCDR1, LCDR2, and LCDR3 comprises an amino acid sequence of SEQ ID NOS: 15959, 9494, and 12671, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6534, 18437, and 5995, respectively and the LCDR1, LCDR2, and LCDR3 comprises an amino acid sequence of SEQ ID NOS: 15761, 3982, and 12471, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9797, and 17662, respectively and the LCDR1, LCDR2, and LCDR3 comprises an amino acid sequence of SEQ ID NOS: 15941, 9494, and 12676, respectively. having a LCDR1, LCDR2, and LCDR3, wherein the HCDR1, HCDR2, and HCDR3 comprises an amino acid sequence of SEQ ID NOS: 10680, 18344, and 6021, respectively and the LCDR1, LCDR2, and LCDR3 comprises an amino acid sequence of SEQ ID NOS: 15873, 9498, and 12525, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11621, 18301, and 9357, respectively and the LCDR1, LCDR2, and LCDR3 comprises an amino acid sequence of SEQ ID NOS:15605, 10727, and 12845, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17003, 9818, and 10920, respectively and the LCDR1, LCDR2, and LCDR3 comprises an amino acid sequence of SEQ ID NOS:15895, 10724, and 15592, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16999, 9819, and 4224, respectively and the LCDR1, LCDR2, and LCDR3 comprises an amino acid sequence of SEQ ID NOS: 15639, 9480, and 12707, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10942, 18424, and 4491, respectively and the LCDR1, LCDR2, and LCDR3 comprises an amino acid sequence of SEQ ID NOS:16131, 9477, and 12533, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 17809, and 6109, respectively and the LCDR1, LCDR2, and LCDR3 comprises an amino acid sequence of SEQ ID NOS: 16322, 3982, and 12957, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17441, 18371, and 10177, respectively and the LCDR1, LCDR2, and LCDR3 comprises an amino acid sequence of SEQ ID NOS: 15864, 9490, and 12502, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11623, 9707, and 10489, respectively and the LCDR1, LCDR2, and LCDR3 comprises an amino acid sequence of SEQ ID NOS: 18258, 9503, and 12749, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 10002, and 10203, respectively and the LCDR1, LCDR2, and LCDR3 comprises an amino acid sequence of SEQ ID NOS:16057, 9494, and 12664, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16571, 9733, and 10202, respectively and the LCDR1, LCDR2, and LCDR3 comprises an amino acid sequence of SEQ ID NOS:15982, 9451, and 12671, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10942, 18387, and 4505, respectively and the LCDR1, LCDR2, and LCDR3 comprises an amino acid sequence of SEQ ID NOS: 16130, 6616, and 12491, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9776, and 18216, respectively and the LCDR1, LCDR2, and LCDR3 comprises an amino acid sequence of SEQ ID NOS:15890, 10727, and 12844, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17282, 18392, and 7880, respectively and the LCDR1, LCDR2, and LCDR3 comprises an amino acid sequence of SEQ ID NOS: 16005, 9494, and 12680, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17618, 18335, and 9620, respectively and the LCDR1, LCDR2, and LCDR3 comprises an amino acid sequence of SEQ ID NOS: 15746, 3964, and 11104, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11391, 10767, and 17038, respectively and the LCDR1, LCDR2, and LCDR3 comprises an amino acid sequence of SEQ ID NOS: 16003, 9503, and 12600, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17848, 10544, and 9600, respectively and the LCDR1, LCDR2, and LCDR3 comprises an amino acid sequence of SEQ ID NOS:17966, 9261, and 4371, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16950, and 11723, respectively and the LCDR1, LCDR2, and LCDR3 comprises an amino acid sequence of SEQ ID NOS:17957, 9275, and 10612, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16822, 17598, and 18230, respectively and the LCDR1, LCDR2, and LCDR3 comprises an amino acid sequence of SEQ ID NOS:16118, 3950, and 12487, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16822, 16952, and 6193, respectively and the LCDR1, LCDR2, and LCDR3 comprises an amino acid sequence of SEQ ID NOS:15890, 10727, and 12840, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17857, 18591, and 6329, respectively and the LCDR1, LCDR2, and LCDR3 comprises an amino acid sequence of SEQ ID NOS: 15594, 9490, and 12482, respectively. having a LCDR1, LCDR2, and LCDR3, wherein the HCDR1, HCDR2, and HCDR3 comprises an amino acid sequence of SEQ ID NOS:17263, 9814, and 17002, respectively and the LCDR1, LCDR2, and LCDR3 comprises an amino acid sequence of SEQ ID NOS:9484, 4432, and 12658, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11391, 10767, and 17038, respectively and the LCDR1, LCDR2, and LCDR3 comprises an amino acid sequence of SEQ ID NOS: 16003, 9503, and 12703, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11382, 10539, and 6627, respectively and the LCDR1, LCDR2, and LCDR3 comprises an amino acid sequence of SEQ ID NOS: 15853, 3982, and 12510, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17890, 9388, and 7903, respectively and the LCDR1, LCDR2, and LCDR3 comprises an amino acid sequence of SEQ ID NOS:15966, 16560, and 12508, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 17755, and 10289, respectively and the LCDR1, LCDR2, and LCDR3 comprises an amino acid sequence of SEQ ID NOS: 16036, 9503, and 12725, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11416, 17242, and 6670, respectively and the LCDR1, LCDR2, and LCDR3 comprises an amino acid sequence of SEQ ID NOS: 16643, 6634, and 9426, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16777, 10412, and 11892, respectively and the LCDR1, LCDR2, and LCDR3 comprises an amino acid sequence of SEQ ID NOS: 15893, 9475, and 12493, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16821, 11537, and 6469, respectively and the LCDR1, LCDR2, and LCDR3 comprises an amino acid sequence of SEQ ID NOS: 15827, 9490, and 12492, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 17809, and 4210, respectively and the LCDR1, LCDR2, and LCDR3 comprises an amino acid sequence of SEQ ID NOS:16408, 10987, and 11206, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17644, 10571, and 16207, respectively and the LCDR1, LCDR2, and LCDR3 comprises an amino acid sequence of SEQ ID NOS:15893, 3961, and 12480, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10937, 16865, and 4051, respectively and the LCDR1, LCDR2, and LCDR3 comprises an amino acid sequence of SEQ ID NOS: 16036, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11632, 18005, and 10192, respectively and the LCDR1, LCDR2, and LCDR3 comprises an amino acid sequence of SEQ ID NOS: 16457, 3955, and 12405, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16987, 9913, and 17724, respectively and the LCDR1, LCDR2, and LCDR3 comprises an amino acid sequence of SEQ ID NOS: 16071, 9511, and 12723, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11373, 9800, and 9610, respectively and the LCDR1, LCDR2, and LCDR3 comprises an amino acid sequence of SEQ ID NOS: 15704, 9498, and 12305, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 10571, and 11699, respectively and the LCDR1, LCDR2, and LCDR3 comprises an amino acid sequence of SEQ ID NOS:9633, 4467, and 15560, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11670, 16894, and 6087, respectively and the LCDR1, LCDR2, and LCDR3 comprises an amino acid sequence of SEQ ID NOS: 16071, 9503, and 12867, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4494, 9373, and 7889, respectively and the LCDR1, LCDR2, and LCDR3 comprises an amino acid sequence of SEQ ID NOS: 15670, 3960, and 12830, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10367, 18323, and 16787, respectively and the LCDR1, LCDR2, and LCDR3 comprises an amino acid sequence of SEQ ID NOS: 15893, 3969, and 12524, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10367, 18323, and 7895, respectively and the LCDR1, LCDR2, and LCDR3 comprises an amino acid sequence of SEQ ID NOS: 16061, 9494, and 12692, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4134, and 17879, respectively and the LCDR1, LCDR2, and LCDR3 comprises an amino acid sequence of SEQ ID NOS: 16763, 16329, and 4246, respectively. having a LCDR1, LCDR2, and LCDR3, wherein the HCDR1, HCDR2, and HCDR3 comprises an amino acid sequence of SEQ ID NOS:17600, 9805, and 6155, respectively and the LCDR1, LCDR2, and LCDR3 comprises an amino acid sequence of SEQ ID NOS: 15823, 3969, and 12528, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9988, and 10417, respectively and the LCDR1, LCDR2, and LCDR3 comprises an amino acid sequence of SEQ ID NOS: 15704, 9490, and 12832, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6571, 4327, and 6153, respectively and the LCDR1, LCDR2, and LCDR3 comprises an amino acid sequence of SEQ ID NOS:10907, 18266, and 12921, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16800, 9982, and 10379, respectively and the LCDR1, LCDR2, and LCDR3 comprises an amino acid sequence of SEQ ID NOS: 15772, 9494, and 10465, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16805, 9756, and 17058, respectively and the LCDR1, LCDR2, and LCDR3 comprises an amino acid sequence of SEQ ID NOS: 15701, 3982, and 12095, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10757, 9851, and 4601, respectively and the LCDR1, LCDR2, and LCDR3 comprises an amino acid sequence of SEQ ID NOS:16020, 10262, and 12560, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17610, 18427, and 6746, respectively and the LCDR1, LCDR2, and LCDR3 comprises an amino acid sequence of SEQ ID NOS: 15812, 9490, and 10455, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10752, 18388, and 4505, respectively and the LCDR1, LCDR2, and LCDR3 comprises an amino acid sequence of SEQ ID NOS: 16130, 6616, and 12534, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9973, and 5963, respectively and the LCDR1, LCDR2, and LCDR3 comprises an amino acid sequence of SEQ ID NOS: 17959, 9261, and 17160, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17558, 11038, and 11123, respectively and the LCDR1, LCDR2, and LCDR3 comprises an amino acid sequence of SEQ ID NOS:11774, 4420, and 12563, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10943, 9934, and 4223, respectively and the LCDR1, LCDR2, and LCDR3 comprises an amino acid sequence of SEQ ID NOS: 15609, 4432, and 12641, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11691, 18456, and 6329, respectively and the LCDR1, LCDR2, and LCDR3 comprises an amino acid sequence of SEQ ID NOS: 15595, 3966, and 12489, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11644, 16882, and 5928, respectively and the LCDR1, LCDR2, and LCDR3 comprises an amino acid sequence of SEQ ID NOS: 15986, 4425, and 12409, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16989, 9750, and 16688, respectively and the LCDR1, LCDR2, and LCDR3 comprises an amino acid sequence of SEQ ID NOS: 15691, 3953, and 11093, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17258, 9810, and 6306, respectively and the LCDR1, LCDR2, and LCDR3 comprises an amino acid sequence of SEQ ID NOS: 15764, 3953, and 12550, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17442, 9853, and 6161, respectively and the LCDR1, LCDR2, and LCDR3 comprises an amino acid sequence of SEQ ID NOS: 16055, 9494, and 12709, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11692, 6789, and 10969, respectively and the LCDR1, LCDR2, and LCDR3 comprises an amino acid sequence of SEQ ID NOS: 16716, 5991, and 10282, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6532, 11065, and 4167, respectively and the LCDR1, LCDR2, and LCDR3 comprises an amino acid sequence of SEQ ID NOS: 16706, 11378, and 4289, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17578, 18036, and 16201, respectively and the LCDR1, LCDR2, and LCDR3 comprises an amino acid sequence of SEQ ID NOS: 15930, 10225, and 11979, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16769, 10324, and 18406, respectively and the LCDR1, LCDR2, and LCDR3 comprises an amino acid sequence of SEQ ID NOS: 15593, 10705, and 12022, respectively. having a LCDR1, LCDR2, and LCDR3, wherein the HCDR1, HCDR2, and HCDR3 comprises an amino acid sequence of SEQ ID NOS: 10775, 11034, and 4594, respectively and the LCDR1, LCDR2, and LCDR3 comprises an amino acid sequence of SEQ ID NOS: 11781, 4419, and 12345, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17473, 16236, and 16815, respectively and the LCDR1, LCDR2, and LCDR3 comprises an amino acid sequence of SEQ ID NOS: 15764, 3969, and 12513, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4321, 11500, and 6144, respectively and the LCDR1, LCDR2, and LCDR3 comprises an amino acid sequence of SEQ ID NOS: 16137, 3952, and 12476, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6187, 18562, and 11143, respectively and the LCDR1, LCDR2, and LCDR3 comprises an amino acid sequence of SEQ ID NOS: 15763, 9490, and 12526, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16174, 16932, and 5908, respectively and the LCDR1, LCDR2, and LCDR3 comprises an amino acid sequence of SEQ ID NOS: 16092, 4417, and 10711, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11366, 16249, and 11418, respectively and the LCDR1, LCDR2, and LCDR3 comprises an amino acid sequence of SEQ ID NOS:16341, 10313, and 12548, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17126, 16955, and 10377, respectively and the LCDR1, LCDR2, and LCDR3 comprises an amino acid sequence of SEQ ID NOS: 17925, 10097, and 13570, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17069, 4155, and 6552, respectively and the LCDR1, LCDR2, and LCDR3 comprises an amino acid sequence of SEQ ID NOS: 16037, 4425, and 10711, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16810, 4107, and 7891, respectively and the LCDR1, LCDR2, and LCDR3 comprises an amino acid sequence of SEQ ID NOS: 16045, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17648, 11515, and 6638, respectively and the LCDR1, LCDR2, and LCDR3 comprises an amino acid sequence of SEQ ID NOS: 16740, 11555, and 3990, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11633, 16868, and 18201, respectively and the LCDR1, LCDR2, and LCDR3 comprises an amino acid sequence of SEQ ID NOS: 16071, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11371, 16256, and 7910, respectively and the LCDR1, LCDR2, and LCDR3 comprises an amino acid sequence of SEQ ID NOS:11947, 10022, and 11593, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16194, 18034, and 17735, respectively and the LCDR1, LCDR2, and LCDR3 comprises an amino acid sequence of SEQ ID NOS: 17944, 6313, and 4360, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10115, 17214, and 10693, respectively and the LCDR1, LCDR2, and LCDR3 comprises an amino acid sequence of SEQ ID NOS: 15782, 3953, and 11983, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16823, 18561, and 15608, respectively and the LCDR1, LCDR2, and LCDR3 comprises an amino acid sequence of SEQ ID NOS: 16046, 9503, and 12605, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16619, 9369, and 17450, respectively and the LCDR1, LCDR2, and LCDR3 comprises an amino acid sequence of SEQ ID NOS: 17959, 6312, and 4353, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17628, 16899, and 4615, respectively and the LCDR1, LCDR2, and LCDR3 comprises an amino acid sequence of SEQ ID NOS:15895, 10733, and 12736, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17534, 17818, and 16495, respectively and the LCDR1, LCDR2, and LCDR3 comprises an amino acid sequence of SEQ ID NOS: 15993, 4425, and 11923, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17270, 17769, and 10198, respectively and the LCDR1, LCDR2, and LCDR3 comprises an amino acid sequence of SEQ ID NOS: 17949, 6312, and 4377, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10179, 17213, and 10679, respectively and the LCDR1, LCDR2, and LCDR3 comprises an amino acid sequence of SEQ ID NOS:16487, 3953, and 15591, respectively. having a LCDR1, LCDR2, and LCDR3, wherein the HCDR1, HCDR2, and HCDR3 comprises an amino acid sequence of SEQ ID NOS:17543, 10414, and 18420, respectively and the LCDR1, LCDR2, and LCDR3 comprises an amino acid sequence of SEQ ID NOS:16407, 11166, and 11212, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10349, 6785, and 10035, respectively and the LCDR1, LCDR2, and LCDR3 comprises an amino acid sequence of SEQ ID NOS:10815, 18272, and 12945, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11673, 10677, and 17677, respectively and the LCDR1, LCDR2, and LCDR3 comprises an amino acid sequence of SEQ ID NOS: 16413, 10987, and 11202, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:0, 9791, and 4222, respectively and the LCDR1, LCDR2, and LCDR3 comprises an amino acid sequence of SEQ ID NOS:16123, 10737, and 11993, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17991, 10571, and 15607, respectively and the LCDR1, LCDR2, and LCDR3 comprises an amino acid sequence of SEQ ID NOS:16669, 6628, and 11888, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17991, 10568, and 15607, respectively and the LCDR1, LCDR2, and LCDR3 comprises an amino acid sequence of SEQ ID NOS:16669, 6628, and 11888, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10367, 18323, and 7804, respectively and the LCDR1, LCDR2, and LCDR3 comprises an amino acid sequence of SEQ ID NOS:15816, 10727, and 12823, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10163, 18559, and 16507, respectively and the LCDR1, LCDR2, and LCDR3 comprises an amino acid sequence of SEQ ID NOS: 16041, 4436, and 12999, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10187, 9992, and 10143, respectively and the LCDR1, LCDR2, and LCDR3 comprises an amino acid sequence of SEQ ID NOS: 16049, 9494, and 12671, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16547, 10503, and 10259, respectively and the LCDR1, LCDR2, and LCDR3 comprises an amino acid sequence of SEQ ID NOS:16408, 10987, and 11343, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17261, 9771, and 16532, respectively and the LCDR1, LCDR2, and LCDR3 comprises an amino acid sequence of SEQ ID NOS: 16067, 4417, and 10713, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9961, and 4568, respectively and the LCDR1, LCDR2, and LCDR3 comprises an amino acid sequence of SEQ ID NOS: 16742, 16586, and 4287, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17451, 9994, and 7902, respectively and the LCDR1, LCDR2, and LCDR3 comprises an amino acid sequence of SEQ ID NOS:15939, 16562, and 12650, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11647, 9776, and 5894, respectively and the LCDR1, LCDR2, and LCDR3 comprises an amino acid sequence of SEQ ID NOS: 16045, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 18007, and 6081, respectively and the LCDR1, LCDR2, and LCDR3 comprises an amino acid sequence of SEQ ID NOS: 16045, 9494, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17634, 17729, and 10154, respectively and the LCDR1, LCDR2, and LCDR3 comprises an amino acid sequence of SEQ ID NOS:10890, 18275, and 12917, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16673, 18586, and 6030, respectively and the LCDR1, LCDR2, and LCDR3 comprises an amino acid sequence of SEQ ID NOS: 11789, 4420, and 12579, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4136, and 16283, respectively and the LCDR1, LCDR2, and LCDR3 comprises an amino acid sequence of SEQ ID NOS: 16045, 9503, and 12760, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10681, 16878, and 6463, respectively and the LCDR1, LCDR2, and LCDR3 comprises an amino acid sequence of SEQ ID NOS: 16761, 17586, and 4000, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10950, 11492, and 10609, respectively and the LCDR1, LCDR2, and LCDR3 comprises an amino acid sequence of SEQ ID NOS:10881, 18275, and 13007, respectively. having a LCDR1, LCDR2, and LCDR3, wherein the HCDR1, HCDR2, and HCDR3 comprises an amino acid sequence of SEQ ID NOS: 11669, 4139, and 4025, respectively and the LCDR1, LCDR2, and LCDR3 comprises an amino acid sequence of SEQ ID NOS: 16036, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6531, 9381, and 5942, respectively and the LCDR1, LCDR2, and LCDR3 comprises an amino acid sequence of SEQ ID NOS: 15835, 3969, and 12536, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 17816, and 10383, respectively and the LCDR1, LCDR2, and LCDR3 comprises an amino acid sequence of SEQ ID NOS: 17959, 10305, and 10237, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11414, 18473, and 6124, respectively and the LCDR1, LCDR2, and LCDR3 comprises an amino acid sequence of SEQ ID NOS: 15873, 3969, and 12488, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 11488, and 6110, respectively and the LCDR1, LCDR2, and LCDR3 comprises an amino acid sequence of SEQ ID NOS: 16748, 17007, and 3999, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16570, 9758, and 9602, respectively and the LCDR1, LCDR2, and LCDR3 comprises an amino acid sequence of SEQ ID NOS: 15614, 9488, and 12626, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17259, 9762, and 10019, respectively and the LCDR1, LCDR2, and LCDR3 comprises an amino acid sequence of SEQ ID NOS: 16125, 9494, and 12654, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17649, 11495, and 11152, respectively and the LCDR1, LCDR2, and LCDR3 comprises an amino acid sequence of SEQ ID NOS:10853, 18259, and 10476, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17067, 9760, and 9608, respectively and the LCDR1, LCDR2, and LCDR3 comprises an amino acid sequence of SEQ ID NOS:15951, 9494, and 12626, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6187, 18502, and 4193, respectively and the LCDR1, LCDR2, and LCDR3 comprises an amino acid sequence of SEQ ID NOS:17966, 9261, and 17164, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16991, 9852, and 10064, respectively and the LCDR1, LCDR2, and LCDR3 comprises an amino acid sequence of SEQ ID NOS: 16049, 9494, and 12671, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18042, 17826, and 10676, respectively and the LCDR1, LCDR2, and LCDR3 comprises an amino acid sequence of SEQ ID NOS: 17962, 6331, and 4356, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17262, 10013, and 16634, respectively and the LCDR1, LCDR2, and LCDR3 comprises an amino acid sequence of SEQ ID NOS: 16045, 9503, and 12429, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16196, 9765, and 11736, respectively and the LCDR1, LCDR2, and LCDR3 comprises an amino acid sequence of SEQ ID NOS:10860, 18278, and 12995, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17262, 9758, and 9606, respectively and the LCDR1, LCDR2, and LCDR3 comprises an amino acid sequence of SEQ ID NOS: 16456, 9494, and 12626, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17469, 9744, and 4185, respectively and the LCDR1, LCDR2, and LCDR3 comprises an amino acid sequence of SEQ ID NOS: 16042, 9503, and 12749, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17528, 16890, and 17245, respectively and the LCDR1, LCDR2, and LCDR3 comprises an amino acid sequence of SEQ ID NOS: 16036, 9494, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9776, and 16697, respectively and the LCDR1, LCDR2, and LCDR3 comprises an amino acid sequence of SEQ ID NOS: 16061, 9494, and 12690, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17274, 4136, and 4597, respectively and the LCDR1, LCDR2, and LCDR3 comprises an amino acid sequence of SEQ ID NOS:16642, 10747, and 13031, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17283, 18409, and 9582, respectively and the LCDR1, LCDR2, and LCDR3 comprises an amino acid sequence of SEQ ID NOS:16105, 10731, and 12889, respectively. having a LCDR1, LCDR2, and LCDR3, wherein the HCDR1, HCDR2, and HCDR3 comprises an amino acid sequence of SEQ ID NOS:6577, 11059, and 9540, respectively and the LCDR1, LCDR2, and LCDR3 comprises an amino acid sequence of SEQ ID NOS:18063, 18596, and 11066, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17549, 10582, and 10675, respectively and the LCDR1, LCDR2, and LCDR3 comprises an amino acid sequence of SEQ ID NOS: 17962, 6331, and 4356, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9741, and 4184, respectively and the LCDR1, LCDR2, and LCDR3 comprises an amino acid sequence of SEQ ID NOS: 16045, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17438, 9757, and 9607, respectively and the LCDR1, LCDR2, and LCDR3 comprises an amino acid sequence of SEQ ID NOS: 15995, 9441, and 11994, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17606, 17730, and 10155, respectively and the LCDR1, LCDR2, and LCDR3 comprises an amino acid sequence of SEQ ID NOS:10872, 18275, and 12919, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 11011, and 9674, respectively and the LCDR1, LCDR2, and LCDR3 comprises an amino acid sequence of SEQ ID NOS: 16034, 9503, and 10459, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17441, 9715, and 10442, respectively and the LCDR1, LCDR2, and LCDR3 comprises an amino acid sequence of SEQ ID NOS: 15725, 9441, and 12620, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6511, 17582, and 5965, respectively and the LCDR1, LCDR2, and LCDR3 comprises an amino acid sequence of SEQ ID NOS:10879, 18275, and 12370, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16799, 9702, and 9614, respectively and the LCDR1, LCDR2, and LCDR3 comprises an amino acid sequence of SEQ ID NOS: 15980, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11386, 9983, and 17050, respectively and the LCDR1, LCDR2, and LCDR3 comprises an amino acid sequence of SEQ ID NOS:16145, 9494, and 12645, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6556, 10256, and 11163, respectively and the LCDR1, LCDR2, and LCDR3 comprises an amino acid sequence of SEQ ID NOS:15822, 10722, and 12881, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16175, 16895, and 5950, respectively and the LCDR1, LCDR2, and LCDR3 comprises an amino acid sequence of SEQ ID NOS: 15879, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11370, 16253, and 17832, respectively and the LCDR1, LCDR2, and LCDR3 comprises an amino acid sequence of SEQ ID NOS:17913, 6629, and 13588, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10368, 6782, and 10193, respectively and the LCDR1, LCDR2, and LCDR3 comprises an amino acid sequence of SEQ ID NOS: 15893, 3969, and 12519, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4136, and 18284, respectively and the LCDR1, LCDR2, and LCDR3 comprises an amino acid sequence of SEQ ID NOS: 16322, 3982, and 12957, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9802, and 10196, respectively and the LCDR1, LCDR2, and LCDR3 comprises an amino acid sequence of SEQ ID NOS: 10832, 9267, and 11317, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 17778, and 10165, respectively and the LCDR1, LCDR2, and LCDR3 comprises an amino acid sequence of SEQ ID NOS:10890, 18275, and 12971, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11560, 10540, and 9540, respectively and the LCDR1, LCDR2, and LCDR3 comprises an amino acid sequence of SEQ ID NOS:18060, 18596, and 17788, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6570, 4127, and 18405, respectively and the LCDR1, LCDR2, and LCDR3 comprises an amino acid sequence of SEQ ID NOS: 15647, 3982, and 12859, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6510, 9379, and 10993, respectively and the LCDR1, LCDR2, and LCDR3 comprises an amino acid sequence of SEQ ID NOS: 10223, 6212, and 11053, respectively. having a LCDR1, LCDR2, and LCDR3, wherein the HCDR1, HCDR2, and HCDR3 comprises an amino acid sequence of SEQ ID NOS:6578, 18547, and 6147, respectively and the LCDR1, LCDR2, and LCDR3 comprises an amino acid sequence of SEQ ID NOS:9633, 18448, and 15562, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17467, 11446, and 11544, respectively and the LCDR1, LCDR2, and LCDR3 comprises an amino acid sequence of SEQ ID NOS:10856, 18261, and 12927, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9754, and 10144, respectively and the LCDR1, LCDR2, and LCDR3 comprises an amino acid sequence of SEQ ID NOS: 16026, 9494, and 12030, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11370, 16250, and 6055, respectively and the LCDR1, LCDR2, and LCDR3 comprises an amino acid sequence of SEQ ID NOS:16408, 10987, and 11211, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17453, 16257, and 18113, respectively and the LCDR1, LCDR2, and LCDR3 comprises an amino acid sequence of SEQ ID NOS: 16022, 9503, and 10458, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 17784, and 4585, respectively and the LCDR1, LCDR2, and LCDR3 comprises an amino acid sequence of SEQ ID NOS: 10839, 9267, and 11313, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9776, and 10267, respectively and the LCDR1, LCDR2, and LCDR3 comprises an amino acid sequence of SEQ ID NOS: 17959, 6318, and 17197, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10935, 10318, and 10441, respectively and the LCDR1, LCDR2, and LCDR3 comprises an amino acid sequence of SEQ ID NOS:16061, 9494, and 12671, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10362, 11545, and 4459, respectively and the LCDR1, LCDR2, and LCDR3 comprises an amino acid sequence of SEQ ID NOS:15778, 10730, and 12874, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9838, and 10232, respectively and the LCDR1, LCDR2, and LCDR3 comprises an amino acid sequence of SEQ ID NOS: 15956, 9480, and 12684, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17141, 16954, and 5910, respectively and the LCDR1, LCDR2, and LCDR3 comprises an amino acid sequence of SEQ ID NOS:15890, 10727, and 12846, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11622, 6755, and 16635, respectively and the LCDR1, LCDR2, and LCDR3 comprises an amino acid sequence of SEQ ID NOS:16024, 10261, and 12429, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17608, 18012, and 4037, respectively and the LCDR1, LCDR2, and LCDR3 comprises an amino acid sequence of SEQ ID NOS: 15790, 9472, and 13016, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9817, and 10517, respectively and the LCDR1, LCDR2, and LCDR3 comprises an amino acid sequence of SEQ ID NOS:16102, 10732, and 12851, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11677, 16244, and 11727, respectively and the LCDR1, LCDR2, and LCDR3 comprises an amino acid sequence of SEQ ID NOS:10870, 18275, and 12952, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17432, 18531, and 17708, respectively and the LCDR1, LCDR2, and LCDR3 comprises an amino acid sequence of SEQ ID NOS: 17966, 6717, and 4368, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16954, and 12091, respectively and the LCDR1, LCDR2, and LCDR3 comprises an amino acid sequence of SEQ ID NOS:16408, 10987, and 11201, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 4146, and 7815, respectively and the LCDR1, LCDR2, and LCDR3 comprises an amino acid sequence of SEQ ID NOS: 15893, 3969, and 12523, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17558, 11037, and 16623, respectively and the LCDR1, LCDR2, and LCDR3 comprises an amino acid sequence of SEQ ID NOS:16061, 9494, and 12691, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9776, and 10941, respectively and the LCDR1, LCDR2, and LCDR3 comprises an amino acid sequence of SEQ ID NOS:16659, 11900, and 11884, respectively. having a LCDR1, LCDR2, and LCDR3, wherein the HCDR1, HCDR2, and HCDR3 comprises an amino acid sequence of SEQ ID NOS: 17621, 16867, and 6544, respectively and the LCDR1, LCDR2, and LCDR3 comprises an amino acid sequence of SEQ ID NOS: 16071, 9503, and 12725, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17068, 9876, and 11955, respectively and the LCDR1, LCDR2, and LCDR3 comprises an amino acid sequence of SEQ ID NOS: 15881, 9503, and 12599, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17208, 18581, and 6183, respectively and the LCDR1, LCDR2, and LCDR3 comprises an amino acid sequence of SEQ ID NOS: 15760, 3975, and 10451, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17848, 10544, and 6569, respectively and the LCDR1, LCDR2, and LCDR3 comprises an amino acid sequence of SEQ ID NOS: 15893, 3969, and 12520, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11663, 11018, and 16521, respectively and the LCDR1, LCDR2, and LCDR3 comprises an amino acid sequence of SEQ ID NOS: 15980, 9503, and 12796, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18360, and 6047, respectively and the LCDR1, LCDR2, and LCDR3 comprises an amino acid sequence of SEQ ID NOS: 16759, 16331, and 4008, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10367, 18323, and 18288, respectively and the LCDR1, LCDR2, and LCDR3 comprises an amino acid sequence of SEQ ID NOS: 15890, 10727, and 12845, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17603, 18341, and 17699, respectively and the LCDR1, LCDR2, and LCDR3 comprises an amino acid sequence of SEQ ID NOS:15711, 3963, and 12335, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18360, and 9645, respectively and the LCDR1, LCDR2, and LCDR3 comprises an amino acid sequence of SEQ ID NOS:10788, 10932, and 11263, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17141, 16954, and 6726, respectively and the LCDR1, LCDR2, and LCDR3 comprises an amino acid sequence of SEQ ID NOS: 15893, 3969, and 12512, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16529, 4153, and 16931, respectively and the LCDR1, LCDR2, and LCDR3 comprises an amino acid sequence of SEQ ID NOS: 16011, 9502, and 12780, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 16919, and 4231, respectively and the LCDR1, LCDR2, and LCDR3 comprises an amino acid sequence of SEQ ID NOS: 17550, 6629, and 13586, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 17809, and 5921, respectively and the LCDR1, LCDR2, and LCDR3 comprises an amino acid sequence of SEQ ID NOS: 16045, 9503, and 12788, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17600, 9821, and 4212, respectively and the LCDR1, LCDR2, and LCDR3 comprises an amino acid sequence of SEQ ID NOS: 17875, 6328, and 17182, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10337, 9790, and 11968, respectively and the LCDR1, LCDR2, and LCDR3 comprises an amino acid sequence of SEQ ID NOS: 15918, 9494, and 12610, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 11488, and 6480, respectively and the LCDR1, LCDR2, and LCDR3 comprises an amino acid sequence of SEQ ID NOS: 16067, 4425, and 10715, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4136, and 5912, respectively and the LCDR1, LCDR2, and LCDR3 comprises an amino acid sequence of SEQ ID NOS: 17959, 6312, and 17199, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9861, and 11973, respectively and the LCDR1, LCDR2, and LCDR3 comprises an amino acid sequence of SEQ ID NOS: 16061, 9503, and 12700, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16954, and 17458, respectively and the LCDR1, LCDR2, and LCDR3 comprises an amino acid sequence of SEQ ID NOS: 15890, 10727, and 12831, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 16919, and 4484, respectively and the LCDR1, LCDR2, and LCDR3 comprises an amino acid sequence of SEQ ID NOS: 16322, 3982, and 12958, respectively. having a LCDR1, LCDR2, and LCDR3, wherein the HCDR1, HCDR2, and HCDR3 comprises an amino acid sequence of SEQ ID NOS:6578, 18531, and 5919, respectively and the LCDR1, LCDR2, and LCDR3 comprises an amino acid sequence of SEQ ID NOS: 16067, 4425, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17270, 17778, and 10755, respectively and the LCDR1, LCDR2, and LCDR3 comprises an amino acid sequence of SEQ ID NOS:16659, 11900, and 11886, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17432, 18531, and 18281, respectively and the LCDR1, LCDR2, and LCDR3 comprises an amino acid sequence of SEQ ID NOS:15713, 3982, and 12389, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17432, 18531, and 6192, respectively and the LCDR1, LCDR2, and LCDR3 comprises an amino acid sequence of SEQ ID NOS: 16736, 7859, and 10276, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16692, 18569, and 4215, respectively and the LCDR1, LCDR2, and LCDR3 comprises an amino acid sequence of SEQ ID NOS: 15893, 3969, and 12519, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16692, 18586, and 7874, respectively and the LCDR1, LCDR2, and LCDR3 comprises an amino acid sequence of SEQ ID NOS: 16045, 9503, and 12787, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17446, 4136, and 6121, respectively and the LCDR1, LCDR2, and LCDR3 comprises an amino acid sequence of SEQ ID NOS:16408, 10987, and 11210, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17447, 16896, and 6553, respectively and the LCDR1, LCDR2, and LCDR3 comprises an amino acid sequence of SEQ ID NOS: 16045, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18101, 9717, and 17219, respectively and the LCDR1, LCDR2, and LCDR3 comprises an amino acid sequence of SEQ ID NOS:15810, 4425, and 12713, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16713, 16824, and 10381, respectively and the LCDR1, LCDR2, and LCDR3 comprises an amino acid sequence of SEQ ID NOS:15952, 4425, and 12440, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17535, 10571, and 5964, respectively and the LCDR1, LCDR2, and LCDR3 comprises an amino acid sequence of SEQ ID NOS: 16061, 9494, and 12686, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17614, 18557, and 4392, respectively and the LCDR1, LCDR2, and LCDR3 comprises an amino acid sequence of SEQ ID NOS: 15958, 9503, and 12450, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6530, 9911, and 18107, respectively and the LCDR1, LCDR2, and LCDR3 comprises an amino acid sequence of SEQ ID NOS: 15655, 3978, and 12945, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17004, 16980, and 13594, respectively and the LCDR1, LCDR2, and LCDR3 comprises an amino acid sequence of SEQ ID NOS: 15736, 9352, and 12504, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16796, 16939, and 9690, respectively and the LCDR1, LCDR2, and LCDR3 comprises an amino acid sequence of SEQ ID NOS: 16759, 16331, and 4005, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17028, 17823, and 10164, respectively and the LCDR1, LCDR2, and LCDR3 comprises an amino acid sequence of SEQ ID NOS:16659, 11900, and 11869, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9971, and 4027, respectively and the LCDR1, LCDR2, and LCDR3 comprises an amino acid sequence of SEQ ID NOS: 16061, 9494, and 12666, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17856, 9437, and 18229, respectively and the LCDR1, LCDR2, and LCDR3 comprises an amino acid sequence of SEQ ID NOS:16662, 11901, and 11862, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6578, 18553, and 9559, respectively and the LCDR1, LCDR2, and LCDR3 comprises an amino acid sequence of SEQ ID NOS:15895, 10727, and 12829, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 10571, and 16182, respectively and the LCDR1, LCDR2, and LCDR3 comprises an amino acid sequence of SEQ ID NOS: 16759, 16331, and 4008, respectively. having a LCDR1, LCDR2, and LCDR3, wherein the HCDR1, HCDR2, and HCDR3 comprises an amino acid sequence of SEQ ID NOS:18100, 9716, and 17120, respectively and the LCDR1, LCDR2, and LCDR3 comprises an amino acid sequence of SEQ ID NOS: 15960, 9494, and 12627, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11677, 12038, and 4462, respectively and the LCDR1, LCDR2, and LCDR3 comprises an amino acid sequence of SEQ ID NOS:10845, 16677, and 11336, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17284, 18415, and 10210, respectively and the LCDR1, LCDR2, and LCDR3 comprises an amino acid sequence of SEQ ID NOS:10890, 18275, and 12994, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17605, 9948, and 17046, respectively and the LCDR1, LCDR2, and LCDR3 comprises an amino acid sequence of SEQ ID NOS: 17948, 6245, and 17175, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10367, 18323, and 6017, respectively and the LCDR1, LCDR2, and LCDR3 comprises an amino acid sequence of SEQ ID NOS: 16749, 17007, and 3997, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17446, 4136, and 16622, respectively and the LCDR1, LCDR2, and LCDR3 comprises an amino acid sequence of SEQ ID NOS: 17959, 6312, and 17197, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 11026, and 6011, respectively and the LCDR1, LCDR2, and LCDR3 comprises an amino acid sequence of SEQ ID NOS:15890, 10697, and 12826, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17558, 11037, and 16350, respectively and the LCDR1, LCDR2, and LCDR3 comprises an amino acid sequence of SEQ ID NOS: 17966, 6717, and 4367, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9873, and 11967, respectively and the LCDR1, LCDR2, and LCDR3 comprises an amino acid sequence of SEQ ID NOS: 16048, 9501, and 12700, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16796, 16939, and 16349, respectively and the LCDR1, LCDR2, and LCDR3 comprises an amino acid sequence of SEQ ID NOS:15890, 10727, and 12831, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16813, 16910, and 10066, respectively and the LCDR1, LCDR2, and LCDR3 comprises an amino acid sequence of SEQ ID NOS:15757, 6214, and 12447, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9859, and 11971, respectively and the LCDR1, LCDR2, and LCDR3 comprises an amino acid sequence of SEQ ID NOS: 15792, 9503, and 12599, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16531, 18307, and 6189, respectively and the LCDR1, LCDR2, and LCDR3 comprises an amino acid sequence of SEQ ID NOS: 17935, 6327, and 11603, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4060, 9711, and 17012, respectively and the LCDR1, LCDR2, and LCDR3 comprises an amino acid sequence of SEQ ID NOS: 16014, 9494, and 12700, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4060, 9932, and 17011, respectively and the LCDR1, LCDR2, and LCDR3 comprises an amino acid sequence of SEQ ID NOS: 16008, 9494, and 12631, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11650, 9434, and 17533, respectively and the LCDR1, LCDR2, and LCDR3 comprises an amino acid sequence of SEQ ID NOS: 16076, 4413, and 10443, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17557, 11146, and 16344, respectively and the LCDR1, LCDR2, and LCDR3 comprises an amino acid sequence of SEQ ID NOS: 15890, 10695, and 12593, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 9388, and 4471, respectively and the LCDR1, LCDR2, and LCDR3 comprises an amino acid sequence of SEQ ID NOS: 15893, 3969, and 12516, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16779, 16311, and 7912, respectively and the LCDR1, LCDR2, and LCDR3 comprises an amino acid sequence of SEQ ID NOS:9632, 16162, and 15565, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10335, 9870, and 11975, respectively and the LCDR1, LCDR2, and LCDR3 comprises an amino acid sequence of SEQ ID NOS: 16049, 9503, and 12719, respectively. having a LCDR1, LCDR2, and LCDR3, wherein the HCDR1, HCDR2, and HCDR3 comprises an amino acid sequence of SEQ ID NOS: 17274, 4136, and 6562, respectively and the LCDR1, LCDR2, and LCDR3 comprises an amino acid sequence of SEQ ID NOS:11949, 10024, and 11584, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9672, 4159, and 17022, respectively and the LCDR1, LCDR2, and LCDR3 comprises an amino acid sequence of SEQ ID NOS: 17959, 6244, and 17157, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17270, 4146, and 18043, respectively and the LCDR1, LCDR2, and LCDR3 comprises an amino acid sequence of SEQ ID NOS: 15711, 3969, and 12336, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16631, 11426, and 4293, respectively and the LCDR1, LCDR2, and LCDR3 comprises an amino acid sequence of SEQ ID NOS:16467, 15521, and 11269, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17135, 16826, and 11041, respectively and the LCDR1, LCDR2, and LCDR3 comprises an amino acid sequence of SEQ ID NOS: 15980, 3983, and 12857, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17633, 18336, and 17565, respectively and the LCDR1, LCDR2, and LCDR3 comprises an amino acid sequence of SEQ ID NOS:10916, 18272, and 12907, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17432, 18531, and 13626, respectively and the LCDR1, LCDR2, and LCDR3 comprises an amino acid sequence of SEQ ID NOS:16067, 4425, and 12412, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17558, 11037, and 4169, respectively and the LCDR1, LCDR2, and LCDR3 comprises an amino acid sequence of SEQ ID NOS:15890, 10727, and 12849, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 10571, and 10375, respectively and the LCDR1, LCDR2, and LCDR3 comprises an amino acid sequence of SEQ ID NOS:16045, 9503, and 12769, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9860, and 11974, respectively and the LCDR1, LCDR2, and LCDR3 comprises an amino acid sequence of SEQ ID NOS: 15792, 9503, and 12599, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16692, 18569, and 10209, respectively and the LCDR1, LCDR2, and LCDR3 comprises an amino acid sequence of SEQ ID NOS:10890, 18275, and 12982, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10338, 10303, and 11954, respectively and the LCDR1, LCDR2, and LCDR3 comprises an amino acid sequence of SEQ ID NOS:16067, 4417, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10367, 18323, and 4593, respectively and the LCDR1, LCDR2, and LCDR3 comprises an amino acid sequence of SEQ ID NOS:16408, 10987, and 11202, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10368, 6781, and 16600, respectively and the LCDR1, LCDR2, and LCDR3 comprises an amino acid sequence of SEQ ID NOS: 16061, 9480, and 12349, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17432, 18531, and 6167, respectively and the LCDR1, LCDR2, and LCDR3 comprises an amino acid sequence of SEQ ID NOS: 16067, 4425, and 12434, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11377, 16172, and 9366, respectively and the LCDR1, LCDR2, and LCDR3 comprises an amino acid sequence of SEQ ID NOS: 15869, 4141, and 13785, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17884, 18509, and 17681, respectively and the LCDR1, LCDR2, and LCDR3 comprises an amino acid sequence of SEQ ID NOS:9595, 4465, and 15570, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17210, 18306, and 4600, respectively and the LCDR1, LCDR2, and LCDR3 comprises an amino acid sequence of SEQ ID NOS: 15686, 9498, and 12535, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:7820, 9562, and 10592, respectively and the LCDR1, LCDR2, and LCDR3 comprises an amino acid sequence of SEQ ID NOS:17924, 10094, and 13576, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11417, 16825, and 11984, respectively and the LCDR1, LCDR2, and LCDR3 comprises an amino acid sequence of SEQ ID NOS: 15645, 17227, and 12347, respectively. having a LCDR1, LCDR2, and LCDR3, wherein the HCDR1, HCDR2, and HCDR3 comprises an amino acid sequence of SEQ ID NOS:17270, 9407, and 6713, respectively and the LCDR1, LCDR2, and LCDR3 comprises an amino acid sequence of SEQ ID NOS: 16049, 4431, and 12003, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11637, 18410, and 6473, respectively and the LCDR1, LCDR2, and LCDR3 comprises an amino acid sequence of SEQ ID NOS: 16717, 9535, and 17232, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16779, 16311, and 4219, respectively and the LCDR1, LCDR2, and LCDR3 comprises an amino acid sequence of SEQ ID NOS:15890, 10727, and 12809, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17856, 18585, and 18141, respectively and the LCDR1, LCDR2, and LCDR3 comprises an amino acid sequence of SEQ ID NOS: 16067, 4425, and 12424, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16692, 18569, and 16601, respectively and the LCDR1, LCDR2, and LCDR3 comprises an amino acid sequence of SEQ ID NOS: 11949, 10024, and 11588, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17838, 16277, and 6080, respectively and the LCDR1, LCDR2, and LCDR3 comprises an amino acid sequence of SEQ ID NOS:15890, 10727, and 13593, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17651, 11439, and 4292, respectively and the LCDR1, LCDR2, and LCDR3 comprises an amino acid sequence of SEQ ID NOS: 16077, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6576, 18332, and 10236, respectively and the LCDR1, LCDR2, and LCDR3 comprises an amino acid sequence of SEQ ID NOS: 16726, 16851, and 3991, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6504, 18381, and 10183, respectively and the LCDR1, LCDR2, and LCDR3 comprises an amino acid sequence of SEQ ID NOS: 16061, 4431, and 12692, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16692, 18569, and 10286, respectively and the LCDR1, LCDR2, and LCDR3 comprises an amino acid sequence of SEQ ID NOS:11949, 10024, and 11586, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17029, 16942, and 6095, respectively and the LCDR1, LCDR2, and LCDR3 comprises an amino acid sequence of SEQ ID NOS: 15905, 9490, and 12483, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17856, 18575, and 6603, respectively and the LCDR1, LCDR2, and LCDR3 comprises an amino acid sequence of SEQ ID NOS: 15893, 3969, and 12519, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17432, 18531, and 11157, respectively and the LCDR1, LCDR2, and LCDR3 comprises an amino acid sequence of SEQ ID NOS: 15691, 3969, and 11083, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16692, 18569, and 6122, respectively and the LCDR1, LCDR2, and LCDR3 comprises an amino acid sequence of SEQ ID NOS: 17959, 6318, and 17190, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17554, 9706, and 17122, respectively and the LCDR1, LCDR2, and LCDR3 comprises an amino acid sequence of SEQ ID NOS: 15741, 4436, and 12621, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11671, 9996, and 16960, respectively and the LCDR1, LCDR2, and LCDR3 comprises an amino acid sequence of SEQ ID NOS:16359, 10976, and 11252, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 17741, and 6091, respectively and the LCDR1, LCDR2, and LCDR3 comprises an amino acid sequence of SEQ ID NOS: 17950, 5445, and 17191, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17291, 4065, and 9453, respectively and the LCDR1, LCDR2, and LCDR3 comprises an amino acid sequence of SEQ ID NOS: 16061, 9494, and 12678, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 10571, and 10981, respectively and the LCDR1, LCDR2, and LCDR3 comprises an amino acid sequence of SEQ ID NOS:10890, 18275, and 12983, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18639, 9808, and 11764, respectively and the LCDR1, LCDR2, and LCDR3 comprises an amino acid sequence of SEQ ID NOS: 16053, 4054, and 12879, respectively. having a LCDR1, LCDR2, and LCDR3, wherein the HCDR1, HCDR2, and HCDR3 comprises an amino acid sequence of SEQ ID NOS: 11370, 16250, and 5931, respectively and the LCDR1, LCDR2, and LCDR3 comprises an amino acid sequence of SEQ ID NOS:10890, 18275, and 12970, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10101, 6782, and 9673, respectively and the LCDR1, LCDR2, and LCDR3 comprises an amino acid sequence of SEQ ID NOS: 11789, 4420, and 12575, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16692, 18586, and 4168, respectively and the LCDR1, LCDR2, and LCDR3 comprises an amino acid sequence of SEQ ID NOS: 15711, 3969, and 12335, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17838, 16277, and 6100, respectively and the LCDR1, LCDR2, and LCDR3 comprises an amino acid sequence of SEQ ID NOS: 18093, 17720, and 9444, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17015, 16499, and 10091, respectively and the LCDR1, LCDR2, and LCDR3 comprises an amino acid sequence of SEQ ID NOS:17959, 6318, and 17198, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10367, 18323, and 17834, respectively and the LCDR1, LCDR2, and LCDR3 comprises an amino acid sequence of SEQ ID NOS: 15893, 3969, and 12519, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17856, 18575, and 4045, respectively and the LCDR1, LCDR2, and LCDR3 comprises an amino acid sequence of SEQ ID NOS: 11789, 4420, and 12575, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 11497, and 4292, respectively and the LCDR1, LCDR2, and LCDR3 comprises an amino acid sequence of SEQ ID NOS:16423, 15521, and 11266, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16680, 16227, and 10045, respectively and the LCDR1, LCDR2, and LCDR3 comprises an amino acid sequence of SEQ ID NOS:16049, 9494, and 12698, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17856, 18585, and 18232, respectively and the LCDR1, LCDR2, and LCDR3 comprises an amino acid sequence of SEQ ID NOS: 17959, 6312, and 4370, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17141, 16911, and 9516, respectively and the LCDR1, LCDR2, and LCDR3 comprises an amino acid sequence of SEQ ID NOS: 16759, 16334, and 4013, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11370, 16250, and 6078, respectively and the LCDR1, LCDR2, and LCDR3 comprises an amino acid sequence of SEQ ID NOS: 17959, 6318, and 17193, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17272, 18342, and 4163, respectively and the LCDR1, LCDR2, and LCDR3 comprises an amino acid sequence of SEQ ID NOS: 16045, 9503, and 12787, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10306, 16508, and 11415, respectively and the LCDR1, LCDR2, and LCDR3 comprises an amino acid sequence of SEQ ID NOS: 16774, 11403, and 4234, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11370, 16252, and 10061, respectively and the LCDR1, LCDR2, and LCDR3 comprises an amino acid sequence of SEQ ID NOS:6180, 6207, and 11050, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16169, 16872, and 16914, respectively and the LCDR1, LCDR2, and LCDR3 comprises an amino acid sequence of SEQ ID NOS: 16007, 9503, and 12856, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 17778, and 6025, respectively and the LCDR1, LCDR2, and LCDR3 comprises an amino acid sequence of SEQ ID NOS:10890, 18275, and 12975, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16954, and 12374, respectively and the LCDR1, LCDR2, and LCDR3 comprises an amino acid sequence of SEQ ID NOS: 16045, 9503, and 12783, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17446, 4136, and 18618, respectively and the LCDR1, LCDR2, and LCDR3 comprises an amino acid sequence of SEQ ID NOS:15890, 10727, and 12819, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16692, 18569, and 11164, respectively and the LCDR1, LCDR2, and LCDR3 comprises an amino acid sequence of SEQ ID NOS: 16061, 9494, and 12657, respectively. having a LCDR1, LCDR2, and LCDR3, wherein the HCDR1, HCDR2, and HCDR3 comprises an amino acid sequence of SEQ ID NOS:17558, 9413, and 11661, respectively and the LCDR1, LCDR2, and LCDR3 comprises an amino acid sequence of SEQ ID NOS: 15710, 3980, and 12384, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16602, 17820, and 4241, respectively and the LCDR1, LCDR2, and LCDR3 comprises an amino acid sequence of SEQ ID NOS:15819, 10908, and 12014, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4226, 9871, and 11976, respectively and the LCDR1, LCDR2, and LCDR3 comprises an amino acid sequence of SEQ ID NOS: 16058, 3983, and 12700, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4038, 11501, and 4309, respectively and the LCDR1, LCDR2, and LCDR3 comprises an amino acid sequence of SEQ ID NOS:10220, 15522, and 11281, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11631, 18342, and 4268, respectively and the LCDR1, LCDR2, and LCDR3 comprises an amino acid sequence of SEQ ID NOS: 16067, 4425, and 12430, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17856, 18585, and 6568, respectively and the LCDR1, LCDR2, and LCDR3 comprises an amino acid sequence of SEQ ID NOS: 16736, 7859, and 10275, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9776, and 7885, respectively and the LCDR1, LCDR2, and LCDR3 comprises an amino acid sequence of SEQ ID NOS:10890, 18275, and 12979, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17446, 4140, and 11733, respectively and the LCDR1, LCDR2, and LCDR3 comprises an amino acid sequence of SEQ ID NOS: 16045, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11649, 9399, and 4395, respectively and the LCDR1, LCDR2, and LCDR3 comprises an amino acid sequence of SEQ ID NOS: 16045, 9499, and 12946, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16777, 16305, and 10194, respectively and the LCDR1, LCDR2, and LCDR3 comprises an amino acid sequence of SEQ ID NOS:15787, 16557, and 12541, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17635, 10254, and 17629, respectively and the LCDR1, LCDR2, and LCDR3 comprises an amino acid sequence of SEQ ID NOS: 15943, 9503, and 12866, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 9388, and 10308, respectively and the LCDR1, LCDR2, and LCDR3 comprises an amino acid sequence of SEQ ID NOS: 16067, 4425, and 12436, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 11488, and 4213, respectively and the LCDR1, LCDR2, and LCDR3 comprises an amino acid sequence of SEQ ID NOS:16659, 11903, and 11874, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17432, 18531, and 5939, respectively and the LCDR1, LCDR2, and LCDR3 comprises an amino acid sequence of SEQ ID NOS:16428, 10764, and 11223, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18100, 9570, and 17830, respectively and the LCDR1, LCDR2, and LCDR3 comprises an amino acid sequence of SEQ ID NOS: 15903, 6613, and 12023, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 9388, and 18045, respectively and the LCDR1, LCDR2, and LCDR3 comprises an amino acid sequence of SEQ ID NOS: 16061, 9494, and 12677, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 17778, and 6499, respectively and the LCDR1, LCDR2, and LCDR3 comprises an amino acid sequence of SEQ ID NOS:18090, 11073, and 13614, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17005, 16831, and 18287, respectively and the LCDR1, LCDR2, and LCDR3 comprises an amino acid sequence of SEQ ID NOS: 16458, 10727, and 12802, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16786, 16300, and 3945, respectively and the LCDR1, LCDR2, and LCDR3 comprises an amino acid sequence of SEQ ID NOS: 16052, 9494, and 12663, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17849, 17822, and 4186, respectively and the LCDR1, LCDR2, and LCDR3 comprises an amino acid sequence of SEQ ID NOS: 16736, 7859, and 10279, respectively. having a LCDR1, LCDR2, and LCDR3, wherein the HCDR1, HCDR2, and HCDR3 comprises an amino acid sequence of SEQ ID NOS:17263, 9776, and 11682, respectively and the LCDR1, LCDR2, and LCDR3 comprises an amino acid sequence of SEQ ID NOS: 16067, 4425, and 12432, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17600, 9714, and 17120, respectively and the LCDR1, LCDR2, and LCDR3 comprises an amino acid sequence of SEQ ID NOS: 16048, 3973, and 12615, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17842, 11488, and 7812, respectively and the LCDR1, LCDR2, and LCDR3 comprises an amino acid sequence of SEQ ID NOS: 16736, 5977, and 10276, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11632, 10317, and 11565, respectively and the LCDR1, LCDR2, and LCDR3 comprises an amino acid sequence of SEQ ID NOS:16676, 11895, and 11866, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17838, 16277, and 5949, respectively and the LCDR1, LCDR2, and LCDR3 comprises an amino acid sequence of SEQ ID NOS:17924, 10094, and 13579, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17848, 10544, and 6046, respectively and the LCDR1, LCDR2, and LCDR3 comprises an amino acid sequence of SEQ ID NOS: 16061, 9494, and 12690, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10337, 9734, and 11968, respectively and the LCDR1, LCDR2, and LCDR3 comprises an amino acid sequence of SEQ ID NOS: 16061, 9494, and 12697, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17856, 18585, and 17589, respectively and the LCDR1, LCDR2, and LCDR3 comprises an amino acid sequence of SEQ ID NOS: 16061, 9494, and 12704, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 16919, and 6709, respectively and the LCDR1, LCDR2, and LCDR3 comprises an amino acid sequence of SEQ ID NOS:15890, 10727, and 12835, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 11488, and 7892, respectively and the LCDR1, LCDR2, and LCDR3 comprises an amino acid sequence of SEQ ID NOS:17959, 6318, and 17194, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17444, 18342, and 6190, respectively and the LCDR1, LCDR2, and LCDR3 comprises an amino acid sequence of SEQ ID NOS: 15879, 9503, and 12792, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16525, 9785, and 4161, respectively and the LCDR1, LCDR2, and LCDR3 comprises an amino acid sequence of SEQ ID NOS:15676, 10736, and 12023, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16779, 16311, and 9667, respectively and the LCDR1, LCDR2, and LCDR3 comprises an amino acid sequence of SEQ ID NOS: 16067, 4425, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17463, 9793, and 11759, respectively and the LCDR1, LCDR2, and LCDR3 comprises an amino acid sequence of SEQ ID NOS: 16084, 9492, and 12701, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11406, 16940, and 15589, respectively and the LCDR1, LCDR2, and LCDR3 comprises an amino acid sequence of SEQ ID NOS:16023, 10230, and 12618, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17848, 10544, and 6102, respectively and the LCDR1, LCDR2, and LCDR3 comprises an amino acid sequence of SEQ ID NOS: 16067, 4425, and 12432, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9776, and 7893, respectively and the LCDR1, LCDR2, and LCDR3 comprises an amino acid sequence of SEQ ID NOS:9633, 4467, and 15558, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17441, 18360, and 17538, respectively and the LCDR1, LCDR2, and LCDR3 comprises an amino acid sequence of SEQ ID NOS:10059, 17222, and 18117, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17838, 16277, and 4456, respectively and the LCDR1, LCDR2, and LCDR3 comprises an amino acid sequence of SEQ ID NOS:16659, 11900, and 11881, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16529, 9573, and 4579, respectively and the LCDR1, LCDR2, and LCDR3 comprises an amino acid sequence of SEQ ID NOS:15988, 10264, and 12772, respectively. having a LCDR1, LCDR2, and LCDR3, wherein the HCDR1, HCDR2, and HCDR3 comprises an amino acid sequence of SEQ ID NOS:16790, 11580, and 12308, respectively and the LCDR1, LCDR2, and LCDR3 comprises an amino acid sequence of SEQ ID NOS:15618, 9657, and 12467, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9679, 18080, and 6703, respectively and the LCDR1, LCDR2, and LCDR3 comprises an amino acid sequence of SEQ ID NOS: 15735, 6716, and 12589, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9776, and 11728, respectively and the LCDR1, LCDR2, and LCDR3 comprises an amino acid sequence of SEQ ID NOS: 16061, 9494, and 12677, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 16919, and 6082, respectively and the LCDR1, LCDR2, and LCDR3 comprises an amino acid sequence of SEQ ID NOS:15890, 10727, and 12829, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6510, 9379, and 18248, respectively and the LCDR1, LCDR2, and LCDR3 comprises an amino acid sequence of SEQ ID NOS:16408, 10987, and 11209, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4177, 16216, and 17076, respectively and the LCDR1, LCDR2, and LCDR3 comprises an amino acid sequence of SEQ ID NOS:16410, 10985, and 11197, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17838, 16277, and 4618, respectively and the LCDR1, LCDR2, and LCDR3 comprises an amino acid sequence of SEQ ID NOS: 15691, 3969, and 11094, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17446, 4136, and 10105, respectively and the LCDR1, LCDR2, and LCDR3 comprises an amino acid sequence of SEQ ID NOS:16642, 10747, and 13030, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11643, 10542, and 10345, respectively and the LCDR1, LCDR2, and LCDR3 comprises an amino acid sequence of SEQ ID NOS:17959, 6244, and 11602, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17279, 17253, and 5944, respectively and the LCDR1, LCDR2, and LCDR3 comprises an amino acid sequence of SEQ ID NOS: 16045, 9472, and 12887, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18100, 9704, and 17218, respectively and the LCDR1, LCDR2, and LCDR3 comprises an amino acid sequence of SEQ ID NOS: 16091, 9492, and 12701, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 17767, and 6037, respectively and the LCDR1, LCDR2, and LCDR3 comprises an amino acid sequence of SEQ ID NOS: 16736, 7859, and 10273, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17602, 18337, and 6022, respectively and the LCDR1, LCDR2, and LCDR3 comprises an amino acid sequence of SEQ ID NOS: 15770, 3953, and 12479, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18100, 9703, and 17121, respectively and the LCDR1, LCDR2, and LCDR3 comprises an amino acid sequence of SEQ ID NOS: 15740, 4413, and 12685, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16796, 16939, and 10434, respectively and the LCDR1, LCDR2, and LCDR3 comprises an amino acid sequence of SEQ ID NOS: 16736, 7859, and 10278, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11627, 9883, and 16913, respectively and the LCDR1, LCDR2, and LCDR3 comprises an amino acid sequence of SEQ ID NOS: 15950, 9503, and 12780, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10188, 9875, and 11972, respectively and the LCDR1, LCDR2, and LCDR3 comprises an amino acid sequence of SEQ ID NOS: 15926, 9503, and 12655, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16796, 16939, and 10956, respectively and the LCDR1, LCDR2, and LCDR3 comprises an amino acid sequence of SEQ ID NOS:16659, 11900, and 11880, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17446, 4136, and 17563, respectively and the LCDR1, LCDR2, and LCDR3 comprises an amino acid sequence of SEQ ID NOS:16659, 11900, and 11876, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 11511, and 4291, respectively and the LCDR1, LCDR2, and LCDR3 comprises an amino acid sequence of SEQ ID NOS:16420, 10926, and 11270, respectively. having a LCDR1, LCDR2, and LCDR3, wherein the HCDR1, HCDR2, and HCDR3 comprises an amino acid sequence of SEQ ID NOS:4324, 18325, and 6714, respectively and the LCDR1, LCDR2, and LCDR3 comprises an amino acid sequence of SEQ ID NOS: 16061, 3974, and 12674, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17463, 9879, and 11761, respectively and the LCDR1, LCDR2, and LCDR3 comprises an amino acid sequence of SEQ ID NOS: 16014, 9480, and 12701, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16779, 16311, and 10026, respectively and the LCDR1, LCDR2, and LCDR3 comprises an amino acid sequence of SEQ ID NOS: 16642, 10747, and 13026, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16692, 18569, and 4021, respectively and the LCDR1, LCDR2, and LCDR3 comprises an amino acid sequence of SEQ ID NOS: 16061, 9494, and 12677, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6145, 16320, and 17539, respectively and the LCDR1, LCDR2, and LCDR3 comprises an amino acid sequence of SEQ ID NOS: 16061, 9494, and 12671, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 17809, and 16789, respectively and the LCDR1, LCDR2, and LCDR3 comprises an amino acid sequence of SEQ ID NOS:17959, 6312, and 17185, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6548, 18025, and 5899, respectively and the LCDR1, LCDR2, and LCDR3 comprises an amino acid sequence of SEQ ID NOS: 17931, 7982, and 4336, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10367, 18323, and 6020, respectively and the LCDR1, LCDR2, and LCDR3 comprises an amino acid sequence of SEQ ID NOS: 15713, 3982, and 13000, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16516, 9807, and 17833, respectively and the LCDR1, LCDR2, and LCDR3 comprises an amino acid sequence of SEQ ID NOS:10882, 18275, and 12997, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17141, 16954, and 6178, respectively and the LCDR1, LCDR2, and LCDR3 comprises an amino acid sequence of SEQ ID NOS: 15893, 3969, and 12522, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16954, and 11978, respectively and the LCDR1, LCDR2, and LCDR3 comprises an amino acid sequence of SEQ ID NOS:15805, 18441, and 10448, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11561, 10577, and 6248, respectively and the LCDR1, LCDR2, and LCDR3 comprises an amino acid sequence of SEQ ID NOS: 16036, 9503, and 12764, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17558, 11037, and 16502, respectively and the LCDR1, LCDR2, and LCDR3 comprises an amino acid sequence of SEQ ID NOS: 16750, 17007, and 3997, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16796, 16939, and 10989, respectively and the LCDR1, LCDR2, and LCDR3 comprises an amino acid sequence of SEQ ID NOS: 16045, 9503, and 12799, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16692, 18569, and 9616, respectively and the LCDR1, LCDR2, and LCDR3 comprises an amino acid sequence of SEQ ID NOS:11949, 10024, and 11590, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11383, 18529, and 6182, respectively and the LCDR1, LCDR2, and LCDR3 comprises an amino acid sequence of SEQ ID NOS: 15603, 3951, and 10453, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17848, 10544, and 4612, respectively and the LCDR1, LCDR2, and LCDR3 comprises an amino acid sequence of SEQ ID NOS:11949, 10024, and 11588, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17883, 10549, and 10171, respectively and the LCDR1, LCDR2, and LCDR3 comprises an amino acid sequence of SEQ ID NOS: 15615, 16352, and 11080, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17463, 9855, and 11757, respectively and the LCDR1, LCDR2, and LCDR3 comprises an amino acid sequence of SEQ ID NOS: 15984, 9480, and 12697, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6576, 18310, and 10293, respectively and the LCDR1, LCDR2, and LCDR3 comprises an amino acid sequence of SEQ ID NOS:18088, 11073, and 13617, respectively. having a LCDR1, LCDR2, and LCDR3, wherein the HCDR1, HCDR2, and HCDR3 comprises an amino acid sequence of SEQ ID NOS:17263, 9776, and 17559, respectively and the LCDR1, LCDR2, and LCDR3 comprises an amino acid sequence of SEQ ID NOS: 16045, 9503, and 12784, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11673, 10535, and 10504, respectively and the LCDR1, LCDR2, and LCDR3 comprises an amino acid sequence of SEQ ID NOS:10894, 18276, and 12962, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11651, 4074, and 18624, respectively and the LCDR1, LCDR2, and LCDR3 comprises an amino acid sequence of SEQ ID NOS: 10533, 5989, and 9509, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11660, 16896, and 4509, respectively and the LCDR1, LCDR2, and LCDR3 comprises an amino acid sequence of SEQ ID NOS:15980, 17868, and 12868, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4242, 10402, and 16363, respectively and the LCDR1, LCDR2, and LCDR3 comprises an amino acid sequence of SEQ ID NOS: 15765, 6616, and 12033, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16529, 9576, and 4580, respectively and the LCDR1, LCDR2, and LCDR3 comprises an amino acid sequence of SEQ ID NOS: 15988, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17068, 9877, and 11973, respectively and the LCDR1, LCDR2, and LCDR3 comprises an amino acid sequence of SEQ ID NOS: 16044, 9503, and 12599, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9776, and 4211, respectively and the LCDR1, LCDR2, and LCDR3 comprises an amino acid sequence of SEQ ID NOS: 16067, 4425, and 12438, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 4146, and 6794, respectively and the LCDR1, LCDR2, and LCDR3 comprises an amino acid sequence of SEQ ID NOS: 15713, 3982, and 12387, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17848, 10544, and 11707, respectively and the LCDR1, LCDR2, and LCDR3 comprises an amino acid sequence of SEQ ID NOS:10890, 18275, and 12992, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17648, 11498, and 4290, respectively and the LCDR1, LCDR2, and LCDR3 comprises an amino acid sequence of SEQ ID NOS:16377, 10926, and 11268, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16171, 16871, and 16931, respectively and the LCDR1, LCDR2, and LCDR3 comprises an amino acid sequence of SEQ ID NOS: 16079, 9503, and 12856, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 17809, and 5951, respectively and the LCDR1, LCDR2, and LCDR3 comprises an amino acid sequence of SEQ ID NOS:16642, 10747, and 13025, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17432, 18514, and 10228, respectively and the LCDR1, LCDR2, and LCDR3 comprises an amino acid sequence of SEQ ID NOS: 15890, 10727, and 12836, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18104, 9705, and 17034, respectively and the LCDR1, LCDR2, and LCDR3 comprises an amino acid sequence of SEQ ID NOS: 15722, 4425, and 12597, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 17778, and 6728, respectively and the LCDR1, LCDR2, and LCDR3 comprises an amino acid sequence of SEQ ID NOS:10890, 18275, and 12988, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10367, 18323, and 17698, respectively and the LCDR1, LCDR2, and LCDR3 comprises an amino acid sequence of SEQ ID NOS: 15890, 10727, and 12831, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10188, 9869, and 11977, respectively and the LCDR1, LCDR2, and LCDR3 comprises an amino acid sequence of SEQ ID NOS: 16059, 9503, and 12559, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9776, and 18142, respectively and the LCDR1, LCDR2, and LCDR3 comprises an amino acid sequence of SEQ ID NOS: 16751, 17007, and 3999, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17279, 9862, and 11795, respectively and the LCDR1, LCDR2, and LCDR3 comprises an amino acid sequence of SEQ ID NOS: 15984, 4436, and 12700, respectively. having a LCDR1, LCDR2, and LCDR3, wherein the HCDR1, HCDR2, and HCDR3 comprises an amino acid sequence of SEQ ID NOS:17284, 18415, and 10180, respectively and the LCDR1, LCDR2, and LCDR3 comprises an amino acid sequence of SEQ ID NOS: 16045, 9503, and 12767, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11649, 17249, and 11517, respectively and the LCDR1, LCDR2, and LCDR3 comprises an amino acid sequence of SEQ ID NOS:16666, 11894, and 13607, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9971, and 5900, respectively and the LCDR1, LCDR2, and LCDR3 comprises an amino acid sequence of SEQ ID NOS: 15713, 3982, and 13004, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11599, 11547, and 5960, respectively and the LCDR1, LCDR2, and LCDR3 comprises an amino acid sequence of SEQ ID NOS: 17959, 6244, and 11576, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17463, 9787, and 11842, respectively and the LCDR1, LCDR2, and LCDR3 comprises an amino acid sequence of SEQ ID NOS: 15733, 9658, and 12656, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17270, 9406, and 6713, respectively and the LCDR1, LCDR2, and LCDR3 comprises an amino acid sequence of SEQ ID NOS: 15944, 4432, and 11997, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11363, 16295, and 17032, respectively and the LCDR1, LCDR2, and LCDR3 comprises an amino acid sequence of SEQ ID NOS: 16061, 4426, and 12596, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4318, 10304, and 11762, respectively and the LCDR1, LCDR2, and LCDR3 comprises an amino acid sequence of SEQ ID NOS: 16050, 9480, and 12701, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17624, 5443, and 6554, respectively and the LCDR1, LCDR2, and LCDR3 comprises an amino acid sequence of SEQ ID NOS: 15879, 9503, and 12725, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 16919, and 5927, respectively and the LCDR1, LCDR2, and LCDR3 comprises an amino acid sequence of SEQ ID NOS:17924, 10094, and 13582, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11362, 9409, and 17032, respectively and the LCDR1, LCDR2, and LCDR3 comprises an amino acid sequence of SEQ ID NOS: 15732, 4426, and 12710, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17446, 4136, and 18234, respectively and the LCDR1, LCDR2, and LCDR3 comprises an amino acid sequence of SEQ ID NOS:15890, 10727, and 12845, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11364, 11028, and 17032, respectively and the LCDR1, LCDR2, and LCDR3 comprises an amino acid sequence of SEQ ID NOS:15731, 4426, and 12635, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17432, 18517, and 10141, respectively and the LCDR1, LCDR2, and LCDR3 comprises an amino acid sequence of SEQ ID NOS:16075, 10727, and 12019, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11685, 16847, and 17044, respectively and the LCDR1, LCDR2, and LCDR3 comprises an amino acid sequence of SEQ ID NOS:4052, 10084, and 13568, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9863, and 11760, respectively and the LCDR1, LCDR2, and LCDR3 comprises an amino acid sequence of SEQ ID NOS: 16023, 9503, and 12700, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17680, 11428, and 4294, respectively and the LCDR1, LCDR2, and LCDR3 comprises an amino acid sequence of SEQ ID NOS:16368, 10926, and 11260, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17432, 18531, and 17661, respectively and the LCDR1, LCDR2, and LCDR3 comprises an amino acid sequence of SEQ ID NOS:10830, 9267, and 11318, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4136, and 17040, respectively and the LCDR1, LCDR2, and LCDR3 comprises an amino acid sequence of SEQ ID NOS: 15701, 3957, and 12989, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 17778, and 6064, respectively and the LCDR1, LCDR2, and LCDR3 comprises an amino acid sequence of SEQ ID NOS:10890, 18275, and 12985, respectively. having a LCDR1, LCDR2, and LCDR3, wherein the HCDR1, HCDR2, and HCDR3 comprises an amino acid sequence of SEQ ID NOS:17274, 4136, and 17707, respectively and the LCDR1, LCDR2, and LCDR3 comprises an amino acid sequence of SEQ ID NOS: 11789, 4420, and 12575, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6510, 9378, and 5936, respectively and the LCDR1, LCDR2, and LCDR3 comprises an amino acid sequence of SEQ ID NOS: 15955, 4425, and 12428, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9669, 18033, and 4040, respectively and the LCDR1, LCDR2, and LCDR3 comprises an amino acid sequence of SEQ ID NOS:16642, 10747, and 13031, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 10571, and 17215, respectively and the LCDR1, LCDR2, and LCDR3 comprises an amino acid sequence of SEQ ID NOS: 15890, 10727, and 12825, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6187, 18500, and 9543, respectively and the LCDR1, LCDR2, and LCDR3 comprises an amino acid sequence of SEQ ID NOS: 15871, 6611, and 12018, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6576, 16285, and 6154, respectively and the LCDR1, LCDR2, and LCDR3 comprises an amino acid sequence of SEQ ID NOS:10790, 18273, and 12951, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16954, and 16703, respectively and the LCDR1, LCDR2, and LCDR3 comprises an amino acid sequence of SEQ ID NOS: 16736, 7859, and 10277, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11635, 9811, and 17037, respectively and the LCDR1, LCDR2, and LCDR3 comprises an amino acid sequence of SEQ ID NOS: 15928, 4425, and 12413, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17432, 18531, and 6668, respectively and the LCDR1, LCDR2, and LCDR3 comprises an amino acid sequence of SEQ ID NOS: 16061, 9494, and 12694, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4452, 16251, and 9364, respectively and the LCDR1, LCDR2, and LCDR3 comprises an amino acid sequence of SEQ ID NOS:4239, 11608, and 11061, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 9391, and 6065, respectively and the LCDR1, LCDR2, and LCDR3 comprises an amino acid sequence of SEQ ID NOS: 11789, 4429, and 12556, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10344, 18516, and 10069, respectively and the LCDR1, LCDR2, and LCDR3 comprises an amino acid sequence of SEQ ID NOS: 15890, 10727, and 12820, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17447, 16891, and 9345, respectively and the LCDR1, LCDR2, and LCDR3 comprises an amino acid sequence of SEQ ID NOS: 16045, 3983, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11651, 16904, and 6119, respectively and the LCDR1, LCDR2, and LCDR3 comprises an amino acid sequence of SEQ ID NOS: 16019, 4425, and 10711, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 10571, and 17146, respectively and the LCDR1, LCDR2, and LCDR3 comprises an amino acid sequence of SEQ ID NOS: 15704, 3982, and 12096, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17611, 4131, and 16934, respectively and the LCDR1, LCDR2, and LCDR3 comprises an amino acid sequence of SEQ ID NOS: 16007, 9442, and 12856, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10368, 6782, and 10130, respectively and the LCDR1, LCDR2, and LCDR3 comprises an amino acid sequence of SEQ ID NOS: 15893, 3969, and 12514, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16517, 9792, and 17828, respectively and the LCDR1, LCDR2, and LCDR3 comprises an amino acid sequence of SEQ ID NOS: 16071, 9499, and 12946, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11370, 16250, and 6522, respectively and the LCDR1, LCDR2, and LCDR3 comprises an amino acid sequence of SEQ ID NOS: 11789, 4420, and 12580, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6571, 18428, and 11683, respectively and the LCDR1, LCDR2, and LCDR3 comprises an amino acid sequence of SEQ ID NOS: 15653, 9497, and 12456, respectively. having a LCDR1, LCDR2, and LCDR3, wherein the HCDR1, HCDR2, and HCDR3 comprises an amino acid sequence of SEQ ID NOS:11367, 10390, and 17581, respectively and the LCDR1, LCDR2, and LCDR3 comprises an amino acid sequence of SEQ ID NOS:16671, 10747, and 18613, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 10571, and 18222, respectively and the LCDR1, LCDR2, and LCDR3 comprises an amino acid sequence of SEQ ID NOS: 16067, 4425, and 12425, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9776, and 6160, respectively and the LCDR1, LCDR2, and LCDR3 comprises an amino acid sequence of SEQ ID NOS:10890, 18275, and 12984, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11670, 16886, and 6060, respectively and the LCDR1, LCDR2, and LCDR3 comprises an amino acid sequence of SEQ ID NOS: 15879, 3983, and 12947, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6564, 16841, and 4570, respectively and the LCDR1, LCDR2, and LCDR3 comprises an amino acid sequence of SEQ ID NOS:9587, 18447, and 15572, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6578, 18531, and 4506, respectively and the LCDR1, LCDR2, and LCDR3 comprises an amino acid sequence of SEQ ID NOS:17924, 10094, and 13578, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16798, 9880, and 10129, respectively and the LCDR1, LCDR2, and LCDR3 comprises an amino acid sequence of SEQ ID NOS: 16047, 9494, and 12622, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17535, 10571, and 7957, respectively and the LCDR1, LCDR2, and LCDR3 comprises an amino acid sequence of SEQ ID NOS: 15711, 3969, and 12334, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16954, and 16187, respectively and the LCDR1, LCDR2, and LCDR3 comprises an amino acid sequence of SEQ ID NOS:9633, 4467, and 15559, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11672, 11171, and 10591, respectively and the LCDR1, LCDR2, and LCDR3 comprises an amino acid sequence of SEQ ID NOS:10807, 18275, and 12937, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11632, 18001, and 16192, respectively and the LCDR1, LCDR2, and LCDR3 comprises an amino acid sequence of SEQ ID NOS:16400, 10987, and 11212, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9776, and 4396, respectively and the LCDR1, LCDR2, and LCDR3 comprises an amino acid sequence of SEQ ID NOS: 15713, 3982, and 12392, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17272, 4150, and 6069, respectively and the LCDR1, LCDR2, and LCDR3 comprises an amino acid sequence of SEQ ID NOS:9629, 5985, and 15567, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6510, 9379, and 17688, respectively and the LCDR1, LCDR2, and LCDR3 comprises an amino acid sequence of SEQ ID NOS:16428, 10764, and 11222, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17432, 18521, and 10218, respectively and the LCDR1, LCDR2, and LCDR3 comprises an amino acid sequence of SEQ ID NOS: 16061, 9494, and 12699, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9776, and 18221, respectively and the LCDR1, LCDR2, and LCDR3 comprises an amino acid sequence of SEQ ID NOS:15890, 10727, and 12831, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16526, 9785, and 17829, respectively and the LCDR1, LCDR2, and LCDR3 comprises an amino acid sequence of SEQ ID NOS:15890, 10737, and 12023, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 17778, and 4567, respectively and the LCDR1, LCDR2, and LCDR3 comprises an amino acid sequence of SEQ ID NOS: 10830, 9267, and 11319, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 11488, and 4048, respectively and the LCDR1, LCDR2, and LCDR3 comprises an amino acid sequence of SEQ ID NOS:16428, 10764, and 11227, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9872, and 11758, respectively and the LCDR1, LCDR2, and LCDR3 comprises an amino acid sequence of SEQ ID NOS: 15984, 9503, and 12700, respectively. having a LCDR1, LCDR2, and LCDR3, wherein the HCDR1, HCDR2, and HCDR3 comprises an amino acid sequence of SEQ ID NOS:17443, 17809, and 17710, respectively and the LCDR1, LCDR2, and LCDR3 comprises an amino acid sequence of SEQ ID NOS:17959, 6318, and 17192, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 10571, and 17596, respectively and the LCDR1, LCDR2, and LCDR3 comprises an amino acid sequence of SEQ ID NOS: 16045, 9503, and 12767, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17068, 9878, and 11969, respectively and the LCDR1, LCDR2, and LCDR3 comprises an amino acid sequence of SEQ ID NOS: 15979, 9472, and 12700, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17284, 18415, and 18108, respectively and the LCDR1, LCDR2, and LCDR3 comprises an amino acid sequence of SEQ ID NOS: 16045, 9494, and 12665, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17274, 4136, and 9592, respectively and the LCDR1, LCDR2, and LCDR3 comprises an amino acid sequence of SEQ ID NOS:16659, 11900, and 11871, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17270, 17778, and 7813, respectively and the LCDR1, LCDR2, and LCDR3 comprises an amino acid sequence of SEQ ID NOS: 17959, 6312, and 4376, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10338, 9732, and 11968, respectively and the LCDR1, LCDR2, and LCDR3 comprises an amino acid sequence of SEQ ID NOS: 16061, 9494, and 12701, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10354, 9788, and 11763, respectively and the LCDR1, LCDR2, and LCDR3 comprises an amino acid sequence of SEQ ID NOS: 16059, 9503, and 12594, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17535, 10571, and 7899, respectively and the LCDR1, LCDR2, and LCDR3 comprises an amino acid sequence of SEQ ID NOS: 16061, 9494, and 12695, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16582, 11017, and 6725, respectively and the LCDR1, LCDR2, and LCDR3 comprises an amino acid sequence of SEQ ID NOS: 15876, 3983, and 12755, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 16919, and 4178, respectively and the LCDR1, LCDR2, and LCDR3 comprises an amino acid sequence of SEQ ID NOS: 11789, 4420, and 12578, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4306, 9933, and 17010, respectively and the LCDR1, LCDR2, and LCDR3 comprises an amino acid sequence of SEQ ID NOS: 15845, 9494, and 12631, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17272, 18342, and 10255, respectively and the LCDR1, LCDR2, and LCDR3 comprises an amino acid sequence of SEQ ID NOS:15713, 3982, and 13006, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17620, 9398, and 4504, respectively and the LCDR1, LCDR2, and LCDR3 comprises an amino acid sequence of SEQ ID NOS: 16067, 4425, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16605, 11431, and 12301, respectively and the LCDR1, LCDR2, and LCDR3 comprises an amino acid sequence of SEQ ID NOS:15673, 6216, and 12457, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4503, 18394, and 10086, respectively and the LCDR1, LCDR2, and LCDR3 comprises an amino acid sequence of SEQ ID NOS:10850, 10931, and 11259, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17885, 10006, and 5975, respectively and the LCDR1, LCDR2, and LCDR3 comprises an amino acid sequence of SEQ ID NOS: 15699, 3981, and 11988, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16818, 16264, and 10039, respectively and the LCDR1, LCDR2, and LCDR3 comprises an amino acid sequence of SEQ ID NOS: 16357, 16480, and 11279, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 10571, and 17061, respectively and the LCDR1, LCDR2, and LCDR3 comprises an amino acid sequence of SEQ ID NOS: 16061, 9494, and 12676, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17284, 18415, and 4220, respectively and the LCDR1, LCDR2, and LCDR3 comprises an amino acid sequence of SEQ ID NOS: 17959, 6312, and 17197, respectively. having a LCDR1, LCDR2, and LCDR3, wherein the HCDR1, HCDR2, and HCDR3 comprises an amino acid sequence of SEQ ID NOS: 17838, 10397, and 5924, respectively and the LCDR1, LCDR2, and LCDR3 comprises an amino acid sequence of SEQ ID NOS:10890, 18275, and 12972, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16954, and 11708, respectively and the LCDR1, LCDR2, and LCDR3 comprises an amino acid sequence of SEQ ID NOS:16061, 9494, and 12671, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 17809, and 6240, respectively and the LCDR1, LCDR2, and LCDR3 comprises an amino acid sequence of SEQ ID NOS: 16067, 4425, and 10711, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16954, and 10416, respectively and the LCDR1, LCDR2, and LCDR3 comprises an amino acid sequence of SEQ ID NOS:17959, 6318, and 17191, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4306, 9850, and 17013, respectively and the LCDR1, LCDR2, and LCDR3 comprises an amino acid sequence of SEQ ID NOS: 15846, 9480, and 12700, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11646, 18380, and 9647, respectively and the LCDR1, LCDR2, and LCDR3 comprises an amino acid sequence of SEQ ID NOS: 16491, 9503, and 12640, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16692, 18569, and 10219, respectively and the LCDR1, LCDR2, and LCDR3 comprises an amino acid sequence of SEQ ID NOS:9633, 18448, and 15563, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11688, 11533, and 10421, respectively and the LCDR1, LCDR2, and LCDR3 comprises an amino acid sequence of SEQ ID NOS: 16052, 9494, and 12637, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16630, 16897, and 6555, respectively and the LCDR1, LCDR2, and LCDR3 comprises an amino acid sequence of SEQ ID NOS: 15957, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10368, 6782, and 11714, respectively and the LCDR1, LCDR2, and LCDR3 comprises an amino acid sequence of SEQ ID NOS:16091, 9494, and 12671, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17547, 18308, and 10098, respectively and the LCDR1, LCDR2, and LCDR3 comprises an amino acid sequence of SEQ ID NOS: 15701, 4256, and 10477, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:7820, 9562, and 18213, respectively and the LCDR1, LCDR2, and LCDR3 comprises an amino acid sequence of SEQ ID NOS: 16067, 4425, and 12443, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6578, 18531, and 10178, respectively and the LCDR1, LCDR2, and LCDR3 comprises an amino acid sequence of SEQ ID NOS: 16045, 9503, and 12768, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17432, 18531, and 6561, respectively and the LCDR1, LCDR2, and LCDR3 comprises an amino acid sequence of SEQ ID NOS: 11789, 4420, and 12581, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17856, 18589, and 4502, respectively and the LCDR1, LCDR2, and LCDR3 comprises an amino acid sequence of SEQ ID NOS: 17942, 6312, and 4378, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11648, 18386, and 6108, respectively and the LCDR1, LCDR2, and LCDR3 comprises an amino acid sequence of SEQ ID NOS:16381, 10763, and 11263, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10682, 18467, and 7956, respectively and the LCDR1, LCDR2, and LCDR3 comprises an amino acid sequence of SEQ ID NOS:9662, 5985, and 11940, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16990, 10001, and 10437, respectively and the LCDR1, LCDR2, and LCDR3 comprises an amino acid sequence of SEQ ID NOS: 10830, 9267, and 11325, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 9388, and 6136, respectively and the LCDR1, LCDR2, and LCDR3 comprises an amino acid sequence of SEQ ID NOS: 11789, 4420, and 12573, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 10571, and 18143, respectively and the LCDR1, LCDR2, and LCDR3 comprises an amino acid sequence of SEQ ID NOS: 10830, 9267, and 11320, respectively. having a LCDR1, LCDR2, and LCDR3, wherein the HCDR1, HCDR2, and HCDR3 comprises an amino acid sequence of SEQ ID NOS: 17270, 17777, and 6485, respectively and the LCDR1, LCDR2, and LCDR3 comprises an amino acid sequence of SEQ ID NOS: 16045, 9503, and 12770, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17856, 16303, and 10323, respectively and the LCDR1, LCDR2, and LCDR3 comprises an amino acid sequence of SEQ ID NOS: 16715, 16326, and 4008, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16595, 16228, and 4623, respectively and the LCDR1, LCDR2, and LCDR3 comprises an amino acid sequence of SEQ ID NOS:10898, 18275, and 12960, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4130, and 10136, respectively and the LCDR1, LCDR2, and LCDR3 comprises an amino acid sequence of SEQ ID NOS: 16045, 9503, and 12604, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4457, 18311, and 11569, respectively and the LCDR1, LCDR2, and LCDR3 comprises an amino acid sequence of SEQ ID NOS:10802, 18275, and 10480, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6146, 18488, and 4574, respectively and the LCDR1, LCDR2, and LCDR3 comprises an amino acid sequence of SEQ ID NOS: 15852, 3962, and 12481, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18359, and 6101, respectively and the LCDR1, LCDR2, and LCDR3 comprises an amino acid sequence of SEQ ID NOS:16432, 10926, and 11291, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16603, 16225, and 4394, respectively and the LCDR1, LCDR2, and LCDR3 comprises an amino acid sequence of SEQ ID NOS:9359, 11895, and 11865, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17027, 17823, and 4463, respectively and the LCDR1, LCDR2, and LCDR3 comprises an amino acid sequence of SEQ ID NOS: 15704, 3969, and 12803, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 9388, and 6149, respectively and the LCDR1, LCDR2, and LCDR3 comprises an amino acid sequence of SEQ ID NOS:16427, 10926, and 11276, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 9388, and 4617, respectively and the LCDR1, LCDR2, and LCDR3 comprises an amino acid sequence of SEQ ID NOS:16408, 10987, and 11208, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 17778, and 4646, respectively and the LCDR1, LCDR2, and LCDR3 comprises an amino acid sequence of SEQ ID NOS: 15713, 3982, and 12391, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17855, 16304, and 6693, respectively and the LCDR1, LCDR2, and LCDR3 comprises an amino acid sequence of SEQ ID NOS: 16045, 9503, and 12780, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11385, 4068, and 10288, respectively and the LCDR1, LCDR2, and LCDR3 comprises an amino acid sequence of SEQ ID NOS: 15897, 3967, and 12521, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9568, 5444, and 4478, respectively and the LCDR1, LCDR2, and LCDR3 comprises an amino acid sequence of SEQ ID NOS:16767, 4173, and 4015, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17991, 10566, and 10429, respectively and the LCDR1, LCDR2, and LCDR3 comprises an amino acid sequence of SEQ ID NOS:4237, 17225, and 11060, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11413, 18632, and 6061, respectively and the LCDR1, LCDR2, and LCDR3 comprises an amino acid sequence of SEQ ID NOS:15635, 17863, and 12474, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17838, 16277, and 7888, respectively and the LCDR1, LCDR2, and LCDR3 comprises an amino acid sequence of SEQ ID NOS:9633, 18448, and 15560, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11633, 10551, and 4183, respectively and the LCDR1, LCDR2, and LCDR3 comprises an amino acid sequence of SEQ ID NOS:10890, 18271, and 12942, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17432, 18555, and 5898, respectively and the LCDR1, LCDR2, and LCDR3 comprises an amino acid sequence of SEQ ID NOS:18090, 11069, and 13623, respectively. having a LCDR1, LCDR2, and LCDR3, wherein the HCDR1, HCDR2, and HCDR3 comprises an amino acid sequence of SEQ ID NOS:17842, 11491, and 10975, respectively and the LCDR1, LCDR2, and LCDR3 comprises an amino acid sequence of SEQ ID NOS:10905, 18275, and 12959, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17606, 9753, and 6097, respectively and the LCDR1, LCDR2, and LCDR3 comprises an amino acid sequence of SEQ ID NOS:16463, 10987, and 11254, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16796, 11430, and 11706, respectively and the LCDR1, LCDR2, and LCDR3 comprises an amino acid sequence of SEQ ID NOS:10811, 18275, and 12896, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 17778, and 4622, respectively and the LCDR1, LCDR2, and LCDR3 comprises an amino acid sequence of SEQ ID NOS:11949, 10024, and 11585, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16692, 18586, and 4312, respectively and the LCDR1, LCDR2, and LCDR3 comprises an amino acid sequence of SEQ ID NOS: 16067, 4425, and 12420, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10935, 9846, and 10240, respectively and the LCDR1, LCDR2, and LCDR3 comprises an amino acid sequence of SEQ ID NOS:16062, 10261, and 12641, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17855, 16220, and 7832, respectively and the LCDR1, LCDR2, and LCDR3 comprises an amino acid sequence of SEQ ID NOS:10806, 18275, and 12987, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4136, and 10957, respectively and the LCDR1, LCDR2, and LCDR3 comprises an amino acid sequence of SEQ ID NOS: 16061, 9494, and 12696, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4633, 18303, and 6009, respectively and the LCDR1, LCDR2, and LCDR3 comprises an amino acid sequence of SEQ ID NOS: 16023, 9494, and 12643, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11679, 11513, and 9473, respectively and the LCDR1, LCDR2, and LCDR3 comprises an amino acid sequence of SEQ ID NOS:10792, 18275, and 12985, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10779, 9795, and 6166, respectively and the LCDR1, LCDR2, and LCDR3 comprises an amino acid sequence of SEQ ID NOS: 16061, 4432, and 12713, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9968, and 16682, respectively and the LCDR1, LCDR2, and LCDR3 comprises an amino acid sequence of SEQ ID NOS:16671, 10747, and 18615, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17655, 18558, and 4474, respectively and the LCDR1, LCDR2, and LCDR3 comprises an amino acid sequence of SEQ ID NOS:16356, 10263, and 12474, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 16919, and 6665, respectively and the LCDR1, LCDR2, and LCDR3 comprises an amino acid sequence of SEQ ID NOS: 17959, 9268, and 17188, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 11488, and 6164, respectively and the LCDR1, LCDR2, and LCDR3 comprises an amino acid sequence of SEQ ID NOS: 10830, 9267, and 11322, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11642, 18533, and 9557, respectively and the LCDR1, LCDR2, and LCDR3 comprises an amino acid sequence of SEQ ID NOS:15847, 10737, and 12834, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11642, 16924, and 10290, respectively and the LCDR1, LCDR2, and LCDR3 comprises an amino acid sequence of SEQ ID NOS: 15597, 4436, and 12725, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10227, 11010, and 10481, respectively and the LCDR1, LCDR2, and LCDR3 comprises an amino acid sequence of SEQ ID NOS:15817, 10727, and 12811, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17028, 17823, and 6120, respectively and the LCDR1, LCDR2, and LCDR3 comprises an amino acid sequence of SEQ ID NOS:15890, 10727, and 12825, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17856, 16306, and 10099, respectively and the LCDR1, LCDR2, and LCDR3 comprises an amino acid sequence of SEQ ID NOS: 16736, 7860, and 10274, respectively. having a LCDR1, LCDR2, and LCDR3, wherein the HCDR1, HCDR2, and HCDR3 comprises an amino acid sequence of SEQ ID NOS:6507, 9971, and 10175, respectively and the LCDR1, LCDR2, and LCDR3 comprises an amino acid sequence of SEQ ID NOS: 17959, 9268, and 17190, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17843, 18049, and 4602, respectively and the LCDR1, LCDR2, and LCDR3 comprises an amino acid sequence of SEQ ID NOS:10878, 18275, and 12916, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10368, 6737, and 10132, respectively and the LCDR1, LCDR2, and LCDR3 comprises an amino acid sequence of SEQ ID NOS:15659, 17663, and 10466, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17839, 11508, and 10526, respectively and the LCDR1, LCDR2, and LCDR3 comprises an amino acid sequence of SEQ ID NOS: 15674, 10370, and 11079, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11640, 17590, and 18283, respectively and the LCDR1, LCDR2, and LCDR3 comprises an amino acid sequence of SEQ ID NOS:15871, 10737, and 12810, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17608, 18030, and 6175, respectively and the LCDR1, LCDR2, and LCDR3 comprises an amino acid sequence of SEQ ID NOS: 16045, 4436, and 12946, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16796, 16939, and 10253, respectively and the LCDR1, LCDR2, and LCDR3 comprises an amino acid sequence of SEQ ID NOS: 16061, 9494, and 12669, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17266, 9728, and 11120, respectively and the LCDR1, LCDR2, and LCDR3 comprises an amino acid sequence of SEQ ID NOS: 15748, 9494, and 12005, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10368, 6782, and 7898, respectively and the LCDR1, LCDR2, and LCDR3 comprises an amino acid sequence of SEQ ID NOS: 16061, 9494, and 12688, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17432, 18523, and 11372, respectively and the LCDR1, LCDR2, and LCDR3 comprises an amino acid sequence of SEQ ID NOS:17936, 6721, and 4361, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6580, 16301, and 10302, respectively and the LCDR1, LCDR2, and LCDR3 comprises an amino acid sequence of SEQ ID NOS:11950, 10024, and 11592, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16990, 9854, and 11191, respectively and the LCDR1, LCDR2, and LCDR3 comprises an amino acid sequence of SEQ ID NOS: 10826, 9267, and 11338, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 9370, and 6053, respectively and the LCDR1, LCDR2, and LCDR3 comprises an amino acid sequence of SEQ ID NOS:15890, 10704, and 10469, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9836, and 10483, respectively and the LCDR1, LCDR2, and LCDR3 comprises an amino acid sequence of SEQ ID NOS: 15883, 4426, and 12625, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17432, 18531, and 6056, respectively and the LCDR1, LCDR2, and LCDR3 comprises an amino acid sequence of SEQ ID NOS:17924, 10094, and 13587, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10367, 18323, and 7858, respectively and the LCDR1, LCDR2, and LCDR3 comprises an amino acid sequence of SEQ ID NOS: 16061, 9494, and 12668, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17558, 11039, and 17545, respectively and the LCDR1, LCDR2, and LCDR3 comprises an amino acid sequence of SEQ ID NOS: 16067, 4425, and 12426, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10367, 18323, and 10298, respectively and the LCDR1, LCDR2, and LCDR3 comprises an amino acid sequence of SEQ ID NOS: 16408, 10987, and 11203, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 10571, and 17657, respectively and the LCDR1, LCDR2, and LCDR3 comprises an amino acid sequence of SEQ ID NOS: 15890, 10727, and 12839, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11413, 18469, and 6123, respectively and the LCDR1, LCDR2, and LCDR3 comprises an amino acid sequence of SEQ ID NOS:15636, 17862, and 12540, respectively. having a LCDR1, LCDR2, and LCDR3, wherein the HCDR1, HCDR2, and HCDR3 comprises an amino acid sequence of SEQ ID NOS: 17466, 16919, and 6623, respectively and the LCDR1, LCDR2, and LCDR3 comprises an amino acid sequence of SEQ ID NOS: 15713, 3982, and 13003, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6578, 18531, and 9609, respectively and the LCDR1, LCDR2, and LCDR3 comprises an amino acid sequence of SEQ ID NOS: 16061, 9494, and 12670, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 18636, 18355, and 4191, respectively and the LCDR1, LCDR2, and LCDR3 comprises an amino acid sequence of SEQ ID NOS: 10808, 9267, and 11329, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6530, 9915, and 4621, respectively and the LCDR1, LCDR2, and LCDR3 comprises an amino acid sequence of SEQ ID NOS:9629, 4470, and 15557, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17840, 11503, and 6163, respectively and the LCDR1, LCDR2, and LCDR3 comprises an amino acid sequence of SEQ ID NOS:18072, 18596, and 11179, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16633, 16313, and 10195, respectively and the LCDR1, LCDR2, and LCDR3 comprises an amino acid sequence of SEQ ID NOS: 16045, 9503, and 12782, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17432, 18531, and 16551, respectively and the LCDR1, LCDR2, and LCDR3 comprises an amino acid sequence of SEQ ID NOS:16122, 6615, and 12848, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 16971, and 4227, respectively and the LCDR1, LCDR2, and LCDR3 comprises an amino acid sequence of SEQ ID NOS:16114, 10703, and 12601, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16575, 18474, and 10523, respectively and the LCDR1, LCDR2, and LCDR3 comprises an amino acid sequence of SEQ ID NOS:4057, 6312, and 4374, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6571, 18323, and 11390, respectively and the LCDR1, LCDR2, and LCDR3 comprises an amino acid sequence of SEQ ID NOS:17946, 6331, and 4347, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9776, and 4061, respectively and the LCDR1, LCDR2, and LCDR3 comprises an amino acid sequence of SEQ ID NOS: 17959, 9268, and 17196, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18360, and 6696, respectively and the LCDR1, LCDR2, and LCDR3 comprises an amino acid sequence of SEQ ID NOS: 17959, 9268, and 17190, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4457, 18384, and 11570, respectively and the LCDR1, LCDR2, and LCDR3 comprises an amino acid sequence of SEQ ID NOS:10802, 18275, and 10480, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11678, 11548, and 4573, respectively and the LCDR1, LCDR2, and LCDR3 comprises an amino acid sequence of SEQ ID NOS:15813, 17226, and 12555, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11411, 11472, and 6031, respectively and the LCDR1, LCDR2, and LCDR3 comprises an amino acid sequence of SEQ ID NOS:10798, 18275, and 12959, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17534, 10568, and 16168, respectively and the LCDR1, LCDR2, and LCDR3 comprises an amino acid sequence of SEQ ID NOS:16018, 4425, and 12427, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11690, 16222, and 5916, respectively and the LCDR1, LCDR2, and LCDR3 comprises an amino acid sequence of SEQ ID NOS:16659, 11900, and 11884, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11582, 9784, and 7915, respectively and the LCDR1, LCDR2, and LCDR3 comprises an amino acid sequence of SEQ ID NOS: 10828, 9267, and 11319, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11690, 16318, and 10341, respectively and the LCDR1, LCDR2, and LCDR3 comprises an amino acid sequence of SEQ ID NOS: 10787, 9267, and 11315, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17856, 16219, and 7955, respectively and the LCDR1, LCDR2, and LCDR3 comprises an amino acid sequence of SEQ ID NOS: 16737, 16331, and 4012, respectively. having a LCDR1, LCDR2, and LCDR3, wherein the HCDR1, HCDR2, and HCDR3 comprises an amino acid sequence of SEQ ID NOS:11642, 18600, and 10149, respectively and the LCDR1, LCDR2, and LCDR3 comprises an amino acid sequence of SEQ ID NOS:15861, 10727, and 12800, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16990, 9881, and 18627, respectively and the LCDR1, LCDR2, and LCDR3 comprises an amino acid sequence of SEQ ID NOS: 10830, 9272, and 11300, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11677, 17246, and 10060, respectively and the LCDR1, LCDR2, and LCDR3 comprises an amino acid sequence of SEQ ID NOS: 15767, 9555, and 10438, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18360, and 6692, respectively and the LCDR1, LCDR2, and LCDR3 comprises an amino acid sequence of SEQ ID NOS: 17550, 6629, and 13584, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17432, 18532, and 4402, respectively and the LCDR1, LCDR2, and LCDR3 comprises an amino acid sequence of SEQ ID NOS:16114, 11791, and 12818, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 17778, and 6171, respectively and the LCDR1, LCDR2, and LCDR3 comprises an amino acid sequence of SEQ ID NOS: 17550, 6629, and 13585, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4136, and 11075, respectively and the LCDR1, LCDR2, and LCDR3 comprises an amino acid sequence of SEQ ID NOS: 17967, 9268, and 17186, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11650, 17809, and 9638, respectively and the LCDR1, LCDR2, and LCDR3 comprises an amino acid sequence of SEQ ID NOS: 16056, 9503, and 12731, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10368, 6782, and 9624, respectively and the LCDR1, LCDR2, and LCDR3 comprises an amino acid sequence of SEQ ID NOS: 16025, 9494, and 12862, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16795, 11520, and 9586, respectively and the LCDR1, LCDR2, and LCDR3 comprises an amino acid sequence of SEQ ID NOS:10900, 18272, and 12992, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6529, 9698, and 5892, respectively and the LCDR1, LCDR2, and LCDR3 comprises an amino acid sequence of SEQ ID NOS: 15984, 9494, and 12278, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11673, 10565, and 17223, respectively and the LCDR1, LCDR2, and LCDR3 comprises an amino acid sequence of SEQ ID NOS:9485, 4393, and 12708, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9971, and 4599, respectively and the LCDR1, LCDR2, and LCDR3 comprises an amino acid sequence of SEQ ID NOS: 11789, 4420, and 12574, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17646, 16262, and 6335, respectively and the LCDR1, LCDR2, and LCDR3 comprises an amino acid sequence of SEQ ID NOS: 15958, 9504, and 12013, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17855, 16306, and 18111, respectively and the LCDR1, LCDR2, and LCDR3 comprises an amino acid sequence of SEQ ID NOS:16659, 11903, and 11879, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17432, 18515, and 4408, respectively and the LCDR1, LCDR2, and LCDR3 comprises an amino acid sequence of SEQ ID NOS:15871, 11794, and 11929, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17856, 18560, and 6217, respectively and the LCDR1, LCDR2, and LCDR3 comprises an amino acid sequence of SEQ ID NOS: 15812, 4255, and 12472, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17474, 9768, and 10135, respectively and the LCDR1, LCDR2, and LCDR3 comprises an amino acid sequence of SEQ ID NOS: 16067, 4425, and 12431, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 17748, and 6113, respectively and the LCDR1, LCDR2, and LCDR3 comprises an amino acid sequence of SEQ ID NOS: 17950, 9268, and 11601, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17478, 9828, and 9356, respectively and the LCDR1, LCDR2, and LCDR3 comprises an amino acid sequence of SEQ ID NOS:10835, 10983, and 11193, respectively. having a LCDR1, LCDR2, and LCDR3, wherein the HCDR1, HCDR2, and HCDR3 comprises an amino acid sequence of SEQ ID NOS:17263, 9763, and 17682, respectively and the LCDR1, LCDR2, and LCDR3 comprises an amino acid sequence of SEQ ID NOS: 16150, 6308, and 12418, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17432, 18531, and 7919, respectively and the LCDR1, LCDR2, and LCDR3 comprises an amino acid sequence of SEQ ID NOS: 16045, 9503, and 12768, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17641, 12036, and 4589, respectively and the LCDR1, LCDR2, and LCDR3 comprises an amino acid sequence of SEQ ID NOS:18086, 10590, and 13610, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17647, 16848, and 10116, respectively and the LCDR1, LCDR2, and LCDR3 comprises an amino acid sequence of SEQ ID NOS:10801, 18275, and 12963, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16796, 16939, and 11717, respectively and the LCDR1, LCDR2, and LCDR3 comprises an amino acid sequence of SEQ ID NOS: 16067, 4425, and 10709, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17847, 17740, and 16191, respectively and the LCDR1, LCDR2, and LCDR3 comprises an amino acid sequence of SEQ ID NOS: 15685, 3969, and 12805, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 16853, and 5448, respectively and the LCDR1, LCDR2, and LCDR3 comprises an amino acid sequence of SEQ ID NOS: 17960, 6722, and 4344, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16796, 16939, and 9520, respectively and the LCDR1, LCDR2, and LCDR3 comprises an amino acid sequence of SEQ ID NOS: 16045, 9503, and 12777, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17648, 16840, and 10995, respectively and the LCDR1, LCDR2, and LCDR3 comprises an amino acid sequence of SEQ ID NOS:10886, 18275, and 12913, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10367, 18323, and 4164, respectively and the LCDR1, LCDR2, and LCDR3 comprises an amino acid sequence of SEQ ID NOS:15713, 3982, and 12390, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17888, 9574, and 6104, respectively and the LCDR1, LCDR2, and LCDR3 comprises an amino acid sequence of SEQ ID NOS: 16056, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17842, 11487, and 17725, respectively and the LCDR1, LCDR2, and LCDR3 comprises an amino acid sequence of SEQ ID NOS: 15890, 10727, and 12837, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17272, 17760, and 6479, respectively and the LCDR1, LCDR2, and LCDR3 comprises an amino acid sequence of SEQ ID NOS: 16736, 5977, and 10279, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17208, 18460, and 4439, respectively and the LCDR1, LCDR2, and LCDR3 comprises an amino acid sequence of SEQ ID NOS: 15633, 3977, and 12544, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18360, and 4490, respectively and the LCDR1, LCDR2, and LCDR3 comprises an amino acid sequence of SEQ ID NOS: 16045, 9503, and 12789, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6500, 9974, and 5966, respectively and the LCDR1, LCDR2, and LCDR3 comprises an amino acid sequence of SEQ ID NOS:17960, 6717, and 4365, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11623, 9806, and 4187, respectively and the LCDR1, LCDR2, and LCDR3 comprises an amino acid sequence of SEQ ID NOS: 16494, 4428, and 12354, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16603, 18578, and 4440, respectively and the LCDR1, LCDR2, and LCDR3 comprises an amino acid sequence of SEQ ID NOS: 15596, 3953, and 12484, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17028, 17823, and 9648, respectively and the LCDR1, LCDR2, and LCDR3 comprises an amino acid sequence of SEQ ID NOS: 15713, 3982, and 13005, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11374, 11471, and 10054, respectively and the LCDR1, LCDR2, and LCDR3 comprises an amino acid sequence of SEQ ID NOS:10869, 18275, and 12965, respectively. having a LCDR1, LCDR2, and LCDR3, wherein the HCDR1, HCDR2, and HCDR3 comprises an amino acid sequence of SEQ ID NOS: 11422, 17771, and 6137, respectively and the LCDR1, LCDR2, and LCDR3 comprises an amino acid sequence of SEQ ID NOS: 16054, 9494, and 10464, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17856, 16224, and 6731, respectively and the LCDR1, LCDR2, and LCDR3 comprises an amino acid sequence of SEQ ID NOS: 16045, 9503, and 12758, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10347, 18333, and 16775, respectively and the LCDR1, LCDR2, and LCDR3 comprises an amino acid sequence of SEQ ID NOS: 16045, 9503, and 12780, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 9388, and 5922, respectively and the LCDR1, LCDR2, and LCDR3 comprises an amino acid sequence of SEQ ID NOS:10059, 17222, and 18118, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17432, 18498, and 4627, respectively and the LCDR1, LCDR2, and LCDR3 comprises an amino acid sequence of SEQ ID NOS:15890, 10727, and 12820, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9542, 16220, and 4616, respectively and the LCDR1, LCDR2, and LCDR3 comprises an amino acid sequence of SEQ ID NOS:16642, 10756, and 13336, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6530, 9918, and 6620, respectively and the LCDR1, LCDR2, and LCDR3 comprises an amino acid sequence of SEQ ID NOS:4056, 10094, and 13590, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9966, and 5442, respectively and the LCDR1, LCDR2, and LCDR3 comprises an amino acid sequence of SEQ ID NOS:17945, 6717, and 4362, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10531, 18506, and 9538, respectively and the LCDR1, LCDR2, and LCDR3 comprises an amino acid sequence of SEQ ID NOS: 16114, 6607, and 12833, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18360, and 9338, respectively and the LCDR1, LCDR2, and LCDR3 comprises an amino acid sequence of SEQ ID NOS: 16045, 9503, and 12787, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16791, 11427, and 6642, respectively and the LCDR1, LCDR2, and LCDR3 comprises an amino acid sequence of SEQ ID NOS: 15680, 3969, and 12337, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18360, and 5999, respectively and the LCDR1, LCDR2, and LCDR3 comprises an amino acid sequence of SEQ ID NOS: 16752, 17007, and 4003, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6528, 9380, and 6018, respectively and the LCDR1, LCDR2, and LCDR3 comprises an amino acid sequence of SEQ ID NOS: 15985, 9480, and 12460, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16597, 4172, and 6730, respectively and the LCDR1, LCDR2, and LCDR3 comprises an amino acid sequence of SEQ ID NOS:15851, 3969, and 12801, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:7820, 9562, and 10238, respectively and the LCDR1, LCDR2, and LCDR3 comprises an amino acid sequence of SEQ ID NOS: 16061, 9494, and 12673, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16522, 18522, and 9849, respectively and the LCDR1, LCDR2, and LCDR3 comprises an amino acid sequence of SEQ ID NOS:11946, 10029, and 11594, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10368, 6782, and 4300, respectively and the LCDR1, LCDR2, and LCDR3 comprises an amino acid sequence of SEQ ID NOS: 16045, 9503, and 12795, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6530, 9919, and 6038, respectively and the LCDR1, LCDR2, and LCDR3 comprises an amino acid sequence of SEQ ID NOS: 15914, 4425, and 12426, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16610, 9775, and 6170, respectively and the LCDR1, LCDR2, and LCDR3 comprises an amino acid sequence of SEQ ID NOS: 16045, 9503, and 12584, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17474, 16231, and 6008, respectively and the LCDR1, LCDR2, and LCDR3 comprises an amino acid sequence of SEQ ID NOS:17924, 10094, and 13583, respectively. having a LCDR1, LCDR2, and LCDR3, wherein the HCDR1, HCDR2, and HCDR3 comprises an amino acid sequence of SEQ ID NOS:17263, 9804, and 9621, respectively and the LCDR1, LCDR2, and LCDR3 comprises an amino acid sequence of SEQ ID NOS: 16125, 4432, and 12697, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17274, 4136, and 4023, respectively and the LCDR1, LCDR2, and LCDR3 comprises an amino acid sequence of SEQ ID NOS:17905, 16330, and 16564, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18252, 9368, and 11713, respectively and the LCDR1, LCDR2, and LCDR3 comprises an amino acid sequence of SEQ ID NOS:10868, 18275, and 12912, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17463, 9737, and 9618, respectively and the LCDR1, LCDR2, and LCDR3 comprises an amino acid sequence of SEQ ID NOS: 16061, 9494, and 12679, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6520, 18413, and 10142, respectively and the LCDR1, LCDR2, and LCDR3 comprises an amino acid sequence of SEQ ID NOS: 17959, 9277, and 17109, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9676, 10552, and 4407, respectively and the LCDR1, LCDR2, and LCDR3 comprises an amino acid sequence of SEQ ID NOS: 17951, 10311, and 4335, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17838, 16293, and 4313, respectively and the LCDR1, LCDR2, and LCDR3 comprises an amino acid sequence of SEQ ID NOS:17959, 9268, and 17181, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6521, 9914, and 18250, respectively and the LCDR1, LCDR2, and LCDR3 comprises an amino acid sequence of SEQ ID NOS: 16061, 4432, and 12677, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16800, 9989, and 10159, respectively and the LCDR1, LCDR2, and LCDR3 comprises an amino acid sequence of SEQ ID NOS:15931, 7929, and 12462, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16177, 10251, and 9581, respectively and the LCDR1, LCDR2, and LCDR3 comprises an amino acid sequence of SEQ ID NOS: 16089, 4431, and 12625, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17456, 18546, and 4468, respectively and the LCDR1, LCDR2, and LCDR3 comprises an amino acid sequence of SEQ ID NOS:9632, 16163, and 15566, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16702, 18576, and 17133, respectively and the LCDR1, LCDR2, and LCDR3 comprises an amino acid sequence of SEQ ID NOS:16659, 6628, and 11878, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17006, 10670, and 4243, respectively and the LCDR1, LCDR2, and LCDR3 comprises an amino acid sequence of SEQ ID NOS: 16037, 4432, and 12667, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17612, 18019, and 16523, respectively and the LCDR1, LCDR2, and LCDR3 comprises an amino acid sequence of SEQ ID NOS:18093, 17719, and 9446, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6564, 11466, and 11149, respectively and the LCDR1, LCDR2, and LCDR3 comprises an amino acid sequence of SEQ ID NOS:18069, 18629, and 11178, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 11015, and 6165, respectively and the LCDR1, LCDR2, and LCDR3 comprises an amino acid sequence of SEQ ID NOS:16661, 11903, and 13609, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17216, 10579, and 10672, respectively and the LCDR1, LCDR2, and LCDR3 comprises an amino acid sequence of SEQ ID NOS: 17918, 6235, and 4355, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16817, 10574, and 16472, respectively and the LCDR1, LCDR2, and LCDR3 comprises an amino acid sequence of SEQ ID NOS:15967, 4425, and 12591, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17113, 9822, and 6067, respectively and the LCDR1, LCDR2, and LCDR3 comprises an amino acid sequence of SEQ ID NOS: 10826, 9272, and 11237, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11678, 11463, and 11150, respectively and the LCDR1, LCDR2, and LCDR3 comprises an amino acid sequence of SEQ ID NOS:18066, 18629, and 11175, respectively. having a LCDR1, LCDR2, and LCDR3, wherein the HCDR1, HCDR2, and HCDR3 comprises an amino acid sequence of SEQ ID NOS: 17270, 17778, and 6029, respectively and the LCDR1, LCDR2, and LCDR3 comprises an amino acid sequence of SEQ ID NOS: 16124, 4432, and 12697, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17279, 9947, and 4334, respectively and the LCDR1, LCDR2, and LCDR3 comprises an amino acid sequence of SEQ ID NOS: 16124, 4432, and 12697, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16528, 10407, and 5904, respectively and the LCDR1, LCDR2, and LCDR3 comprises an amino acid sequence of SEQ ID NOS:15860, 10224, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16990, 9839, and 17551, respectively and the LCDR1, LCDR2, and LCDR3 comprises an amino acid sequence of SEQ ID NOS: 16049, 6214, and 11995, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11424, 11509, and 6625, respectively and the LCDR1, LCDR2, and LCDR3 comprises an amino acid sequence of SEQ ID NOS:17907, 10093, and 17235, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11597, 18011, and 6702, respectively and the LCDR1, LCDR2, and LCDR3 comprises an amino acid sequence of SEQ ID NOS: 15861, 6615, and 12838, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17474, 9888, and 9533, respectively and the LCDR1, LCDR2, and LCDR3 comprises an amino acid sequence of SEQ ID NOS: 10827, 9272, and 11235, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9686, 18327, and 6052, respectively and the LCDR1, LCDR2, and LCDR3 comprises an amino acid sequence of SEQ ID NOS: 15964, 9499, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17131, 17256, and 16812, respectively and the LCDR1, LCDR2, and LCDR3 comprises an amino acid sequence of SEQ ID NOS:10901, 18275, and 12032, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17896, 11434, and 9687, respectively and the LCDR1, LCDR2, and LCDR3 comprises an amino acid sequence of SEQ ID NOS:11775, 4441, and 11987, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16519, 4138, and 5906, respectively and the LCDR1, LCDR2, and LCDR3 comprises an amino acid sequence of SEQ ID NOS:15887, 10225, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:3948, 10389, and 17897, respectively and the LCDR1, LCDR2, and LCDR3 comprises an amino acid sequence of SEQ ID NOS:15806, 11791, and 12729, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16864, 16944, and 17658, respectively and the LCDR1, LCDR2, and LCDR3 comprises an amino acid sequence of SEQ ID NOS: 15796, 13595, and 10998, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17449, 18385, and 6467, respectively and the LCDR1, LCDR2, and LCDR3 comprises an amino acid sequence of SEQ ID NOS:9632, 16154, and 10506, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17455, 18535, and 5948, respectively and the LCDR1, LCDR2, and LCDR3 comprises an amino acid sequence of SEQ ID NOS: 16699, 10773, and 4010, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16799, 9730, and 9611, respectively and the LCDR1, LCDR2, and LCDR3 comprises an amino acid sequence of SEQ ID NOS: 15989, 9503, and 12767, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10071, 9940, and 4206, respectively and the LCDR1, LCDR2, and LCDR3 comprises an amino acid sequence of SEQ ID NOS:16403, 10961, and 11205, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17623, 18490, and 5954, respectively and the LCDR1, LCDR2, and LCDR3 comprises an amino acid sequence of SEQ ID NOS:18046, 16958, and 11188, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10685, 18389, and 4680, respectively and the LCDR1, LCDR2, and LCDR3 comprises an amino acid sequence of SEQ ID NOS: 16728, 7863, and 10281, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6184, 4666, and 6059, respectively and the LCDR1, LCDR2, and LCDR3 comprises an amino acid sequence of SEQ ID NOS: 15606, 6214, and 12590, respectively. having a LCDR1, LCDR2, and LCDR3, wherein the HCDR1, HCDR2, and HCDR3 comprises an amino acid sequence of SEQ ID NOS: 11597, 18010, and 6701, respectively and the LCDR1, LCDR2, and LCDR3 comprises an amino acid sequence of SEQ ID NOS: 15861, 6615, and 12737, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6502, 9965, and 4029, respectively and the LCDR1, LCDR2, and LCDR3 comprises an amino acid sequence of SEQ ID NOS: 15786, 9503, and 12791, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17028, 17823, and 6688, respectively and the LCDR1, LCDR2, and LCDR3 comprises an amino acid sequence of SEQ ID NOS:17924, 10094, and 13581, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17149, 16276, and 17899, respectively and the LCDR1, LCDR2, and LCDR3 comprises an amino acid sequence of SEQ ID NOS: 17950, 8079, and 17575, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17006, 10669, and 4236, respectively and the LCDR1, LCDR2, and LCDR3 comprises an amino acid sequence of SEQ ID NOS: 16111, 4436, and 12667, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11653, 9888, and 18133, respectively and the LCDR1, LCDR2, and LCDR3 comprises an amino acid sequence of SEQ ID NOS:15695, 10737, and 12603, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10684, 18401, and 5974, respectively and the LCDR1, LCDR2, and LCDR3 comprises an amino acid sequence of SEQ ID NOS: 16732, 5977, and 7954, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16571, 10312, and 10182, respectively and the LCDR1, LCDR2, and LCDR3 comprises an amino acid sequence of SEQ ID NOS:16061, 4432, and 12715, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9593, 5449, and 11119, respectively and the LCDR1, LCDR2, and LCDR3 comprises an amino acid sequence of SEQ ID NOS:16418, 10926, and 11192, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11155, 18461, and 9348, respectively and the LCDR1, LCDR2, and LCDR3 comprises an amino acid sequence of SEQ ID NOS:17959, 9277, and 4384, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10368, 6782, and 6551, respectively and the LCDR1, LCDR2, and LCDR3 comprises an amino acid sequence of SEQ ID NOS:16659, 11900, and 11884, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6548, 18463, and 9348, respectively and the LCDR1, LCDR2, and LCDR3 comprises an amino acid sequence of SEQ ID NOS: 17985, 9277, and 4384, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17441, 9778, and 6234, respectively and the LCDR1, LCDR2, and LCDR3 comprises an amino acid sequence of SEQ ID NOS: 15715, 3969, and 11089, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16618, 9910, and 18218, respectively and the LCDR1, LCDR2, and LCDR3 comprises an amino acid sequence of SEQ ID NOS:15884, 10742, and 12602, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11633, 17252, and 17066, respectively and the LCDR1, LCDR2, and LCDR3 comprises an amino acid sequence of SEQ ID NOS: 16045, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10934, 9926, and 10107, respectively and the LCDR1, LCDR2, and LCDR3 comprises an amino acid sequence of SEQ ID NOS: 15883, 4436, and 12697, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16208, 16972, and 5920, respectively and the LCDR1, LCDR2, and LCDR3 comprises an amino acid sequence of SEQ ID NOS: 16453, 9503, and 12725, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10362, 16873, and 4483, respectively and the LCDR1, LCDR2, and LCDR3 comprises an amino acid sequence of SEQ ID NOS:17908, 10093, and 17239, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17462, 18465, and 9354, respectively and the LCDR1, LCDR2, and LCDR3 comprises an amino acid sequence of SEQ ID NOS: 17959, 9277, and 4384, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16800, 9692, and 17039, respectively and the LCDR1, LCDR2, and LCDR3 comprises an amino acid sequence of SEQ ID NOS: 16049, 9494, and 12865, respectively. having a LCDR1, LCDR2, and LCDR3, wherein the HCDR1, HCDR2, and HCDR3 comprises an amino acid sequence of SEQ ID NOS:11169, 9761, and 6066, respectively and the LCDR1, LCDR2, and LCDR3 comprises an amino acid sequence of SEQ ID NOS:15859, 10225, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17440, 9843, and 10160, respectively and the LCDR1, LCDR2, and LCDR3 comprises an amino acid sequence of SEQ ID NOS: 16012, 6214, and 10385, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11674, 16294, and 4498, respectively and the LCDR1, LCDR2, and LCDR3 comprises an amino acid sequence of SEQ ID NOS: 17938, 6316, and 17139, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 10571, and 10418, respectively and the LCDR1, LCDR2, and LCDR3 comprises an amino acid sequence of SEQ ID NOS: 16067, 4425, and 12428, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11632, 4128, and 6482, respectively and the LCDR1, LCDR2, and LCDR3 comprises an amino acid sequence of SEQ ID NOS:18067, 18594, and 11357, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10367, 18323, and 6581, respectively and the LCDR1, LCDR2, and LCDR3 comprises an amino acid sequence of SEQ ID NOS: 16061, 9494, and 12682, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11646, 17804, and 6094, respectively and the LCDR1, LCDR2, and LCDR3 comprises an amino acid sequence of SEQ ID NOS:15890, 10727, and 12845, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10934, 9930, and 10143, respectively and the LCDR1, LCDR2, and LCDR3 comprises an amino acid sequence of SEQ ID NOS: 16049, 4432, and 12365, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16610, 4069, and 9612, respectively and the LCDR1, LCDR2, and LCDR3 comprises an amino acid sequence of SEQ ID NOS: 15684, 4454, and 12735, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17272, 18342, and 18285, respectively and the LCDR1, LCDR2, and LCDR3 comprises an amino acid sequence of SEQ ID NOS:15691, 3969, and 11083, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11614, 16243, and 5909, respectively and the LCDR1, LCDR2, and LCDR3 comprises an amino acid sequence of SEQ ID NOS:16408, 18286, and 11192, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17544, 10580, and 11132, respectively and the LCDR1, LCDR2, and LCDR3 comprises an amino acid sequence of SEQ ID NOS: 17951, 6243, and 4356, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4451, 16246, and 9449, respectively and the LCDR1, LCDR2, and LCDR3 comprises an amino acid sequence of SEQ ID NOS:9636, 16161, and 15555, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11677, 16261, and 6049, respectively and the LCDR1, LCDR2, and LCDR3 comprises an amino acid sequence of SEQ ID NOS: 16725, 10770, and 4059, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17262, 9772, and 11729, respectively and the LCDR1, LCDR2, and LCDR3 comprises an amino acid sequence of SEQ ID NOS:15822, 10705, and 12021, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11406, 17813, and 11724, respectively and the LCDR1, LCDR2, and LCDR3 comprises an amino acid sequence of SEQ ID NOS:15716, 3949, and 12386, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16926, 16943, and 10520, respectively and the LCDR1, LCDR2, and LCDR3 comprises an amino acid sequence of SEQ ID NOS:15871, 15520, and 12806, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6565, 18477, and 4026, respectively and the LCDR1, LCDR2, and LCDR3 comprises an amino acid sequence of SEQ ID NOS:16386, 15512, and 11221, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17284, 18415, and 6234, respectively and the LCDR1, LCDR2, and LCDR3 comprises an amino acid sequence of SEQ ID NOS:16659, 11900, and 11875, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9971, and 6232, respectively and the LCDR1, LCDR2, and LCDR3 comprises an amino acid sequence of SEQ ID NOS: 17959, 6312, and 4369, respectively. having a LCDR1, LCDR2, and LCDR3, wherein the HCDR1, HCDR2, and HCDR3 comprises an amino acid sequence of SEQ ID NOS: 17654, 10541, and 4590, respectively and the LCDR1, LCDR2, and LCDR3 comprises an amino acid sequence of SEQ ID NOS: 16714, 10770, and 4264, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17600, 9770, and 16963, respectively and the LCDR1, LCDR2, and LCDR3 comprises an amino acid sequence of SEQ ID NOS: 15870, 4432, and 12676, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 16919, and 6540, respectively and the LCDR1, LCDR2, and LCDR3 comprises an amino acid sequence of SEQ ID NOS: 17959, 6312, and 17195, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6546, 11001, and 18605, respectively and the LCDR1, LCDR2, and LCDR3 comprises an amino acid sequence of SEQ ID NOS:16408, 10987, and 11215, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10933, 9979, and 10109, respectively and the LCDR1, LCDR2, and LCDR3 comprises an amino acid sequence of SEQ ID NOS: 16119, 6326, and 12558, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10780, 9393, and 4267, respectively and the LCDR1, LCDR2, and LCDR3 comprises an amino acid sequence of SEQ ID NOS: 15998, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17634, 18544, and 9353, respectively and the LCDR1, LCDR2, and LCDR3 comprises an amino acid sequence of SEQ ID NOS: 17959, 9277, and 17202, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16520, 16890, and 4397, respectively and the LCDR1, LCDR2, and LCDR3 comprises an amino acid sequence of SEQ ID NOS: 16030, 4425, and 10711, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11655, 18630, and 7894, respectively and the LCDR1, LCDR2, and LCDR3 comprises an amino acid sequence of SEQ ID NOS: 11789, 4406, and 12571, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 10009, and 4207, respectively and the LCDR1, LCDR2, and LCDR3 comprises an amino acid sequence of SEQ ID NOS: 16089, 4432, and 12689, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17279, 9946, and 4217, respectively and the LCDR1, LCDR2, and LCDR3 comprises an amino acid sequence of SEQ ID NOS: 16061, 4432, and 12718, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17568, 18495, and 5992, respectively and the LCDR1, LCDR2, and LCDR3 comprises an amino acid sequence of SEQ ID NOS:18076, 17715, and 11184, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16800, 9693, and 17036, respectively and the LCDR1, LCDR2, and LCDR3 comprises an amino acid sequence of SEQ ID NOS: 16027, 9494, and 12664, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17436, 9999, and 10030, respectively and the LCDR1, LCDR2, and LCDR3 comprises an amino acid sequence of SEQ ID NOS: 16049, 9494, and 12680, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:5441, 16976, and 10955, respectively and the LCDR1, LCDR2, and LCDR3 comprises an amino acid sequence of SEQ ID NOS:15820, 11779, and 10996, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11410, 4664, and 4628, respectively and the LCDR1, LCDR2, and LCDR3 comprises an amino acid sequence of SEQ ID NOS: 15606, 6214, and 12591, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17555, 16217, and 17595, respectively and the LCDR1, LCDR2, and LCDR3 comprises an amino acid sequence of SEQ ID NOS: 16721, 6628, and 10280, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17648, 16845, and 4508, respectively and the LCDR1, LCDR2, and LCDR3 comprises an amino acid sequence of SEQ ID NOS:16422, 10763, and 11275, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10362, 18006, and 4062, respectively and the LCDR1, LCDR2, and LCDR3 comprises an amino acid sequence of SEQ ID NOS: 16036, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9742, and 10151, respectively and the LCDR1, LCDR2, and LCDR3 comprises an amino acid sequence of SEQ ID NOS: 16089, 9494, and 12671, respectively. having a LCDR1, LCDR2, and LCDR3, wherein the HCDR1, HCDR2, and HCDR3 comprises an amino acid sequence of SEQ ID NOS: 16989, 9840, and 4044, respectively and the LCDR1, LCDR2, and LCDR3 comprises an amino acid sequence of SEQ ID NOS:15958, 10102, and 12767, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6547, 18462, and 9349, respectively and the LCDR1, LCDR2, and LCDR3 comprises an amino acid sequence of SEQ ID NOS: 17983, 9279, and 4383, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17442, 18379, and 6140, respectively and the LCDR1, LCDR2, and LCDR3 comprises an amino acid sequence of SEQ ID NOS: 15704, 9477, and 11094, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11409, 11435, and 9688, respectively and the LCDR1, LCDR2, and LCDR3 comprises an amino acid sequence of SEQ ID NOS: 11782, 4418, and 12562, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11381, 10571, and 11113, respectively and the LCDR1, LCDR2, and LCDR3 comprises an amino acid sequence of SEQ ID NOS: 15737, 10727, and 12331, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17639, 16969, and 10498, respectively and the LCDR1, LCDR2, and LCDR3 comprises an amino acid sequence of SEQ ID NOS: 16036, 9503, and 12724, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9874, and 17060, respectively and the LCDR1, LCDR2, and LCDR3 comprises an amino acid sequence of SEQ ID NOS: 16061, 4432, and 12692, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11665, 9823, and 7805, respectively and the LCDR1, LCDR2, and LCDR3 comprises an amino acid sequence of SEQ ID NOS:10906, 18275, and 12612, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17889, 11147, and 16515, respectively and the LCDR1, LCDR2, and LCDR3 comprises an amino acid sequence of SEQ ID NOS:16428, 10765, and 11348, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17474, 9708, and 10110, respectively and the LCDR1, LCDR2, and LCDR3 comprises an amino acid sequence of SEQ ID NOS: 16049, 4432, and 12705, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:7856, 6791, and 6551, respectively and the LCDR1, LCDR2, and LCDR3 comprises an amino acid sequence of SEQ ID NOS: 16043, 9494, and 12704, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17456, 18546, and 10287, respectively and the LCDR1, LCDR2, and LCDR3 comprises an amino acid sequence of SEQ ID NOS: 17950, 6331, and 4348, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17856, 18598, and 9598, respectively and the LCDR1, LCDR2, and LCDR3 comprises an amino acid sequence of SEQ ID NOS: 16764, 10772, and 4266, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10367, 18323, and 17546, respectively and the LCDR1, LCDR2, and LCDR3 comprises an amino acid sequence of SEQ ID NOS:16659, 11900, and 11879, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10257, 6745, and 5918, respectively and the LCDR1, LCDR2, and LCDR3 comprises an amino acid sequence of SEQ ID NOS: 16072, 4437, and 12661, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17609, 4135, and 17588, respectively and the LCDR1, LCDR2, and LCDR3 comprises an amino acid sequence of SEQ ID NOS: 16783, 10758, and 3989, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11631, 4137, and 9532, respectively and the LCDR1, LCDR2, and LCDR3 comprises an amino acid sequence of SEQ ID NOS:15890, 10727, and 12806, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17649, 11455, and 11077, respectively and the LCDR1, LCDR2, and LCDR3 comprises an amino acid sequence of SEQ ID NOS:10819, 6635, and 11241, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6565, 4669, and 4625, respectively and the LCDR1, LCDR2, and LCDR3 comprises an amino acid sequence of SEQ ID NOS: 10827, 9272, and 11239, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17017, 16498, and 9571, respectively and the LCDR1, LCDR2, and LCDR3 comprises an amino acid sequence of SEQ ID NOS:17941, 10320, and 17201, respectively. having a LCDR1, LCDR2, and LCDR3, wherein the HCDR1, HCDR2, and HCDR3 comprises an amino acid sequence of SEQ ID NOS:16598, 10614, and 11115, respectively and the LCDR1, LCDR2, and LCDR3 comprises an amino acid sequence of SEQ ID NOS:16103, 10913, and 12010, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9743, and 9613, respectively and the LCDR1, LCDR2, and LCDR3 comprises an amino acid sequence of SEQ ID NOS: 15701, 4430, and 12826, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17465, 18464, and 9355, respectively and the LCDR1, LCDR2, and LCDR3 comprises an amino acid sequence of SEQ ID NOS: 17959, 9277, and 4384, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17266, 9889, and 4595, respectively and the LCDR1, LCDR2, and LCDR3 comprises an amino acid sequence of SEQ ID NOS: 16061, 6176, and 12585, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16475, 10576, and 5969, respectively and the LCDR1, LCDR2, and LCDR3 comprises an amino acid sequence of SEQ ID NOS: 17958, 9275, and 17201, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17475, 16912, and 17858, respectively and the LCDR1, LCDR2, and LCDR3 comprises an amino acid sequence of SEQ ID NOS:16148, 9503, and 12588, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11604, 10493, and 10683, respectively and the LCDR1, LCDR2, and LCDR3 comprises an amino acid sequence of SEQ ID NOS:15890, 10727, and 12841, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17441, 18338, and 18050, respectively and the LCDR1, LCDR2, and LCDR3 comprises an amino acid sequence of SEQ ID NOS: 17904, 10095, and 17238, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16624, 18342, and 18215, respectively and the LCDR1, LCDR2, and LCDR3 comprises an amino acid sequence of SEQ ID NOS:16428, 10764, and 11228, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4493, 9374, and 6489, respectively and the LCDR1, LCDR2, and LCDR3 comprises an amino acid sequence of SEQ ID NOS:15664, 17587, and 11084, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11577, 18363, and 6096, respectively and the LCDR1, LCDR2, and LCDR3 comprises an amino acid sequence of SEQ ID NOS:16384, 10926, and 11275, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17432, 18518, and 4022, respectively and the LCDR1, LCDR2, and LCDR3 comprises an amino acid sequence of SEQ ID NOS: 10710, 6617, and 11087, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17856, 16309, and 18443, respectively and the LCDR1, LCDR2, and LCDR3 comprises an amino acid sequence of SEQ ID NOS: 15897, 3965, and 12473, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11367, 10392, and 18056, respectively and the LCDR1, LCDR2, and LCDR3 comprises an amino acid sequence of SEQ ID NOS:16647, 16164, and 13033, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 18451, 17803, and 6036, respectively and the LCDR1, LCDR2, and LCDR3 comprises an amino acid sequence of SEQ ID NOS: 15682, 4434, and 12950, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11626, 17753, and 9519, respectively and the LCDR1, LCDR2, and LCDR3 comprises an amino acid sequence of SEQ ID NOS: 16114, 6611, and 12846, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6565, 17998, and 17134, respectively and the LCDR1, LCDR2, and LCDR3 comprises an amino acid sequence of SEQ ID NOS:10797, 18280, and 12941, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 17809, and 16178, respectively and the LCDR1, LCDR2, and LCDR3 comprises an amino acid sequence of SEQ ID NOS:15713, 3982, and 12394, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16553, 10390, and 10585, respectively and the LCDR1, LCDR2, and LCDR3 comprises an amino acid sequence of SEQ ID NOS: 16638, 10966, and 13035, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4039, 5447, and 11952, respectively and the LCDR1, LCDR2, and LCDR3 comprises an amino acid sequence of SEQ ID NOS: 16136, 3956, and 13010, respectively. having a LCDR1, LCDR2, and LCDR3, wherein the HCDR1, HCDR2, and HCDR3 comprises an amino acid sequence of SEQ ID NOS: 17537, 18471, and 6619, respectively and the LCDR1, LCDR2, and LCDR3 comprises an amino acid sequence of SEQ ID NOS: 16061, 9491, and 12693, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17474, 10295, and 16626, respectively and the LCDR1, LCDR2, and LCDR3 comprises an amino acid sequence of SEQ ID NOS:15623, 16141, and 11106, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16535, 10008, and 18217, respectively and the LCDR1, LCDR2, and LCDR3 comprises an amino acid sequence of SEQ ID NOS:16106, 3974, and 12693, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11637, 18403, and 10131, respectively and the LCDR1, LCDR2, and LCDR3 comprises an amino acid sequence of SEQ ID NOS: 16488, 4425, and 11103, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17608, 16858, and 6210, respectively and the LCDR1, LCDR2, and LCDR3 comprises an amino acid sequence of SEQ ID NOS: 16094, 4425, and 10711, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4453, 16247, and 11042, respectively and the LCDR1, LCDR2, and LCDR3 comprises an amino acid sequence of SEQ ID NOS:15908, 16558, and 12498, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17273, 5440, and 5945, respectively and the LCDR1, LCDR2, and LCDR3 comprises an amino acid sequence of SEQ ID NOS: 16011, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17009, 16497, and 5895, respectively and the LCDR1, LCDR2, and LCDR3 comprises an amino acid sequence of SEQ ID NOS: 15625, 6215, and 12632, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17843, 11488, and 17676, respectively and the LCDR1, LCDR2, and LCDR3 comprises an amino acid sequence of SEQ ID NOS:10864, 18275, and 12894, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17856, 11033, and 11068, respectively and the LCDR1, LCDR2, and LCDR3 comprises an amino acid sequence of SEQ ID NOS:15828, 9490, and 12527, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11367, 10393, and 18057, respectively and the LCDR1, LCDR2, and LCDR3 comprises an amino acid sequence of SEQ ID NOS:16642, 16164, and 13033, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11650, 17762, and 5986, respectively and the LCDR1, LCDR2, and LCDR3 comprises an amino acid sequence of SEQ ID NOS: 15825, 9499, and 12946, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 18545, and 6185, respectively and the LCDR1, LCDR2, and LCDR3 comprises an amino acid sequence of SEQ ID NOS: 16061, 9494, and 12675, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17132, 16828, and 10974, respectively and the LCDR1, LCDR2, and LCDR3 comprises an amino acid sequence of SEQ ID NOS:16134, 9506, and 12529, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:7820, 9562, and 4047, respectively and the LCDR1, LCDR2, and LCDR3 comprises an amino acid sequence of SEQ ID NOS:16642, 10747, and 13031, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4136, and 10994, respectively and the LCDR1, LCDR2, and LCDR3 comprises an amino acid sequence of SEQ ID NOS:16642, 10747, and 13028, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17447, 9387, and 11056, respectively and the LCDR1, LCDR2, and LCDR3 comprises an amino acid sequence of SEQ ID NOS: 17916, 4179, and 17233, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16822, 18581, and 5990, respectively and the LCDR1, LCDR2, and LCDR3 comprises an amino acid sequence of SEQ ID NOS: 16117, 3982, and 10450, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16797, 9394, and 10153, respectively and the LCDR1, LCDR2, and LCDR3 comprises an amino acid sequence of SEQ ID NOS: 15973, 3983, and 12868, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16569, 9376, and 6233, respectively and the LCDR1, LCDR2, and LCDR3 comprises an amino acid sequence of SEQ ID NOS:16438, 10926, and 11271, respectively. having a LCDR1, LCDR2, and LCDR3, wherein the HCDR1, HCDR2, and HCDR3 comprises an amino acid sequence of SEQ ID NOS:11574, 9372, and 10021, respectively and the LCDR1, LCDR2, and LCDR3 comprises an amino acid sequence of SEQ ID NOS:10869, 18275, and 12923, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17852, 18320, and 10028, respectively and the LCDR1, LCDR2, and LCDR3 comprises an amino acid sequence of SEQ ID NOS:10915, 18275, and 10479, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11664, 16925, and 6198, respectively and the LCDR1, LCDR2, and LCDR3 comprises an amino acid sequence of SEQ ID NOS: 15848, 3982, and 12519, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17028, 17823, and 17846, respectively and the LCDR1, LCDR2, and LCDR3 comprises an amino acid sequence of SEQ ID NOS:16659, 11903, and 11863, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16337, 16849, and 11111, respectively and the LCDR1, LCDR2, and LCDR3 comprises an amino acid sequence of SEQ ID NOS:10880, 18275, and 12360, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16591, 18481, and 9597, respectively and the LCDR1, LCDR2, and LCDR3 comprises an amino acid sequence of SEQ ID NOS:18075, 18603, and 11177, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17068, 9944, and 10201, respectively and the LCDR1, LCDR2, and LCDR3 comprises an amino acid sequence of SEQ ID NOS: 16062, 9494, and 12706, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4136, and 4571, respectively and the LCDR1, LCDR2, and LCDR3 comprises an amino acid sequence of SEQ ID NOS: 16061, 9494, and 12661, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16957, 7846, and 6116, respectively and the LCDR1, LCDR2, and LCDR3 comprises an amino acid sequence of SEQ ID NOS: 16063, 9487, and 12000, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17276, 17757, and 4214, respectively and the LCDR1, LCDR2, and LCDR3 comprises an amino acid sequence of SEQ ID NOS: 15976, 9495, and 12948, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6510, 17746, and 17986, respectively and the LCDR1, LCDR2, and LCDR3 comprises an amino acid sequence of SEQ ID NOS:16442, 15521, and 11288, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17220, 10575, and 6044, respectively and the LCDR1, LCDR2, and LCDR3 comprises an amino acid sequence of SEQ ID NOS: 10223, 6199, and 11057, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10368, 6782, and 9463, respectively and the LCDR1, LCDR2, and LCDR3 comprises an amino acid sequence of SEQ ID NOS: 16067, 4425, and 10717, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10939, 18112, and 16788, respectively and the LCDR1, LCDR2, and LCDR3 comprises an amino acid sequence of SEQ ID NOS:18422, 15521, and 11245, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 18439, and 9660, respectively and the LCDR1, LCDR2, and LCDR3 comprises an amino acid sequence of SEQ ID NOS:16440, 11612, and 11265, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17459, 18497, and 6667, respectively and the LCDR1, LCDR2, and LCDR3 comprises an amino acid sequence of SEQ ID NOS: 16768, 16585, and 4288, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16202, 18480, and 9530, respectively and the LCDR1, LCDR2, and LCDR3 comprises an amino acid sequence of SEQ ID NOS:16415, 10926, and 11273, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11658, 11480, and 16964, respectively and the LCDR1, LCDR2, and LCDR3 comprises an amino acid sequence of SEQ ID NOS:15784, 10918, and 12882, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16951, and 17993, respectively and the LCDR1, LCDR2, and LCDR3 comprises an amino acid sequence of SEQ ID NOS:11945, 10024, and 11591, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6508, 4105, and 4391, respectively and the LCDR1, LCDR2, and LCDR3 comprises an amino acid sequence of SEQ ID NOS:16470, 10987, and 11327, respectively. having a LCDR1, LCDR2, and LCDR3, wherein the HCDR1, HCDR2, and HCDR3 comprises an amino acid sequence of SEQ ID NOS:16170, 4390, and 4458, respectively and the LCDR1, LCDR2, and LCDR3 comprises an amino acid sequence of SEQ ID NOS:16438, 10926, and 11261, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9566, 6788, and 10161, respectively and the LCDR1, LCDR2, and LCDR3 comprises an amino acid sequence of SEQ ID NOS:15910, 16142, and 10467, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9988, and 4043, respectively and the LCDR1, LCDR2, and LCDR3 comprises an amino acid sequence of SEQ ID NOS: 16061, 3974, and 12685, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17649, 11476, and 6517, respectively and the LCDR1, LCDR2, and LCDR3 comprises an amino acid sequence of SEQ ID NOS:15627, 10696, and 12738, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 18538, and 0, respectively and the LCDR1, LCDR2, and LCDR3 comprises an amino acid sequence of SEQ ID NOS:16060, 10262, and 12671, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10350, 18478, and 5925, respectively and the LCDR1, LCDR2, and LCDR3 comprises an amino acid sequence of SEQ ID NOS:16367, 10926, and 11258, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10774, 17999, and 11148, respectively and the LCDR1, LCDR2, and LCDR3 comprises an amino acid sequence of SEQ ID NOS:18073, 18596, and 11358, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17646, 16260, and 9459, respectively and the LCDR1, LCDR2, and LCDR3 comprises an amino acid sequence of SEQ ID NOS:4240, 17231, and 11058, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 11473, and 6515, respectively and the LCDR1, LCDR2, and LCDR3 comprises an amino acid sequence of SEQ ID NOS:15890, 10696, and 11928, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18099, 9809, and 10978, respectively and the LCDR1, LCDR2, and LCDR3 comprises an amino acid sequence of SEQ ID NOS: 10784, 9272, and 11299, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16524, 18373, and 6461, respectively and the LCDR1, LCDR2, and LCDR3 comprises an amino acid sequence of SEQ ID NOS: 16446, 9266, and 11287, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 11478, and 6516, respectively and the LCDR1, LCDR2, and LCDR3 comprises an amino acid sequence of SEQ ID NOS:16140, 10744, and 11934, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11636, 9912, and 6032, respectively and the LCDR1, LCDR2, and LCDR3 comprises an amino acid sequence of SEQ ID NOS:16416, 18275, and 12964, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16804, 16883, and 6481, respectively and the LCDR1, LCDR2, and LCDR3 comprises an amino acid sequence of SEQ ID NOS:16711, 10092, and 18228, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9834, and 18599, respectively and the LCDR1, LCDR2, and LCDR3 comprises an amino acid sequence of SEQ ID NOS: 15599, 4425, and 12368, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10423, 11029, and 16811, respectively and the LCDR1, LCDR2, and LCDR3 comprises an amino acid sequence of SEQ ID NOS: 15992, 9494, and 12566, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11654, 18364, and 6130, respectively and the LCDR1, LCDR2, and LCDR3 comprises an amino acid sequence of SEQ ID NOS: 17142, 6208, and 11145, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17619, 18351, and 4455, respectively and the LCDR1, LCDR2, and LCDR3 comprises an amino acid sequence of SEQ ID NOS:16373, 10926, and 11258, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17432, 18525, and 6138, respectively and the LCDR1, LCDR2, and LCDR3 comprises an amino acid sequence of SEQ ID NOS: 16036, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17648, 11479, and 6514, respectively and the LCDR1, LCDR2, and LCDR3 comprises an amino acid sequence of SEQ ID NOS:15753, 10912, and 11933, respectively. having a LCDR1, LCDR2, and LCDR3, wherein the HCDR1, HCDR2, and HCDR3 comprises an amino acid sequence of SEQ ID NOS:11623, 10211, and 18608, respectively and the LCDR1, LCDR2, and LCDR3 comprises an amino acid sequence of SEQ ID NOS:15829, 10735, and 12817, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17270, 17778, and 7881, respectively and the LCDR1, LCDR2, and LCDR3 comprises an amino acid sequence of SEQ ID NOS: 15713, 3982, and 12949, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6500, 11020, and 17835, respectively and the LCDR1, LCDR2, and LCDR3 comprises an amino acid sequence of SEQ ID NOS:16408, 10987, and 11198, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11386, 9774, and 5917, respectively and the LCDR1, LCDR2, and LCDR3 comprises an amino acid sequence of SEQ ID NOS: 17950, 6312, and 17190, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16542, 11493, and 17687, respectively and the LCDR1, LCDR2, and LCDR3 comprises an amino acid sequence of SEQ ID NOS:10855, 18264, and 12931, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4248, 16513, and 6720, respectively and the LCDR1, LCDR2, and LCDR3 comprises an amino acid sequence of SEQ ID NOS:16355, 10924, and 11283, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17535, 10571, and 11129, respectively and the LCDR1, LCDR2, and LCDR3 comprises an amino acid sequence of SEQ ID NOS:17924, 10094, and 13581, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10513, 18304, and 5953, respectively and the LCDR1, LCDR2, and LCDR3 comprises an amino acid sequence of SEQ ID NOS:16441, 17593, and 11258, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17632, 11546, and 11051, respectively and the LCDR1, LCDR2, and LCDR3 comprises an amino acid sequence of SEQ ID NOS:18425, 18275, and 12928, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16794, 16939, and 10378, respectively and the LCDR1, LCDR2, and LCDR3 comprises an amino acid sequence of SEQ ID NOS:17959, 6331, and 17190, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11679, 18000, and 11148, respectively and the LCDR1, LCDR2, and LCDR3 comprises an amino acid sequence of SEQ ID NOS:18073, 18596, and 11360, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17842, 11488, and 11062, respectively and the LCDR1, LCDR2, and LCDR3 comprises an amino acid sequence of SEQ ID NOS: 16408, 10987, and 11345, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 11477, and 6513, respectively and the LCDR1, LCDR2, and LCDR3 comprises an amino acid sequence of SEQ ID NOS:15738, 10696, and 11931, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16784, 16308, and 16970, respectively and the LCDR1, LCDR2, and LCDR3 comprises an amino acid sequence of SEQ ID NOS:16093, 9503, and 12814, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17028, 17824, and 6114, respectively and the LCDR1, LCDR2, and LCDR3 comprises an amino acid sequence of SEQ ID NOS: 16076, 4425, and 10707, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16998, 9830, and 4276, respectively and the LCDR1, LCDR2, and LCDR3 comprises an amino acid sequence of SEQ ID NOS:16650, 10747, and 13022, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17854, 16514, and 6719, respectively and the LCDR1, LCDR2, and LCDR3 comprises an amino acid sequence of SEQ ID NOS:16438, 10926, and 11284, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17148, 18059, and 11148, respectively and the LCDR1, LCDR2, and LCDR3 comprises an amino acid sequence of SEQ ID NOS:18071, 18596, and 11180, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11678, 11505, and 4496, respectively and the LCDR1, LCDR2, and LCDR3 comprises an amino acid sequence of SEQ ID NOS:15744, 17866, and 11936, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11669, 16881, and 6478, respectively and the LCDR1, LCDR2, and LCDR3 comprises an amino acid sequence of SEQ ID NOS: 16762, 5987, and 17673, respectively. having a LCDR1, LCDR2, and LCDR3, wherein the HCDR1, HCDR2, and HCDR3 comprises an amino acid sequence of SEQ ID NOS: 17854, 16512, and 6719, respectively and the LCDR1, LCDR2, and LCDR3 comprises an amino acid sequence of SEQ ID NOS:16342, 10926, and 11285, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10367, 18329, and 10554, respectively and the LCDR1, LCDR2, and LCDR3 comprises an amino acid sequence of SEQ ID NOS:4057, 6238, and 18625, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11421, 10559, and 5897, respectively and the LCDR1, LCDR2, and LCDR3 comprises an amino acid sequence of SEQ ID NOS: 11785, 4420, and 12576, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16518, 17742, and 6010, respectively and the LCDR1, LCDR2, and LCDR3 comprises an amino acid sequence of SEQ ID NOS:16138, 10698, and 12609, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16195, 18305, and 5956, respectively and the LCDR1, LCDR2, and LCDR3 comprises an amino acid sequence of SEQ ID NOS: 16135, 9503, and 12767, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17000, 18395, and 4577, respectively and the LCDR1, LCDR2, and LCDR3 comprises an amino acid sequence of SEQ ID NOS:16433, 15521, and 11287, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18638, 17807, and 10147, respectively and the LCDR1, LCDR2, and LCDR3 comprises an amino acid sequence of SEQ ID NOS: 17966, 6312, and 4380, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16538, 4148, and 11076, respectively and the LCDR1, LCDR2, and LCDR3 comprises an amino acid sequence of SEQ ID NOS: 15704, 3976, and 12826, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17070, 18404, and 5937, respectively and the LCDR1, LCDR2, and LCDR3 comprises an amino acid sequence of SEQ ID NOS:16029, 10697, and 12095, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18382, and 4576, respectively and the LCDR1, LCDR2, and LCDR3 comprises an amino acid sequence of SEQ ID NOS:16445, 15521, and 11287, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11630, 18431, and 10360, respectively and the LCDR1, LCDR2, and LCDR3 comprises an amino acid sequence of SEQ ID NOS: 16383, 10926, and 11272, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6529, 11031, and 10113, respectively and the LCDR1, LCDR2, and LCDR3 comprises an amino acid sequence of SEQ ID NOS: 16727, 5977, and 10268, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16588, 6792, and 6538, respectively and the LCDR1, LCDR2, and LCDR3 comprises an amino acid sequence of SEQ ID NOS: 16082, 9494, and 12704, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17556, 17787, and 6007, respectively and the LCDR1, LCDR2, and LCDR3 comprises an amino acid sequence of SEQ ID NOS: 17960, 6239, and 4359, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11365, 11438, and 6042, respectively and the LCDR1, LCDR2, and LCDR3 comprises an amino acid sequence of SEQ ID NOS:16436, 10926, and 11295, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16351, 18508, and 17874, respectively and the LCDR1, LCDR2, and LCDR3 comprises an amino acid sequence of SEQ ID NOS:16481, 3969, and 11095, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17606, 18018, and 10358, respectively and the LCDR1, LCDR2, and LCDR3 comprises an amino acid sequence of SEQ ID NOS: 17902, 10094, and 13567, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16575, 18580, and 7901, respectively and the LCDR1, LCDR2, and LCDR3 comprises an amino acid sequence of SEQ ID NOS:15766, 11695, and 12532, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17891, 18492, and 17048, respectively and the LCDR1, LCDR2, and LCDR3 comprises an amino acid sequence of SEQ ID NOS: 15879, 9502, and 12724, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10694, 17764, and 9518, respectively and the LCDR1, LCDR2, and LCDR3 comprises an amino acid sequence of SEQ ID NOS:15847, 10705, and 11935, respectively. having a LCDR1, LCDR2, and LCDR3, wherein the HCDR1, HCDR2, and HCDR3 comprises an amino acid sequence of SEQ ID NOS:17643, 10573, and 10485, respectively and the LCDR1, LCDR2, and LCDR3 comprises an amino acid sequence of SEQ ID NOS: 10833, 9267, and 11312, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 9411, and 10529, respectively and the LCDR1, LCDR2, and LCDR3 comprises an amino acid sequence of SEQ ID NOS:9641, 4464, and 15573, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6509, 9977, and, respectively and the LCDR1, LCDR2, and LCDR3 comprises an amino acid sequence of SEQ ID NOS: 15620, 3974, and 12791, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17130, 7803, and 7928, respectively and the LCDR1, LCDR2, and LCDR3 comprises an amino acid sequence of SEQ ID NOS:9639, 6632, and 15569, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4667, 10388, and 17548, respectively and the LCDR1, LCDR2, and LCDR3 comprises an amino acid sequence of SEQ ID NOS: 16482, 3944, and 12478, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11407, 11436, and 17695, respectively and the LCDR1, LCDR2, and LCDR3 comprises an amino acid sequence of SEQ ID NOS:17982, 9277, and 17111, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11650, 9408, and 4398, respectively and the LCDR1, LCDR2, and LCDR3 comprises an amino acid sequence of SEQ ID NOS:15793, 10718, and 12011, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11673, 17817, and 4171, respectively and the LCDR1, LCDR2, and LCDR3 comprises an amino acid sequence of SEQ ID NOS:4058, 6333, and 17207, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6519, 11014, and 4507, respectively and the LCDR1, LCDR2, and LCDR3 comprises an amino acid sequence of SEQ ID NOS:16394, 10990, and 11202, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18360, and 9529, respectively and the LCDR1, LCDR2, and LCDR3 comprises an amino acid sequence of SEQ ID NOS:15713, 3982, and 12388, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6529, 11032, and 10114, respectively and the LCDR1, LCDR2, and LCDR3 comprises an amino acid sequence of SEQ ID NOS: 16736, 5977, and 10269, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11650, 17806, and 10139, respectively and the LCDR1, LCDR2, and LCDR3 comprises an amino acid sequence of SEQ ID NOS:9633, 4472, and 15554, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16527, 17749, and 5932, respectively and the LCDR1, LCDR2, and LCDR3 comprises an amino acid sequence of SEQ ID NOS:16396, 10987, and 11202, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16518, 18195, and 6148, respectively and the LCDR1, LCDR2, and LCDR3 comprises an amino acid sequence of SEQ ID NOS:16029, 10697, and 12878, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17114, 17786, and 6006, respectively and the LCDR1, LCDR2, and LCDR3 comprises an amino acid sequence of SEQ ID NOS: 17964, 6239, and 4388, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17441, 9824, and 10291, respectively and the LCDR1, LCDR2, and LCDR3 comprises an amino acid sequence of SEQ ID NOS:10890, 18275, and 12977, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6510, 9415, and 18254, respectively and the LCDR1, LCDR2, and LCDR3 comprises an amino acid sequence of SEQ ID NOS: 15887, 9503, and 12793, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6529, 11032, and 10114, respectively and the LCDR1, LCDR2, and LCDR3 comprises an amino acid sequence of SEQ ID NOS: 16736, 5977, and 10269, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11641, 6735, and 10979, respectively and the LCDR1, LCDR2, and LCDR3 comprises an amino acid sequence of SEQ ID NOS:15631, 10241, and 12342, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 17806, and 10140, respectively and the LCDR1, LCDR2, and LCDR3 comprises an amino acid sequence of SEQ ID NOS:9631, 4467, and 15554, respectively. having a LCDR1, LCDR2, and LCDR3, wherein the HCDR1, HCDR2, and HCDR3 comprises an amino acid sequence of SEQ ID NOS:17279, 9741, and 17241, respectively and the LCDR1, LCDR2, and LCDR3 comprises an amino acid sequence of SEQ ID NOS: 15701, 3982, and 12095, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17278, 18339, and 5957, respectively and the LCDR1, LCDR2, and LCDR3 comprises an amino acid sequence of SEQ ID NOS: 17962, 6244, and 4350, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17891, 18492, and 9422, respectively and the LCDR1, LCDR2, and LCDR3 comprises an amino acid sequence of SEQ ID NOS: 15879, 9502, and 12724, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17621, 17793, and 4629, respectively and the LCDR1, LCDR2, and LCDR3 comprises an amino acid sequence of SEQ ID NOS: 16492, 4425, and 12448, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16524, 18378, and 5903, respectively and the LCDR1, LCDR2, and LCDR3 comprises an amino acid sequence of SEQ ID NOS:16471, 11611, and 11257, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16690, 9412, and 7927, respectively and the LCDR1, LCDR2, and LCDR3 comprises an amino acid sequence of SEQ ID NOS:16642, 16164, and 13334, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11633, 16973, and 17860, respectively and the LCDR1, LCDR2, and LCDR3 comprises an amino acid sequence of SEQ ID NOS:9637, 4472, and 15564, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11641, 6736, and 10980, respectively and the LCDR1, LCDR2, and LCDR3 comprises an amino acid sequence of SEQ ID NOS:16339, 10244, and 12340, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4630, 11013, and 10226, respectively and the LCDR1, LCDR2, and LCDR3 comprises an amino acid sequence of SEQ ID NOS:10819, 9267, and 11331, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 9950, and 11375, respectively and the LCDR1, LCDR2, and LCDR3 comprises an amino acid sequence of SEQ ID NOS:17970, 9261, and 4342, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11650, 17806, and 10138, respectively and the LCDR1, LCDR2, and LCDR3 comprises an amino acid sequence of SEQ ID NOS:9588, 5984, and 15556, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11638, 17785, and 4507, respectively and the LCDR1, LCDR2, and LCDR3 comprises an amino acid sequence of SEQ ID NOS:16397, 10990, and 11202, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11633, 16876, and 6464, respectively and the LCDR1, LCDR2, and LCDR3 comprises an amino acid sequence of SEQ ID NOS: 16718, 11555, and 4286, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11620, 16278, and 4020, respectively and the LCDR1, LCDR2, and LCDR3 comprises an amino acid sequence of SEQ ID NOS:16655, 16505, and 11740, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17558, 11009, and 16348, respectively and the LCDR1, LCDR2, and LCDR3 comprises an amino acid sequence of SEQ ID NOS:10844, 18275, and 12939, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17842, 11488, and 15581, respectively and the LCDR1, LCDR2, and LCDR3 comprises an amino acid sequence of SEQ ID NOS: 15893, 3969, and 12517, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9964, and 9546, respectively and the LCDR1, LCDR2, and LCDR3 comprises an amino acid sequence of SEQ ID NOS: 15611, 9503, and 12766, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9776, and 16820, respectively and the LCDR1, LCDR2, and LCDR3 comprises an amino acid sequence of SEQ ID NOS:17924, 10094, and 13579, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9579, 16274, and 11722, respectively and the LCDR1, LCDR2, and LCDR3 comprises an amino acid sequence of SEQ ID NOS: 17915, 6629, and 13332, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11650, 9408, and 4398, respectively and the LCDR1, LCDR2, and LCDR3 comprises an amino acid sequence of SEQ ID NOS:15948, 10729, and 12860, respectively. having a LCDR1, LCDR2, and LCDR3, wherein the HCDR1, HCDR2, and HCDR3 comprises an amino acid sequence of SEQ ID NOS:4634, 16284, and 11681, respectively and the LCDR1, LCDR2, and LCDR3 comprises an amino acid sequence of SEQ ID NOS: 15675, 3982, and 12552, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17553, 6790, and 6169, respectively and the LCDR1, LCDR2, and LCDR3 comprises an amino acid sequence of SEQ ID NOS: 15971, 9488, and 11986, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11423, 18476, and 10512, respectively and the LCDR1, LCDR2, and LCDR3 comprises an amino acid sequence of SEQ ID NOS:10878, 18275, and 12930, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17466, 18496, and 17051, respectively and the LCDR1, LCDR2, and LCDR3 comprises an amino acid sequence of SEQ ID NOS:16128, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11668, 16902, and 11966, respectively and the LCDR1, LCDR2, and LCDR3 comprises an amino acid sequence of SEQ ID NOS: 15638, 9503, and 12366, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17642, 11137, and 17056, respectively and the LCDR1, LCDR2, and LCDR3 comprises an amino acid sequence of SEQ ID NOS: 17933, 6331, and 10235, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17880, 18249, and 8076, respectively and the LCDR1, LCDR2, and LCDR3 comprises an amino acid sequence of SEQ ID NOS: 15958, 9503, and 12891, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6505, 9415, and 17728, respectively and the LCDR1, LCDR2, and LCDR3 comprises an amino acid sequence of SEQ ID NOS: 15887, 9503, and 12733, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17891, 18492, and 9423, respectively and the LCDR1, LCDR2, and LCDR3 comprises an amino acid sequence of SEQ ID NOS: 15758, 9503, and 12586, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17432, 18520, and 18144, respectively and the LCDR1, LCDR2, and LCDR3 comprises an amino acid sequence of SEQ ID NOS:17892, 18629, and 11182, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6529, 11032, and 10114, respectively and the LCDR1, LCDR2, and LCDR3 comprises an amino acid sequence of SEQ ID NOS: 16736, 5968, and 10269, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16692, 9414, and 6179, respectively and the LCDR1, LCDR2, and LCDR3 comprises an amino acid sequence of SEQ ID NOS: 16085, 9503, and 12587, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17607, 17783, and 7909, respectively and the LCDR1, LCDR2, and LCDR3 comprises an amino acid sequence of SEQ ID NOS: 16045, 9464, and 12886, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11352, 4151, and 5914, respectively and the LCDR1, LCDR2, and LCDR3 comprises an amino acid sequence of SEQ ID NOS: 15711, 3969, and 12469, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16954, and 10508, respectively and the LCDR1, LCDR2, and LCDR3 comprises an amino acid sequence of SEQ ID NOS: 16045, 9503, and 12790, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16527, 17750, and 5933, respectively and the LCDR1, LCDR2, and LCDR3 comprises an amino acid sequence of SEQ ID NOS:16395, 10987, and 11202, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9976, and 9603, respectively and the LCDR1, LCDR2, and LCDR3 comprises an amino acid sequence of SEQ ID NOS: 15637, 4435, and 12761, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17275, 16898, and 6462, respectively and the LCDR1, LCDR2, and LCDR3 comprises an amino acid sequence of SEQ ID NOS:16722, 10074, and 17667, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18638, 17811, and 10145, respectively and the LCDR1, LCDR2, and LCDR3 comprises an amino acid sequence of SEQ ID NOS: 17968, 9275, and 4359, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11650, 17770, and 5915, respectively and the LCDR1, LCDR2, and LCDR3 comprises an amino acid sequence of SEQ ID NOS:11951, 10024, and 11589, respectively. having a LCDR1, LCDR2, and LCDR3, wherein the HCDR1, HCDR2, and HCDR3 comprises an amino acid sequence of SEQ ID NOS:10494, 16981, and 6012, respectively and the LCDR1, LCDR2, and LCDR3 comprises an amino acid sequence of SEQ ID NOS: 16014, 9488, and 12884, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6529, 11032, and 10112, respectively and the LCDR1, LCDR2, and LCDR3 comprises an amino acid sequence of SEQ ID NOS: 16736, 5977, and 10268, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17843, 11506, and 6043, respectively and the LCDR1, LCDR2, and LCDR3 comprises an amino acid sequence of SEQ ID NOS:16434, 10923, and 11287, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16803, 18340, and 5957, respectively and the LCDR1, LCDR2, and LCDR3 comprises an amino acid sequence of SEQ ID NOS: 17898, 6244, and 4349, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11650, 17806, and 10137, respectively and the LCDR1, LCDR2, and LCDR3 comprises an amino acid sequence of SEQ ID NOS:9628, 4467, and 15554, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6530, 9375, and 16580, respectively and the LCDR1, LCDR2, and LCDR3 comprises an amino acid sequence of SEQ ID NOS:16435, 10926, and 11271, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17621, 17801, and 7909, respectively and the LCDR1, LCDR2, and LCDR3 comprises an amino acid sequence of SEQ ID NOS: 16045, 9464, and 12864, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11626, 18430, and 10361, respectively and the LCDR1, LCDR2, and LCDR3 comprises an amino acid sequence of SEQ ID NOS: 16383, 10926, and 11272, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6566, 11462, and 6197, respectively and the LCDR1, LCDR2, and LCDR3 comprises an amino acid sequence of SEQ ID NOS:16391, 10927, and 11275, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11647, 18433, and 5929, respectively and the LCDR1, LCDR2, and LCDR3 comprises an amino acid sequence of SEQ ID NOS: 16444, 9266, and 11272, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17621, 17774, and 4511, respectively and the LCDR1, LCDR2, and LCDR3 comprises an amino acid sequence of SEQ ID NOS: 16067, 4423, and 12417, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10944, 18390, and 5930, respectively and the LCDR1, LCDR2, and LCDR3 comprises an amino acid sequence of SEQ ID NOS: 16443, 9481, and 11257, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11650, 17805, and 10146, respectively and the LCDR1, LCDR2, and LCDR3 comprises an amino acid sequence of SEQ ID NOS: 17966, 9261, and 4342, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17272, 18339, and 5957, respectively and the LCDR1, LCDR2, and LCDR3 comprises an amino acid sequence of SEQ ID NOS: 17962, 6244, and 4350, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10365, 16273, and 16705, respectively and the LCDR1, LCDR2, and LCDR3 comprises an amino acid sequence of SEQ ID NOS:17978, 6715, and 4381, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17441, 6747, and 16820, respectively and the LCDR1, LCDR2, and LCDR3 comprises an amino acid sequence of SEQ ID NOS: 15777, 3974, and 12006, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10515, 18299, and 7814, respectively and the LCDR1, LCDR2, and LCDR3 comprises an amino acid sequence of SEQ ID NOS:16431, 10762, and 11347, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4492, 9954, and 9452, respectively and the LCDR1, LCDR2, and LCDR3 comprises an amino acid sequence of SEQ ID NOS: 15611, 9503, and 12766, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17599, 18298, and 10234, respectively and the LCDR1, LCDR2, and LCDR3 comprises an amino acid sequence of SEQ ID NOS:16379, 15521, and 11292, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 4681, and 6028, respectively and the LCDR1, LCDR2, and LCDR3 comprises an amino acid sequence of SEQ ID NOS: 16687, 16995, and 4002, respectively. having a LCDR1, LCDR2, and LCDR3, wherein the HCDR1, HCDR2, and HCDR3 comprises an amino acid sequence of SEQ ID NOS: 17443, 17765, and 5446, respectively and the LCDR1, LCDR2, and LCDR3 comprises an amino acid sequence of SEQ ID NOS:16139, 17865, and 12507, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16990, 9835, and 17047, respectively and the LCDR1, LCDR2, and LCDR3 comprises an amino acid sequence of SEQ ID NOS:17930, 10229, and 13566, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17130, 6782, and 10593, respectively and the LCDR1, LCDR2, and LCDR3 comprises an amino acid sequence of SEQ ID NOS:16404, 10987, and 11207, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17257, 16939, and 10322, respectively and the LCDR1, LCDR2, and LCDR3 comprises an amino acid sequence of SEQ ID NOS: 11777, 4420, and 11989, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17258, 9739, and 4272, respectively and the LCDR1, LCDR2, and LCDR3 comprises an amino acid sequence of SEQ ID NOS: 16045, 9494, and 12852, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11420, 16909, and 6126, respectively and the LCDR1, LCDR2, and LCDR3 comprises an amino acid sequence of SEQ ID NOS: 15769, 9501, and 12946, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16517, 6754, and 10754, respectively and the LCDR1, LCDR2, and LCDR3 comprises an amino acid sequence of SEQ ID NOS: 15899, 3970, and 12497, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17856, 16311, and 6003, respectively and the LCDR1, LCDR2, and LCDR3 comprises an amino acid sequence of SEQ ID NOS: 11786, 9476, and 12575, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17270, 4147, and 16358, respectively and the LCDR1, LCDR2, and LCDR3 comprises an amino acid sequence of SEQ ID NOS:17966, 9261, and 4386, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17849, 11499, and 4295, respectively and the LCDR1, LCDR2, and LCDR3 comprises an amino acid sequence of SEQ ID NOS:16424, 10926, and 11267, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6578, 18531, and 16186, respectively and the LCDR1, LCDR2, and LCDR3 comprises an amino acid sequence of SEQ ID NOS: 17928, 9680, and 13574, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6566, 11462, and 6181, respectively and the LCDR1, LCDR2, and LCDR3 comprises an amino acid sequence of SEQ ID NOS:16424, 10929, and 11274, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10690, 18008, and 11711, respectively and the LCDR1, LCDR2, and LCDR3 comprises an amino acid sequence of SEQ ID NOS: 16056, 4426, and 12027, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17433, 16933, and 17211, respectively and the LCDR1, LCDR2, and LCDR3 comprises an amino acid sequence of SEQ ID NOS:16665, 11900, and 11890, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17560, 17243, and 4162, respectively and the LCDR1, LCDR2, and LCDR3 comprises an amino acid sequence of SEQ ID NOS:15713, 16561, and 12375, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10692, 18013, and 18055, respectively and the LCDR1, LCDR2, and LCDR3 comprises an amino acid sequence of SEQ ID NOS: 15876, 9503, and 12780, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17856, 16226, and 6002, respectively and the LCDR1, LCDR2, and LCDR3 comprises an amino acid sequence of SEQ ID NOS: 11776, 9476, and 12575, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9971, and 4575, respectively and the LCDR1, LCDR2, and LCDR3 comprises an amino acid sequence of SEQ ID NOS: 15698, 3982, and 12097, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11690, 18590, and 6330, respectively and the LCDR1, LCDR2, and LCDR3 comprises an amino acid sequence of SEQ ID NOS: 15754, 3963, and 12470, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17274, 18016, and 4030, respectively and the LCDR1, LCDR2, and LCDR3 comprises an amino acid sequence of SEQ ID NOS:17924, 10094, and 13575, respectively. having a LCDR1, LCDR2, and LCDR3, wherein the HCDR1, HCDR2, and HCDR3 comprises an amino acid sequence of SEQ ID NOS:6578, 18550, and 11705, respectively and the LCDR1, LCDR2, and LCDR3 comprises an amino acid sequence of SEQ ID NOS:15798, 18440, and 10447, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17435, 10319, and 4274, respectively and the LCDR1, LCDR2, and LCDR3 comprises an amino acid sequence of SEQ ID NOS: 16479, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16539, 12037, and 6050, respectively and the LCDR1, LCDR2, and LCDR3 comprises an amino acid sequence of SEQ ID NOS:18054, 18136, and 11889, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6168, 18626, and 4480, respectively and the LCDR1, LCDR2, and LCDR3 comprises an amino acid sequence of SEQ ID NOS:17900, 16158, and 17571, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10691, 18014, and 11710, respectively and the LCDR1, LCDR2, and LCDR3 comprises an amino acid sequence of SEQ ID NOS: 15961, 4432, and 12885, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17856, 16223, and 6005, respectively and the LCDR1, LCDR2, and LCDR3 comprises an amino acid sequence of SEQ ID NOS: 11787, 9476, and 12575, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17274, 18024, and 6088, respectively and the LCDR1, LCDR2, and LCDR3 comprises an amino acid sequence of SEQ ID NOS: 15990, 4422, and 12437, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6578, 18531, and 10044, respectively and the LCDR1, LCDR2, and LCDR3 comprises an amino acid sequence of SEQ ID NOS:17928, 10094, and 13574, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17446, 10405, and 17694, respectively and the LCDR1, LCDR2, and LCDR3 comprises an amino acid sequence of SEQ ID NOS:15942, 9472, and 12749, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16176, 16209, and 6486, respectively and the LCDR1, LCDR2, and LCDR3 comprises an amino acid sequence of SEQ ID NOS: 16096, 3960, and 12543, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16617, 9984, and 4273, respectively and the LCDR1, LCDR2, and LCDR3 comprises an amino acid sequence of SEQ ID NOS: 15779, 9503, and 12727, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16993, 9392, and 6694, respectively and the LCDR1, LCDR2, and LCDR3 comprises an amino acid sequence of SEQ ID NOS: 16045, 9503, and 12724, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11628, 10056, and 10221, respectively and the LCDR1, LCDR2, and LCDR3 comprises an amino acid sequence of SEQ ID NOS:10891, 18262, and 12992, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10949, 11481, and 6181, respectively and the LCDR1, LCDR2, and LCDR3 comprises an amino acid sequence of SEQ ID NOS:16427, 15513, and 11220, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17616, 16281, and 6001, respectively and the LCDR1, LCDR2, and LCDR3 comprises an amino acid sequence of SEQ ID NOS: 11788, 7913, and 12575, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6570, 16364, and 11731, respectively and the LCDR1, LCDR2, and LCDR3 comprises an amino acid sequence of SEQ ID NOS:16428, 10764, and 11227, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17114, 9386, and 17043, respectively and the LCDR1, LCDR2, and LCDR3 comprises an amino acid sequence of SEQ ID NOS: 15604, 3969, and 11110, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16954, and 10435, respectively and the LCDR1, LCDR2, and LCDR3 comprises an amino acid sequence of SEQ ID NOS:16659, 11900, and 11884, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18621, 9689, and 18220, respectively and the LCDR1, LCDR2, and LCDR3 comprises an amino acid sequence of SEQ ID NOS:16419, 10762, and 17540, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6541, 18504, and 6072, respectively and the LCDR1, LCDR2, and LCDR3 comprises an amino acid sequence of SEQ ID NOS:9365, 7855, and 11127, respectively. having a LCDR1, LCDR2, and LCDR3, wherein the HCDR1, HCDR2, and HCDR3 comprises an amino acid sequence of SEQ ID NOS: 17443, 9390, and 6033, respectively and the LCDR1, LCDR2, and LCDR3 comprises an amino acid sequence of SEQ ID NOS: 15704, 3982, and 12099, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11151, 11464, and 6157, respectively and the LCDR1, LCDR2, and LCDR3 comprises an amino acid sequence of SEQ ID NOS:16424, 10929, and 11274, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17274, 18017, and 6070, respectively and the LCDR1, LCDR2, and LCDR3 comprises an amino acid sequence of SEQ ID NOS: 15986, 4422, and 10711, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6526, 16279, and 6004, respectively and the LCDR1, LCDR2, and LCDR3 comprises an amino acid sequence of SEQ ID NOS: 11780, 4420, and 12575, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4181, 16272, and 7890, respectively and the LCDR1, LCDR2, and LCDR3 comprises an amino acid sequence of SEQ ID NOS: 17924, 9534, and 13578, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16796, 16939, and 18243, respectively and the LCDR1, LCDR2, and LCDR3 comprises an amino acid sequence of SEQ ID NOS:16660, 11900, and 11885, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11557, 16891, and 18449, respectively and the LCDR1, LCDR2, and LCDR3 comprises an amino acid sequence of SEQ ID NOS: 15922, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 18545, and 5896, respectively and the LCDR1, LCDR2, and LCDR3 comprises an amino acid sequence of SEQ ID NOS: 15713, 3982, and 13001, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4635, 11002, and 12236, respectively and the LCDR1, LCDR2, and LCDR3 comprises an amino acid sequence of SEQ ID NOS:16408, 10987, and 11216, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17844, 11189, and 16967, respectively and the LCDR1, LCDR2, and LCDR3 comprises an amino acid sequence of SEQ ID NOS:16753, 17007, and 4194, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10680, 18009, and 18079, respectively and the LCDR1, LCDR2, and LCDR3 comprises an amino acid sequence of SEQ ID NOS:16127, 9461, and 12747, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17989, 18377, and 6105, respectively and the LCDR1, LCDR2, and LCDR3 comprises an amino acid sequence of SEQ ID NOS:16462, 10926, and 11289, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17655, 16280, and 6000, respectively and the LCDR1, LCDR2, and LCDR3 comprises an amino acid sequence of SEQ ID NOS: 11788, 4420, and 12575, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4632, 16365, and 11730, respectively and the LCDR1, LCDR2, and LCDR3 comprises an amino acid sequence of SEQ ID NOS:16383, 10764, and 11217, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17529, 16930, and 5973, respectively and the LCDR1, LCDR2, and LCDR3 comprises an amino acid sequence of SEQ ID NOS: 16045, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10690, 18008, and 11711, respectively and the LCDR1, LCDR2, and LCDR3 comprises an amino acid sequence of SEQ ID NOS: 16056, 4427, and 12029, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17258, 9739, and 10497, respectively and the LCDR1, LCDR2, and LCDR3 comprises an amino acid sequence of SEQ ID NOS: 15962, 3974, and 12713, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17271, 16896, and 6477, respectively and the LCDR1, LCDR2, and LCDR3 comprises an amino acid sequence of SEQ ID NOS: 15856, 4436, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10948, 10569, and 10509, respectively and the LCDR1, LCDR2, and LCDR3 comprises an amino acid sequence of SEQ ID NOS:11789, 4412, and 12582, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6510, 9379, and 4466, respectively and the LCDR1, LCDR2, and LCDR3 comprises an amino acid sequence of SEQ ID NOS: 16045, 9503, and 12797, respectively. having a LCDR1, LCDR2, and LCDR3, wherein the HCDR1, HCDR2, and HCDR3 comprises an amino acid sequence of SEQ ID NOS:6507, 9971, and 6209, respectively and the LCDR1, LCDR2, and LCDR3 comprises an amino acid sequence of SEQ ID NOS: 16067, 4425, and 12437, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11634, 16906, and 6139, respectively and the LCDR1, LCDR2, and LCDR3 comprises an amino acid sequence of SEQ ID NOS: 15970, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17454, 10543, and 4233, respectively and the LCDR1, LCDR2, and LCDR3 comprises an amino acid sequence of SEQ ID NOS:16653, 10747, and 18614, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6565, 11450, and 10346, respectively and the LCDR1, LCDR2, and LCDR3 comprises an amino acid sequence of SEQ ID NOS:10807, 18263, and 12936, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11623, 9759, and 10490, respectively and the LCDR1, LCDR2, and LCDR3 comprises an amino acid sequence of SEQ ID NOS: 16744, 10089, and 3988, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9988, and 9617, respectively and the LCDR1, LCDR2, and LCDR3 comprises an amino acid sequence of SEQ ID NOS: 16002, 9505, and 10461, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17645, 11496, and 17734, respectively and the LCDR1, LCDR2, and LCDR3 comprises an amino acid sequence of SEQ ID NOS: 17926, 10088, and 13572, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11632, 16861, and 6484, respectively and the LCDR1, LCDR2, and LCDR3 comprises an amino acid sequence of SEQ ID NOS:18074, 18596, and 11356, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11656, 16239, and 17033, respectively and the LCDR1, LCDR2, and LCDR3 comprises an amino acid sequence of SEQ ID NOS:16056, 9503, and 10457, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11650, 17812, and 6305, respectively and the LCDR1, LCDR2, and LCDR3 comprises an amino acid sequence of SEQ ID NOS: 11777, 4420, and 12344, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16197, 9938, and 6099, respectively and the LCDR1, LCDR2, and LCDR3 comprises an amino acid sequence of SEQ ID NOS: 15855, 9490, and 12514, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16800, 9710, and 10070, respectively and the LCDR1, LCDR2, and LCDR3 comprises an amino acid sequence of SEQ ID NOS: 15882, 9494, and 12629, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17536, 16214, and 11380, respectively and the LCDR1, LCDR2, and LCDR3 comprises an amino acid sequence of SEQ ID NOS:16656, 10746, and 18610, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11667, 9925, and 10068, respectively and the LCDR1, LCDR2, and LCDR3 comprises an amino acid sequence of SEQ ID NOS: 15719, 3954, and 12732, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16592, 18023, and 7958, respectively and the LCDR1, LCDR2, and LCDR3 comprises an amino acid sequence of SEQ ID NOS: 15856, 4425, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4136, and 6103, respectively and the LCDR1, LCDR2, and LCDR3 comprises an amino acid sequence of SEQ ID NOS:16642, 10747, and 13030, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11369, 16173, and 10327, respectively and the LCDR1, LCDR2, and LCDR3 comprises an amino acid sequence of SEQ ID NOS:15927, 9494, and 12001, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17645, 11496, and 17734, respectively and the LCDR1, LCDR2, and LCDR3 comprises an amino acid sequence of SEQ ID NOS:17926, 10088, and 13571, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 17778, and 7808, respectively and the LCDR1, LCDR2, and LCDR3 comprises an amino acid sequence of SEQ ID NOS: 16054, 9494, and 12690, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11369, 16173, and 10326, respectively and the LCDR1, LCDR2, and LCDR3 comprises an amino acid sequence of SEQ ID NOS:16065, 10314, and 12001, respectively. having a LCDR1, LCDR2, and LCDR3, wherein the HCDR1, HCDR2, and HCDR3 comprises an amino acid sequence of SEQ ID NOS: 17466, 4143, and 7878, respectively and the LCDR1, LCDR2, and LCDR3 comprises an amino acid sequence of SEQ ID NOS: 15904, 4425, and 10725, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:12304, 17819, and 6639, respectively and the LCDR1, LCDR2, and LCDR3 comprises an amino acid sequence of SEQ ID NOS:16644, 10748, and 13034, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11668, 16233, and 17035, respectively and the LCDR1, LCDR2, and LCDR3 comprises an amino acid sequence of SEQ ID NOS: 16067, 4425, and 12408, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17645, 11496, and 17733, respectively and the LCDR1, LCDR2, and LCDR3 comprises an amino acid sequence of SEQ ID NOS: 17926, 10097, and 13565, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17068, 9752, and 9642, respectively and the LCDR1, LCDR2, and LCDR3 comprises an amino acid sequence of SEQ ID NOS: 15974, 4432, and 12683, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17579, 17827, and 11064, respectively and the LCDR1, LCDR2, and LCDR3 comprises an amino acid sequence of SEQ ID NOS: 17979, 6312, and 4373, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16568, 17752, and 10063, respectively and the LCDR1, LCDR2, and LCDR3 comprises an amino acid sequence of SEQ ID NOS:16378, 18275, and 12953, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16673, 18577, and 7875, respectively and the LCDR1, LCDR2, and LCDR3 comprises an amino acid sequence of SEQ ID NOS: 15898, 4281, and 12465, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10351, 17800, and 6014, respectively and the LCDR1, LCDR2, and LCDR3 comprises an amino acid sequence of SEQ ID NOS: 16036, 4404, and 12406, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17441, 18360, and 11715, respectively and the LCDR1, LCDR2, and LCDR3 comprises an amino acid sequence of SEQ ID NOS:16045, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16336, 16298, and 10117, respectively and the LCDR1, LCDR2, and LCDR3 comprises an amino acid sequence of SEQ ID NOS: 16340, 3969, and 12515, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10946, 16892, and 10446, respectively and the LCDR1, LCDR2, and LCDR3 comprises an amino acid sequence of SEQ ID NOS: 16022, 9503, and 12725, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16673, 18587, and 7807, respectively and the LCDR1, LCDR2, and LCDR3 comprises an amino acid sequence of SEQ ID NOS:15932, 15643, and 12452, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17536, 16213, and 11379, respectively and the LCDR1, LCDR2, and LCDR3 comprises an amino acid sequence of SEQ ID NOS:16656, 10746, and 18611, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16990, 9723, and 10199, respectively and the LCDR1, LCDR2, and LCDR3 comprises an amino acid sequence of SEQ ID NOS: 15775, 4436, and 12751, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 17778, and 4461, respectively and the LCDR1, LCDR2, and LCDR3 comprises an amino acid sequence of SEQ ID NOS: 16045, 9494, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9347, 11516, and 18051, respectively and the LCDR1, LCDR2, and LCDR3 comprises an amino acid sequence of SEQ ID NOS:15800, 10930, and 13019, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17140, 4071, and 9358, respectively and the LCDR1, LCDR2, and LCDR3 comprises an amino acid sequence of SEQ ID NOS:17906, 16330, and 16564, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17472, 16235, and 7862, respectively and the LCDR1, LCDR2, and LCDR3 comprises an amino acid sequence of SEQ ID NOS:15657, 10263, and 12355, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17230, 10548, and 9681, respectively and the LCDR1, LCDR2, and LCDR3 comprises an amino acid sequence of SEQ ID NOS:10899, 18275, and 12359, respectively. having a LCDR1, LCDR2, and LCDR3, wherein the HCDR1, HCDR2, and HCDR3 comprises an amino acid sequence of SEQ ID NOS: 16533, 16240, and 9640, respectively and the LCDR1, LCDR2, and LCDR3 comprises an amino acid sequence of SEQ ID NOS: 16045, 9503, and 12012, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17468, 16237, and 6518, respectively and the LCDR1, LCDR2, and LCDR3 comprises an amino acid sequence of SEQ ID NOS: 15888, 6608, and 12410, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11386, 9759, and 10491, respectively and the LCDR1, LCDR2, and LCDR3 comprises an amino acid sequence of SEQ ID NOS: 16744, 10096, and 3988, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17558, 11039, and 4323, respectively and the LCDR1, LCDR2, and LCDR3 comprises an amino acid sequence of SEQ ID NOS:17924, 10094, and 13577, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16692, 18571, and 9604, respectively and the LCDR1, LCDR2, and LCDR3 comprises an amino acid sequence of SEQ ID NOS: 16765, 16331, and 3998, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18361, and 6158, respectively and the LCDR1, LCDR2, and LCDR3 comprises an amino acid sequence of SEQ ID NOS:16441, 10926, and 11277, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 17759, and 5938, respectively and the LCDR1, LCDR2, and LCDR3 comprises an amino acid sequence of SEQ ID NOS: 16030, 6176, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17645, 11496, and 17732, respectively and the LCDR1, LCDR2, and LCDR3 comprises an amino acid sequence of SEQ ID NOS: 16045, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16673, 11532, and 7876, respectively and the LCDR1, LCDR2, and LCDR3 comprises an amino acid sequence of SEQ ID NOS: 15865, 3968, and 12486, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9978, and 10027, respectively and the LCDR1, LCDR2, and LCDR3 comprises an amino acid sequence of SEQ ID NOS: 16038, 9488, and 12623, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16170, 4066, and 7879, respectively and the LCDR1, LCDR2, and LCDR3 comprises an amino acid sequence of SEQ ID NOS:15815, 16143, and 10474, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11632, 17754, and 5439, respectively and the LCDR1, LCDR2, and LCDR3 comprises an amino acid sequence of SEQ ID NOS: 16460, 9502, and 12947, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11673, 10536, and 4341, respectively and the LCDR1, LCDR2, and LCDR3 comprises an amino acid sequence of SEQ ID NOS:15890, 10727, and 11107, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 4152, and 4461, respectively and the LCDR1, LCDR2, and LCDR3 comprises an amino acid sequence of SEQ ID NOS: 16067, 4425, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9682, 16234, and 3942, respectively and the LCDR1, LCDR2, and LCDR3 comprises an amino acid sequence of SEQ ID NOS: 16045, 3983, and 12557, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17471, 16231, and 18455, respectively and the LCDR1, LCDR2, and LCDR3 comprises an amino acid sequence of SEQ ID NOS: 15983, 9503, and 10386, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9684, 18296, and 18446, respectively and the LCDR1, LCDR2, and LCDR3 comprises an amino acid sequence of SEQ ID NOS: 16738, 5977, and 10272, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16610, 10042, and 3946, respectively and the LCDR1, LCDR2, and LCDR3 comprises an amino acid sequence of SEQ ID NOS:15801, 10727, and 12024, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17838, 11456, and 12105, respectively and the LCDR1, LCDR2, and LCDR3 comprises an amino acid sequence of SEQ ID NOS: 16733, 17007, and 4016, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17270, 18302, and 17709, respectively and the LCDR1, LCDR2, and LCDR3 comprises an amino acid sequence of SEQ ID NOS: 16382, 10926, and 11293, respectively. having a LCDR1, LCDR2, and LCDR3, wherein the HCDR1, HCDR2, and HCDR3 comprises an amino acid sequence of SEQ ID NOS:6573, 18321, and 10213, respectively and the LCDR1, LCDR2, and LCDR3 comprises an amino acid sequence of SEQ ID NOS: 16773, 10025, and 4000, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17621, 17778, and 4399, respectively and the LCDR1, LCDR2, and LCDR3 comprises an amino acid sequence of SEQ ID NOS:15948, 10705, and 12861, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17631, 8043, and 11116, respectively and the LCDR1, LCDR2, and LCDR3 comprises an amino acid sequence of SEQ ID NOS: 16016, 9477, and 12501, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11649, 18400, and 10343, respectively and the LCDR1, LCDR2, and LCDR3 comprises an amino acid sequence of SEQ ID NOS: 17953, 6312, and 4337, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17645, 4070, and 4401, respectively and the LCDR1, LCDR2, and LCDR3 comprises an amino acid sequence of SEQ ID NOS:17927, 10088, and 13323, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16534, 16241, and 17564, respectively and the LCDR1, LCDR2, and LCDR3 comprises an amino acid sequence of SEQ ID NOS: 16035, 4425, and 12408, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17292, 18584, and 6676, respectively and the LCDR1, LCDR2, and LCDR3 comprises an amino acid sequence of SEQ ID NOS: 15893, 9498, and 12518, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6578, 10410, and 18137, respectively and the LCDR1, LCDR2, and LCDR3 comprises an amino acid sequence of SEQ ID NOS:10847, 18275, and 12980, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17627, 10547, and 4232, respectively and the LCDR1, LCDR2, and LCDR3 comprises an amino acid sequence of SEQ ID NOS:16671, 10749, and 18614, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17562, 16832, and 18444, respectively and the LCDR1, LCDR2, and LCDR3 comprises an amino acid sequence of SEQ ID NOS:17959, 9262, and 17152, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6501, 4106, and 10185, respectively and the LCDR1, LCDR2, and LCDR3 comprises an amino acid sequence of SEQ ID NOS: 15617, 6604, and 12853, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17446, 16856, and 4170, respectively and the LCDR1, LCDR2, and LCDR3 comprises an amino acid sequence of SEQ ID NOS: 16099, 4425, and 12449, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17604, 9399, and 10584, respectively and the LCDR1, LCDR2, and LCDR3 comprises an amino acid sequence of SEQ ID NOS: 15901, 9503, and 12448, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17645, 11496, and 17734, respectively and the LCDR1, LCDR2, and LCDR3 comprises an amino acid sequence of SEQ ID NOS:17926, 10088, and 13571, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16335, 16297, and 4317, respectively and the LCDR1, LCDR2, and LCDR3 comprises an amino acid sequence of SEQ ID NOS: 15885, 3974, and 12651, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17841, 11437, and 4041, respectively and the LCDR1, LCDR2, and LCDR3 comprises an amino acid sequence of SEQ ID NOS: 16045, 9503, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11619, 9777, and 17059, respectively and the LCDR1, LCDR2, and LCDR3 comprises an amino acid sequence of SEQ ID NOS: 17949, 6318, and 17184, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17580, 17827, and 11063, respectively and the LCDR1, LCDR2, and LCDR3 comprises an amino acid sequence of SEQ ID NOS: 16780, 6242, and 4373, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10940, 11449, and 10687, respectively and the LCDR1, LCDR2, and LCDR3 comprises an amino acid sequence of SEQ ID NOS:10887, 18275, and 12911, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4132, and 10977, respectively and the LCDR1, LCDR2, and LCDR3 comprises an amino acid sequence of SEQ ID NOS:17966, 9261, and 4379, respectively. having a LCDR1, LCDR2, and LCDR3, wherein the HCDR1, HCDR2, and HCDR3 comprises an amino acid sequence of SEQ ID NOS:16536, 16230, and 11055, respectively and the LCDR1, LCDR2, and LCDR3 comprises an amino acid sequence of SEQ ID NOS: 15927, 9494, and 12642, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17114, 17751, and 9619, respectively and the LCDR1, LCDR2, and LCDR3 comprises an amino acid sequence of SEQ ID NOS:10795, 18275, and 12371, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17622, 16922, and 10134, respectively and the LCDR1, LCDR2, and LCDR3 comprises an amino acid sequence of SEQ ID NOS:15916, 9503, and 12725, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6565, 18539, and 17110, respectively and the LCDR1, LCDR2, and LCDR3 comprises an amino acid sequence of SEQ ID NOS: 16036, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17536, 16212, and 11379, respectively and the LCDR1, LCDR2, and LCDR3 comprises an amino acid sequence of SEQ ID NOS:16663, 10746, and 18612, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17275, 4144, and 4296, respectively and the LCDR1, LCDR2, and LCDR3 comprises an amino acid sequence of SEQ ID NOS: 16743, 17007, and 4018, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17646, 16245, and 11617, respectively and the LCDR1, LCDR2, and LCDR3 comprises an amino acid sequence of SEQ ID NOS: 17939, 9268, and 4385, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11679, 16836, and 17859, respectively and the LCDR1, LCDR2, and LCDR3 comprises an amino acid sequence of SEQ ID NOS:10885, 18275, and 12910, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10080, 10563, and, respectively and the LCDR1, LCDR2, and LCDR3 comprises an amino acid sequence of SEQ ID NOS:4238, 6212, and 9416, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6575, 16862, and 11351, respectively and the LCDR1, LCDR2, and LCDR3 comprises an amino acid sequence of SEQ ID NOS:18269, 3982, and 12383, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11598, 11444, and 11100, respectively and the LCDR1, LCDR2, and LCDR3 comprises an amino acid sequence of SEQ ID NOS:10886, 18275, and 12903, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10951, 11447, and 10963, respectively and the LCDR1, LCDR2, and LCDR3 comprises an amino acid sequence of SEQ ID NOS:10877, 18275, and 12611, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11662, 11468, and 11130, respectively and the LCDR1, LCDR2, and LCDR3 comprises an amino acid sequence of SEQ ID NOS:10884, 18268, and 12934, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10368, 6782, and 15585, respectively and the LCDR1, LCDR2, and LCDR3 comprises an amino acid sequence of SEQ ID NOS: 17959, 6318, and 17163, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11559, 10568, and 11114, respectively and the LCDR1, LCDR2, and LCDR3 comprises an amino acid sequence of SEQ ID NOS:10813, 18275, and 12903, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11650, 5893, and 10181, respectively and the LCDR1, LCDR2, and LCDR3 comprises an amino acid sequence of SEQ ID NOS:16149, 17577, and 12546, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11406, 10568, and 11114, respectively and the LCDR1, LCDR2, and LCDR3 comprises an amino acid sequence of SEQ ID NOS:16147, 10705, and 12858, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6673, 10560, and 17026, respectively and the LCDR1, LCDR2, and LCDR3 comprises an amino acid sequence of SEQ ID NOS:16658, 12302, and 11868, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11650, 17792, and 16996, respectively and the LCDR1, LCDR2, and LCDR3 comprises an amino acid sequence of SEQ ID NOS: 15987, 9494, and 10997, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4321, 16842, and 16814, respectively and the LCDR1, LCDR2, and LCDR3 comprises an amino acid sequence of SEQ ID NOS:10871, 18275, and 12926, respectively. having a LCDR1, LCDR2, and LCDR3, wherein the HCDR1, HCDR2, and HCDR3 comprises an amino acid sequence of SEQ ID NOS: 17228, 10422, and 9622, respectively and the LCDR1, LCDR2, and LCDR3 comprises an amino acid sequence of SEQ ID NOS: 15991, 9474, and 12714, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17648, 11457, and 6054, respectively and the LCDR1, LCDR2, and LCDR3 comprises an amino acid sequence of SEQ ID NOS:18087, 11071, and 13613, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17456, 18536, and 5994, respectively and the LCDR1, LCDR2, and LCDR3 comprises an amino acid sequence of SEQ ID NOS:18003, 18597, and 11030, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17069, 9403, and 6071, respectively and the LCDR1, LCDR2, and LCDR3 comprises an amino acid sequence of SEQ ID NOS:15958, 10316, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17291, 17994, and 10111, respectively and the LCDR1, LCDR2, and LCDR3 comprises an amino acid sequence of SEQ ID NOS: 15660, 3969, and 11094, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:7819, 16613, and 9344, respectively and the LCDR1, LCDR2, and LCDR3 comprises an amino acid sequence of SEQ ID NOS: 16409, 9363, and 11344, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11564, 11443, and 11712, respectively and the LCDR1, LCDR2, and LCDR3 comprises an amino acid sequence of SEQ ID NOS:10799, 18275, and 13013, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6563, 10678, and 11125, respectively and the LCDR1, LCDR2, and LCDR3 comprises an amino acid sequence of SEQ ID NOS:10816, 18275, and 12967, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11678, 17997, and 10594, respectively and the LCDR1, LCDR2, and LCDR3 comprises an amino acid sequence of SEQ ID NOS:10816, 18275, and 12918, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11158, 18343, and 10430, respectively and the LCDR1, LCDR2, and LCDR3 comprises an amino acid sequence of SEQ ID NOS:15832, 10310, and 12461, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11647, 10007, and 18138, respectively and the LCDR1, LCDR2, and LCDR3 comprises an amino acid sequence of SEQ ID NOS:15917, 4425, and 12442, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6575, 4329, and 4343, respectively and the LCDR1, LCDR2, and LCDR3 comprises an amino acid sequence of SEQ ID NOS:15661, 16561, and 12382, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11677, 16258, and 17895, respectively and the LCDR1, LCDR2, and LCDR3 comprises an amino acid sequence of SEQ ID NOS: 10053, 17222, and 4165, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11393, 18530, and 9545, respectively and the LCDR1, LCDR2, and LCDR3 comprises an amino acid sequence of SEQ ID NOS:18078, 18596, and 11183, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11412, 11003, and 11168, respectively and the LCDR1, LCDR2, and LCDR3 comprises an amino acid sequence of SEQ ID NOS: 16412, 10960, and 11194, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4635, 10553, and 17128, respectively and the LCDR1, LCDR2, and LCDR3 comprises an amino acid sequence of SEQ ID NOS:10781, 10764, and 11224, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 18466, and 5955, respectively and the LCDR1, LCDR2, and LCDR3 comprises an amino acid sequence of SEQ ID NOS:16425, 10926, and 11272, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17842, 11488, and 18031, respectively and the LCDR1, LCDR2, and LCDR3 comprises an amino acid sequence of SEQ ID NOS: 16640, 6634, and 18620, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11600, 18524, and 6468, respectively and the LCDR1, LCDR2, and LCDR3 comprises an amino acid sequence of SEQ ID NOS: 15776, 4433, and 12459, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16544, 11458, and 17659, respectively and the LCDR1, LCDR2, and LCDR3 comprises an amino acid sequence of SEQ ID NOS:10803, 18273, and 16345, respectively. having a LCDR1, LCDR2, and LCDR3, wherein the HCDR1, HCDR2, and HCDR3 comprises an amino acid sequence of SEQ ID NOS: 17886, 18350, and 9425, respectively and the LCDR1, LCDR2, and LCDR3 comprises an amino acid sequence of SEQ ID NOS: 17975, 6245, and 17204, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4620, 16477, and 4603, respectively and the LCDR1, LCDR2, and LCDR3 comprises an amino acid sequence of SEQ ID NOS:10904, 18275, and 12933, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:7818, 9572, and 9343, respectively and the LCDR1, LCDR2, and LCDR3 comprises an amino acid sequence of SEQ ID NOS: 16409, 9361, and 11272, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 18545, and 4460, respectively and the LCDR1, LCDR2, and LCDR3 comprises an amino acid sequence of SEQ ID NOS: 11789, 4420, and 12577, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4569, 18328, and 17701, respectively and the LCDR1, LCDR2, and LCDR3 comprises an amino acid sequence of SEQ ID NOS: 16056, 9503, and 12644, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11630, 17776, and 9627, respectively and the LCDR1, LCDR2, and LCDR3 comprises an amino acid sequence of SEQ ID NOS:16402, 17717, and 11278, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6571, 11000, and 9666, respectively and the LCDR1, LCDR2, and LCDR3 comprises an amino acid sequence of SEQ ID NOS:16405, 17665, and 11195, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17275, 16974, and 6483, respectively and the LCDR1, LCDR2, and LCDR3 comprises an amino acid sequence of SEQ ID NOS:16745, 18105, and 4011, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:5901, 9565, and 9343, respectively and the LCDR1, LCDR2, and LCDR3 comprises an amino acid sequence of SEQ ID NOS: 16409, 9362, and 11344, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10769, 9858, and 11725, respectively and the LCDR1, LCDR2, and LCDR3 comprises an amino acid sequence of SEQ ID NOS:15847, 10706, and 12015, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6576, 18322, and 10958, respectively and the LCDR1, LCDR2, and LCDR3 comprises an amino acid sequence of SEQ ID NOS:15880, 10706, and 12015, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11412, 11136, and 11167, respectively and the LCDR1, LCDR2, and LCDR3 comprises an amino acid sequence of SEQ ID NOS: 16465, 10990, and 11195, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11424, 11458, and 11937, respectively and the LCDR1, LCDR2, and LCDR3 comprises an amino acid sequence of SEQ ID NOS:16376, 18275, and 10460, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 17778, and 4613, respectively and the LCDR1, LCDR2, and LCDR3 comprises an amino acid sequence of SEQ ID NOS: 16045, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 17798, and 12102, respectively and the LCDR1, LCDR2, and LCDR3 comprises an amino acid sequence of SEQ ID NOS:10891, 18275, and 12973, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17441, 4326, and 10197, respectively and the LCDR1, LCDR2, and LCDR3 comprises an amino acid sequence of SEQ ID NOS: 10716, 9503, and 12798, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17023, 18038, and 10369, respectively and the LCDR1, LCDR2, and LCDR3 comprises an amino acid sequence of SEQ ID NOS:16736, 7864, and 10271, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4251, 10581, and 11355, respectively and the LCDR1, LCDR2, and LCDR3 comprises an amino acid sequence of SEQ ID NOS: 17980, 11610, and 4372, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6545, 18564, and 6697, respectively and the LCDR1, LCDR2, and LCDR3 comprises an amino acid sequence of SEQ ID NOS: 16741, 16332, and 4014, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17262, 9798, and 11694, respectively and the LCDR1, LCDR2, and LCDR3 comprises an amino acid sequence of SEQ ID NOS:15809, 10727, and 12020, respectively. having a LCDR1, LCDR2, and LCDR3, wherein the HCDR1, HCDR2, and HCDR3 comprises an amino acid sequence of SEQ ID NOS: 11687, 18519, and 4475, respectively and the LCDR1, LCDR2, and LCDR3 comprises an amino acid sequence of SEQ ID NOS: 15841, 3962, and 12472, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17260, 18300, and 4411, respectively and the LCDR1, LCDR2, and LCDR3 comprises an amino acid sequence of SEQ ID NOS:10789, 18275, and 12373, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17618, 18358, and 17836, respectively and the LCDR1, LCDR2, and LCDR3 comprises an amino acid sequence of SEQ ID NOS:17959, 6318, and 17195, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11647, 18372, and 6151, respectively and the LCDR1, LCDR2, and LCDR3 comprises an amino acid sequence of SEQ ID NOS: 16101, 4257, and 12551, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17856, 18631, and 10328, respectively and the LCDR1, LCDR2, and LCDR3 comprises an amino acid sequence of SEQ ID NOS:15864, 10671, and 12489, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17441, 18360, and 10519, respectively and the LCDR1, LCDR2, and LCDR3 comprises an amino acid sequence of SEQ ID NOS:16659, 11900, and 11873, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11566, 18470, and 10329, respectively and the LCDR1, LCDR2, and LCDR3 comprises an amino acid sequence of SEQ ID NOS:15749, 10671, and 12532, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17569, 16947, and 11927, respectively and the LCDR1, LCDR2, and LCDR3 comprises an amino acid sequence of SEQ ID NOS:16389, 18256, and 12968, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4324, 18316, and 9655, respectively and the LCDR1, LCDR2, and LCDR3 comprises an amino acid sequence of SEQ ID NOS: 15785, 3953, and 12503, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17024, 16945, and 10374, respectively and the LCDR1, LCDR2, and LCDR3 comprises an amino acid sequence of SEQ ID NOS:15836, 11368, and 12744, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11653, 9738, and 17071, respectively and the LCDR1, LCDR2, and LCDR3 comprises an amino acid sequence of SEQ ID NOS: 15981, 9494, and 12664, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11648, 18391, and 10172, respectively and the LCDR1, LCDR2, and LCDR3 comprises an amino acid sequence of SEQ ID NOS: 16771, 5988, and 10270, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11650, 11012, and 9531, respectively and the LCDR1, LCDR2, and LCDR3 comprises an amino acid sequence of SEQ ID NOS:15728, 10697, and 12875, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11690, 18585, and 10299, respectively and the LCDR1, LCDR2, and LCDR3 comprises an amino acid sequence of SEQ ID NOS: 15688, 11732, and 12338, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11675, 16215, and 10440, respectively and the LCDR1, LCDR2, and LCDR3 comprises an amino acid sequence of SEQ ID NOS: 16733, 18445, and 4001, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16778, 16985, and 15601, respectively and the LCDR1, LCDR2, and LCDR3 comprises an amino acid sequence of SEQ ID NOS:18053, 11070, and 13620, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16785, 16984, and 15600, respectively and the LCDR1, LCDR2, and LCDR3 comprises an amino acid sequence of SEQ ID NOS:18052, 11070, and 13620, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11647, 18432, and 4488, respectively and the LCDR1, LCDR2, and LCDR3 comprises an amino acid sequence of SEQ ID NOS:16353, 10926, and 11280, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11690, 18472, and 4473, respectively and the LCDR1, LCDR2, and LCDR3 comprises an amino acid sequence of SEQ ID NOS: 15802, 3966, and 12488, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4308, 16857, and 6470, respectively and the LCDR1, LCDR2, and LCDR3 comprises an amino acid sequence of SEQ ID NOS:16766, 18102, and 4285, respectively. having a LCDR1, LCDR2, and LCDR3, wherein the HCDR1, HCDR2, and HCDR3 comprises an amino acid sequence of SEQ ID NOS:11655, 18634, and 10325, respectively and the LCDR1, LCDR2, and LCDR3 comprises an amino acid sequence of SEQ ID NOS:15705, 10671, and 12489, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 9695, and 5961, respectively and the LCDR1, LCDR2, and LCDR3 comprises an amino acid sequence of SEQ ID NOS: 16709, 16327, and 4245, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17552, 11039, and 11572, respectively and the LCDR1, LCDR2, and LCDR3 comprises an amino acid sequence of SEQ ID NOS:10883, 18275, and 12968, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4630, 9410, and 10168, respectively and the LCDR1, LCDR2, and LCDR3 comprises an amino acid sequence of SEQ ID NOS:10869, 18275, and 12959, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4310, 9395, and 6084, respectively and the LCDR1, LCDR2, and LCDR3 comprises an amino acid sequence of SEQ ID NOS: 15860, 4425, and 12415, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4311, 18393, and 9580, respectively and the LCDR1, LCDR2, and LCDR3 comprises an amino acid sequence of SEQ ID NOS: 15900, 3953, and 12531, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17656, 18635, and 4479, respectively and the LCDR1, LCDR2, and LCDR3 comprises an amino acid sequence of SEQ ID NOS: 16117, 3964, and 12401, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11408, 16263, and 17250, respectively and the LCDR1, LCDR2, and LCDR3 comprises an amino acid sequence of SEQ ID NOS:16133, 17666, and 12547, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10768, 10537, and 16606, respectively and the LCDR1, LCDR2, and LCDR3 comprises an amino acid sequence of SEQ ID NOS:16652, 11893, and 11864, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11647, 18370, and 6152, respectively and the LCDR1, LCDR2, and LCDR3 comprises an amino acid sequence of SEQ ID NOS:16100, 4257, and 12551, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16374, 17244, and 10371, respectively and the LCDR1, LCDR2, and LCDR3 comprises an amino acid sequence of SEQ ID NOS:15907, 6614, and 12807, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17475, 16830, and 6698, respectively and the LCDR1, LCDR2, and LCDR3 comprises an amino acid sequence of SEQ ID NOS:18081, 18134, and 13619, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17558, 11040, and 11571, respectively and the LCDR1, LCDR2, and LCDR3 comprises an amino acid sequence of SEQ ID NOS:10874, 18275, and 10478, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16793, 10564, and 10307, respectively and the LCDR1, LCDR2, and LCDR3 comprises an amino acid sequence of SEQ ID NOS:9635, 4467, and 15561, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17649, 11429, and 9605, respectively and the LCDR1, LCDR2, and LCDR3 comprises an amino acid sequence of SEQ ID NOS: 15789, 3969, and 12500, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11650, 9420, and 6024, respectively and the LCDR1, LCDR2, and LCDR3 comprises an amino acid sequence of SEQ ID NOS:16437, 10931, and 11271, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9971, and 4586, respectively and the LCDR1, LCDR2, and LCDR3 comprises an amino acid sequence of SEQ ID NOS: 16045, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10759, 4668, and 4469, respectively and the LCDR1, LCDR2, and LCDR3 comprises an amino acid sequence of SEQ ID NOS:17914, 16333, and 16566, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11632, 17821, and 10239, respectively and the LCDR1, LCDR2, and LCDR3 comprises an amino acid sequence of SEQ ID NOS:15702, 3961, and 12944, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17124, 18039, and 17697, respectively and the LCDR1, LCDR2, and LCDR3 comprises an amino acid sequence of SEQ ID NOS:10810, 18275, and 12992, respectively. having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 15771, 9502, and 12743, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6509, 9957, and 4197, respectively and the LCDR1, LCDR2, and LCDR3 comprises an amino acid sequence of SEQ ID NOS:15619, 10264, and 12794, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17278, 17739, and 4297, respectively and the LCDR1, LCDR2, and LCDR3 comprises an amino acid sequence of SEQ ID NOS:9626, 5985, and 15553, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:16104, 9503, and 12867, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:17959, 6327, and 17187, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 10532, 10093, and 17234, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17278, 9741, and 4298, respectively and the LCDR1, LCDR2, and LCDR3 comprises an amino acid sequence of SEQ ID NOS:9625, 5985, and 15553, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17474, 9985, and 18421, respectively and the LCDR1, LCDR2, and LCDR3 comprises an amino acid sequence of SEQ ID NOS: 10828, 9267, and 11310, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16822, 16290, and 4501, respectively and the LCDR1, LCDR2, and LCDR3 comprises an amino acid sequence of SEQ ID NOS: 15880, 3953, and 12519, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17432, 18483, and 17652, respectively and the LCDR1, LCDR2, and LCDR3 comprises an amino acid sequence of SEQ ID NOS:15826, 11791, and 12816, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:16450, 18262, and 12954, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:18048, 18609, and 11177, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:4053, 10076, and 17237, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9739, and 10484, respectively and the LCDR1, LCDR2, and LCDR3 comprises an amino acid sequence of SEQ ID NOS: 10830, 9267, and 11307, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17020, 10294, and 6089, respectively and the LCDR1, LCDR2, and LCDR3 comprises an amino acid sequence of SEQ ID NOS: 11789, 4420, and 12577, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17446, 4271, and 6496, respectively and the LCDR1, LCDR2, and LCDR3 comprises an amino acid sequence of SEQ ID NOS:16754, 17001, and 17671, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18641, 4166, and 10239, respectively and the LCDR1, LCDR2, and LCDR3 comprises an amino acid sequence of SEQ ID NOS:16641, 10747, and 13020, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17053, 11006, and 17911, respectively and the LCDR1, LCDR2, and LCDR3 comprises an amino acid sequence of SEQ ID NOS: 15762, 3986, and 12539, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11678, 11489, and 10239, respectively and the LCDR1, LCDR2, and LCDR3 comprises an amino acid sequence of SEQ ID NOS:10875, 18271, and 12899, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 16735, 10087, and 17672, respectively. having a LCDR1, LCDR2, and LCDR3, wherein the HCDR1, HCDR2, and HCDR3 comprises an amino acid sequence of SEQ ID NOS: 17263, 9812, and 4338, respectively and the LCDR1, LCDR2, and LCDR3 comprises an amino acid sequence of SEQ ID NOS:11790, 10727, and 12016, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17478, 18396, and 16190, respectively and the LCDR1, LCDR2, and LCDR3 comprises an amino acid sequence of SEQ ID NOS:15768, 3974, and 12681, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16567, 4332, and 9675, respectively and the LCDR1, LCDR2, and LCDR3 comprises an amino acid sequence of SEQ ID NOS: 16731, 7859, and 10284, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:15652, 9470, and 11981, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 15708, 3980, and 12395, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16632, 11172, and 4643, respectively and the LCDR1, LCDR2, and LCDR3 comprises an amino acid sequence of SEQ ID NOS: 17952, 9275, and 17172, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11654, 18349, and 4578, respectively and the LCDR1, LCDR2, and LCDR3 comprises an amino acid sequence of SEQ ID NOS: 16371, 9257, and 11279, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17619, 18399, and 4235, respectively and the LCDR1, LCDR2, and LCDR3 comprises an amino acid sequence of SEQ ID NOS: 17961, 9275, and 17173, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:10817, 18275, and 12904, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10938, 9936, and 10239, respectively and the LCDR1, LCDR2, and LCDR3 comprises an amino acid sequence of SEQ ID NOS:16684, 16587, and 17668, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16541, 10400, and 6079, respectively and the LCDR1, LCDR2, and LCDR3 comprises an amino acid sequence of SEQ ID NOS: 15690, 3961, and 11082, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17608, 16850, and 6491, respectively and the LCDR1, LCDR2, and LCDR3 comprises an amino acid sequence of SEQ ID NOS: 16710, 16585, and 4262, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 16085, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6191, 10999, and 4129, respectively and the LCDR1, LCDR2, and LCDR3 comprises an amino acid sequence of SEQ ID NOS: 10830, 9267, and 11272, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17053, 11008, and 17910, respectively and the LCDR1, LCDR2, and LCDR3 comprises an amino acid sequence of SEQ ID NOS: 15843, 3986, and 12539, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17264, 16835, and 16552, respectively and the LCDR1, LCDR2, and LCDR3 comprises an amino acid sequence of SEQ ID NOS:10903, 7981, and 11341, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:16781, 9254, and 17159, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11633, 16880, and 10239, respectively and the LCDR1, LCDR2, and LCDR3 comprises an amino acid sequence of SEQ ID NOS: 16755, 17007, and 17669, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10336, 9776, and 16347, respectively and the LCDR1, LCDR2, and LCDR3 comprises an amino acid sequence of SEQ ID NOS: 15681, 4611, and 12351, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16802, 9942, and 11122, respectively and the LCDR1, LCDR2, and LCDR3 comprises an amino acid sequence of SEQ ID NOS: 16061, 4431, and 12677, respectively. having a LCDR1, LCDR2, and LCDR3, wherein the HCDR1, HCDR2, and HCDR3 comprises an amino acid sequence of SEQ ID NOS:17209, 11536, and 10309, respectively and the LCDR1, LCDR2, and LCDR3 comprises an amino acid sequence of SEQ ID NOS:10830, 9267, and 11321, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17535, 10570, and 4221, respectively and the LCDR1, LCDR2, and LCDR3 comprises an amino acid sequence of SEQ ID NOS:10889, 18275, and 12966, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11154, 9803, and 9656, respectively and the LCDR1, LCDR2, and LCDR3 comprises an amino acid sequence of SEQ ID NOS: 17920, 6244, and 4382, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17848, 10546, and 17065, respectively and the LCDR1, LCDR2, and LCDR3 comprises an amino acid sequence of SEQ ID NOS: 16092, 9494, and 12664, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:5958, 11135, and 4192, respectively and the LCDR1, LCDR2, and LCDR3 comprises an amino acid sequence of SEQ ID NOS: 16045, 9503, and 12779, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16544, 16839, and 11982, respectively and the LCDR1, LCDR2, and LCDR3 comprises an amino acid sequence of SEQ ID NOS:10806, 18275, and 10475, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6045, 16289, and 10176, respectively and the LCDR1, LCDR2, and LCDR3 comprises an amino acid sequence of SEQ ID NOS:10794, 18275, and 12920, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11630, 17763, and 6062, respectively and the LCDR1, LCDR2, and LCDR3 comprises an amino acid sequence of SEQ ID NOS: 16045, 9503, and 12749, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:7811, 16612, and 11704, respectively and the LCDR1, LCDR2, and LCDR3 comprises an amino acid sequence of SEQ ID NOS:16408, 10984, and 11346, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10368, 6780, and 10247, respectively and the LCDR1, LCDR2, and LCDR3 comprises an amino acid sequence of SEQ ID NOS:11946, 10024, and 13327, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6501, 18484, and 9670, respectively and the LCDR1, LCDR2, and LCDR3 comprises an amino acid sequence of SEQ ID NOS:16439, 10926, and 11294, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16961, 10398, and 11117, respectively and the LCDR1, LCDR2, and LCDR3 comprises an amino acid sequence of SEQ ID NOS:10807, 18275, and 13009, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11067, 11016, and 5998, respectively and the LCDR1, LCDR2, and LCDR3 comprises an amino acid sequence of SEQ ID NOS:16414, 10987, and 11199, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11634, 17778, and 6093, respectively and the LCDR1, LCDR2, and LCDR3 comprises an amino acid sequence of SEQ ID NOS: 11784, 4609, and 12398, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17856, 18585, and 11118, respectively and the LCDR1, LCDR2, and LCDR3 comprises an amino acid sequence of SEQ ID NOS:16712, 18105, and 4284, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9776, and 6723, respectively and the LCDR1, LCDR2, and LCDR3 comprises an amino acid sequence of SEQ ID NOS:16712, 18105, and 4284, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9781, and 4333, respectively and the LCDR1, LCDR2, and LCDR3 comprises an amino acid sequence of SEQ ID NOS:15847, 10704, and 12021, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11659, 18507, and 5993, respectively and the LCDR1, LCDR2, and LCDR3 comprises an amino acid sequence of SEQ ID NOS:18047, 18628, and 11186, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11419, 18346, and 10332, respectively and the LCDR1, LCDR2, and LCDR3 comprises an amino acid sequence of SEQ ID NOS:10796, 18267, and 12935, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18641, 4067, and 18219, respectively and the LCDR1, LCDR2, and LCDR3 comprises an amino acid sequence of SEQ ID NOS:16642, 10747, and 13021, respectively. having a LCDR1, LCDR2, and LCDR3, wherein the HCDR1, HCDR2, and HCDR3 comprises an amino acid sequence of SEQ ID NOS:5996, 18312, and 9515, respectively and the LCDR1, LCDR2, and LCDR3 comprises an amino acid sequence of SEQ ID NOS:10869, 18265, and 12932, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6565, 4647, and 4477, respectively and the LCDR1, LCDR2, and LCDR3 comprises an amino acid sequence of SEQ ID NOS: 16760, 11575, and 4284, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9783, and 9421, respectively and the LCDR1, LCDR2, and LCDR3 comprises an amino acid sequence of SEQ ID NOS: 15807, 3953, and 12477, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:15818, 18426, and 12842, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10337, 9991, and 9455, respectively and the LCDR1, LCDR2, and LCDR3 comprises an amino acid sequence of SEQ ID NOS: 16059, 4432, and 11998, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9856, and 6085, respectively and the LCDR1, LCDR2, and LCDR3 comprises an amino acid sequence of SEQ ID NOS:17951, 9261, and 17205, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4634, 18223, and 17240, respectively and the LCDR1, LCDR2, and LCDR3 comprises an amino acid sequence of SEQ ID NOS: 15679, 6324, and 12381, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11652, 9397, and 11165, respectively and the LCDR1, LCDR2, and LCDR3 comprises an amino acid sequence of SEQ ID NOS: 15858, 4436, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:15906, 4436, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17266, 9867, and 4160, respectively and the LCDR1, LCDR2, and LCDR3 comprises an amino acid sequence of SEQ ID NOS: 15834, 3972, and 12350, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 16073, 6606, and 11985, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4228, 18479, and 11563, respectively and the LCDR1, LCDR2, and LCDR3 comprises an amino acid sequence of SEQ ID NOS: 16559, 6628, and 7952, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11690, 4331, and 9665, respectively and the LCDR1, LCDR2, and LCDR3 comprises an amino acid sequence of SEQ ID NOS: 16017, 3983, and 12746, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 16380, 10929, and 11290, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 16022, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10518, 11507, and 18200, respectively and the LCDR1, LCDR2, and LCDR3 comprises an amino acid sequence of SEQ ID NOS:10869, 18275, and 12915, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11388, 16927, and 10239, respectively and the LCDR1, LCDR2, and LCDR3 comprises an amino acid sequence of SEQ ID NOS: 15953, 10700, and 12567, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18383, and 6488, respectively and the LCDR1, LCDR2, and LCDR3 comprises an amino acid sequence of SEQ ID NOS:16421, 10927, and 11282, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18640, 11173, and 18450, respectively and the LCDR1, LCDR2, and LCDR3 comprises an amino acid sequence of SEQ ID NOS: 15646, 3969, and 11090, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17270, 10556, and 10239, respectively and the LCDR1, LCDR2, and LCDR3 comprises an amino acid sequence of SEQ ID NOS:16642, 16157, and 13335, respectively. having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:15919, 4425, and 12867, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10337, 9884, and 9455, respectively and the LCDR1, LCDR2, and LCDR3 comprises an amino acid sequence of SEQ ID NOS: 16064, 4425, and 11991, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11622, 16286, and 4299, respectively and the LCDR1, LCDR2, and LCDR3 comprises an amino acid sequence of SEQ ID NOS:10800, 18275, and 12898, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:5997, 17738, and 10239, respectively and the LCDR1, LCDR2, and LCDR3 comprises an amino acid sequence of SEQ ID NOS:9360, 6459, and 17176, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10511, 16879, and 6490, respectively and the LCDR1, LCDR2, and LCDR3 comprises an amino acid sequence of SEQ ID NOS:16758, 4176, and 4284, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11678, 11454, and 10496, respectively and the LCDR1, LCDR2, and LCDR3 comprises an amino acid sequence of SEQ ID NOS:10876, 18271, and 12908, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9783, and 9421, respectively and the LCDR1, LCDR2, and LCDR3 comprises an amino acid sequence of SEQ ID NOS: 15704, 3982, and 12094, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9935, and 10239, respectively and the LCDR1, LCDR2, and LCDR3 comprises an amino acid sequence of SEQ ID NOS: 10861, 9267, and 11323, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9567, 9885, and 10751, respectively and the LCDR1, LCDR2, and LCDR3 comprises an amino acid sequence of SEQ ID NOS: 15696, 3982, and 12393, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17274, 4142, and 6474, respectively and the LCDR1, LCDR2, and LCDR3 comprises an amino acid sequence of SEQ ID NOS:16719, 17007, and 3995, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:18064, 18596, and 11174, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 16037, 4425, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11648, 18365, and 17781, respectively and the LCDR1, LCDR2, and LCDR3 comprises an amino acid sequence of SEQ ID NOS:16657, 11895, and 13608, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16800, 9943, and 11124, respectively and the LCDR1, LCDR2, and LCDR3 comprises an amino acid sequence of SEQ ID NOS:16361, 10926, and 11330, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10368, 6784, and 10083, respectively and the LCDR1, LCDR2, and LCDR3 comprises an amino acid sequence of SEQ ID NOS:11942, 10023, and 13329, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 15941, 9494, and 10463, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:10806, 18275, and 10475, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 17766, and 10239, respectively and the LCDR1, LCDR2, and LCDR3 comprises an amino acid sequence of SEQ ID NOS:16121, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 16965, and 6472, respectively and the LCDR1, LCDR2, and LCDR3 comprises an amino acid sequence of SEQ ID NOS:18070, 18595, and 11177, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:10865, 18275, and 12914, respectively. having a LCDR1, LCDR2, and LCDR3, wherein the HCDR1, HCDR2, and HCDR3 comprises an amino acid sequence of SEQ ID NOS: 16806, 18570, and 7900, respectively and the LCDR1, LCDR2, and LCDR3 comprises an amino acid sequence of SEQ ID NOS:15840, 17664, and 12494, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17021, 10294, and 6090, respectively and the LCDR1, LCDR2, and LCDR3 comprises an amino acid sequence of SEQ ID NOS: 11789, 4420, and 12577, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9986, and 6241, respectively and the LCDR1, LCDR2, and LCDR3 comprises an amino acid sequence of SEQ ID NOS: 16126, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16543, 18475, and 11353, respectively and the LCDR1, LCDR2, and LCDR3 comprises an amino acid sequence of SEQ ID NOS:10873, 18275, and 12991, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6571, 18290, and 9514, respectively and the LCDR1, LCDR2, and LCDR3 comprises an amino acid sequence of SEQ ID NOS:10886, 18265, and 12929, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 15909, 9503, and 12724, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11690, 6786, and 10246, respectively and the LCDR1, LCDR2, and LCDR3 comprises an amino acid sequence of SEQ ID NOS:6690, 10023, and 13330, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17272, 10297, and 6083, respectively and the LCDR1, LCDR2, and LCDR3 comprises an amino acid sequence of SEQ ID NOS: 15842, 4425, and 10444, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17270, 17772, and 10266, respectively and the LCDR1, LCDR2, and LCDR3 comprises an amino acid sequence of SEQ ID NOS:15999, 10264, and 12767, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10353, 18330, and 18097, respectively and the LCDR1, LCDR2, and LCDR3 comprises an amino acid sequence of SEQ ID NOS:15996, 9494, and 11990, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:16729, 17001, and 3995, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11622, 16288, and 10330, respectively and the LCDR1, LCDR2, and LCDR3 comprises an amino acid sequence of SEQ ID NOS:10805, 18276, and 12935, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17474, 9815, and 4249, respectively and the LCDR1, LCDR2, and LCDR3 comprises an amino acid sequence of SEQ ID NOS:15890, 10737, and 12871, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17461, 9789, and 17057, respectively and the LCDR1, LCDR2, and LCDR3 comprises an amino acid sequence of SEQ ID NOS:15896, 10705, and 12804, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11678, 17821, and 10239, respectively and the LCDR1, LCDR2, and LCDR3 comprises an amino acid sequence of SEQ ID NOS:10814, 18275, and 12978, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10364, 6777, and 10082, respectively and the LCDR1, LCDR2, and LCDR3 comprises an amino acid sequence of SEQ ID NOS:11943, 10023, and 13328, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16796, 16939, and 17221, respectively and the LCDR1, LCDR2, and LCDR3 comprises an amino acid sequence of SEQ ID NOS: 17924, 10094, and 13579, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6150, 17731, and 4129, respectively and the LCDR1, LCDR2, and LCDR3 comprises an amino acid sequence of SEQ ID NOS:16399, 10959, and 11256, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 16855, and 10239, respectively and the LCDR1, LCDR2, and LCDR3 comprises an amino acid sequence of SEQ ID NOS:18065, 18593, and 11176, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11361, 9827, and 17583, respectively and the LCDR1, LCDR2, and LCDR3 comprises an amino acid sequence of SEQ ID NOS:16354, 10987, and 11204, respectively. having a LCDR1, LCDR2, and LCDR3, wherein the HCDR1, HCDR2, and HCDR3 comprises an amino acid sequence of SEQ ID NOS:11630, 9960, and 6194, respectively and the LCDR1, LCDR2, and LCDR3 comprises an amino acid sequence of SEQ ID NOS: 16723, 10911, and 4017, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17262, 16321, and 7877, respectively and the LCDR1, LCDR2, and LCDR3 comprises an amino acid sequence of SEQ ID NOS: 16068, 3982, and 12451, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17617, 18362, and 5913, respectively and the LCDR1, LCDR2, and LCDR3 comprises an amino acid sequence of SEQ ID NOS: 15858, 9503, and 12025, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17614, 18482, and 17653, respectively and the LCDR1, LCDR2, and LCDR3 comprises an amino acid sequence of SEQ ID NOS:15861, 11791, and 12843, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 15821, 4425, and 12763, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 15954, 9503, and 12769, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:16452, 18275, and 12914, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16543, 18475, and 11354, respectively and the LCDR1, LCDR2, and LCDR3 comprises an amino acid sequence of SEQ ID NOS:10873, 18275, and 12991, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11633, 16627, and 10988, respectively and the LCDR1, LCDR2, and LCDR3 comprises an amino acid sequence of SEQ ID NOS:15759, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 16735, 10090, and 4004, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6503, 18345, and 10333, respectively and the LCDR1, LCDR2, and LCDR3 comprises an amino acid sequence of SEQ ID NOS:10793, 18260, and 12898, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:10712, 4425, and 12406, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 17976, 6717, and 4389, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17052, 18459, and 7883, respectively and the LCDR1, LCDR2, and LCDR3 comprises an amino acid sequence of SEQ ID NOS: 16031, 3982, and 12485, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:10804, 18275, and 12994, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9578, and 4340, respectively and the LCDR1, LCDR2, and LCDR3 comprises an amino acid sequence of SEQ ID NOS:15890, 10917, and 12016, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10152, 18412, and 4497, respectively and the LCDR1, LCDR2, and LCDR3 comprises an amino acid sequence of SEQ ID NOS: 17974, 6307, and 17171, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11622, 18347, and 10331, respectively and the LCDR1, LCDR2, and LCDR3 comprises an amino acid sequence of SEQ ID NOS:10805, 18275, and 12935, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9747, and 4230, respectively and the LCDR1, LCDR2, and LCDR3 comprises an amino acid sequence of SEQ ID NOS: 16070, 9503, and 12778, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11650, 11027, and 10239, respectively and the LCDR1, LCDR2, and LCDR3 comprises an amino acid sequence of SEQ ID NOS: 16646, 10747, and 13032, respectively. having a LCDR1, LCDR2, and LCDR3, wherein the HCDR1, HCDR2, and HCDR3 comprises an amino acid sequence of SEQ ID NOS:16571, 9985, and 18421, respectively and the LCDR1, LCDR2, and LCDR3 comprises an amino acid sequence of SEQ ID NOS: 10836, 9267, and 11332, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 17977, 11605, and 16474, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10935, 9723, and 10214, respectively and the LCDR1, LCDR2, and LCDR3 comprises an amino acid sequence of SEQ ID NOS: 10824, 7822, and 11240, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4304, 9783, and 18227, respectively and the LCDR1, LCDR2, and LCDR3 comprises an amino acid sequence of SEQ ID NOS:10058, 17222, and 18116, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:16636, 10771, and 10285, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6509, 9953, and 4195, respectively and the LCDR1, LCDR2, and LCDR3 comprises an amino acid sequence of SEQ ID NOS: 15628, 9503, and 12763, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10368, 18513, and 11939, respectively and the LCDR1, LCDR2, and LCDR3 comprises an amino acid sequence of SEQ ID NOS:9653, 4467, and 15571, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 16000, 9503, and 12586, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:16712, 18105, and 4284, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:15824, 11793, and 12816, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10337, 9990, and 9456, respectively and the LCDR1, LCDR2, and LCDR3 comprises an amino acid sequence of SEQ ID NOS: 16059, 4432, and 12004, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:10869, 18275, and 12986, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11632, 16609, and 6471, respectively and the LCDR1, LCDR2, and LCDR3 comprises an amino acid sequence of SEQ ID NOS:18068, 18596, and 11181, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11634, 17778, and 10239, respectively and the LCDR1, LCDR2, and LCDR3 comprises an amino acid sequence of SEQ ID NOS: 11784, 4609, and 12398, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:16756, 17001, and 3995, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6571, 18314, and 9513, respectively and the LCDR1, LCDR2, and LCDR3 comprises an amino acid sequence of SEQ ID NOS:10846, 18268, and 12929, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:15742, 11791, and 12816, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9339, 9972, and 10239, respectively and the LCDR1, LCDR2, and LCDR3 comprises an amino acid sequence of SEQ ID NOS: 15879, 9503, and 12791, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11634, 11026, and 10239, respectively and the LCDR1, LCDR2, and LCDR3 comprises an amino acid sequence of SEQ ID NOS:11784, 4421, and 12343, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11573, 9909, and 10962, respectively and the LCDR1, LCDR2, and LCDR3 comprises an amino acid sequence of SEQ ID NOS:15795, 10731, and 12873, respectively. having a LCDR1, LCDR2, and LCDR3, wherein the HCDR1, HCDR2, and HCDR3 comprises an amino acid sequence of SEQ ID NOS: 11652, 4270, and 6497, respectively and the LCDR1, LCDR2, and LCDR3 comprises an amino acid sequence of SEQ ID NOS:16720, 10081, and 11133, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:10857, 18257, and 12972, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11385, 10012, and 4339, respectively and the LCDR1, LCDR2, and LCDR3 comprises an amino acid sequence of SEQ ID NOS:15890, 10726, and 11992, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11389, 17779, and 6098, respectively and the LCDR1, LCDR2, and LCDR3 comprises an amino acid sequence of SEQ ID NOS: 11784, 4420, and 12399, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17064, 16829, and 18282, respectively and the LCDR1, LCDR2, and LCDR3 comprises an amino acid sequence of SEQ ID NOS: 10830, 9267, and 11314, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17019, 11534, and 18454, respectively and the LCDR1, LCDR2, and LCDR3 comprises an amino acid sequence of SEQ ID NOS: 16408, 10990, and 11196, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11421, 16905, and 6177, respectively and the LCDR1, LCDR2, and LCDR3 comprises an amino acid sequence of SEQ ID NOS: 15925, 4417, and 12341, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6578, 18547, and 4278, respectively and the LCDR1, LCDR2, and LCDR3 comprises an amino acid sequence of SEQ ID NOS:16712, 18105, and 4284, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:11783, 4416, and 12722, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11679, 11474, and 6040, respectively and the LCDR1, LCDR2, and LCDR3 comprises an amino acid sequence of SEQ ID NOS:16441, 10926, and 11287, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11678, 11469, and 10527, respectively and the LCDR1, LCDR2, and LCDR3 comprises an amino acid sequence of SEQ ID NOS:10804, 18275, and 12897, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4634, 18223, and 17251, respectively and the LCDR1, LCDR2, and LCDR3 comprises an amino acid sequence of SEQ ID NOS: 15678, 6324, and 12378, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11385, 10012, and 4339, respectively and the LCDR1, LCDR2, and LCDR3 comprises an amino acid sequence of SEQ ID NOS:16712, 18105, and 4284, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 15807, 3953, and 12496, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11678, 11490, and 10239, respectively and the LCDR1, LCDR2, and LCDR3 comprises an amino acid sequence of SEQ ID NOS:10875, 18271, and 12900, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11405, 16908, and 4510, respectively and the LCDR1, LCDR2, and LCDR3 comprises an amino acid sequence of SEQ ID NOS: 15902, 9499, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11629, 16877, and 10239, respectively and the LCDR1, LCDR2, and LCDR3 comprises an amino acid sequence of SEQ ID NOS: 16765, 17008, and 3996, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17053, 11007, and 17912, respectively and the LCDR1, LCDR2, and LCDR3 comprises an amino acid sequence of SEQ ID NOS: 15833, 3953, and 12464, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS: 16045, 9507, and 12468, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16962, 17821, and 10239, respectively and the LCDR1, LCDR2, and LCDR3 comprises an amino acid sequence of SEQ ID NOS:18062, 18596, and 11359, respectively. having a LCDR1, LCDR2, and LCDR3, wherein the HCDR1, HCDR2, and HCDR3 comprises an amino acid sequence of SEQ ID NOS:11622, 16287, and 10334, respectively and the LCDR1, LCDR2, and LCDR3 comprises an amino acid sequence of SEQ ID NOS:10800, 18275, and 12898, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17856, 18588, and 10184, respectively and the LCDR1, LCDR2, and LCDR3 comprises an amino acid sequence of SEQ ID NOS: 11789, 4420, and 12572, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:7871, 9963, and 10342, respectively and the LCDR1, LCDR2, and LCDR3 comprises an amino acid sequence of SEQ ID NOS: 16005, 9494, and 12638, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11669, 16890, and 10055, respectively and the LCDR1, LCDR2, and LCDR3 comprises an amino acid sequence of SEQ ID NOS: 16036, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9776, and 16193, respectively and the LCDR1, LCDR2, and LCDR3 comprises an amino acid sequence of SEQ ID NOS:15890, 10727, and 12017, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6509, 9971, and 4064, respectively and the LCDR1, LCDR2, and LCDR3 comprises an amino acid sequence of SEQ ID NOS: 15620, 4436, and 12791, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17129, 18028, and 6013, respectively and the LCDR1, LCDR2, and LCDR3 comprises an amino acid sequence of SEQ ID NOS: 17950, 9261, and 17151, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6573, 18297, and 10014, respectively and the LCDR1, LCDR2, and LCDR3 comprises an amino acid sequence of SEQ ID NOS: 16090, 9503, and 12634, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11629, 4662, and 17054, respectively and the LCDR1, LCDR2, and LCDR3 comprises an amino acid sequence of SEQ ID NOS: 15671, 3982, and 12101, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11690, 16312, and 3947, respectively and the LCDR1, LCDR2, and LCDR3 comprises an amino acid sequence of SEQ ID NOS:10859, 18275, and 12980, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11384, 9725, and 16160, respectively and the LCDR1, LCDR2, and LCDR3 comprises an amino acid sequence of SEQ ID NOS:15861, 10727, and 12890, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 16919, and 9664, respectively and the LCDR1, LCDR2, and LCDR3 comprises an amino acid sequence of SEQ ID NOS:18090, 11073, and 13616, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6558, 9697, and 6111, respectively and the LCDR1, LCDR2, and LCDR3 comprises an amino acid sequence of SEQ ID NOS: 15844, 3969, and 11925, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17534, 10567, and 16180, respectively and the LCDR1, LCDR2, and LCDR3 comprises an amino acid sequence of SEQ ID NOS:17878, 6633, and 17156, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 18637, 18368, and 6493, respectively and the LCDR1, LCDR2, and LCDR3 comprises an amino acid sequence of SEQ ID NOS:15750, 10986, and 12453, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6577, 18468, and 4190, respectively and the LCDR1, LCDR2, and LCDR3 comprises an amino acid sequence of SEQ ID NOS:10852, 18271, and 12031, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17882, 9899, and 9591, respectively and the LCDR1, LCDR2, and LCDR3 comprises an amino acid sequence of SEQ ID NOS: 16078, 4417, and 12446, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17601, 12039, and 6074, respectively and the LCDR1, LCDR2, and LCDR3 comprises an amino acid sequence of SEQ ID NOS:15797, 10731, and 12017, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10783, 11453, and 10315, respectively and the LCDR1, LCDR2, and LCDR3 comprises an amino acid sequence of SEQ ID NOS: 15743, 11792, and 12883, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16608, 9741, and 10169, respectively and the LCDR1, LCDR2, and LCDR3 comprises an amino acid sequence of SEQ ID NOS: 16056, 9503, and 12787, respectively. having a LCDR1, LCDR2, and LCDR3, wherein the HCDR1, HCDR2, and HCDR3 comprises an amino acid sequence of SEQ ID NOS:16779, 16311, and 17597, respectively and the LCDR1, LCDR2, and LCDR3 comprises an amino acid sequence of SEQ ID NOS:16408, 10987, and 11214, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11657, 10545, and 6027, respectively and the LCDR1, LCDR2, and LCDR3 comprises an amino acid sequence of SEQ ID NOS:16700, 11902, and 18622, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17432, 10411, and 4587, respectively and the LCDR1, LCDR2, and LCDR3 comprises an amino acid sequence of SEQ ID NOS: 17954, 9258, and 17179, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11631, 17594, and 6173, respectively and the LCDR1, LCDR2, and LCDR3 comprises an amino acid sequence of SEQ ID NOS: 16056, 3983, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17439, 17808, and 18106, respectively and the LCDR1, LCDR2, and LCDR3 comprises an amino acid sequence of SEQ ID NOS:16670, 11902, and 11941, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17650, 16221, and 18139, respectively and the LCDR1, LCDR2, and LCDR3 comprises an amino acid sequence of SEQ ID NOS:15912, 9503, and 12364, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11392, 18554, and 6695, respectively and the LCDR1, LCDR2, and LCDR3 comprises an amino acid sequence of SEQ ID NOS: 16056, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17838, 16259, and 5962, respectively and the LCDR1, LCDR2, and LCDR3 comprises an amino acid sequence of SEQ ID NOS:16639, 10747, and 13024, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17440, 9799, and 10964, respectively and the LCDR1, LCDR2, and LCDR3 comprises an amino acid sequence of SEQ ID NOS: 10830, 9260, and 11170, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17018, 9720, and 9521, respectively and the LCDR1, LCDR2, and LCDR3 comprises an amino acid sequence of SEQ ID NOS:10830, 7821, and 11337, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11393, 18543, and 17700, respectively and the LCDR1, LCDR2, and LCDR3 comprises an amino acid sequence of SEQ ID NOS:15961, 9503, and 12613, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17621, 17747, and 11953, respectively and the LCDR1, LCDR2, and LCDR3 comprises an amino acid sequence of SEQ ID NOS: 15853, 16557, and 12542, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11623, 9844, and 11906, respectively and the LCDR1, LCDR2, and LCDR3 comprises an amino acid sequence of SEQ ID NOS: 15924, 4425, and 12441, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9685, 4253, and 6247, respectively and the LCDR1, LCDR2, and LCDR3 comprises an amino acid sequence of SEQ ID NOS:15713, 16144, and 12390, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16954, and 17873, respectively and the LCDR1, LCDR2, and LCDR3 comprises an amino acid sequence of SEQ ID NOS: 17924, 10094, and 13578, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9796, and 11970, respectively and the LCDR1, LCDR2, and LCDR3 comprises an amino acid sequence of SEQ ID NOS: 16045, 9503, and 12759, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6527, 6744, and 10502, respectively and the LCDR1, LCDR2, and LCDR3 comprises an amino acid sequence of SEQ ID NOS:17566, 10922, and 11229, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17466, 18537, and 16599, respectively and the LCDR1, LCDR2, and LCDR3 comprises an amino acid sequence of SEQ ID NOS: 16045, 9503, and 12947, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11633, 4279, and 11702, respectively and the LCDR1, LCDR2, and LCDR3 comprises an amino acid sequence of SEQ ID NOS: 16036, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9685, 4254, and 6246, respectively and the LCDR1, LCDR2, and LCDR3 comprises an amino acid sequence of SEQ ID NOS:15707, 16554, and 12382, respectively. having a LCDR1, LCDR2, and LCDR3, wherein the HCDR1, HCDR2, and HCDR3 comprises an amino acid sequence of SEQ ID NOS:7809, 10037, and 10032, respectively and the LCDR1, LCDR2, and LCDR3 comprises an amino acid sequence of SEQ ID NOS:16464, 10990, and 11251, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6509, 9971, and 4063, respectively and the LCDR1, LCDR2, and LCDR3 comprises an amino acid sequence of SEQ ID NOS: 15620, 4436, and 12791, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11650, 10562, and 10103, respectively and the LCDR1, LCDR2, and LCDR3 comprises an amino acid sequence of SEQ ID NOS:17950, 6237, and 17177, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6525, 16211, and 6729, respectively and the LCDR1, LCDR2, and LCDR3 comprises an amino acid sequence of SEQ ID NOS:16408, 10985, and 11328, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11623, 9782, and 11905, respectively and the LCDR1, LCDR2, and LCDR3 comprises an amino acid sequence of SEQ ID NOS: 16024, 9480, and 10719, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17887, 9898, and 9590, respectively and the LCDR1, LCDR2, and LCDR3 comprises an amino acid sequence of SEQ ID NOS: 16078, 4417, and 12446, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16324, 16884, and 17217, respectively and the LCDR1, LCDR2, and LCDR3 comprises an amino acid sequence of SEQ ID NOS: 15668, 3969, and 12333, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:7806, 9564, and 10433, respectively and the LCDR1, LCDR2, and LCDR3 comprises an amino acid sequence of SEQ ID NOS: 16009, 4425, and 12439, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10057, 9563, and 10432, respectively and the LCDR1, LCDR2, and LCDR3 comprises an amino acid sequence of SEQ ID NOS: 16067, 4425, and 12423, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11652, 9396, and 10157, respectively and the LCDR1, LCDR2, and LCDR3 comprises an amino acid sequence of SEQ ID NOS:16036, 9471, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17264, 9829, and 16530, respectively and the LCDR1, LCDR2, and LCDR3 comprises an amino acid sequence of SEQ ID NOS: 16061, 9480, and 12649, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10681, 4108, and 4325, respectively and the LCDR1, LCDR2, and LCDR3 comprises an amino acid sequence of SEQ ID NOS: 15694, 3971, and 11086, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11647, 10296, and 18289, respectively and the LCDR1, LCDR2, and LCDR3 comprises an amino acid sequence of SEQ ID NOS: 15911, 10731, and 12876, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4136, and 18131, respectively and the LCDR1, LCDR2, and LCDR3 comprises an amino acid sequence of SEQ ID NOS: 17959, 6312, and 4375, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16986, 9825, and 10492, respectively and the LCDR1, LCDR2, and LCDR3 comprises an amino acid sequence of SEQ ID NOS: 15616, 3982, and 12850, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17634, 18503, and 17705, respectively and the LCDR1, LCDR2, and LCDR3 comprises an amino acid sequence of SEQ ID NOS: 15998, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9709, and 9454, respectively and the LCDR1, LCDR2, and LCDR3 comprises an amino acid sequence of SEQ ID NOS: 15717, 3982, and 12095, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 10555, and 6706, respectively and the LCDR1, LCDR2, and LCDR3 comprises an amino acid sequence of SEQ ID NOS: 16045, 9503, and 12753, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11653, 9773, and 17903, respectively and the LCDR1, LCDR2, and LCDR3 comprises an amino acid sequence of SEQ ID NOS:15890, 10731, and 12606, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10348, 6778, and 11078, respectively and the LCDR1, LCDR2, and LCDR3 comprises an amino acid sequence of SEQ ID NOS: 10825, 9267, and 11326, respectively. having a LCDR1, LCDR2, and LCDR3, wherein the HCDR1, HCDR2, and HCDR3 comprises an amino acid sequence of SEQ ID NOS:17018, 9721, and 9522, respectively and the LCDR1, LCDR2, and LCDR3 comprises an amino acid sequence of SEQ ID NOS: 10830, 9267, and 11337, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17432, 18531, and 17723, respectively and the LCDR1, LCDR2, and LCDR3 comprises an amino acid sequence of SEQ ID NOS: 16061, 9494, and 12659, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6186, 11005, and 6712, respectively and the LCDR1, LCDR2, and LCDR3 comprises an amino acid sequence of SEQ ID NOS:16417, 10982, and 11342, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16990, 9801, and 10206, respectively and the LCDR1, LCDR2, and LCDR3 comprises an amino acid sequence of SEQ ID NOS: 10786, 9267, and 11317, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17045, 12040, and 6073, respectively and the LCDR1, LCDR2, and LCDR3 comprises an amino acid sequence of SEQ ID NOS:15734, 10740, and 12028, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17534, 10571, and 16167, respectively and the LCDR1, LCDR2, and LCDR3 comprises an amino acid sequence of SEQ ID NOS: 15972, 4422, and 10445, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9987, and 9615, respectively and the LCDR1, LCDR2, and LCDR3 comprises an amino acid sequence of SEQ ID NOS:16406, 11126, and 11200, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6531, 9694, and 9560, respectively and the LCDR1, LCDR2, and LCDR3 comprises an amino acid sequence of SEQ ID NOS: 17966, 9278, and 4364, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6508, 9956, and 4175, respectively and the LCDR1, LCDR2, and LCDR3 comprises an amino acid sequence of SEQ ID NOS: 15610, 9503, and 12766, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11609, 10401, and 6718, respectively and the LCDR1, LCDR2, and LCDR3 comprises an amino acid sequence of SEQ ID NOS:15712, 3987, and 12332, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11622, 9927, and 16583, respectively and the LCDR1, LCDR2, and LCDR3 comprises an amino acid sequence of SEQ ID NOS: 10829, 9272, and 11303, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17856, 18585, and 9848, respectively and the LCDR1, LCDR2, and LCDR3 comprises an amino acid sequence of SEQ ID NOS:10890, 18275, and 12996, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11689, 18592, and 16604, respectively and the LCDR1, LCDR2, and LCDR3 comprises an amino acid sequence of SEQ ID NOS: 16772, 16331, and 4009, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 18637, 18376, and 6492, respectively and the LCDR1, LCDR2, and LCDR3 comprises an amino acid sequence of SEQ ID NOS:15747, 11144, and 12453, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10368, 6677, and 9569, respectively and the LCDR1, LCDR2, and LCDR3 comprises an amino acid sequence of SEQ ID NOS: 16734, 5977, and 10283, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6188, 9256, and 12407, respectively and the LCDR1, LCDR2, and LCDR3 comprises an amino acid sequence of SEQ ID NOS:10892, 18275, and 12938, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17990, 16893, and 17049, respectively and the LCDR1, LCDR2, and LCDR3 comprises an amino acid sequence of SEQ ID NOS: 16069, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11623, 9941, and 11550, respectively and the LCDR1, LCDR2, and LCDR3 comprises an amino acid sequence of SEQ ID NOS: 15691, 4282, and 11096, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6512, 9419, and 11716, respectively and the LCDR1, LCDR2, and LCDR3 comprises an amino acid sequence of SEQ ID NOS: 17932, 6307, and 16978, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10367, 18323, and 9457, respectively and the LCDR1, LCDR2, and LCDR3 comprises an amino acid sequence of SEQ ID NOS: 15704, 3982, and 12098, respectively. having a LCDR1, LCDR2, and LCDR3, wherein the HCDR1, HCDR2, and HCDR3 comprises an amino acid sequence of SEQ ID NOS: 10365, 18294, and 6542, respectively and the LCDR1, LCDR2, and LCDR3 comprises an amino acid sequence of SEQ ID NOS: 18094, 17718, and 9443, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17262, 9900, and 16589, respectively and the LCDR1, LCDR2, and LCDR3 comprises an amino acid sequence of SEQ ID NOS: 10829, 9267, and 11232, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16816, 10568, and 16155, respectively and the LCDR1, LCDR2, and LCDR3 comprises an amino acid sequence of SEQ ID NOS: 15724, 4425, and 11102, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17018, 9720, and 9521, respectively and the LCDR1, LCDR2, and LCDR3 comprises an amino acid sequence of SEQ ID NOS: 10830, 9267, and 11337, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17987, 9998, and 7861, respectively and the LCDR1, LCDR2, and LCDR3 comprises an amino acid sequence of SEQ ID NOS: 10828, 9267, and 11340, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6186, 11004, and 6711, respectively and the LCDR1, LCDR2, and LCDR3 comprises an amino acid sequence of SEQ ID NOS:16455, 10990, and 11342, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10355, 18540, and 17703, respectively and the LCDR1, LCDR2, and LCDR3 comprises an amino acid sequence of SEQ ID NOS: 15980, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17466, 18541, and 17704, respectively and the LCDR1, LCDR2, and LCDR3 comprises an amino acid sequence of SEQ ID NOS:18270, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16610, 9931, and 4204, respectively and the LCDR1, LCDR2, and LCDR3 comprises an amino acid sequence of SEQ ID NOS: 16045, 9503, and 12780, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:7806, 10036, and 10031, respectively and the LCDR1, LCDR2, and LCDR3 comprises an amino acid sequence of SEQ ID NOS:10834, 10990, and 11251, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16681, 18572, and 5941, respectively and the LCDR1, LCDR2, and LCDR3 comprises an amino acid sequence of SEQ ID NOS: 15844, 3969, and 12530, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9806, and 4269, respectively and the LCDR1, LCDR2, and LCDR3 comprises an amino acid sequence of SEQ ID NOS:16372, 10925, and 11218, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9701, and 4216, respectively and the LCDR1, LCDR2, and LCDR3 comprises an amino acid sequence of SEQ ID NOS: 16089, 9480, and 12653, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17477, 16834, and 6174, respectively and the LCDR1, LCDR2, and LCDR3 comprises an amino acid sequence of SEQ ID NOS: 15940, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9955, and 16966, respectively and the LCDR1, LCDR2, and LCDR3 comprises an amino acid sequence of SEQ ID NOS:15622, 10224, and 12766, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11642, 12048, and 9646, respectively and the LCDR1, LCDR2, and LCDR3 comprises an amino acid sequence of SEQ ID NOS: 15830, 4425, and 12444, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9731, and 9526, respectively and the LCDR1, LCDR2, and LCDR3 comprises an amino acid sequence of SEQ ID NOS: 16061, 9494, and 12639, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11651, 18004, and 5934, respectively and the LCDR1, LCDR2, and LCDR3 comprises an amino acid sequence of SEQ ID NOS: 15975, 4417, and 10711, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11623, 9708, and 10204, respectively and the LCDR1, LCDR2, and LCDR3 comprises an amino acid sequence of SEQ ID NOS: 16028, 9494, and 12627, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16573, 9842, and 10970, respectively and the LCDR1, LCDR2, and LCDR3 comprises an amino acid sequence of SEQ ID NOS:9486, 4415, and 12635, respectively. having a LCDR1, LCDR2, and LCDR3, wherein the HCDR1, HCDR2, and HCDR3 comprises an amino acid sequence of SEQ ID NOS:11578, 18534, and 17705, respectively and the LCDR1, LCDR2, and LCDR3 comprises an amino acid sequence of SEQ ID NOS: 15879, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16816, 11519, and 16476, respectively and the LCDR1, LCDR2, and LCDR3 comprises an amino acid sequence of SEQ ID NOS:16032, 6608, and 12421, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17620, 17773, and 16607, respectively and the LCDR1, LCDR2, and LCDR3 comprises an amino acid sequence of SEQ ID NOS: 17871, 6624, and 13564, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6506, 4660, and 11568, respectively and the LCDR1, LCDR2, and LCDR3 comprises an amino acid sequence of SEQ ID NOS: 15720, 9498, and 12101, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10689, 18549, and 10260, respectively and the LCDR1, LCDR2, and LCDR3 comprises an amino acid sequence of SEQ ID NOS: 15854, 9503, and 12769, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17642, 11522, and 16469, respectively and the LCDR1, LCDR2, and LCDR3 comprises an amino acid sequence of SEQ ID NOS: 16033, 4425, and 12445, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11625, 10011, and 4024, respectively and the LCDR1, LCDR2, and LCDR3 comprises an amino acid sequence of SEQ ID NOS: 15691, 3969, and 11094, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17466, 16919, and 10245, respectively and the LCDR1, LCDR2, and LCDR3 comprises an amino acid sequence of SEQ ID NOS:18077, 18596, and 11185, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10681, 16859, and 16548, respectively and the LCDR1, LCDR2, and LCDR3 comprises an amino acid sequence of SEQ ID NOS:15689, 3969, and 11099, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17630, 9402, and 6115, respectively and the LCDR1, LCDR2, and LCDR3 comprises an amino acid sequence of SEQ ID NOS:11703, 6333, and 4387, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17615, 4280, and 5943, respectively and the LCDR1, LCDR2, and LCDR3 comprises an amino acid sequence of SEQ ID NOS: 16045, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16618, 9751, and 11376, respectively and the LCDR1, LCDR2, and LCDR3 comprises an amino acid sequence of SEQ ID NOS: 16049, 9494, and 12687, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9776, and 10106, respectively and the LCDR1, LCDR2, and LCDR3 comprises an amino acid sequence of SEQ ID NOS: 16061, 9494, and 12660, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 16954, and 16704, respectively and the LCDR1, LCDR2, and LCDR3 comprises an amino acid sequence of SEQ ID NOS: 16045, 9503, and 12776, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4305, 9712, and 3943, respectively and the LCDR1, LCDR2, and LCDR3 comprises an amino acid sequence of SEQ ID NOS: 17959, 9275, and 17162, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16549, 18572, and 5940, respectively and the LCDR1, LCDR2, and LCDR3 comprises an amino acid sequence of SEQ ID NOS: 15844, 3969, and 12518, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17637, 17778, and 4489, respectively and the LCDR1, LCDR2, and LCDR3 comprises an amino acid sequence of SEQ ID NOS: 17947, 9275, and 4345, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17837, 11470, and 7884, respectively and the LCDR1, LCDR2, and LCDR3 comprises an amino acid sequence of SEQ ID NOS:15913, 10728, and 12742, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9727, and 17116, respectively and the LCDR1, LCDR2, and LCDR3 comprises an amino acid sequence of SEQ ID NOS: 10830, 9269, and 11311, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17466, 18494, and 5923, respectively and the LCDR1, LCDR2, and LCDR3 comprises an amino acid sequence of SEQ ID NOS: 16045, 9503, and 12775, respectively. having a LCDR1, LCDR2, and LCDR3, wherein the HCDR1, HCDR2, and HCDR3 comprises an amino acid sequence of SEQ ID NOS:10077, 17797, and 16188, respectively and the LCDR1, LCDR2, and LCDR3 comprises an amino acid sequence of SEQ ID NOS:17973, 9271, and 17574, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16608, 9748, and 10380, respectively and the LCDR1, LCDR2, and LCDR3 comprises an amino acid sequence of SEQ ID NOS: 10902, 9267, and 11324, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17030, 16949, and 10265, respectively and the LCDR1, LCDR2, and LCDR3 comprises an amino acid sequence of SEQ ID NOS:16659, 11895, and 11882, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16537, 16903, and 10157, respectively and the LCDR1, LCDR2, and LCDR3 comprises an amino acid sequence of SEQ ID NOS: 16069, 9472, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16779, 16307, and 16179, respectively and the LCDR1, LCDR2, and LCDR3 comprises an amino acid sequence of SEQ ID NOS:15721, 3958, and 12361, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17855, 16302, and 7924, respectively and the LCDR1, LCDR2, and LCDR3 comprises an amino acid sequence of SEQ ID NOS: 15872, 3985, and 12549, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17842, 16846, and 18226, respectively and the LCDR1, LCDR2, and LCDR3 comprises an amino acid sequence of SEQ ID NOS: 15860, 4425, and 12369, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16152, 16979, and 18438, respectively and the LCDR1, LCDR2, and LCDR3 comprises an amino acid sequence of SEQ ID NOS: 15788, 4425, and 12403, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17479, 17758, and 18214, respectively and the LCDR1, LCDR2, and LCDR3 comprises an amino acid sequence of SEQ ID NOS:17956, 6460, and 17189, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6507, 9971, and 4063, respectively and the LCDR1, LCDR2, and LCDR3 comprises an amino acid sequence of SEQ ID NOS:15620, 4436, and 12791, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17634, 18548, and 17702, respectively and the LCDR1, LCDR2, and LCDR3 comprises an amino acid sequence of SEQ ID NOS: 15624, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17457, 9847, and 10583, respectively and the LCDR1, LCDR2, and LCDR3 comprises an amino acid sequence of SEQ ID NOS: 16052, 9493, and 12671, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 11488, and 6143, respectively and the LCDR1, LCDR2, and LCDR3 comprises an amino acid sequence of SEQ ID NOS: 16045, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9831, and 10189, respectively and the LCDR1, LCDR2, and LCDR3 comprises an amino acid sequence of SEQ ID NOS:11596, 10928, and 11231, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9837, and 4229, respectively and the LCDR1, LCDR2, and LCDR3 comprises an amino acid sequence of SEQ ID NOS: 10851, 9267, and 11340, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10365, 18429, and 6543, respectively and the LCDR1, LCDR2, and LCDR3 comprises an amino acid sequence of SEQ ID NOS: 18093, 17721, and 9450, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 17768, and 6452, respectively and the LCDR1, LCDR2, and LCDR3 comprises an amino acid sequence of SEQ ID NOS:15945, 10720, and 12356, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18352, and 4055, respectively and the LCDR1, LCDR2, and LCDR3 comprises an amino acid sequence of SEQ ID NOS:16659, 11900, and 11877, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 4661, and 16679, respectively and the LCDR1, LCDR2, and LCDR3 comprises an amino acid sequence of SEQ ID NOS: 16045, 3983, and 12815, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17432, 18563, and 17861, respectively and the LCDR1, LCDR2, and LCDR3 comprises an amino acid sequence of SEQ ID NOS:16668, 11904, and 11872, respectively. having a LCDR1, LCDR2, and LCDR3, wherein the HCDR1, HCDR2, and HCDR3 comprises an amino acid sequence of SEQ ID NOS: 17449, 9780, and 4209, respectively and the LCDR1, LCDR2, and LCDR3 comprises an amino acid sequence of SEQ ID NOS:15866, 10727, and 12602, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 18637, 18369, and 6495, respectively and the LCDR1, LCDR2, and LCDR3 comprises an amino acid sequence of SEQ ID NOS:15751, 11144, and 12453, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10935, 10085, and 4028, respectively and the LCDR1, LCDR2, and LCDR3 comprises an amino acid sequence of SEQ ID NOS: 16045, 9503, and 12748, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6530, 9916, and 6026, respectively and the LCDR1, LCDR2, and LCDR3 comprises an amino acid sequence of SEQ ID NOS: 15704, 3969, and 12827, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17476, 16834, and 6174, respectively and the LCDR1, LCDR2, and LCDR3 comprises an amino acid sequence of SEQ ID NOS: 16083, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10190, 18291, and 17714, respectively and the LCDR1, LCDR2, and LCDR3 comprises an amino acid sequence of SEQ ID NOS: 15963, 10727, and 12466, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11686, 9405, and 16616, respectively and the LCDR1, LCDR2, and LCDR3 comprises an amino acid sequence of SEQ ID NOS: 15629, 4258, and 12277, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10365, 18295, and 6542, respectively and the LCDR1, LCDR2, and LCDR3 comprises an amino acid sequence of SEQ ID NOS: 18093, 17718, and 9445, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11632, 9923, and 16166, respectively and the LCDR1, LCDR2, and LCDR3 comprises an amino acid sequence of SEQ ID NOS: 18092, 10968, and 7953, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6509, 9958, and 4175, respectively and the LCDR1, LCDR2, and LCDR3 comprises an amino acid sequence of SEQ ID NOS:15612, 9500, and 12766, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11673, 17814, and 16183, respectively and the LCDR1, LCDR2, and LCDR3 comprises an amino acid sequence of SEQ ID NOS: 17876, 6626, and 17572, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 18637, 18376, and 6492, respectively and the LCDR1, LCDR2, and LCDR3 comprises an amino acid sequence of SEQ ID NOS:15751, 11144, and 12453, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11647, 18366, and 6476, respectively and the LCDR1, LCDR2, and LCDR3 comprises an amino acid sequence of SEQ ID NOS:15877, 11144, and 12495, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6508, 10000, and 4605, respectively and the LCDR1, LCDR2, and LCDR3 comprises an amino acid sequence of SEQ ID NOS: 16045, 9501, and 12745, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 4136, and 11697, respectively and the LCDR1, LCDR2, and LCDR3 comprises an amino acid sequence of SEQ ID NOS:16659, 11900, and 11884, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9751, and 11376, respectively and the LCDR1, LCDR2, and LCDR3 comprises an amino acid sequence of SEQ ID NOS: 16061, 9494, and 12628, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17261, 10004, and 17689, respectively and the LCDR1, LCDR2, and LCDR3 comprises an amino acid sequence of SEQ ID NOS: 16061, 9494, and 12633, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11666, 9962, and 9585, respectively and the LCDR1, LCDR2, and LCDR3 comprises an amino acid sequence of SEQ ID NOS: 16726, 11556, and 4244, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4182, 18313, and 18145, respectively and the LCDR1, LCDR2, and LCDR3 comprises an amino acid sequence of SEQ ID NOS: 17963, 9275, and 11128, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9512, 4145, and 17660, respectively and the LCDR1, LCDR2, and LCDR3 comprises an amino acid sequence of SEQ ID NOS: 16061, 9494, and 10462, respectively. having a LCDR1, LCDR2, and LCDR3, wherein the HCDR1, HCDR2, and HCDR3 comprises an amino acid sequence of SEQ ID NOS:10945, 9766, and 16997, respectively and the LCDR1, LCDR2, and LCDR3 comprises an amino acid sequence of SEQ ID NOS: 16001, 9494, and 12720, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10777, 16860, and 6498, respectively and the LCDR1, LCDR2, and LCDR3 comprises an amino acid sequence of SEQ ID NOS:18061, 18606, and 11177, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4182, 18319, and 18145, respectively and the LCDR1, LCDR2, and LCDR3 comprises an amino acid sequence of SEQ ID NOS: 17965, 9275, and 9427, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10359, 16917, and 6125, respectively and the LCDR1, LCDR2, and LCDR3 comprises an amino acid sequence of SEQ ID NOS: 16045, 9480, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17144, 16953, and 16338, respectively and the LCDR1, LCDR2, and LCDR3 comprises an amino acid sequence of SEQ ID NOS: 17959, 6312, and 4357, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17608, 18027, and 5926, respectively and the LCDR1, LCDR2, and LCDR3 comprises an amino acid sequence of SEQ ID NOS: 16067, 4425, and 10711, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17881, 10207, and 11567, respectively and the LCDR1, LCDR2, and LCDR3 comprises an amino acid sequence of SEQ ID NOS:16654, 6636, and 11881, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9718, and 17115, respectively and the LCDR1, LCDR2, and LCDR3 comprises an amino acid sequence of SEQ ID NOS: 15929, 9494, and 10439, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6530, 9916, and 9550, respectively and the LCDR1, LCDR2, and LCDR3 comprises an amino acid sequence of SEQ ID NOS:16045, 10264, and 12813, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16621, 9764, and 6015, respectively and the LCDR1, LCDR2, and LCDR3 comprises an amino acid sequence of SEQ ID NOS: 15664, 9489, and 11109, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6574, 18565, and 18146, respectively and the LCDR1, LCDR2, and LCDR3 comprises an amino acid sequence of SEQ ID NOS: 16459, 4436, and 12771, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:18100, 9868, and 16503, respectively and the LCDR1, LCDR2, and LCDR3 comprises an amino acid sequence of SEQ ID NOS: 16049, 9494, and 12671, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11649, 18374, and 6475, respectively and the LCDR1, LCDR2, and LCDR3 comprises an amino acid sequence of SEQ ID NOS:15868, 11144, and 12463, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10944, 9702, and 10191, respectively and the LCDR1, LCDR2, and LCDR3 comprises an amino acid sequence of SEQ ID NOS: 15947, 3974, and 12648, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11391, 10005, and 10062, respectively and the LCDR1, LCDR2, and LCDR3 comprises an amino acid sequence of SEQ ID NOS:17950, 9261, and 17167, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16473, 11442, and 18607, respectively and the LCDR1, LCDR2, and LCDR3 comprises an amino acid sequence of SEQ ID NOS: 15691, 3982, and 11092, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10753, 17796, and 9424, respectively and the LCDR1, LCDR2, and LCDR3 comprises an amino acid sequence of SEQ ID NOS:15934, 10738, and 11924, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11633, 16915, and 6200, respectively and the LCDR1, LCDR2, and LCDR3 comprises an amino acid sequence of SEQ ID NOS:15662, 16556, and 12981, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6509, 9958, and 17434, respectively and the LCDR1, LCDR2, and LCDR3 comprises an amino acid sequence of SEQ ID NOS: 15630, 9503, and 12794, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9814, and 6117, respectively and the LCDR1, LCDR2, and LCDR3 comprises an amino acid sequence of SEQ ID NOS: 16493, 9503, and 12757, respectively. having a LCDR1, LCDR2, and LCDR3, wherein the HCDR1, HCDR2, and HCDR3 comprises an amino acid sequence of SEQ ID NOS:17991, 11523, and 16218, respectively and the LCDR1, LCDR2, and LCDR3 comprises an amino acid sequence of SEQ ID NOS: 15997, 9464, and 12785, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6579, 18579, and 17055, respectively and the LCDR1, LCDR2, and LCDR3 comprises an amino acid sequence of SEQ ID NOS: 15664, 3953, and 11081, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16819, 11461, and 6466, respectively and the LCDR1, LCDR2, and LCDR3 comprises an amino acid sequence of SEQ ID NOS:15936, 11778, and 12739, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16696, 16937, and 4485, respectively and the LCDR1, LCDR2, and LCDR3 comprises an amino acid sequence of SEQ ID NOS:10843, 18277, and 12925, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17992, 18292, and 18202, respectively and the LCDR1, LCDR2, and LCDR3 comprises an amino acid sequence of SEQ ID NOS:10231, 6218, and 11043, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4619, 9971, and 4636, respectively and the LCDR1, LCDR2, and LCDR3 comprises an amino acid sequence of SEQ ID NOS: 15649, 3969, and 12554, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17262, 9767, and 10243, respectively and the LCDR1, LCDR2, and LCDR3 comprises an amino acid sequence of SEQ ID NOS: 16040, 9503, and 10468, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17141, 11527, and 11773, respectively and the LCDR1, LCDR2, and LCDR3 comprises an amino acid sequence of SEQ ID NOS:17958, 6332, and 17178, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16620, 18493, and 17706, respectively and the LCDR1, LCDR2, and LCDR3 comprises an amino acid sequence of SEQ ID NOS:15946, 9503, and 12725, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17062, 9833, and 10321, respectively and the LCDR1, LCDR2, and LCDR3 comprises an amino acid sequence of SEQ ID NOS: 10830, 9265, and 11316, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6578, 18512, and 18140, respectively and the LCDR1, LCDR2, and LCDR3 comprises an amino acid sequence of SEQ ID NOS:16449, 18274, and 12959, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10367, 18323, and 6618, respectively and the LCDR1, LCDR2, and LCDR3 comprises an amino acid sequence of SEQ ID NOS:18077, 18596, and 11185, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9438, 10248, and 17138, respectively and the LCDR1, LCDR2, and LCDR3 comprises an amino acid sequence of SEQ ID NOS: 16039, 9494, and 12002, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17460, 9746, and 16550, respectively and the LCDR1, LCDR2, and LCDR3 comprises an amino acid sequence of SEQ ID NOS: 16189, 9503, and 12765, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16796, 11518, and 9921, respectively and the LCDR1, LCDR2, and LCDR3 comprises an amino acid sequence of SEQ ID NOS: 17924, 4180, and 13569, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17446, 18501, and 11726, respectively and the LCDR1, LCDR2, and LCDR3 comprises an amino acid sequence of SEQ ID NOS: 16036, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17441, 9779, and 16792, respectively and the LCDR1, LCDR2, and LCDR3 comprises an amino acid sequence of SEQ ID NOS: 15691, 3969, and 11098, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17275, 17756, and 5935, respectively and the LCDR1, LCDR2, and LCDR3 comprises an amino acid sequence of SEQ ID NOS: 16045, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11673, 10568, and 16184, respectively and the LCDR1, LCDR2, and LCDR3 comprises an amino acid sequence of SEQ ID NOS:17877, 6626, and 17158, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4307, 9577, and 6086, respectively and the LCDR1, LCDR2, and LCDR3 comprises an amino acid sequence of SEQ ID NOS: 15964, 4424, and 10711, respectively. having a LCDR1, LCDR2, and LCDR3, wherein the HCDR1, HCDR2, and HCDR3 comprises an amino acid sequence of SEQ ID NOS:11650, 10562, and 10103, respectively and the LCDR1, LCDR2, and LCDR3 comprises an amino acid sequence of SEQ ID NOS:17981, 6237, and 17177, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9945, and 6162, respectively and the LCDR1, LCDR2, and LCDR3 comprises an amino acid sequence of SEQ ID NOS: 10830, 9267, and 11334, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4322, 11483, and 6672, respectively and the LCDR1, LCDR2, and LCDR3 comprises an amino acid sequence of SEQ ID NOS:18085, 18224, and 13625, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17634, 18539, and 17702, respectively and the LCDR1, LCDR2, and LCDR3 comprises an amino acid sequence of SEQ ID NOS: 16045, 10264, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11650, 17802, and 5905, respectively and the LCDR1, LCDR2, and LCDR3 comprises an amino acid sequence of SEQ ID NOS:16408, 10985, and 11253, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16799, 10065, and 11735, respectively and the LCDR1, LCDR2, and LCDR3 comprises an amino acid sequence of SEQ ID NOS: 15958, 9503, and 12353, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 18637, 18375, and 6492, respectively and the LCDR1, LCDR2, and LCDR3 comprises an amino acid sequence of SEQ ID NOS:15751, 11144, and 12454, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9438, 10249, and 17137, respectively and the LCDR1, LCDR2, and LCDR3 comprises an amino acid sequence of SEQ ID NOS: 16080, 9494, and 12614, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11632, 9924, and 6107, respectively and the LCDR1, LCDR2, and LCDR3 comprises an amino acid sequence of SEQ ID NOS:10893, 18275, and 12372, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17991, 17794, and 11112, respectively and the LCDR1, LCDR2, and LCDR3 comprises an amino acid sequence of SEQ ID NOS:15933, 10737, and 12455, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11652, 18489, and 17679, respectively and the LCDR1, LCDR2, and LCDR3 comprises an amino acid sequence of SEQ ID NOS: 16360, 10928, and 11350, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 16885, and 10157, respectively and the LCDR1, LCDR2, and LCDR3 comprises an amino acid sequence of SEQ ID NOS: 16036, 9472, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16546, 18408, and 10613, respectively and the LCDR1, LCDR2, and LCDR3 comprises an amino acid sequence of SEQ ID NOS: 16782, 6244, and 4366, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17466, 18539, and 17705, respectively and the LCDR1, LCDR2, and LCDR3 comprises an amino acid sequence of SEQ ID NOS: 16045, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17018, 9722, and 9525, respectively and the LCDR1, LCDR2, and LCDR3 comprises an amino acid sequence of SEQ ID NOS: 10830, 9267, and 11337, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17443, 17809, and 6533, respectively and the LCDR1, LCDR2, and LCDR3 comprises an amino acid sequence of SEQ ID NOS:18077, 18596, and 11187, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17626, 18633, and 4409, respectively and the LCDR1, LCDR2, and LCDR3 comprises an amino acid sequence of SEQ ID NOS:9671, 4464, and 15501, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11676, 11484, and 10973, respectively and the LCDR1, LCDR2, and LCDR3 comprises an amino acid sequence of SEQ ID NOS:10854, 18271, and 12902, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16692, 18583, and 17696, respectively and the LCDR1, LCDR2, and LCDR3 comprises an amino acid sequence of SEQ ID NOS:16659, 11900, and 11874, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11622, 9769, and 11734, respectively and the LCDR1, LCDR2, and LCDR3 comprises an amino acid sequence of SEQ ID NOS: 15958, 9503, and 12773, respectively. having a LCDR1, LCDR2, and LCDR3, wherein the HCDR1, HCDR2, and HCDR3 comprises an amino acid sequence of SEQ ID NOS:16809, 9708, and 10505, respectively and the LCDR1, LCDR2, and LCDR3 comprises an amino acid sequence of SEQ ID NOS:16006, 10262, and 12702, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17441, 9857, and 11719, respectively and the LCDR1, LCDR2, and LCDR3 comprises an amino acid sequence of SEQ ID NOS:15890, 10705, and 12021, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17262, 9786, and 10242, respectively and the LCDR1, LCDR2, and LCDR3 comprises an amino acid sequence of SEQ ID NOS: 16045, 9503, and 10473, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4619, 9959, and 4314, respectively and the LCDR1, LCDR2, and LCDR3 comprises an amino acid sequence of SEQ ID NOS: 15621, 9501, and 12762, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11650, 10561, and 10104, respectively and the LCDR1, LCDR2, and LCDR3 comprises an amino acid sequence of SEQ ID NOS:17958, 6312, and 17174, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17534, 11526, and 16468, respectively and the LCDR1, LCDR2, and LCDR3 comprises an amino acid sequence of SEQ ID NOS: 15999, 4400, and 10723, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17270, 17778, and 16691, respectively and the LCDR1, LCDR2, and LCDR3 comprises an amino acid sequence of SEQ ID NOS: 17924, 10094, and 13579, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6536, 9377, and 17674, respectively and the LCDR1, LCDR2, and LCDR3 comprises an amino acid sequence of SEQ ID NOS:16362, 16510, and 11219, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11652, 9399, and 10157, respectively and the LCDR1, LCDR2, and LCDR3 comprises an amino acid sequence of SEQ ID NOS: 16036, 9472, and 12724, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17263, 9729, and 4277, respectively and the LCDR1, LCDR2, and LCDR3 comprises an amino acid sequence of SEQ ID NOS: 10830, 9267, and 11309, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10233, 16299, and 4250, respectively and the LCDR1, LCDR2, and LCDR3 comprises an amino acid sequence of SEQ ID NOS:15895, 10699, and 10162, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17476, 16833, and 6174, respectively and the LCDR1, LCDR2, and LCDR3 comprises an amino acid sequence of SEQ ID NOS: 16083, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16199, 9575, and 11696, respectively and the LCDR1, LCDR2, and LCDR3 comprises an amino acid sequence of SEQ ID NOS: 16074, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18352, and 9541, respectively and the LCDR1, LCDR2, and LCDR3 comprises an amino acid sequence of SEQ ID NOS: 16759, 16331, and 4006, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10365, 18324, and 3984, respectively and the LCDR1, LCDR2, and LCDR3 comprises an amino acid sequence of SEQ ID NOS:10896, 18275, and 13011, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17274, 18021, and 6127, respectively and the LCDR1, LCDR2, and LCDR3 comprises an amino acid sequence of SEQ ID NOS: 15598, 6214, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10359, 16887, and 5959, respectively and the LCDR1, LCDR2, and LCDR3 comprises an amino acid sequence of SEQ ID NOS: 16036, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10376, 10015, and 7907, respectively and the LCDR1, LCDR2, and LCDR3 comprises an amino acid sequence of SEQ ID NOS: 15874, 3969, and 10454, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17264, 9702, and 11579, respectively and the LCDR1, LCDR2, and LCDR3 comprises an amino acid sequence of SEQ ID NOS: 16005, 9494, and 12662, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16540, 18020, and 9652, respectively and the LCDR1, LCDR2, and LCDR3 comprises an amino acid sequence of SEQ ID NOS: 16113, 9480, and 12772, respectively. having a LCDR1, LCDR2, and LCDR3, wherein the HCDR1, HCDR2, and HCDR3 comprises an amino acid sequence of SEQ ID NOS: 11632, 9937, and 4626, respectively and the LCDR1, LCDR2, and LCDR3 comprises an amino acid sequence of SEQ ID NOS: 15858, 9503, and 12367, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16988, 9995, and 11581, respectively and the LCDR1, LCDR2, and LCDR3 comprises an amino acid sequence of SEQ ID NOS: 16757, 17007, and 3992, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11630, 17780, and 4481, respectively and the LCDR1, LCDR2, and LCDR3 comprises an amino acid sequence of SEQ ID NOS:16651, 10746, and 13339, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17842, 4665, and 5978, respectively and the LCDR1, LCDR2, and LCDR3 comprises an amino acid sequence of SEQ ID NOS:18084, 11074, and 13622, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6579, 18457, and 16956, respectively and the LCDR1, LCDR2, and LCDR3 comprises an amino acid sequence of SEQ ID NOS: 15691, 3969, and 11081, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 16889, and 10166, respectively and the LCDR1, LCDR2, and LCDR3 comprises an amino acid sequence of SEQ ID NOS: 16036, 9503, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17018, 9721, and 9521, respectively and the LCDR1, LCDR2, and LCDR3 comprises an amino acid sequence of SEQ ID NOS: 10830, 9267, and 11335, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16786, 11035, and 10258, respectively and the LCDR1, LCDR2, and LCDR3 comprises an amino acid sequence of SEQ ID NOS:16659, 11900, and 11870, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17470, 16310, and 10292, respectively and the LCDR1, LCDR2, and LCDR3 comprises an amino acid sequence of SEQ ID NOS:16490, 16558, and 12545, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17224, 9882, and 9589, respectively and the LCDR1, LCDR2, and LCDR3 comprises an amino acid sequence of SEQ ID NOS:15895, 10727, and 12877, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17263, 9740, and 10018, respectively and the LCDR1, LCDR2, and LCDR3 comprises an amino acid sequence of SEQ ID NOS: 15700, 3982, and 13017, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17619, 9764, and 10170, respectively and the LCDR1, LCDR2, and LCDR3 comprises an amino acid sequence of SEQ ID NOS:16640, 10747, and 13023, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 18637, 18369, and 6494, respectively and the LCDR1, LCDR2, and LCDR3 comprises an amino acid sequence of SEQ ID NOS:15751, 11144, and 12453, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 18637, 18375, and 6494, respectively and the LCDR1, LCDR2, and LCDR3 comprises an amino acid sequence of SEQ ID NOS:15750, 11144, and 12454, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16695, 18582, and 10522, respectively and the LCDR1, LCDR2, and LCDR3 comprises an amino acid sequence of SEQ ID NOS: 11788, 4420, and 12569, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11623, 9726, and 17117, respectively and the LCDR1, LCDR2, and LCDR3 comprises an amino acid sequence of SEQ ID NOS: 10830, 9267, and 11316, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6523, 16863, and 9458, respectively and the LCDR1, LCDR2, and LCDR3 comprises an amino acid sequence of SEQ ID NOS:16426, 10764, and 18197, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17440, 18397, and 11721, respectively and the LCDR1, LCDR2, and LCDR3 comprises an amino acid sequence of SEQ ID NOS:17929, 6312, and 17166, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11632, 9922, and 6106, respectively and the LCDR1, LCDR2, and LCDR3 comprises an amino acid sequence of SEQ ID NOS:10842, 18267, and 12372, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6557, 11021, and 9650, respectively and the LCDR1, LCDR2, and LCDR3 comprises an amino acid sequence of SEQ ID NOS: 15713, 3982, and 13002, respectively. having a LCDR1, LCDR2, and LCDR3, wherein the HCDR1, HCDR2, and HCDR3 comprises an amino acid sequence of SEQ ID NOS: 11651, 10016, and 7907, respectively and the LCDR1, LCDR2, and LCDR3 comprises an amino acid sequence of SEQ ID NOS: 15875, 3969, and 10452, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17609, 18016, and 11701, respectively and the LCDR1, LCDR2, and LCDR3 comprises an amino acid sequence of SEQ ID NOS: 15977, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:5997, 10586, and 9201, respectively and the LCDR1, LCDR2, and LCDR3 comprises an amino acid sequence of SEQ ID NOS: 15756, 3979, and 12475, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17269, 4663, and 17054, respectively and the LCDR1, LCDR2, and LCDR3 comprises an amino acid sequence of SEQ ID NOS: 15677, 3982, and 12100, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11647, 18367, and 4588, respectively and the LCDR1, LCDR2, and LCDR3 comprises an amino acid sequence of SEQ ID NOS:16112, 10697, and 12831, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11642, 18551, and 4049, respectively and the LCDR1, LCDR2, and LCDR3 comprises an amino acid sequence of SEQ ID NOS: 16036, 9503, and 12778, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17636, 9845, and 11718, respectively and the LCDR1, LCDR2, and LCDR3 comprises an amino acid sequence of SEQ ID NOS:16428, 10764, and 11225, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 17799, and 6772, respectively and the LCDR1, LCDR2, and LCDR3 comprises an amino acid sequence of SEQ ID NOS: 16390, 9503, and 12886, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10948, 17795, and 9424, respectively and the LCDR1, LCDR2, and LCDR3 comprises an amino acid sequence of SEQ ID NOS:15933, 10738, and 12455, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17634, 16923, and 9547, respectively and the LCDR1, LCDR2, and LCDR3 comprises an amino acid sequence of SEQ ID NOS:16067, 4425, and 12419, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10366, 16270, and 9203, respectively and the LCDR1, LCDR2, and LCDR3 comprises an amino acid sequence of SEQ ID NOS: 15706, 3979, and 11980, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 16888, and 16959, respectively and the LCDR1, LCDR2, and LCDR3 comprises an amino acid sequence of SEQ ID NOS: 16086, 4436, and 12366, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17530, 17778, and 11720, respectively and the LCDR1, LCDR2, and LCDR3 comprises an amino acid sequence of SEQ ID NOS: 15895, 10727, and 12863, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16778, 17995, and 18247, respectively and the LCDR1, LCDR2, and LCDR3 comprises an amino acid sequence of SEQ ID NOS:17959, 6307, and 17197, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17845, 17744, and 10148, respectively and the LCDR1, LCDR2, and LCDR3 comprises an amino acid sequence of SEQ ID NOS:16637, 16164, and 13027, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16518, 18026, and 4584, respectively and the LCDR1, LCDR2, and LCDR3 comprises an amino acid sequence of SEQ ID NOS:11944, 10020, and 11583, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17229, 18041, and 17722, respectively and the LCDR1, LCDR2, and LCDR3 comprises an amino acid sequence of SEQ ID NOS: 16685, 16584, and 3997, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16165, 11607, and 10427, respectively and the LCDR1, LCDR2, and LCDR3 comprises an amino acid sequence of SEQ ID NOS: 15669, 17867, and 12397, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11540, 16323, and 9561, respectively and the LCDR1, LCDR2, and LCDR3 comprises an amino acid sequence of SEQ ID NOS:16675, 12300, and 11883, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11673, 10571, and 10965, respectively and the LCDR1, LCDR2, and LCDR3 comprises an amino acid sequence of SEQ ID NOS: 16398, 10967, and 11349, respectively. having a LCDR1, LCDR2, and LCDR3, wherein the HCDR1, HCDR2, and HCDR3 comprises an amino acid sequence of SEQ ID NOS:17655, 6779, and 12041, respectively and the LCDR1, LCDR2, and LCDR3 comprises an amino acid sequence of SEQ ID NOS: 15713, 3982, and 12363, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10368, 6783, and 16366, respectively and the LCDR1, LCDR2, and LCDR3 comprises an amino acid sequence of SEQ ID NOS: 17972, 6318, and 17200, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17475, 16888, and 9649, respectively and the LCDR1, LCDR2, and LCDR3 comprises an amino acid sequence of SEQ ID NOS:16151, 9494, and 12774, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11554, 18417, and 4252, respectively and the LCDR1, LCDR2, and LCDR3 comprises an amino acid sequence of SEQ ID NOS: 16724, 9549, and 3994, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16819, 11485, and 16982, respectively and the LCDR1, LCDR2, and LCDR3 comprises an amino acid sequence of SEQ ID NOS: 15666, 10750, and 12740, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18436, and 5970, respectively and the LCDR1, LCDR2, and LCDR3 comprises an amino acid sequence of SEQ ID NOS: 15648, 3982, and 12402, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11650, 10557, and 7896, respectively and the LCDR1, LCDR2, and LCDR3 comprises an amino acid sequence of SEQ ID NOS:15839, 10727, and 12601, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17272, 18342, and 17901, respectively and the LCDR1, LCDR2, and LCDR3 comprises an amino acid sequence of SEQ ID NOS: 15704, 3969, and 11088, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6671, 9975, and 4604, respectively and the LCDR1, LCDR2, and LCDR3 comprises an amino acid sequence of SEQ ID NOS: 15937, 9951, and 12855, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17127, 18032, and 16282, respectively and the LCDR1, LCDR2, and LCDR3 comprises an amino acid sequence of SEQ ID NOS:10866, 18278, and 12357, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17064, 16948, and 10372, respectively and the LCDR1, LCDR2, and LCDR3 comprises an amino acid sequence of SEQ ID NOS: 15887, 9503, and 12583, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17064, 10017, and 10373, respectively and the LCDR1, LCDR2, and LCDR3 comprises an amino acid sequence of SEQ ID NOS: 15887, 9503, and 12583, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6509, 9952, and 4174, respectively and the LCDR1, LCDR2, and LCDR3 comprises an amino acid sequence of SEQ ID NOS: 10701, 9503, and 12352, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16518, 18231, and 18212, respectively and the LCDR1, LCDR2, and LCDR3 comprises an amino acid sequence of SEQ ID NOS:15861, 10743, and 12608, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17270, 18302, and 6600, respectively and the LCDR1, LCDR2, and LCDR3 comprises an amino acid sequence of SEQ ID NOS: 16045, 9503, and 12787, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10368, 6782, and 13015, respectively and the LCDR1, LCDR2, and LCDR3 comprises an amino acid sequence of SEQ ID NOS: 16061, 9494, and 12671, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6511, 9404, and 4495, respectively and the LCDR1, LCDR2, and LCDR3 comprises an amino acid sequence of SEQ ID NOS: 15894, 4425, and 11101, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10935, 9713, and 10158, respectively and the LCDR1, LCDR2, and LCDR3 comprises an amino acid sequence of SEQ ID NOS: 15701, 3982, and 12303, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6520, 18419, and 10156, respectively and the LCDR1, LCDR2, and LCDR3 comprises an amino acid sequence of SEQ ID NOS:15651, 6309, and 12416, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:9579, 16268, and 6112, respectively and the LCDR1, LCDR2, and LCDR3 comprises an amino acid sequence of SEQ ID NOS: 15687, 3954, and 12376, respectively. having a LCDR1, LCDR2, and LCDR3, wherein the HCDR1, HCDR2, and HCDR3 comprises an amino acid sequence of SEQ ID NOS: 11680, 18499, and 4188, respectively and the LCDR1, LCDR2, and LCDR3 comprises an amino acid sequence of SEQ ID NOS:10863, 18272, and 12592, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17853, 11616, and 13014, respectively and the LCDR1, LCDR2, and LCDR3 comprises an amino acid sequence of SEQ ID NOS: 16642, 10749, and 13331, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11638, 16852, and 18096, respectively and the LCDR1, LCDR2, and LCDR3 comprises an amino acid sequence of SEQ ID NOS:15665, 3982, and 13018, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11650, 10558, and 7897, respectively and the LCDR1, LCDR2, and LCDR3 comprises an amino acid sequence of SEQ ID NOS:15839, 10727, and 12601, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10365, 16271, and 9200, respectively and the LCDR1, LCDR2, and LCDR3 comprises an amino acid sequence of SEQ ID NOS: 15713, 3949, and 11088, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:4476, 10567, and 11048, respectively and the LCDR1, LCDR2, and LCDR3 comprises an amino acid sequence of SEQ ID NOS:15862, 10727, and 12009, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11684, 16267, and 7920, respectively and the LCDR1, LCDR2, and LCDR3 comprises an amino acid sequence of SEQ ID NOS: 15658, 9498, and 12377, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17474, 10761, and 16590, respectively and the LCDR1, LCDR2, and LCDR3 comprises an amino acid sequence of SEQ ID NOS: 16061, 9494, and 12624, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10337, 16238, and 17041, respectively and the LCDR1, LCDR2, and LCDR3 comprises an amino acid sequence of SEQ ID NOS:16429, 10926, and 11275, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16529, 10403, and 6051, respectively and the LCDR1, LCDR2, and LCDR3 comprises an amino acid sequence of SEQ ID NOS:15791, 9501, and 12772, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18434, and 5972, respectively and the LCDR1, LCDR2, and LCDR3 comprises an amino acid sequence of SEQ ID NOS: 15650, 3982, and 12538, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6500, 9967, and 4606, respectively and the LCDR1, LCDR2, and LCDR3 comprises an amino acid sequence of SEQ ID NOS: 15729, 9503, and 12750, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11561, 17737, and 9651, respectively and the LCDR1, LCDR2, and LCDR3 comprises an amino acid sequence of SEQ ID NOS: 17959, 6331, and 4352, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17534, 10568, and 16153, respectively and the LCDR1, LCDR2, and LCDR3 comprises an amino acid sequence of SEQ ID NOS: 15999, 4425, and 12434, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17856, 16319, and 9202, respectively and the LCDR1, LCDR2, and LCDR3 comprises an amino acid sequence of SEQ ID NOS: 16045, 9503, and 12782, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6465, 6787, and 16343, respectively and the LCDR1, LCDR2, and LCDR3 comprises an amino acid sequence of SEQ ID NOS:16694, 11903, and 11861, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17869, 16255, and 16370, respectively and the LCDR1, LCDR2, and LCDR3 comprises an amino acid sequence of SEQ ID NOS:9634, 16330, and 10507, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16593, 16496, and 17851, respectively and the LCDR1, LCDR2, and LCDR3 comprises an amino acid sequence of SEQ ID NOS:15895, 10731, and 12741, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6576, 16506, and 9596, respectively and the LCDR1, LCDR2, and LCDR3 comprises an amino acid sequence of SEQ ID NOS:17971, 6312, and 4354, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11650, 4260, and 4275, respectively and the LCDR1, LCDR2, and LCDR3 comprises an amino acid sequence of SEQ ID NOS: 16648, 6628, and 18619, respectively. having a LCDR1, LCDR2, and LCDR3, wherein the HCDR1, HCDR2, and HCDR3 comprises an amino acid sequence of SEQ ID NOS:17263, 9940, and 10150, respectively and the LCDR1, LCDR2, and LCDR3 comprises an amino acid sequence of SEQ ID NOS: 15935, 9494, and 12647, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11689, 18568, and 4189, respectively and the LCDR1, LCDR2, and LCDR3 comprises an amino acid sequence of SEQ ID NOS:10791, 18256, and 12975, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17437, 18348, and 7857, respectively and the LCDR1, LCDR2, and LCDR3 comprises an amino acid sequence of SEQ ID NOS: 15693, 3982, and 12402, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11630, 18244, and 9346, respectively and the LCDR1, LCDR2, and LCDR3 comprises an amino acid sequence of SEQ ID NOS:16419, 18510, and 11262, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17143, 16827, and 10954, respectively and the LCDR1, LCDR2, and LCDR3 comprises an amino acid sequence of SEQ ID NOS: 11948, 10024, and 11587, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10935, 16229, and 9510, respectively and the LCDR1, LCDR2, and LCDR3 comprises an amino acid sequence of SEQ ID NOS:16428, 10764, and 11226, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17618, 16291, and 11131, respectively and the LCDR1, LCDR2, and LCDR3 comprises an amino acid sequence of SEQ ID NOS: 16683, 16328, and 4007, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6530, 16935, and 4225, respectively and the LCDR1, LCDR2, and LCDR3 comprises an amino acid sequence of SEQ ID NOS: 16056, 9503, and 12725, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10935, 16296, and 6691, respectively and the LCDR1, LCDR2, and LCDR3 comprises an amino acid sequence of SEQ ID NOS:9483, 4417, and 10470, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16802, 10300, and 4203, respectively and the LCDR1, LCDR2, and LCDR3 comprises an amino acid sequence of SEQ ID NOS:18089, 18132, and 13618, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10033, 16265, and 7906, respectively and the LCDR1, LCDR2, and LCDR3 comprises an amino acid sequence of SEQ ID NOS: 15697, 9498, and 11980, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17064, 11528, and 17585, respectively and the LCDR1, LCDR2, and LCDR3 comprises an amino acid sequence of SEQ ID NOS: 10785, 9272, and 11233, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17265, 17745, and 6063, respectively and the LCDR1, LCDR2, and LCDR3 comprises an amino acid sequence of SEQ ID NOS: 15642, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17838, 16254, and 10217, respectively and the LCDR1, LCDR2, and LCDR3 comprises an amino acid sequence of SEQ ID NOS: 15799, 10731, and 11108, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16198, 10043, and 9508, respectively and the LCDR1, LCDR2, and LCDR3 comprises an amino acid sequence of SEQ ID NOS: 16089, 9494, and 12561, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11404, 10041, and 11049, respectively and the LCDR1, LCDR2, and LCDR3 comprises an amino acid sequence of SEQ ID NOS: 15895, 10739, and 11932, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6571, 18309, and 6689, respectively and the LCDR1, LCDR2, and LCDR3 comprises an amino acid sequence of SEQ ID NOS: 16005, 9494, and 12348, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10368, 16511, and 4410, respectively and the LCDR1, LCDR2, and LCDR3 comprises an amino acid sequence of SEQ ID NOS: 16061, 6320, and 12685, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11690, 11606, and 10428, respectively and the LCDR1, LCDR2, and LCDR3 comprises an amino acid sequence of SEQ ID NOS:16132, 17867, and 12396, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10514, 9385, and 9551, respectively and the LCDR1, LCDR2, and LCDR3 comprises an amino acid sequence of SEQ ID NOS: 17959, 9275, and 17154, respectively. having a LCDR1, LCDR2, and LCDR3, wherein the HCDR1, HCDR2, and HCDR3 comprises an amino acid sequence of SEQ ID NOS: 18253, 11025, and 6129, respectively and the LCDR1, LCDR2, and LCDR3 comprises an amino acid sequence of SEQ ID NOS: 15938, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17648, 16844, and 4500, respectively and the LCDR1, LCDR2, and LCDR3 comprises an amino acid sequence of SEQ ID NOS:16427, 16509, and 11275, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17636, 16231, and 11698, respectively and the LCDR1, LCDR2, and LCDR3 comprises an amino acid sequence of SEQ ID NOS: 16396, 10987, and 11209, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17073, 18334, and 6034, respectively and the LCDR1, LCDR2, and LCDR3 comprises an amino acid sequence of SEQ ID NOS:10888, 18272, and 12969, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17431, 18527, and 6048, respectively and the LCDR1, LCDR2, and LCDR3 comprises an amino acid sequence of SEQ ID NOS: 15876, 9503, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17064, 11535, and 18435, respectively and the LCDR1, LCDR2, and LCDR3 comprises an amino acid sequence of SEQ ID NOS: 10822, 9267, and 11339, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17284, 18407, and 9643, respectively and the LCDR1, LCDR2, and LCDR3 comprises an amino acid sequence of SEQ ID NOS:15871, 10697, and 13012, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:11678, 16837, and 10495, respectively and the LCDR1, LCDR2, and LCDR3 comprises an amino acid sequence of SEQ ID NOS:10895, 18275, and 12956, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17856, 18575, and 10991, respectively and the LCDR1, LCDR2, and LCDR3 comprises an amino acid sequence of SEQ ID NOS:10848, 18256, and 12726, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 11634, 10550, and 4199, respectively and the LCDR1, LCDR2, and LCDR3 comprises an amino acid sequence of SEQ ID NOS:15861, 10731, and 12026, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10368, 4682, and 10436, respectively and the LCDR1, LCDR2, and LCDR3 comprises an amino acid sequence of SEQ ID NOS:10878, 18275, and 12974, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16625, 9794, and 17711, respectively and the LCDR1, LCDR2, and LCDR3 comprises an amino acid sequence of SEQ ID NOS: 16146, 3969, and 12385, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17291, 9388, and 4581, respectively and the LCDR1, LCDR2, and LCDR3 comprises an amino acid sequence of SEQ ID NOS:16649, 10747, and 13029, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:10212, 16832, and 11700, respectively and the LCDR1, LCDR2, and LCDR3 comprises an amino acid sequence of SEQ ID NOS:10897, 18275, and 12993, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17466, 16920, and 17712, respectively and the LCDR1, LCDR2, and LCDR3 comprises an amino acid sequence of SEQ ID NOS:17972, 9261, and 17161, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17274, 9939, and 4614, respectively and the LCDR1, LCDR2, and LCDR3 comprises an amino acid sequence of SEQ ID NOS: 16039, 9494, and 12672, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6571, 16266, and 9594, respectively and the LCDR1, LCDR2, and LCDR3 comprises an amino acid sequence of SEQ ID NOS: 17940, 6331, and 4358, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6572, 16269, and 7920, respectively and the LCDR1, LCDR2, and LCDR3 comprises an amino acid sequence of SEQ ID NOS: 15672, 9498, and 12377, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16692, 18586, and 6732, respectively and the LCDR1, LCDR2, and LCDR3 comprises an amino acid sequence of SEQ ID NOS:10890, 18275, and 12826, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6531, 9917, and 9623, respectively and the LCDR1, LCDR2, and LCDR3 comprises an amino acid sequence of SEQ ID NOS:15780, 10734, and 12730, respectively. having a LCDR1, LCDR2, and LCDR3, wherein the HCDR1, HCDR2, and HCDR3 comprises an amino acid sequence of SEQ ID NOS: 11410, 11549, and 7882, respectively and the LCDR1, LCDR2, and LCDR3 comprises an amino acid sequence of SEQ ID NOS:10910, 18275, and 12924, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17016, 16500, and 6487, respectively and the LCDR1, LCDR2, and LCDR3 comprises an amino acid sequence of SEQ ID NOS: 17969, 6628, and 4351, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17850, 17823, and 17541, respectively and the LCDR1, LCDR2, and LCDR3 comprises an amino acid sequence of SEQ ID NOS:16672, 10747, and 18617, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17625, 16983, and 7823, respectively and the LCDR1, LCDR2, and LCDR3 comprises an amino acid sequence of SEQ ID NOS: 16081, 9503, and 12778, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6509, 9970, and 4196, respectively and the LCDR1, LCDR2, and LCDR3 comprises an amino acid sequence of SEQ ID NOS: 15613, 9503, and 12794, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 16678, 16938, and 9460, respectively and the LCDR1, LCDR2, and LCDR3 comprises an amino acid sequence of SEQ ID NOS:16107, 10741, and 12008, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17443, 17810, and 6172, respectively and the LCDR1, LCDR2, and LCDR3 comprises an amino acid sequence of SEQ ID NOS:9630, 4467, and 15559, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:16801, 10760, and 10424, respectively and the LCDR1, LCDR2, and LCDR3 comprises an amino acid sequence of SEQ ID NOS:10223, 6212, and 11052, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 17441, 18434, and 5971, respectively and the LCDR1, LCDR2, and LCDR3 comprises an amino acid sequence of SEQ ID NOS: 15650, 3982, and 12538, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17063, 11432, and 17584, respectively and the LCDR1, LCDR2, and LCDR3 comprises an amino acid sequence of SEQ ID NOS:10828, 9272, and 11233, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:6505, 10408, and 6141, respectively and the LCDR1, LCDR2, and LCDR3 comprises an amino acid sequence of SEQ ID NOS: 15978, 9472, and 12775, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10680, 17761, and 6039, respectively and the LCDR1, LCDR2, and LCDR3 comprises an amino acid sequence of SEQ ID NOS: 15632, 4413, and 12433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 10937, 10408, and 5902, respectively and the LCDR1, LCDR2, and LCDR3 comprises an amino acid sequence of SEQ ID NOS: 15975, 4425, and 12433, respectively.

The disclosure also describes a heavy and light chain comprising the sequences of SEQ ID NO:8200 and 7658; SEQ ID NO:8199 and 7657; SEQ ID NO:8234 and 6918; SEQ ID NO:8233 and 6917; SEQ ID NO:8280 and 6876; SEQ ID NO:8279 and 6875; SEQ ID NO:8332 and 6918; SEQ ID NO:8331 and 6917; SEQ ID NO:8623 and 7460; SEQ ID NO:8622 and 7459; SEQ ID NO:8228 and 7504; SEQ ID NO:8227 and 7503; SEQ ID NO:8440 and 7676; SEQ ID NO:8439 and 7675; SEQ ID NO:8637 and 6972; SEQ ID NO:8636 and 6971; SEQ ID NO:8192 and 6974; SEQ ID NO:8191 and 6973; SEQ ID NO:8254 and 6900; SEQ ID NO:8253 and 6899; SEQ ID NO:8182 and 6969; SEQ ID NO:8181 and 6968; SEQ ID NO:8148 and 7490; SEQ ID NO:8147 and 7489; SEQ ID NO:8300 and 7634; SEQ ID NO:8299 and 7633; SEQ ID NO:8302 and 7788; SEQ ID NO:8301 and 7787; SEQ ID NO:8264 and 7770; SEQ ID NO:8263 and 7769; SEQ ID NO:13811 and 7380; SEQ ID NO:13810 and 7379; SEQ ID NO: 14223 and 13409; SEQ ID NO: 14222 and 13408; SEQ ID NO: 13776 and 6802; SEQ ID NO: 13775 and 6801; SEQ ID NO:14333 and 11746; SEQ ID NO: 14332 and 11745; SEQ ID NO: 14925 and 5481; SEQ ID NO: 14924 and 5480; SEQ ID NO:8834 and 6294; SEQ ID NO:8833 and 6293; SEQ ID NO:7992 and 12248; SEQ ID NO:7991 and 12247; SEQ ID NO:8418 and 11854; SEQ ID NO:8417 and 11853; SEQ ID NO:15498 and 4816; SEQ ID NO:15497 and 4815; SEQ ID NO:15517 and 7046; SEQ ID NO:15516 and 7045; SEQ ID NO:15474 and 5645; SEQ ID NO:15473 and 5644; SEQ ID NO: 13986 and 4764; SEQ ID NO: 13985 and 4763; SEQ ID NO: 12147 and 4080; SEQ ID NO: 12146 and 4079; SEQ ID NO:14551 and 6751; SEQ ID NO: 14550 and 6750; SEQ ID NO:13694 and 7458; SEQ ID NO: 13693 and 7457; SEQ ID NO: 15243 and 7398; SEQ ID NO:15242 and 7397; SEQ ID NO:8625 and 17404; SEQ ID NO:8624 and 17403; SEQ ID NO:9554 and 13174; SEQ ID NO:9553 and 13173; SEQ ID NO: 15235 and 5794; SEQ ID NO: 15234 and 5793; SEQ ID NO:8874 and 6848; SEQ ID NO:8873 and 6847; SEQ ID NO:8943 and 5465; SEQ ID NO:8942 and 5464; SEQ ID NO:6286 and 4675; SEQ ID NO:6285 and 4674; SEQ ID NO: 14241 and 5455; SEQ ID NO: 14240 and 5454; SEQ ID NO:7976 and 4776; SEQ ID NO:7975 and 4775; SEQ ID NO:9125 and 7162; SEQ ID NO:9124 and 7161; SEQ ID NO:8651 and 17338; SEQ ID NO:8650 and 17337; SEQ ID NO:13789 and 4931; SEQ ID NO:13788 and 4930; SEQ ID NO:13682 and 7040; SEQ ID NO:13681 and 7039; SEQ ID NO:8838 and 18242; SEQ ID NO:8837 and 18241; SEQ ID NO: 12195 and 16629; SEQ ID NO: 12194 and 16628; SEQ ID NO:9056 and 4800; SEQ ID NO:9055 and 4799; SEQ ID NO: 14077 and 5173; SEQ ID NO: 14076 and 5172; SEQ ID NO: 14152 and 5657; SEQ ID NO: 14151 and 5656; SEQ ID NO:15343 and 7122; SEQ ID NO:15342 and 7121; SEQ ID NO:8284 and 6992; SEQ ID NO:8283 and 6991; SEQ ID NO:8000 and 10666; SEQ ID NO:7999 and 10665; SEQ ID NO:13819 and 7030; SEQ ID NO:13818 and 7029; SEQ ID NO:8998 and 4726; SEQ ID NO:8997 and 4725; SEQ ID NO:9153 and 13246; SEQ ID NO:9152 and 13245; SEQ ID NO:12281 and 7080; SEQ ID NO:12280 and 7079; SEQ ID NO:12193 and 17268; SEQ ID NO: 12192 and 17267; SEQ ID NO:6280 and 13391; SEQ ID NO:6279 and 13390; SEQ ID NO:14527 and 7798; SEQ ID NO:14526 and 7797; SEQ ID NO:14073 and 5107; SEQ ID NO:14072 and 5106; SEQ ID NO:12076 and 13377; SEQ ID NO: 12075 and 13376; SEQ ID NO:8500 and 4927; SEQ ID NO:8499 and 4926; SEQ ID NO:8593 and 13506; SEQ ID NO:8592 and 13505; SEQ ID NO:13724 and 12270; SEQ ID NO:13723 and 12269; SEQ ID NO:8428 and 13216; SEQ ID NO:8427 and 13215; SEQ ID NO:8994 and 13508; SEQ ID NO:8993 and 13507; SEQ ID NO:7970 and 6454; SEQ ID NO:7969 and 6453; SEQ ID NO:12135 and 7554; SEQ ID NO:12134 and 7553; SEQ ID NO:13823 and 7146; SEQ ID NO:13822 and 7145; SEQ ID NO: 12246 and 5889; SEQ ID NO: 12245 and 5888; SEQ ID NO:6266 and 6872; SEQ ID NO:6265 and 6871; SEQ ID NO:18194 and 5539; SEQ ID NO:18193 and 5538; SEQ ID NO:12141 and 17416; SEQ ID NO:12140 and 17415; SEQ ID NO:14891 and 5792; SEQ ID NO:14890 and 5791; SEQ ID NO:6272 and 5463; SEQ ID NO:6271 and 5462; SEQ ID NO:9183 and 4553; SEQ ID NO:9182 and 4552; SEQ ID NO:15319 and 5875; SEQ ID NO:15318 and 5874; SEQ ID NO:15466 and 7370; SEQ ID NO: 15465 and 7369; SEQ ID NO: 15295 and 7926; SEQ ID NO: 15294 and 7925; SEQ ID NO:8088 and 4551; SEQ ID NO:8087 and 4550; SEQ ID NO:14264 and 6920; SEQ ID NO: 14263 and 6917; SEQ ID NO: 14383 and 7605; SEQ ID NO: 14382 and 7604; SEQ ID NO: 14327 and 13039; SEQ ID NO:14326 and 13038; SEQ ID NO:15301 and 5311; SEQ ID NO:15300 and 5310; SEQ ID NO:15524 and 17517; SEQ ID NO:15523 and 17516; SEQ ID NO:8438 and 5513; SEQ ID NO:8437 and 5512; SEQ ID NO:8502 and 5565; SEQ ID NO:8501 and 5564; SEQ ID NO:8012 and 4557; SEQ ID NO:8011 and 4556; SEQ ID NO:15169 and 4774; SEQ ID NO:15168 and 4773; SEQ ID NO:14557 and 4112; SEQ ID NO: 14556 and 4111; SEQ ID NO: 14247 and 6934; SEQ ID NO: 14246 and 6933; SEQ ID NO: 13847 and 7160; SEQ ID NO: 13846 and 7159; SEQ ID NO:8537 and 4974; SEQ ID NO:8527 and 4972; SEQ ID NO:8547 and 17392; SEQ ID NO:8546 and 17391; SEQ ID NO:18168 and 5881; SEQ ID NO:18167 and 5880; SEQ ID NO:8360 and 6826; SEQ ID NO:8359 and 6825; SEQ ID NO:13835 and 18115; SEQ ID NO:13834 and 18114; SEQ ID NO: 14294 and 11750; SEQ ID NO:14293 and 11749; SEQ ID NO:8992 and 4549; SEQ ID NO:8991 and 4548; SEQ ID NO:14079 and 4748; SEQ ID NO:14078 and 4747; SEQ ID NO:14803 and 4561; SEQ ID NO:14802 and 4560; SEQ ID NO:8462 and 7368; SEQ ID NO:8461 and 7367; SEQ ID NO:13805 and 7532; SEQ ID NO: 13804 and 7531; SEQ ID NO: 14923 and 5814; SEQ ID NO: 14922 and 5813; SEQ ID NO:8504 and 12324; SEQ ID NO:8503 and 12323; SEQ ID NO: 12307 and 17487; SEQ ID NO: 12306 and 17486; SEQ ID NO:9018 and 7597; SEQ ID NO:9017 and 7596; SEQ ID NO:8452 and 13435; SEQ ID NO:8451 and 13434; SEQ ID NO:8842 and 4519; SEQ ID NO:8841 and 4518; SEQ ID NO:14237 and 6850; SEQ ID NO:14236 and 6849; SEQ ID NO:14859 and 7310; SEQ ID NO: 14858 and 7309; SEQ ID NO:13813 and 7318; SEQ ID NO:13812 and 7317; SEQ ID NO: 14209 and 13357; SEQ ID NO: 14208 and 13356; SEQ ID NO:9066 and 17400; SEQ ID NO:9065 and 17399; SEQ ID NO: 14621 and 4078; SEQ ID NO: 14620 and 4077; SEQ ID NO: 15059 and 5383; SEQ ID NO:15058 and 5382; SEQ ID NO:8152 and 5395; SEQ ID NO:8151 and 5394; SEQ ID NO:15032 and 5747; SEQ ID NO:15031 and 5736; SEQ ID NO:7968 and 6458; SEQ ID NO:7967 and 6457; SEQ ID NO:8778 and 6852; SEQ ID NO:8777 and 6851; SEQ ID NO:18176 and 13248; SEQ ID NO:18175 and 13247; SEQ ID NO:13815 and 7028; SEQ ID NO:13814 and 7027; SEQ ID NO:12211 and 5343; SEQ ID NO:12210 and 5342; SEQ ID NO: 14861 and 7048; SEQ ID NO: 14860 and 7047; SEQ ID NO:9109 and 6894; SEQ ID NO:9108 and 6893; SEQ ID NO:15275 and 7438; SEQ ID NO:15274 and 7437; SEQ ID NO:13712 and 5275; SEQ ID NO:13711 and 5274; SEQ ID NO: 14847 and 5800; SEQ ID NO: 14846 and 5799; SEQ ID NO:8631 and 5113; SEQ ID NO:8630 and 5112; SEQ ID NO:14795 and 7070; SEQ ID NO:14794 and 7069; SEQ ID NO:9264 and 5239; SEQ ID NO:9263 and 5238; SEQ ID NO:8104 and 9283; SEQ ID NO:8103 and 9282; SEQ ID NO:12149 and 4088; SEQ ID NO: 12148 and 4087; SEQ ID NO: 12143 and 4086; SEQ ID NO: 12142 and 4085; SEQ ID NO:8166 and 5309; SEQ ID NO:8165 and 5308; SEQ ID NO:13732 and 18128; SEQ ID NO:13731 and 18127; SEQ ID NO: 14075 and 5109; SEQ ID NO: 14074 and 5108; SEQ ID NO:8794 and 7486; SEQ ID NO:8793 and 7485; SEQ ID NO:8036 and 5559; SEQ ID NO:8035 and 5558; SEQ ID NO:14627 and 7951; SEQ ID NO: 14626 and 7950; SEQ ID NO:11920 and 7756; SEQ ID NO:11919 and 7755; SEQ ID NO: 13762 and 6800; SEQ ID NO:13761 and 6799; SEQ ID NO:8020 and 5345; SEQ ID NO:8019 and 5344; SEQ ID NO:15349 and 5775; SEQ ID NO:15348 and 5774; SEQ ID NO: 14053 and 13272; SEQ ID NO: 14052 and 13271; SEQ ID NO: 14071 and 5187; SEQ ID NO: 14070 and 5186; SEQ ID NO:13831 and 7366; SEQ ID NO:13830 and 7365; SEQ ID NO:9213 and 13395; SEQ ID NO:9212 and 13394; SEQ ID NO:15237 and 13111; SEQ ID NO:15236 and 13110; SEQ ID NO:9141 and 7424; SEQ ID NO:9140 and 7423; SEQ ID NO:13837 and 7032; SEQ ID NO:13836 and 7031; SEQ ID NO:13827 and 6443; SEQ ID NO:13826 and 6442; SEQ ID NO: 15117 and 5778; SEQ ID NO:15116 and 5776; SEQ ID NO:6298 and 5451; SEQ ID NO:6297 and 5450; SEQ ID NO:8585 and 6451; SEQ ID NO:8584 and 6450; SEQ ID NO: 14525 and 7800; SEQ ID NO: 14524 and 7799; SEQ ID NO:13803 and 6761; SEQ ID NO: 13802 and 6760; SEQ ID NO: 11908 and 4858; SEQ ID NO: 11907 and 4857; SEQ ID NO: 15511 and 6449; SEQ ID NO:15510 and 6448; SEQ ID NO:14339 and 5251; SEQ ID NO:14338 and 5250; SEQ ID NO:14935 and 5804; SEQ ID NO:14934 and 5803; SEQ ID NO:14553 and 13057; SEQ ID NO: 14552 and 13056; SEQ ID NO: 14693 and 5610; SEQ ID NO: 14692 and 5607; SEQ ID NO: 14245 and 6822; SEQ ID NO: 14244 and 6821; SEQ ID NO: 12161 and 12266; SEQ ID NO: 12160 and 12265; SEQ ID NO:14569 and 6196; SEQ ID NO:14568 and 6195; SEQ ID NO:12175 and 13230; SEQ ID NO: 12170 and 13229; SEQ ID NO:13853 and 7474; SEQ ID NO:13852 and 7473; SEQ ID NO:14619 and 4092; SEQ ID NO:14618 and 4091; SEQ ID NO:12113 and 6838; SEQ ID NO:12112 and 6837; SEQ ID NO:8996 and 7466; SEQ ID NO:8995 and 7465; SEQ ID NO:13632 and 5483; SEQ ID NO:13631 and 5482; SEQ ID NO:9169 and 4513; SEQ ID NO:9168 and 4512; SEQ ID NO:14899 and 7190; SEQ ID NO:14898 and 7189; SEQ ID NO:13841 and 7174; SEQ ID NO:13840 and 7173; SEQ ID NO: 13875 and 5436; SEQ ID NO:13874 and 5435; SEQ ID NO: 14869 and 6759; SEQ ID NO: 14868 and 6758; SEQ ID NO: 13754 and 12320; SEQ ID NO:13753 and 12319; SEQ ID NO:8010 and 7006; SEQ ID NO:8009 and 7005; SEQ ID NO:14887 and 5790; SEQ ID NO: 14886 and 5789; SEQ ID NO: 14176 and 7258; SEQ ID NO: 14175 and 7257; SEQ ID NO: 15163 and 5816; SEQ ID NO:15162 and 5815; SEQ ID NO:8611 and 5399; SEQ ID NO:8610 and 5398; SEQ ID NO:15119 and 5777; SEQ ID NO:15118 and 5776; SEQ ID NO:8782 and 13073; SEQ ID NO:8781 and 13072; SEQ ID NO:7990 and 4967; SEQ ID NO:7989 and 4966; SEQ ID NO:15472 and 10652; SEQ ID NO:15471 and 10651; SEQ ID NO:15247 and 7008; SEQ ID NO:15246 and 7007; SEQ ID NO:13821 and 7020; SEQ ID NO:13820 and 7019; SEQ ID NO:13807 and 7034; SEQ ID NO: 13806 and 7033; SEQ ID NO:8808 and 4541; SEQ ID NO:8807 and 4540; SEQ ID NO:15345 and 6224; SEQ ID NO:15344 and 6223; SEQ ID NO:9042 and 5183; SEQ ID NO:9041 and 5182; SEQ ID NO:14284 and 13101; SEQ ID NO:14283 and 13100; SEQ ID NO:9173 and 18238; SEQ ID NO:9172 and 18237; SEQ ID NO:8672 and 7710; SEQ ID NO:8671 and 7709; SEQ ID NO:13968 and 4766; SEQ ID NO:13967 and 4765; SEQ ID NO:13817 and 7038; SEQ ID NO:13816 and 7037; SEQ ID NO: 14917 and 7392; SEQ ID NO:14916 and 7391; SEQ ID NO:13843 and 7210; SEQ ID NO:13842 and 7209; SEQ ID NO:14270 and 7110; SEQ ID NO:14269 and 7109; SEQ ID NO:14893 and 10664; SEQ ID NO:14892 and 10663; SEQ ID NO:9151 and 17402; SEQ ID NO:9150 and 17401; SEQ ID NO:9155 and 6890; SEQ ID NO:9154 and 6889; SEQ ID NO: 13644 and 7164; SEQ ID NO:13643 and 7163; SEQ ID NO: 12203 and 13065; SEQ ID NO: 12202 and 13064; SEQ ID NO:15123 and 7120; SEQ ID NO:15122 and 7119; SEQ ID NO:7844 and 6375; SEQ ID NO:7843 and 6374; SEQ ID NO:13984 and 4764; SEQ ID NO: 13983 and 4763; SEQ ID NO:7980 and 10662; SEQ ID NO:7979 and 10661; SEQ ID NO:15458 and 4736; SEQ ID NO:15457 and 4735; SEQ ID NO:13839 and 7094; SEQ ID NO:13838 and 7093; SEQ ID NO:8840 and 4555; SEQ ID NO:8839 and 4554; SEQ ID NO: 14325 and 4782; SEQ ID NO: 14324 and 4781; SEQ ID NO:13825 and 5475; SEQ ID NO:13824 and 5474; SEQ ID NO:8972 and 13236; SEQ ID NO:8971 and 13235; SEQ ID NO:9064 and 6818; SEQ ID NO:9063 and 6817; SEQ ID NO:8208 and 6385; SEQ ID NO:8207 and 6384; SEQ ID NO:9123 and 6982; SEQ ID NO:9122 and 6981; SEQ ID NO:13833 and 7036; SEQ ID NO:13832 and 7035; SEQ ID NO:9147 and 7396; SEQ ID NO:9146 and 7395; SEQ ID NO:14617 and 4090; SEQ ID NO: 14616 and 4089; SEQ ID NO:13829 and 7372; SEQ ID NO:13828 and 7371; SEQ ID NO:15299 and 5780; SEQ ID NO:15298 and 5779; SEQ ID NO:18148 and 7320; SEQ ID NO:18147 and 7319; SEQ ID NO:13642 and 7166; SEQ ID NO: 13641 and 7165; SEQ ID NO: 14631 and 5147; SEQ ID NO: 14630 and 5146; SEQ ID NO:15503 and 4836; SEQ ID NO:15502 and 4835; SEQ ID NO:11897 and 7140; SEQ ID NO:11896 and 7139; SEQ ID NO:15450 and 5133; SEQ ID NO:15449 and 5132; SEQ ID NO:15091 and 7022; SEQ ID NO:15090 and 7021; SEQ ID NO:13787 and 4844; SEQ ID NO:13786 and 4843; SEQ ID NO: 14625 and 5195; SEQ ID NO: 14624 and 5194; SEQ ID NO: 15283 and 5349; SEQ ID NO: 15282 and 5348; SEQ ID NO: 15049 and 7078; SEQ ID NO: 15048 and 7077; SEQ ID NO:12047 and 5095; SEQ ID NO:12046 and 5094; SEQ ID NO: 14463 and 17791; SEQ ID NO:14460 and 17789; SEQ ID NO:15492 and 4806; SEQ ID NO:15491 and 4805; SEQ ID NO:15223 and 7686; SEQ ID NO:15222 and 7685; SEQ ID NO: 14791 and 7230; SEQ ID NO: 14790 and 7229; SEQ ID NO:13778 and 7348; SEQ ID NO:13777 and 7347; SEQ ID NO:18209 and 4852; SEQ ID NO:18208 and 4851; SEQ ID NO:14998 and 5333; SEQ ID NO:14984 and 5327; SEQ ID NO:15335 and 7168; SEQ ID NO:15334 and 7167; SEQ ID NO:11756 and 5438; SEQ ID NO:11755 and 5437; SEQ ID NO: 14563 and 7376; SEQ ID NO: 14562 and 7375; SEQ ID NO: 14733 and 13176; SEQ ID NO: 14724 and 13175; SEQ ID NO: 12163 and 13244; SEQ ID NO: 12162 and 13243; SEQ ID NO:13907 and 5093; SEQ ID NO: 13906 and 5092; SEQ ID NO: 14004 and 5332; SEQ ID NO: 14003 and 5330; SEQ ID NO: 13994 and 4919; SEQ ID NO: 13993 and 4918; SEQ ID NO: 15257 and 6840; SEQ ID NO:15256 and 6839; SEQ ID NO:14563 and 7378; SEQ ID NO:14562 and 7377; SEQ ID NO:14581 and 4842; SEQ ID NO:14580 and 4841; SEQ ID NO: 13634 and 5087; SEQ ID NO:13633 and 5086; SEQ ID NO:8340 and 7436; SEQ ID NO:8339 and 7435; SEQ ID NO:14144 and 17295; SEQ ID NO: 14143 and 17294; SEQ ID NO: 13668 and 5071; SEQ ID NO:13667 and 5070; SEQ ID NO: 13903 and 4872; SEQ ID NO:13902 and 4871; SEQ ID NO: 14469 and 5742; SEQ ID NO: 14458 and 5741; SEQ ID NO:9105 and 4860; SEQ ID NO:9104 and 4859; SEQ ID NO:8326 and 6998; SEQ ID NO:8325 and 6997; SEQ ID NO:6583 and 5453; SEQ ID NO:6582 and 5452; SEQ ID NO:13696 and 7728; SEQ ID NO:13695 and 7727; SEQ ID NO: 14809 and 4983; SEQ ID NO: 14808 and 4982; SEQ ID NO:9080 and 17505; SEQ ID NO:9074 and 17504; SEQ ID NO:6315 and 7774; SEQ ID NO:6314 and 7773; SEQ ID NO:8022 and 4937; SEQ ID NO:8021 and 4936; SEQ ID NO:14675 and 5069; SEQ ID NO:14664 and 5058; SEQ ID NO:14672 and 7287; SEQ ID NO:14665 and 7268; SEQ ID NO:8306 and 13361; SEQ ID NO:8305 and 13360; SEQ ID NO:15039 and 5027; SEQ ID NO: 15038 and 5026; SEQ ID NO: 15159 and 4976; SEQ ID NO:15158 and 4975; SEQ ID NO: 12299 and 4638; SEQ ID NO:12298 and 4637; SEQ ID NO:11899 and 7092; SEQ ID NO:11898 and 7091; SEQ ID NO:15519 and 4963; SEQ ID NO:15518 and 4962; SEQ ID NO:15229 and 7180; SEQ ID NO:15228 and 7179; SEQ ID NO:14736 and 5025; SEQ ID NO: 14735 and 5024; SEQ ID NO:18211 and 4880; SEQ ID NO:18210 and 4879; SEQ ID NO:8909 and 13252; SEQ ID NO:8908 and 13251; SEQ ID NO: 12090 and 4828; SEQ ID NO: 12089 and 4827; SEQ ID NO: 13728 and 7018; SEQ ID NO: 13727 and 7017; SEQ ID NO: 13956 and 4921; SEQ ID NO:13955 and 4920; SEQ ID NO:8224 and 6886; SEQ ID NO:8223 and 6885; SEQ ID NO:15436 and 6206; SEQ ID NO:15435 and 6205; SEQ ID NO: 14967 and 5009; SEQ ID NO: 14966 and 5008; SEQ ID NO: 14817 and 7228; SEQ ID NO: 14816 and 7227; SEQ ID NO:14217 and 13494; SEQ ID NO: 14216 and 13493; SEQ ID NO:14321 and 13449; SEQ ID NO:14320 and 13448; SEQ ID NO:12117 and 7044; SEQ ID NO:12116 and 7043; SEQ ID NO:12151 and 5225; SEQ ID NO:12150 and 5224; SEQ ID NO:8474 and 5137; SEQ ID NO:8473 and 5136; SEQ ID NO:13716 and 5135; SEQ ID NO:13715 and 5134; SEQ ID NO:6278 and 5097; SEQ ID NO:6277 and 5096; SEQ ID NO: 14239 and 5007; SEQ ID NO: 14238 and 5006; SEQ ID NO:8308 and 10618; SEQ ID NO:8307 and 10617; SEQ ID NO:6589 and 5101; SEQ ID NO:6588 and 5100; SEQ ID NO:12125 and 13527; SEQ ID NO: 12124 and 13526; SEQ ID NO:8230 and 6919; SEQ ID NO:8229 and 6917; SEQ ID NO:8126 and 7644; SEQ ID NO:8125 and 7643; SEQ ID NO:7840 and 13397; SEQ ID NO:7839 and 13396; SEQ ID NO:6252 and 7664; SEQ ID NO:6251 and 7663; SEQ ID NO:8490 and 17084; SEQ ID NO:8489 and 17083; SEQ ID NO:13917 and 13292; SEQ ID NO:13916 and 13291; SEQ ID NO:12227 and 5015; SEQ ID NO: 12226 and 5014; SEQ ID NO: 14006 and 9327; SEQ ID NO: 14005 and 9326; SEQ ID NO: 14337 and 13310; SEQ ID NO: 14336 and 13309; SEQ ID NO:9143 and 5357; SEQ ID NO:9142 and 5356; SEQ ID NO:9062 and 6984; SEQ ID NO:9061 and 6983; SEQ ID NO: 14485 and 13296; SEQ ID NO: 14484 and 13295; SEQ ID NO: 12225 and 5111; SEQ ID NO: 12224 and 5110; SEQ ID NO: 12056 and 5725; SEQ ID NO: 12055 and 5724; SEQ ID NO:13672 and 5549; SEQ ID NO:13671 and 5548; SEQ ID NO:9253 and 5757; SEQ ID NO:9252 and 5756; SEQ ID NO:15416 and 5403; SEQ ID NO:15415 and 5402; SEQ ID NO:9101 and 17356; SEQ ID NO:9100 and 17355; SEQ ID NO:9099 and 17358; SEQ ID NO:9098 and 17357; SEQ ID NO: 15333 and 5253; SEQ ID NO: 15332 and 5252; SEQ ID NO: 14146 and 6622; SEQ ID NO: 14145 and 6621; SEQ ID NO:15217 and 7214; SEQ ID NO:15216 and 7213; SEQ ID NO: 14055 and 5751; SEQ ID NO:14054 and 5750; SEQ ID NO: 13750 and 6930; SEQ ID NO: 13749 and 6929; SEQ ID NO:8886 and 13355; SEQ ID NO:8885 and 13354; SEQ ID NO: 12272 and 9289; SEQ ID NO: 12271 and 9288; SEQ ID NO:15113 and 7730; SEQ ID NO:15112 and 7729; SEQ ID NO:8142 and 9323; SEQ ID NO:8141 and 9322; SEQ ID NO:8176 and 4539; SEQ ID NO:8175 and 4538; SEQ ID NO:14097 and 4911; SEQ ID NO: 14096 and 4905; SEQ ID NO:8248 and 7627; SEQ ID NO:8238 and 7627; SEQ ID NO:8348 and 12322; SEQ ID NO:8347 and 12321; SEQ ID NO:10420 and 5587; SEQ ID NO:10419 and 5586; SEQ ID NO:8278 and 7569; SEQ ID NO:8277 and 7568; SEQ ID NO:8460 and 5029; SEQ ID NO:8459 and 5028; SEQ ID NO:9032 and 13148; SEQ ID NO:9031 and 13147; SEQ ID NO:13998 and 4798; SEQ ID NO: 13997 and 4797; SEQ ID NO:8728 and 13416; SEQ ID NO:8726 and 13413; SEQ ID NO:13744 and 10620; SEQ ID NO:13743 and 10619; SEQ ID NO:15141 and 7334; SEQ ID NO:15140 and 7333; SEQ ID NO:8086 and 9436; SEQ ID NO:8085 and 9435; SEQ ID NO:14799 and 7136; SEQ ID NO: 14798 and 7135; SEQ ID NO: 14243 and 13182; SEQ ID NO: 14242 and 13181; SEQ ID NO:15462 and 7016; SEQ ID NO:15461 and 7015; SEQ ID NO:12088 and 13547; SEQ ID NO:12087 and 13546; SEQ ID NO:15353 and 6922; SEQ ID NO:15352 and 6921; SEQ ID NO:15197 and 5563; SEQ ID NO: 15196 and 5562; SEQ ID NO: 14895 and 7336; SEQ ID NO: 14894 and 7335; SEQ ID NO: 14587 and 7577; SEQ ID NO: 14586 and 7576; SEQ ID NO:9209 and 7560; SEQ ID NO:9208 and 7559; SEQ ID NO:15004 and 7284; SEQ ID NO: 15002 and 7268; SEQ ID NO:8241 and 17332; SEQ ID NO:8238 and 17329; SEQ ID NO: 15255 and 5415; SEQ ID NO: 15254 and 5414; SEQ ID NO:6585 and 11807; SEQ ID NO:6584 and 11806; SEQ ID NO:12050 and 13314; SEQ ID NO:12049 and 13313; SEQ ID NO:15151 and 6767; SEQ ID NO:15150 and 6766; SEQ ID NO: 15233 and 9299; SEQ ID NO: 15232 and 9298; SEQ ID NO:9163 and 4533; SEQ ID NO:9162 and 4532; SEQ ID NO:9016 and 10644; SEQ ID NO:9015 and 10643; SEQ ID NO:15432 and 7084; SEQ ID NO: 15431 and 7083; SEQ ID NO:8024 and 4537; SEQ ID NO:8023 and 4536; SEQ ID NO:15496 and 6765; SEQ ID NO:15495 and 6764; SEQ ID NO:15195 and 7060; SEQ ID NO:15194 and 7059; SEQ ID NO: 15055 and 5259; SEQ ID NO:15054 and 5258; SEQ ID NO:6254 and 7502; SEQ ID NO:6253 and 7501; SEQ ID NO:9181 and 11395; SEQ ID NO:9180 and 11394; SEQ ID NO:14204 and 5065; SEQ ID NO:14201 and 5058; SEQ ID NO:8252 and 17790; SEQ ID NO:8238 and 17789; SEQ ID NO: 14813 and 7794; SEQ ID NO: 14812 and 7793; SEQ ID NO:14453 and 5606; SEQ ID NO: 14450 and 5604; SEQ ID NO:18154 and 11860; SEQ ID NO:18153 and 11859; SEQ ID NO:6270 and 4929; SEQ ID NO:6269 and 4928; SEQ ID NO:8930 and 11850; SEQ ID NO:8929 and 11849; SEQ ID NO:14258 and 17502; SEQ ID NO: 14256 and 17500; SEQ ID NO:8492 and 5557; SEQ ID NO:8491 and 5556; SEQ ID NO:14779 and 7186; SEQ ID NO:14778 and 7185; SEQ ID NO:8519 and 5746; SEQ ID NO:8517 and 5737; SEQ ID NO:15189 and 6994; SEQ ID NO:15188 and 6993; SEQ ID NO:14363 and 5845; SEQ ID NO:14362 and 5844; SEQ ID NO:15003 and 13133; SEQ ID NO:15002 and 13126; SEQ ID NO: 14875 and 7294; SEQ ID NO: 14874 and 7293; SEQ ID NO:8806 and 4790; SEQ ID NO:8805 and 4789; SEQ ID NO:13881 and 7138; SEQ ID NO:13880 and 7137; SEQ ID NO:12171 and 5335; SEQ ID NO: 12170 and 5327; SEQ ID NO: 15285 and 6908; SEQ ID NO: 15284 and 6907; SEQ ID NO:8206 and 7098; SEQ ID NO:8205 and 7097; SEQ ID NO:15087 and 5385; SEQ ID NO:15086 and 5384; SEQ ID NO:8657 and 4531; SEQ ID NO:8656 and 4530; SEQ ID NO:8698 and 13172; SEQ ID NO:8687 and 13171; SEQ ID NO: 12178 and 5738; SEQ ID NO: 12170 and 5737; SEQ ID NO:8669 and 5068; SEQ ID NO:8667 and 5058; SEQ ID NO:12069 and 7286; SEQ ID NO:12063 and 7268; SEQ ID NO: 14994 and 17387; SEQ ID NO: 14984 and 17377; SEQ ID NO:8868 and 7662; SEQ ID NO:8867 and 7661; SEQ ID NO:15355 and 7346; SEQ ID NO: 15354 and 7345; SEQ ID NO: 14138 and 5005; SEQ ID NO: 14137 and 5004; SEQ ID NO: 14732 and 5066; SEQ ID NO: 14725 and 5058; SEQ ID NO:8655 and 9317; SEQ ID NO:8654 and 9316; SEQ ID NO:14708 and 13425; SEQ ID NO:14705 and 13422; SEQ ID NO:14679 and 5334; SEQ ID NO:14665 and 5327; SEQ ID NO:15380 and 5155; SEQ ID NO:15379 and 5154; SEQ ID NO: 14713 and 6367; SEQ ID NO: 14705 and 6366; SEQ ID NO: 12172 and 5059; SEQ ID NO: 12170 and 5058; SEQ ID NO: 8402 and 7768; SEQ ID NO:8401 and 7767; SEQ ID NO:8529 and 11399; SEQ ID NO:8526 and 11398; SEQ ID NO: 14461 and 7627; SEQ ID NO: 14460 and 7615; SEQ ID NO:15041 and 15584; SEQ ID NO:15040 and 15583; SEQ ID NO: 14561 and 7943; SEQ ID NO: 14560 and 7942; SEQ ID NO:8731 and 6960; SEQ ID NO:8726 and 6952; SEQ ID NO:8242 and 13125; SEQ ID NO:8238 and 13120; SEQ ID NO:15404 and 7296; SEQ ID NO:15403 and 7295; SEQ ID NO: 12168 and 5326; SEQ ID NO: 12167 and 5321; SEQ ID NO:8530 and 4158; SEQ ID NO:8527 and 4157; SEQ ID NO: 14252 and 6956; SEQ ID NO: 14250 and 6952; SEQ ID NO:14434 and 17388; SEQ ID NO:14424 and 17375; SEQ ID NO:8700 and 4719; SEQ ID NO:8686 and 4716; SEQ ID NO:8691 and 13479; SEQ ID NO:8687 and 13477; SEQ ID NO:14112 and 5064; SEQ ID NO:14110 and 5058; SEQ ID NO:14107 and 7627; SEQ ID NO: 14104 and 7615; SEQ ID NO:8864 and 5745; SEQ ID NO:8863 and 5737; SEQ ID NO:18152 and 7627; SEQ ID NO:18151 and 7627; SEQ ID NO:15482 and 7052; SEQ ID NO:15481 and 7051; SEQ ID NO: 12104 and 6874; SEQ ID NO: 12103 and 6873; SEQ ID NO:9075 and 7282; SEQ ID NO:9074 and 7268; SEQ ID NO:9217 and 6739; SEQ ID NO:9216 and 6738; SEQ ID NO:8970 and 5127; SEQ ID NO:8969 and 5126; SEQ ID NO:12235 and 4876; SEQ ID NO:12234 and 4875; SEQ ID NO:13923 and 13423; SEQ ID NO:13921 and 13422; SEQ ID NO:8762 and 17376; SEQ ID NO:8759 and 17375; SEQ ID NO:8900 and 7269; SEQ ID NO:8899 and 7268; SEQ ID NO:14174 and 17430; SEQ ID NO:14173 and 17429; SEQ ID NO:14266 and 5351; SEQ ID NO:14265 and 5350; SEQ ID NO:9081 and 13425; SEQ ID NO:9074 and 13422; SEQ ID NO:15490 and 7142; SEQ ID NO:15489 and 7141; SEQ ID NO:6602 and 5713; SEQ ID NO:6601 and 5712; SEQ ID NO:14695 and 5619; SEQ ID NO:14692 and 5607; SEQ ID NO:7974 and 13087; SEQ ID NO:7973 and 13086; SEQ ID NO: 14667 and 13415; SEQ ID NO: 14665 and 13414; SEQ ID NO:8859 and 13124; SEQ ID NO:8857 and 13122; SEQ ID NO: 14479 and 5305; SEQ ID NO: 14478 and 5304; SEQ ID NO:12066 and 13170; SEQ ID NO: 12065 and 13169; SEQ ID NO: 15406 and 7410; SEQ ID NO:15405 and 7409; SEQ ID NO:13928 and 5326; SEQ ID NO: 13921 and 5321; SEQ ID NO:8545 and 10588; SEQ ID NO:8544 and 10587; SEQ ID NO:15400 and 7102; SEQ ID NO:15399 and 7101; SEQ ID NO:15371 and 13069; SEQ ID NO:15370 and 13068; SEQ ID NO: 14937 and 6685; SEQ ID NO:14936 and 6684; SEQ ID NO:14593 and 6687; SEQ ID NO:14592 and 6686; SEQ ID NO:14335 and 6967; SEQ ID NO:14334 and 6966; SEQ ID NO:12082 and 5407; SEQ ID NO:12081 and 5406; SEQ ID NO:14305 and 5062; SEQ ID NO:14304 and 5060; SEQ ID NO:14125 and 17303; SEQ ID NO:14123 and 17302; SEQ ID NO: 15454 and 7216; SEQ ID NO:15453 and 7215; SEQ ID NO:8244 and 17090; SEQ ID NO:8238 and 17089; SEQ ID NO:13768 and 13304; SEQ ID NO: 13767 and 13303; SEQ ID NO:8670 and 5161; SEQ ID NO:8666 and 5160; SEQ ID NO:6268 and 9433; SEQ ID NO:6267 and 9432; SEQ ID NO: 12197 and 7534; SEQ ID NO: 12196 and 7533; SEQ ID NO: 14537 and 4564; SEQ ID NO: 14536 and 4563; SEQ ID NO: 8699 and 6940; SEQ ID NO:8686 and 6939; SEQ ID NO: 12064 and 5336; SEQ ID NO: 12063 and 5321; SEQ ID NO:9077 and 7619; SEQ ID NO:9074 and 7618; SEQ ID NO: 15434 and 7100; SEQ ID NO: 15433 and 7099; SEQ ID NO: 14115 and 5613; SEQ ID NO: 14110 and 5611; SEQ ID NO:15418 and 6932; SEQ ID NO: 15417 and 6931; SEQ ID NO: 14666 and 5739; SEQ ID NO: 14665 and 5737; SEQ ID NO: 14190 and 7252; SEQ ID NO: 14189 and 7251; SEQ ID NO:8690 and 6962; SEQ ID NO:8687 and 6950; SEQ ID NO: 14093 and 4808; SEQ ID NO: 14092 and 4807; SEQ ID NO:8057 and 17527; SEQ ID NO:8056 and 17526; SEQ ID NO:14609 and 4802; SEQ ID NO: 14608 and 4801; SEQ ID NO: 14756 and 13516; SEQ ID NO: 14753 and 13515; SEQ ID NO:13936 and 7082; SEQ ID NO:13935 and 7081; SEQ ID NO: 14503 and 7208; SEQ ID NO: 14502 and 7207; SEQ ID NO: 14777 and 13363; SEQ ID NO:14776 and 13362; SEQ ID NO:14124 and 5320; SEQ ID NO:14123 and 5317; SEQ ID NO: 14023 and 6947; SEQ ID NO: 14019 and 6946; SEQ ID NO: 14119 and 17093; SEQ ID NO:14108 and 17088; SEQ ID NO:8721 and 5337; SEQ ID NO:8718 and 5316; SEQ ID NO:8571 and 7716; SEQ ID NO:8570 and 7715; SEQ ID NO:14744 and 13325; SEQ ID NO:14743 and 13324; SEQ ID NO:14613 and 7360; SEQ ID NO: 14612 and 7359; SEQ ID NO:14117 and 17092; SEQ ID NO:14110 and 17089; SEQ ID NO:8482 and 4866; SEQ ID NO:8481 and 4865; SEQ ID NO: 14017 and 5065; SEQ ID NO:14015 and 5058; SEQ ID NO:8695 and 4102; SEQ ID NO:8687 and 4101; SEQ ID NO:14113 and 13128; SEQ ID NO: 14110 and 13127; SEQ ID NO:15484 and 7090; SEQ ID NO:15483 and 7089; SEQ ID NO:13740 and 18124; SEQ ID NO:13739 and 18123; SEQ ID NO: 14369 and 13097; SEQ ID NO:14368 and 13096; SEQ ID NO:8679 and 7280; SEQ ID NO:8678 and 7275; SEQ ID NO:9079 and 5614; SEQ ID NO:9074 and 5607; SEQ ID NO:15265 and 7226; SEQ ID NO:15264 and 7225; SEQ ID NO:8518 and 5605; SEQ ID NO:8517 and 5604; SEQ ID NO: 14219 and 4874; SEQ ID NO: 14218 and 4873; SEQ ID NO: 14105 and 5159; SEQ ID NO: 14104 and 5158; SEQ ID NO:8722 and 12314; SEQ ID NO:8718 and 12313; SEQ ID NO:14178 and 13134; SEQ ID NO:14177 and 13126; SEQ ID NO:14678 and 5065; SEQ ID NO: 14665 and 5058; SEQ ID NO: 14016 and 4907; SEQ ID NO: 14015 and 4905; SEQ ID NO:6274 and 6439; SEQ ID NO:6273 and 6438; SEQ ID NO:14085 and 7342; SEQ ID NO:14084 and 7341; SEQ ID NO:14024 and 13119; SEQ ID NO:14019 and 13116; SEQ ID NO:12187 and 13421; SEQ ID NO:12186 and 13420; SEQ ID NO:8520 and 13131; SEQ ID NO:8517 and 13126; SEQ ID NO:15375 and 7627; SEQ ID NO:15374 and 7615; SEQ ID NO:18156 and 13553; SEQ ID NO:18155 and 13552; SEQ ID NO:8053 and 11848; SEQ ID NO:8052 and 11847; SEQ ID NO:8130 and 7552; SEQ ID NO:8129 and 7551; SEQ ID NO:14428 and 5609; SEQ ID NO:14425 and 5607; SEQ ID NO:12179 and 7626; SEQ ID NO:12170 and 7615; SEQ ID NO:8862 and 5322; SEQ ID NO:8857 and 5321; SEQ ID NO: 14118 and 7264; SEQ ID NO: 14109 and 7263; SEQ ID NO: 12062 and 4712; SEQ ID NO: 12061 and 4711; SEQ ID NO:8468 and 4750; SEQ ID NO:8467 and 4749; SEQ ID NO:15402 and 7232; SEQ ID NO:15401 and 7231; SEQ ID NO:7842 and 6441; SEQ ID NO:7841 and 6440; SEQ ID NO:15373 and 6953; SEQ ID NO:15372 and 6952; SEQ ID NO:14020 and 13478; SEQ ID NO:14019 and 13477; SEQ ID NO:14991 and 5612; SEQ ID NO:14985 and 5611; SEQ ID NO:8866 and 7646; SEQ ID NO:8865 and 7645; SEQ ID NO:8484 and 7428; SEQ ID NO:8483 and 7427; SEQ ID NO: 14154 and 4840; SEQ ID NO: 14153 and 4839; SEQ ID NO: 13692 and 6776; SEQ ID NO: 13691 and 6775; SEQ ID NO:14310 and 6963; SEQ ID NO: 14304 and 6946; SEQ ID NO:8848 and 17366; SEQ ID NO:8847 and 17365; SEQ ID NO:8688 and 5864; SEQ ID NO:8687 and 5862; SEQ ID NO:15175 and 5209; SEQ ID NO:15174 and 5208; SEQ ID NO:14311 and 7279; SEQ ID NO: 14303 and 7275; SEQ ID NO:14437 and 12261; SEQ ID NO: 14436 and 12260; SEQ ID NO:12131 and 5201; SEQ ID NO:12130 and 5200; SEQ ID NO: 14130 and 7224; SEQ ID NO: 14129 and 7223; SEQ ID NO:8434 and 13482; SEQ ID NO:8433 and 13476; SEQ ID NO:15001 and 6955; SEQ ID NO: 14984 and 6952; SEQ ID NO:15488 and 7204; SEQ ID NO:15487 and 7203; SEQ ID NO:8732 and 13472; SEQ ID NO:8726 and 13471; SEQ ID NO:6262 and 17299; SEQ ID NO:6261 and 17298; SEQ ID NO:8717 and 13520; SEQ ID NO:8716 and 13515; SEQ ID NO: 14730 and 7285; SEQ ID NO: 14726 and 7268; SEQ ID NO:15442 and 7304; SEQ ID NO: 15441 and 7303; SEQ ID NO: 14022 and 7292; SEQ ID NO: 14019 and 7263; SEQ ID NO:8536 and 5328; SEQ ID NO:8527 and 5327; SEQ ID NO:8733 and 13132; SEQ ID NO:8726 and 13126; SEQ ID NO: 15369 and 7496; SEQ ID NO: 15368 and 7495; SEQ ID NO: 15412 and 5417; SEQ ID NO: 15411 and 5416; SEQ ID NO: 14127 and 6957; SEQ ID NO: 14122 and 6952; SEQ ID NO:15069 and 6808; SEQ ID NO:15068 and 6807; SEQ ID NO:9135 and 6812; SEQ ID NO:9134 and 6811; SEQ ID NO:14731 and 6955; SEQ ID NO: 14727 and 6952; SEQ ID NO: 14992 and 17497; SEQ ID NO: 14985 and 17496; SEQ ID NO:14716 and 13601; SEQ ID NO: 14705 and 13600; SEQ ID NO:8719 and 17386; SEQ ID NO:8718 and 17377; SEQ ID NO:8980 and 7422; SEQ ID NO:8979 and 7421; SEQ ID NO:15476 and 5425; SEQ ID NO: 15475 and 5424; SEQ ID NO:16317 and 5409; SEQ ID NO:16316 and 5408; SEQ ID NO: 15000 and 7279; SEQ ID NO: 14984 and 7275; SEQ ID NO:8532 and 5325; SEQ ID NO:8527 and 5317; SEQ ID NO:8931 and 5744; SEQ ID NO:8929 and 5737; SEQ ID NO:15548 and 4677; SEQ ID NO:15547 and 4676; SEQ ID NO:8720 and 4945; SEQ ID NO:8718 and 4944; SEQ ID NO:8858 and 17331; SEQ ID NO:8857 and 17330; SEQ ID NO:15446 and 13055; SEQ ID NO:15445 and 13054; SEQ ID NO:8260 and 7589; SEQ ID NO:8259 and 7588; SEQ ID NO:15480 and 6810; SEQ ID NO:15479 and 6809; SEQ ID NO: 14495 and 13475; SEQ ID NO: 14494 and 13474; SEQ ID NO: 14771 and 5013; SEQ ID NO: 14770 and 5012; SEQ ID NO:15486 and 7050; SEQ ID NO:15485 and 7049; SEQ ID NO:13925 and 13481; SEQ ID NO:13921 and 13476; SEQ ID NO: 8134 and 7520; SEQ ID NO:8133 and 7519; SEQ ID NO:15422 and 7124; SEQ ID NO:15421 and 7123; SEQ ID NO:13926 and 17385; SEQ ID NO:13921 and 17377; SEQ ID NO:8860 and 17382; SEQ ID NO:8857 and 17378; SEQ ID NO:6658 and 9431; SEQ ID NO:6657 and 9430; SEQ ID NO:14547 and 7064; SEQ ID NO:14546 and 7063; SEQ ID NO:15221 and 7178; SEQ ID NO:15220 and 7177; SEQ ID NO:14126 and 17327; SEQ ID NO: 14123 and 17324; SEQ ID NO: 14111 and 7279; SEQ ID NO: 14110 and 7275; SEQ ID NO:8422 and 7260; SEQ ID NO:8421 and 7259; SEQ ID NO:14459 and 13121; SEQ ID NO:14458 and 13120; SEQ ID NO:8366 and 13232; SEQ ID NO:8365 and 13231; SEQ ID NO: 14668 and 4123; SEQ ID NO: 14665 and 4122; SEQ ID NO:15183 and 5485; SEQ ID NO:15182 and 5484; SEQ ID NO:12169 and 5067; SEQ ID NO: 12166 and 5058; SEQ ID NO: 12177 and 7004; SEQ ID NO: 12170 and 7003; SEQ ID NO:8581 and 7462; SEQ ID NO:8580 and 7461; SEQ ID NO: 12067 and 13415; SEQ ID NO: 12065 and 13414; SEQ ID NO:13927 and 7627; SEQ ID NO:13920 and 7614; SEQ ID NO:14114 and 17094; SEQ ID NO:14110 and 17087; SEQ ID NO:13996 and 4812; SEQ ID NO:13995 and 4811; SEQ ID NO:14729 and 17093; SEQ ID NO: 14728 and 17088; SEQ ID NO:15507 and 4096; SEQ ID NO:15506 and 4095; SEQ ID NO: 13702 and 6743; SEQ ID NO: 13701 and 6742; SEQ ID NO:14659 and 12256; SEQ ID NO:14658 and 12255; SEQ ID NO: 14996 and 7630; SEQ ID NO: 14984 and 7627; SEQ ID NO:9117 and 5631; SEQ ID NO:9116 and 5630; SEQ ID NO:8986 and 13447; SEQ ID NO:8985 and 13446; SEQ ID NO:8380 and 7536; SEQ ID NO:8379 and 7535; SEQ ID NO:8370 and 5203; SEQ ID NO:8369 and 5202; SEQ ID NO:8448 and 7540; SEQ ID NO:8447 and 7539; SEQ ID NO:15426 and 7352; SEQ ID NO:15425 and 7351; SEQ ID NO: 14987 and 6964; SEQ ID NO: 14985 and 6946; SEQ ID NO:8668 and 4717; SEQ ID NO:8667 and 4716; SEQ ID NO: 14734 and 5618; SEQ ID NO:14723 and 5607; SEQ ID NO:8404 and 6343; SEQ ID NO:8403 and 6342; SEQ ID NO:8599 and 7666; SEQ ID NO:8598 and 7665; SEQ ID NO:14462 and 17326; SEQ ID NO: 14460 and 17325; SEQ ID NO:8822 and 5185; SEQ ID NO:8821 and 5184; SEQ ID NO:15478 and 9291; SEQ ID NO: 15477 and 9290; SEQ ID NO: 14432 and 5617; SEQ ID NO: 14425 and 5607; SEQ ID NO:14677 and 5326; SEQ ID NO: 14664 and 5317; SEQ ID NO: 15430 and 7234; SEQ ID NO: 15429 and 7233; SEQ ID NO: 14997 and 13416; SEQ ID NO: 14985 and 13413; SEQ ID NO:15420 and 7154; SEQ ID NO:15419 and 7153; SEQ ID NO: 14694 and 7616; SEQ ID NO: 14692 and 7615; SEQ ID NO:6282 and 17315; SEQ ID NO:6281 and 17314; SEQ ID NO: 8905 and 4118; SEQ ID NO:8901 and 4117; SEQ ID NO:8456 and 13113; SEQ ID NO:8455 and 13112; SEQ ID NO:15219 and 6774; SEQ ID NO:15218 and 6773; SEQ ID NO: 14377 and 7362; SEQ ID NO:14376 and 7361; SEQ ID NO:13630 and 9301; SEQ ID NO:13629 and 9300; SEQ ID NO:15297 and 7220; SEQ ID NO:15296 and 7219; SEQ ID NO:8458 and 7500; SEQ ID NO:8457 and 7499; SEQ ID NO:8531 and 13522; SEQ ID NO:8527 and 13515; SEQ ID NO: 14341 and 7088; SEQ ID NO: 14340 and 7087; SEQ ID NO:8861 and 5334; SEQ ID NO:8857 and 5327; SEQ ID NO: 14483 and 7086; SEQ ID NO: 14482 and 7085; SEQ ID NO:8810 and 5381; SEQ ID NO:8809 and 5380; SEQ ID NO:8661 and 15550; SEQ ID NO:8660 and 15549; SEQ ID NO: 15424 and 7184; SEQ ID NO: 15423 and 7183; SEQ ID NO:7838 and 6437; SEQ ID NO:7837 and 6436; SEQ ID NO:8697 and 5830; SEQ ID NO:8687 and 5828; SEQ ID NO:8249 and 4961; SEQ ID NO:8238 and 4960; SEQ ID NO: 14430 and 5616; SEQ ID NO: 14425 and 5607; SEQ ID NO:8250 and 4907; SEQ ID NO:8238 and 4905; SEQ ID NO:8917 and 6832; SEQ ID NO:8916 and 6831; SEQ ID NO:13919 and 17406; SEQ ID NO: 13918 and 17405; SEQ ID NO:9084 and 5324; SEQ ID NO:9074 and 5321; SEQ ID NO:14262 and 5293; SEQ ID NO:14261 and 5292; SEQ ID NO:8846 and 5517; SEQ ID NO:8845 and 5516; SEQ ID NO:12185 and 13480; SEQ ID NO: 12166 and 13476; SEQ ID NO: 14929 and 6796; SEQ ID NO: 14928 and 6795; SEQ ID NO:8693 and 7288; SEQ ID NO:8687 and 7263; SEQ ID NO:8512 and 13604; SEQ ID NO:8511 and 13603; SEQ ID NO:14315 and 4915; SEQ ID NO:14314 and 4914; SEQ ID NO:8639 and 5323; SEQ ID NO:8638 and 5321; SEQ ID NO:8118 and 7587; SEQ ID NO:8117 and 7586; SEQ ID NO:8870 and 6904; SEQ ID NO:8869 and 6903; SEQ ID NO:9083 and 4979; SEQ ID NO:9074 and 4978; SEQ ID NO:8132 and 7550; SEQ ID NO:8131 and 7549; SEQ ID NO:14203 and 5061; SEQ ID NO:14201 and 5060; SEQ ID NO: 15325 and 7430; SEQ ID NO: 15324 and 7429; SEQ ID NO:8521 and 4912; SEQ ID NO:8517 and 4905; SEQ ID NO:15279 and 10525; SEQ ID NO:15278 and 10524; SEQ ID NO:15530 and 17419; SEQ ID NO: 15529 and 17418; SEQ ID NO:9086 and 6948; SEQ ID NO:9074 and 6946; SEQ ID NO:14989 and 5615; SEQ ID NO:14985 and 5607; SEQ ID NO:8412 and 7454; SEQ ID NO:8411 and 7453; SEQ ID NO:9177 and 17485; SEQ ID NO:9176 and 17484; SEQ ID NO:14251 and 13519; SEQ ID NO:14250 and 13518; SEQ ID NO: 15145 and 4671; SEQ ID NO:15144 and 4670; SEQ ID NO:9087 and 5157; SEQ ID NO:9073 and 5156; SEQ ID NO: 12180 and 17498; SEQ ID NO:12166 and 17496; SEQ ID NO:9121 and 5501; SEQ ID NO:9120 and 5500; SEQ ID NO:9161 and 10658; SEQ ID NO:9160 and 10657; SEQ ID NO: 14988 and 4723; SEQ ID NO: 14985 and 4721; SEQ ID NO: 14441 and 17481; SEQ ID NO: 14440 and 17480; SEQ ID NO:8928 and 5863; SEQ ID NO:8926 and 5862; SEQ ID NO:8685 and 7291; SEQ ID NO:8684 and 7263; SEQ ID NO: 14999 and 5326; SEQ ID NO:14985 and 5321; SEQ ID NO:14951 and 5179; SEQ ID NO:14950 and 5178; SEQ ID NO:14426 and 5831; SEQ ID NO: 14425 and 5828; SEQ ID NO:8727 and 5865; SEQ ID NO:8726 and 5862; SEQ ID NO:15438 and 7152; SEQ ID NO:15437 and 7151; SEQ ID NO:14465 and 13130; SEQ ID NO:14460 and 13127; SEQ ID NO:9088 and 7628; SEQ ID NO:9073 and 7627; SEQ ID NO:15428 and 7150; SEQ ID NO:15427 and 7149; SEQ ID NO: 14688 and 7595; SEQ ID NO: 14687 and 7594; SEQ ID NO:8245 and 17379; SEQ ID NO:8238 and 17378; SEQ ID NO:14433 and 13308; SEQ ID NO: 14425 and 13305; SEQ ID NO:15444 and 7302; SEQ ID NO:15443 and 7301; SEQ ID NO: 13760 and 7056; SEQ ID NO:13759 and 7055; SEQ ID NO:9076 and 7289; SEQ ID NO:9074 and 7263; SEQ ID NO: 14231 and 7492; SEQ ID NO:14230 and 7491; SEQ ID NO:8528 and 4910; SEQ ID NO:8527 and 4905; SEQ ID NO:15303 and 6683; SEQ ID NO:15302 and 6682; SEQ ID NO: 15376 and 4126; SEQ ID NO:15374 and 4124; SEQ ID NO:8947 and 6936; SEQ ID NO:8946 and 6935; SEQ ID NO:13662 and 4834; SEQ ID NO:13661 and 4833; SEQ ID NO: 15311 and 6341; SEQ ID NO:15310 and 6340; SEQ ID NO:14785 and 5153; SEQ ID NO: 14784 and 5152; SEQ ID NO:9337 and 6355; SEQ ID NO:9336 and 6354; SEQ ID NO:9082 and 7278; SEQ ID NO:9074 and 7275; SEQ ID NO: 14690 and 13124; SEQ ID NO: 14689 and 13122; SEQ ID NO:8715 and 5608; SEQ ID NO:8714 and 5607; SEQ ID NO:12176 and 7274; SEQ ID NO: 12170 and 7268; SEQ ID NO:14464 and 6959; SEQ ID NO:14460 and 6952; SEQ ID NO:12173 and 13129; SEQ ID NO:12170 and 13127; SEQ ID NO: 14941 and 6681; SEQ ID NO:14940 and 6680; SEQ ID NO:15227 and 7338; SEQ ID NO:15226 and 7337; SEQ ID NO: 14116 and 17503; SEQ ID NO: 14110 and 17499; SEQ ID NO: 13911 and 7356; SEQ ID NO:13910 and 7355; SEQ ID NO: 8328 and 7524; SEQ ID NO:8327 and 7523; SEQ ID NO: 14196 and 7324; SEQ ID NO: 14195 and 7323; SEQ ID NO:15365 and 4738; SEQ ID NO:15364 and 4737; SEQ ID NO:14757 and 6965; SEQ ID NO:14753 and 6941; SEQ ID NO:14255 and 7629; SEQ ID NO: 14250 and 7627; SEQ ID NO:8692 and 4913; SEQ ID NO:8687 and 4905; SEQ ID NO: 14027 and 13081; SEQ ID NO: 14026 and 13080; SEQ ID NO:13883 and 6365; SEQ ID NO:13882 and 6364; SEQ ID NO:8583 and 17483; SEQ ID NO:8582 and 17482; SEQ ID NO:14857 and 5826; SEQ ID NO: 14856 and 5825; SEQ ID NO: 14308 and 4904; SEQ ID NO: 14304 and 4903; SEQ ID NO:9085 and 5832; SEQ ID NO:9074 and 5828; SEQ ID NO:14423 and 7620; SEQ ID NO:14422 and 7618; SEQ ID NO:14014 and 13383; SEQ ID NO:14013 and 13382; SEQ ID NO: 13948 and 5635; SEQ ID NO:13947 and 5634; SEQ ID NO:8674 and 7854; SEQ ID NO:8673 and 7853; SEQ ID NO: 14645 and 4521; SEQ ID NO: 14644 and 4520; SEQ ID NO:8555 and 5041; SEQ ID NO:8554 and 5040; SEQ ID NO: 14703 and 6407; SEQ ID NO: 14702 and 6406; SEQ ID NO: 14035 and 17362; SEQ ID NO: 14034 and 17361; SEQ ID NO:8758 and 4971; SEQ ID NO:8757 and 4970; SEQ ID NO: 14309 and 6399; SEQ ID NO: 14304 and 6398; SEQ ID NO: 14306 and 5743; SEQ ID NO: 14304 and 5737; SEQ ID NO: 14427 and 4722; SEQ ID NO: 14425 and 4721; SEQ ID NO:14008 and 7780; SEQ ID NO: 14007 and 7779; SEQ ID NO: 14787 and 5077; SEQ ID NO: 14786 and 5076; SEQ ID NO:11957 and 13433; SEQ ID NO:11956 and 13432; SEQ ID NO:9091 and 18246; SEQ ID NO:9090 and 18245; SEQ ID NO:13944 and 4830; SEQ ID NO:13943 and 4829; SEQ ID NO:8723 and 17501; SEQ ID NO:8718 and 17500; SEQ ID NO:8222 and 5603; SEQ ID NO:8221 and 5602; SEQ ID NO:8432 and 12264; SEQ ID NO:8431 and 12263; SEQ ID NO:9191 and 5641; SEQ ID NO:9190 and 5640; SEQ ID NO:6258 and 5761; SEQ ID NO:6257 and 5760; SEQ ID NO: 14164 and 5543; SEQ ID NO: 14163 and 5542; SEQ ID NO:14452 and 17091; SEQ ID NO:14450 and 17089; SEQ ID NO: 14103 and 6943; SEQ ID NO: 14102 and 6942; SEQ ID NO: 14827 and 7062; SEQ ID NO: 14826 and 7061; SEQ ID NO: 13982 and 5531; SEQ ID NO:13981 and 5530; SEQ ID NO:8676 and 7290; SEQ ID NO:8675 and 7263; SEQ ID NO: 14647 and 7182; SEQ ID NO: 14646 and 7181; SEQ ID NO:6296 and 5519; SEQ ID NO:6295 and 5518; SEQ ID NO:14945 and 7250; SEQ ID NO:14944 and 7249; SEQ ID NO:8894 and 17420; SEQ ID NO:8893 and 17417; SEQ ID NO:13636 and 5103; SEQ ID NO:13635 and 5102; SEQ ID NO:8535 and 13140; SEQ ID NO:8527 and 13139; SEQ ID NO:8730 and 5833; SEQ ID NO:8726 and 5827; SEQ ID NO:12240 and 5269; SEQ ID NO:12239 and 5268; SEQ ID NO:8579 and 7700; SEQ ID NO:8578 and 7699; SEQ ID NO:9245 and 5255; SEQ ID NO:9244 and 5254; SEQ ID NO:9020 and 5324; SEQ ID NO:9019 and 5321; SEQ ID NO:14049 and 13484; SEQ ID NO:14048 and 13483; SEQ ID NO:8907 and 13143; SEQ ID NO:8899 and 13142; SEQ ID NO:8028 and 4535; SEQ ID NO:8027 and 4534; SEQ ID NO: 14188 and 4742; SEQ ID NO:14187 and 4741; SEQ ID NO:8424 and 4084; SEQ ID NO:8423 and 4083; SEQ ID NO:8764 and 5297; SEQ ID NO:8763 and 5296; SEQ ID NO:8346 and 7694; SEQ ID NO:8345 and 7693; SEQ ID NO:13924 and 7271; SEQ ID NO:13921 and 7268; SEQ ID NO: 15177 and 7939; SEQ ID NO: 15176 and 7938; SEQ ID NO: 14200 and 7283; SEQ ID NO: 14197 and 7268; SEQ ID NO:8776 and 13071; SEQ ID NO:8775 and 13070; SEQ ID NO: 13628 and 17096; SEQ ID NO:13627 and 17095; SEQ ID NO:14585 and 7412; SEQ ID NO:14584 and 7411; SEQ ID NO:11912 and 4746; SEQ ID NO:11911 and 4745; SEQ ID NO:14783 and 7116; SEQ ID NO:14782 and 7115; SEQ ID NO:8689 and 13525; SEQ ID NO:8687 and 13524; SEQ ID NO: 14671 and 7270; SEQ ID NO: 14665 and 7268; SEQ ID NO: 12072 and 6949; SEQ ID NO: 12070 and 6946; SEQ ID NO: 14674 and 5740; SEQ ID NO:14665 and 5737; SEQ ID NO:9089 and 5331; SEQ ID NO:9073 and 5330; SEQ ID NO:13960 and 4832; SEQ ID NO:13959 and 4831; SEQ ID NO:8534 and 4125; SEQ ID NO:8527 and 4124; SEQ ID NO: 14253 and 7272; SEQ ID NO: 14250 and 7268; SEQ ID NO:13748 and 5853; SEQ ID NO:13747 and 5852; SEQ ID NO:6595 and 17511; SEQ ID NO:6594 and 17510; SEQ ID NO:8788 and 11739; SEQ ID NO:8787 and 11738; SEQ ID NO: 13905 and 7607; SEQ ID NO: 13904 and 7606; SEQ ID NO:8696 and 5401; SEQ ID NO:8687 and 5400; SEQ ID NO:8549 and 5177; SEQ ID NO:8548 and 5176; SEQ ID NO: 14095 and 13280; SEQ ID NO: 14094 and 13279; SEQ ID NO:13772 and 13294; SEQ ID NO:13771 and 13293; SEQ ID NO:14986 and 13144; SEQ ID NO:14985 and 13141; SEQ ID NO:14710 and 13143; SEQ ID NO: 14705 and 13142; SEQ ID NO:15392 and 5503; SEQ ID NO:15391 and 5502; SEQ ID NO:8784 and 7068; SEQ ID NO:8783 and 7067; SEQ ID NO:9195 and 4547; SEQ ID NO:9194 and 4546; SEQ ID NO:9133 and 6902; SEQ ID NO:9132 and 6901; SEQ ID NO:13885 and 17387; SEQ ID NO:13884 and 17377; SEQ ID NO:15099 and 4642; SEQ ID NO:15098 and 4641; SEQ ID NO:13954 and 5765; SEQ ID NO:13953 and 5764; SEQ ID NO:13988 and 17248; SEQ ID NO: 13987 and 17247; SEQ ID NO:8790 and 5277; SEQ ID NO:8789 and 5276; SEQ ID NO:15267 and 10668; SEQ ID NO: 15266 and 10667; SEQ ID NO:8961 and 6816; SEQ ID NO:8960 and 6815; SEQ ID NO:14709 and 11401; SEQ ID NO: 14705 and 11400; SEQ ID NO:8704 and 5391; SEQ ID NO:8703 and 5390; SEQ ID NO:14431 and 11402; SEQ ID NO:14425 and 11400; SEQ ID NO:8677 and 13268; SEQ ID NO:8675 and 13267; SEQ ID NO:14415 and 7654; SEQ ID NO:14414 and 7653; SEQ ID NO:14192 and 6741; SEQ ID NO: 14191 and 6740; SEQ ID NO:13901 and 5637; SEQ ID NO:13900 and 5636; SEQ ID NO:8935 and 6806; SEQ ID NO:8934 and 6805; SEQ ID NO:9058 and 6842; SEQ ID NO:9057 and 6841; SEQ ID NO:8904 and 4906; SEQ ID NO:8901 and 4905; SEQ ID NO:8589 and 5495; SEQ ID NO:8588 and 5494; SEQ ID NO:13964 and 17368; SEQ ID NO:13963 and 17367; SEQ ID NO:8488 and 5299; SEQ ID NO:8487 and 5298; SEQ ID NO:13899 and 4838; SEQ ID NO:13898 and 4837; SEQ ID NO:15028 and 6954; SEQ ID NO:15027 and 6952; SEQ ID NO: 14447 and 13095; SEQ ID NO: 14446 and 13094; SEQ ID NO: 15035 and 5693; SEQ ID NO: 15034 and 5692; SEQ ID NO: 15259 and 7406; SEQ ID NO: 15258 and 7405; SEQ ID NO:8694 and 7617; SEQ ID NO:8687 and 7615; SEQ ID NO: 14539 and 12276; SEQ ID NO:14538 and 12275; SEQ ID NO:8573 and 5521; SEQ ID NO:8572 and 5520; SEQ ID NO:13930 and 6958; SEQ ID NO:13920 and 6952; SEQ ID NO: 14655 and 4762; SEQ ID NO: 14654 and 4761; SEQ ID NO:7998 and 13059; SEQ ID NO:7997 and 13058; SEQ ID NO:13922 and 7625; SEQ ID NO:13921 and 7618; SEQ ID NO:9187 and 4566; SEQ ID NO:9186 and 4565; SEQ ID NO: 14680 and 4720; SEQ ID NO: 14664 and 4716; SEQ ID NO:8945 and 6753; SEQ ID NO:8944 and 6752; SEQ ID NO:8725 and 5329; SEQ ID NO:8724 and 5327; SEQ ID NO: 14300 and 13473; SEQ ID NO: 14299 and 13470; SEQ ID NO: 14045 and 4925; SEQ ID NO: 14044 and 4924; SEQ ID NO: 14706 and 7627; SEQ ID NO: 14705 and 7615; SEQ ID NO:8933 and 13158; SEQ ID NO:8932 and 13157; SEQ ID NO:15071 and 4094; SEQ ID NO:15070 and 4093; SEQ ID NO: 14000 and 4923; SEQ ID NO:13999 and 4922; SEQ ID NO:8761 and 4119; SEQ ID NO:8759 and 4115; SEQ ID NO:8613 and 4545; SEQ ID NO:8612 and 4544; SEQ ID NO:12231 and 7054; SEQ ID NO:12230 and 7053; SEQ ID NO:13966 and 7613; SEQ ID NO:13965 and 7612; SEQ ID NO: 14746 and 7601; SEQ ID NO: 14745 and 7600; SEQ ID NO: 14307 and 13602; SEQ ID NO: 14304 and 13600; SEQ ID NO:8368 and 4752; SEQ ID NO:8367 and 4751; SEQ ID NO:13958 and 16577; SEQ ID NO:13957 and 16576; SEQ ID NO:8955 and 13502; SEQ ID NO:8954 and 13501; SEQ ID NO:8911 and 13083; SEQ ID NO:8910 and 13082; SEQ ID NO: 12209 and 5393; SEQ ID NO: 12208 and 5392; SEQ ID NO:14711 and 7631; SEQ ID NO: 14705 and 7627; SEQ ID NO: 14051 and 5149; SEQ ID NO:14050 and 5148; SEQ ID NO:14707 and 13417; SEQ ID NO:14705 and 13412; SEQ ID NO:8450 and 9297; SEQ ID NO:8449 and 9296; SEQ ID NO:14319 and 5199; SEQ ID NO:14318 and 5198; SEQ ID NO: 14755 and 7276; SEQ ID NO: 14753 and 7275; SEQ ID NO:8006 and 17082; SEQ ID NO:8005 and 17081; SEQ ID NO:14198 and 7632; SEQ ID NO:14197 and 7627; SEQ ID NO:8963 and 6679; SEQ ID NO:8962 and 6678; SEQ ID NO:14807 and 7603; SEQ ID NO: 14806 and 7602; SEQ ID NO:15033 and 13523; SEQ ID NO:15031 and 13513; SEQ ID NO:14575 and 7332; SEQ ID NO:14574 and 7331; SEQ ID NO:8239 and 11748; SEQ ID NO:8238 and 11747; SEQ ID NO:14323 and 5885; SEQ ID NO: 14322 and 5884; SEQ ID NO: 15135 and 7298; SEQ ID NO: 15134 and 7297; SEQ ID NO: 13758 and 13260; SEQ ID NO:13757 and 13259; SEQ ID NO:14286 and 13298; SEQ ID NO:14285 and 13297; SEQ ID NO:8436 and 13156; SEQ ID NO:8435 and 13155; SEQ ID NO: 8965 and 7243; SEQ ID NO:8964 and 7242; SEQ ID NO:13770 and 7132; SEQ ID NO: 13769 and 7131; SEQ ID NO:15191 and 6804; SEQ ID NO: 15190 and 6803; SEQ ID NO:8430 and 17305; SEQ ID NO:8429 and 17304; SEQ ID NO: 13889 and 17370; SEQ ID NO: 13888 and 17369; SEQ ID NO: 15329 and 7344; SEQ ID NO: 15328 and 7343; SEQ ID NO:8342 and 12312; SEQ ID NO:8341 and 12311; SEQ ID NO:8951 and 11799; SEQ ID NO:8950 and 11798; SEQ ID NO:8065 and 17396; SEQ ID NO:8064 and 17395; SEQ ID NO: 12078 and 13288; SEQ ID NO:12077 and 13287; SEQ ID NO:9107 and 6878; SEQ ID NO:9106 and 6877; SEQ ID NO: 15215 and 4640; SEQ ID NO:15214 and 4639; SEQ ID NO:8004 and 11813; SEQ ID NO:8003 and 11812; SEQ ID NO: 14429 and 7330; SEQ ID NO: 14425 and 7329; SEQ ID NO:15153 and 7330; SEQ ID NO:15152 and 7329; SEQ ID NO:6648 and 7776; SEQ ID NO:6647 and 7775; SEQ ID NO: 13845 and 10624; SEQ ID NO:13844 and 10623; SEQ ID NO:8647 and 13051; SEQ ID NO:8646 and 13050; SEQ ID NO:8304 and 5207; SEQ ID NO:8303 and 5206; SEQ ID NO:14841 and 5810; SEQ ID NO:14840 and 5809; SEQ ID NO:15396 and 7736; SEQ ID NO:15395 and 7735; SEQ ID NO: 12127 and 5639; SEQ ID NO: 12126 and 5638; SEQ ID NO:7887 and 6204; SEQ ID NO:7886 and 6203; SEQ ID NO:6646 and 7504; SEQ ID NO:6645 and 7503; SEQ ID NO:15532 and 5217; SEQ ID NO:15531 and 5216; SEQ ID NO:12295 and 5433; SEQ ID NO: 12294 and 5432; SEQ ID NO:15309 and 17307; SEQ ID NO:15308 and 17306; SEQ ID NO:8160 and 13375; SEQ ID NO:8159 and 13374; SEQ ID NO:15464 and 7947; SEQ ID NO:15463 and 7946; SEQ ID NO:13726 and 5773; SEQ ID NO: 13725 and 5772; SEQ ID NO:8740 and 11803; SEQ ID NO:8739 and 11802; SEQ ID NO: 11959 and 13488; SEQ ID NO: 11958 and 13487; SEQ ID NO:8082 and 5471; SEQ ID NO:8081 and 5470; SEQ ID NO:8296 and 5283; SEQ ID NO:8295 and 5282; SEQ ID NO:8557 and 7831; SEQ ID NO:8556 and 7830; SEQ ID NO:8760 and 13521; SEQ ID NO:8759 and 13515; SEQ ID NO:8605 and 13053; SEQ ID NO:8604 and 13052; SEQ ID NO:14577 and 9295; SEQ ID NO:14576 and 9294; SEQ ID NO:14573 and 5219; SEQ ID NO:14572 and 5218; SEQ ID NO:15500 and 13463; SEQ ID NO: 15499 and 13462; SEQ ID NO: 14523 and 7240; SEQ ID NO: 14522 and 7239; SEQ ID NO: 12219 and 5703; SEQ ID NO: 12218 and 5702; SEQ ID NO:8982 and 13150; SEQ ID NO:8981 and 13149; SEQ ID NO:14199 and 17387; SEQ ID NO:14197 and 17377; SEQ ID NO:9000 and 13202; SEQ ID NO:8999 and 13201; SEQ ID NO:15020 and 5227; SEQ ID NO: 15019 and 5226; SEQ ID NO:15057 and 5175; SEQ ID NO:15056 and 5174; SEQ ID NO:7836 and 7202; SEQ ID NO:7835 and 7201; SEQ ID NO:14871 and 7114; SEQ ID NO:14870 and 7113; SEQ ID NO:9281 and 7782; SEQ ID NO:9280 and 7781; SEQ ID NO:8002 and 13047; SEQ ID NO:8001 and 13046; SEQ ID NO:8978 and 13152; SEQ ID NO: 8977 and 13151; SEQ ID NO:14851 and 7026; SEQ ID NO:14850 and 7025; SEQ ID NO:15327 and 7112; SEQ ID NO:15326 and 7111; SEQ ID NO:14811 and 7176; SEQ ID NO:14810 and 7175; SEQ ID NO:8774 and 13075; SEQ ID NO:8773 and 13074; SEQ ID NO:9078 and 6951; SEQ ID NO:9074 and 6950; SEQ ID NO:8144 and 11815; SEQ ID NO:8143 and 11814; SEQ ID NO:14669 and 7281; SEQ ID NO: 14665 and 7268; SEQ ID NO: 14505 and 5334; SEQ ID NO: 14504 and 5327; SEQ ID NO:14873 and 7206; SEQ ID NO:14872 and 7205; SEQ ID NO:15205 and 4814; SEQ ID NO:15204 and 4813; SEQ ID NO:15378 and 4102; SEQ ID NO: 15374 and 4101; SEQ ID NO:8506 and 10656; SEQ ID NO:8505 and 10655; SEQ ID NO: 12052 and 13276; SEQ ID NO:12051 and 13275; SEQ ID NO:6264 and 17281; SEQ ID NO:6263 and 17280; SEQ ID NO:9239 and 13387; SEQ ID NO:9238 and 13386; SEQ ID NO: 14971 and 5169; SEQ ID NO: 14970 and 5168; SEQ ID NO:9137 and 10596; SEQ ID NO:9136 and 10595; SEQ ID NO: 12297 and 5291; SEQ ID NO: 12296 and 5290; SEQ ID NO:8844 and 6357; SEQ ID NO:8843 and 6356; SEQ ID NO:14691 and 17381; SEQ ID NO:14689 and 17378; SEQ ID NO:8906 and 13118; SEQ ID NO:8899 and 13117; SEQ ID NO:11922 and 13381; SEQ ID NO:11921 and 13380; SEQ ID NO:15037 and 7010; SEQ ID NO:15036 and 7009; SEQ ID NO:8533 and 13123; SEQ ID NO:8527 and 13122; SEQ ID NO:9131 and 5749; SEQ ID NO:9130 and 5736; SEQ ID NO:15440 and 7386; SEQ ID NO:15439 and 7385; SEQ ID NO:8146 and 7750; SEQ ID NO:8145 and 7749; SEQ ID NO:8621 and 13146; SEQ ID NO:8620 and 13145; SEQ ID NO:8324 and 6910; SEQ ID NO:8323 and 6909; SEQ ID NO: 14039 and 4900; SEQ ID NO: 14038 and 4899; SEQ ID NO: 15155 and 7866; SEQ ID NO: 15154 and 7865; SEQ ID NO:15085 and 7246; SEQ ID NO: 15084 and 7245; SEQ ID NO:8454 and 11819; SEQ ID NO:8453 and 11818; SEQ ID NO:15341 and 7937; SEQ ID NO:15340 and 7936; SEQ ID NO:15357 and 7218; SEQ ID NO:15356 and 7217; SEQ ID NO: 12289 and 5257; SEQ ID NO: 12288 and 5256; SEQ ID NO:8595 and 7072; SEQ ID NO:8594 and 7071; SEQ ID NO:15546 and 13457; SEQ ID NO:15545 and 13456; SEQ ID NO:8818 and 6391; SEQ ID NO:8817 and 6390; SEQ ID NO:6650 and 7566; SEQ ID NO:6649 and 7565; SEQ ID NO:15137 and 7358; SEQ ID NO:15136 and 7357; SEQ ID NO:13640 and 7704; SEQ ID NO: 13639 and 7703; SEQ ID NO:8988 and 13208; SEQ ID NO:8987 and 13207; SEQ ID NO:15337 and 4786; SEQ ID NO: 15336 and 4785; SEQ ID NO:9167 and 6202; SEQ ID NO:9166 and 6201; SEQ ID NO:9097 and 5233; SEQ ID NO:9096 and 5232; SEQ ID NO:8178 and 7446; SEQ ID NO:8177 and 7445; SEQ ID NO:15143 and 7244; SEQ ID NO:15142 and 7241; SEQ ID NO:15261 and 5689; SEQ ID NO: 15260 and 5688; SEQ ID NO:12058 and 5873; SEQ ID NO: 12057 and 5872; SEQ ID NO: 14839 and 5469; SEQ ID NO:14838 and 5468; SEQ ID NO: 14069 and 7198; SEQ ID NO: 14068 and 7197; SEQ ID NO:8746 and 13091; SEQ ID NO:8745 and 13090; SEQ ID NO: 14172 and 13429; SEQ ID NO:14171 and 13428; SEQ ID NO:14676 and 17384; SEQ ID NO:14665 and 17377; SEQ ID NO:14057 and 7312; SEQ ID NO:14056 and 7311; SEQ ID NO:8122 and 13439; SEQ ID NO:8121 and 13438; SEQ ID NO:8214 and 5319; SEQ ID NO:8213 and 5318; SEQ ID NO:8744 and 5802; SEQ ID NO:8743 and 5801; SEQ ID NO:8597 and 5812; SEQ ID NO:8596 and 5811; SEQ ID NO: 14180 and 13079; SEQ ID NO: 14179 and 13078; SEQ ID NO:9044 and 5387; SEQ ID NO:9043 and 5386; SEQ ID NO: 14981 and 4959; SEQ ID NO: 14980 and 4958; SEQ ID NO:8984 and 13154; SEQ ID NO:8983 and 13153; SEQ ID NO: 15047 and 10626; SEQ ID NO: 15046 and 10625; SEQ ID NO:8426 and 13212; SEQ ID NO:8425 and 13211; SEQ ID NO:8128 and 9331; SEQ ID NO:8127 and 9330; SEQ ID NO:15536 and 5181; SEQ ID NO:15535 and 5180; SEQ ID NO: 14742 and 13045; SEQ ID NO: 14741 and 13044; SEQ ID NO:15367 and 5869; SEQ ID NO:15366 and 5868; SEQ ID NO:8464 and 4098; SEQ ID NO:8463 and 4097; SEQ ID NO:15313 and 6433; SEQ ID NO:15312 and 6432; SEQ ID NO:11047 and 7931; SEQ ID NO:11046 and 7930; SEQ ID NO:14012 and 5075; SEQ ID NO:14011 and 5074; SEQ ID NO:15534 and 17348; SEQ ID NO:15533 and 17347; SEQ ID NO: 18453 and 4114; SEQ ID NO:18452 and 4113; SEQ ID NO:14298 and 5397; SEQ ID NO: 14297 and 5396; SEQ ID NO:6276 and 4525; SEQ ID NO:6275 and 4524; SEQ ID NO: 14468 and 4724; SEQ ID NO: 14466 and 4715; SEQ ID NO:15538 and 17317; SEQ ID NO:15537 and 17316; SEQ ID NO: 13849 and 4684; SEQ ID NO:13848 and 4683; SEQ ID NO:14031 and 7256; SEQ ID NO:14030 and 7255; SEQ ID NO:14901 and 5431; SEQ ID NO:14900 and 5430; SEQ ID NO:14953 and 7328; SEQ ID NO: 14952 and 7327; SEQ ID NO: 15394 and 7802; SEQ ID NO:15393 and 7801; SEQ ID NO:8190 and 6830; SEQ ID NO:8189 and 6829; SEQ ID NO:8514 and 5083; SEQ ID NO:8513 and 5082; SEQ ID NO:8312 and 7708; SEQ ID NO:8311 and 7707; SEQ ID NO:14168 and 7074; SEQ ID NO:14167 and 7073; SEQ ID NO:8084 and 5673; SEQ ID NO:8083 and 5672; SEQ ID NO:13887 and 4796; SEQ ID NO:13886 and 4795; SEQ ID NO:15544 and 17340; SEQ ID NO: 15543 and 17339; SEQ ID NO: 14497 and 7702; SEQ ID NO:14496 and 7701; SEQ ID NO:8713 and 7277; SEQ ID NO:8711 and 7275; SEQ ID NO: 12207 and 5121; SEQ ID NO: 12206 and 5120; SEQ ID NO:14754 and 17332; SEQ ID NO: 14753 and 17329; SEQ ID NO:8246 and 17328; SEQ ID NO:8238 and 17324; SEQ ID NO: 14292 and 13049; SEQ ID NO:14291 and 13048; SEQ ID NO:13940 and 4846; SEQ ID NO:13939 and 4845; SEQ ID NO:8047 and 7444; SEQ ID NO:8046 and 7443; SEQ ID NO:14329 and 6413; SEQ ID NO:14328 and 6412; SEQ ID NO:8444 and 5675; SEQ ID NO:8443 and 5674; SEQ ID NO: 13690 and 5891; SEQ ID NO: 13689 and 5890; SEQ ID NO:8525 and 5039; SEQ ID NO:8524 and 5038; SEQ ID NO:8478 and 17390; SEQ ID NO:8477 and 17389; SEQ ID NO:8830 and 5585; SEQ ID NO:8829 and 5584; SEQ ID NO:8567 and 11821; SEQ ID NO:8566 and 11820; SEQ ID NO: 15129 and 7306; SEQ ID NO: 15128 and 7305; SEQ ID NO:8240 and 7267; SEQ ID NO:8238 and 7263; SEQ ID NO:14067 and 7941; SEQ ID NO: 14066 and 7940; SEQ ID NO: 14509 and 7456; SEQ ID NO: 14508 and 7455; SEQ ID NO:8164 and 6421; SEQ ID NO:8163 and 6420; SEQ ID NO:8040 and 11844; SEQ ID NO:8039 and 11843; SEQ ID NO: 14202 and 6961; SEQ ID NO:14201 and 6952; SEQ ID NO:8032 and 18240; SEQ ID NO:8031 and 18239; SEQ ID NO:8949 and 6415; SEQ ID NO:8948 and 6414; SEQ ID NO:8408 and 9429; SEQ ID NO:8407 and 9428; SEQ ID NO:8059 and 6425; SEQ ID NO:8058 and 6424; SEQ ID NO:9006 and 5413; SEQ ID NO:9005 and 5412; SEQ ID NO:12165 and 17080; SEQ ID NO: 12164 and 17079; SEQ ID NO:8049 and 5755; SEQ ID NO:8048 and 5754; SEQ ID NO:14317 and 4559; SEQ ID NO:14316 and 4558; SEQ ID NO:12043 and 5421; SEQ ID NO:12042 and 5420; SEQ ID NO: 15293 and 7248; SEQ ID NO: 15292 and 7247; SEQ ID NO:8384 and 5215; SEQ ID NO:8383 and 5214; SEQ ID NO:6660 and 7236; SEQ ID NO:6659 and 7235; SEQ ID NO:15363 and 6359; SEQ ID NO: 15362 and 6358; SEQ ID NO:9189 and 11835; SEQ ID NO:9188 and 11834; SEQ ID NO:8742 and 13606; SEQ ID NO:8741 and 13605; SEQ ID NO:8396 and 5341; SEQ ID NO:8395 and 5340; SEQ ID NO: 14843 and 5763; SEQ ID NO: 14842 and 5762; SEQ ID NO:14603 and 6447; SEQ ID NO: 14602 and 6446; SEQ ID NO:8394 and 5405; SEQ ID NO:8393 and 5404; SEQ ID NO:8286 and 5681; SEQ ID NO:8285 and 5680; SEQ ID NO:8338 and 13445; SEQ ID NO:8337 and 13444; SEQ ID NO:15109 and 6834; SEQ ID NO:15108 and 6833; SEQ ID NO:8212 and 6757; SEQ ID NO:8211 and 6756; SEQ ID NO:13746 and 11858; SEQ ID NO:13745 and 11857; SEQ ID NO:15359 and 6373; SEQ ID NO:15358 and 6372; SEQ ID NO:9197 and 6996; SEQ ID NO:9196 and 6995; SEQ ID NO:9175 and 5167; SEQ ID NO:9174 and 5166; SEQ ID NO: 14919 and 5213; SEQ ID NO:14918 and 5212; SEQ ID NO:14439 and 4121; SEQ ID NO:14438 and 4120; SEQ ID NO:9012 and 5735; SEQ ID NO:9011 and 5734; SEQ ID NO:14927 and 13206; SEQ ID NO:14926 and 13205; SEQ ID NO: 17684 and 5555; SEQ ID NO: 17683 and 5554; SEQ ID NO: 13972 and 4444; SEQ ID NO:13971 and 4443; SEQ ID NO:9072 and 13521; SEQ ID NO:9071 and 13515; SEQ ID NO:14579 and 6361; SEQ ID NO:14578 and 6360; SEQ ID NO:8014 and 5883; SEQ ID NO:8013 and 5882; SEQ ID NO:13915 and 13353; SEQ ID NO:13914 and 13352; SEQ ID NO:8659 and 11833; SEQ ID NO:8658 and 11832; SEQ ID NO:8494 and 5731; SEQ ID NO:8493 and 5730; SEQ ID NO:8398 and 5235; SEQ ID NO:8397 and 5234; SEQ ID NO:14136 and 7440; SEQ ID NO: 14135 and 7439; SEQ ID NO:8780 and 6970; SEQ ID NO:8779 and 6968; SEQ ID NO:15207 and 16808; SEQ ID NO:15206 and 16807; SEQ ID NO:14156 and 18126; SEQ ID NO: 14155 and 18125; SEQ ID NO:8603 and 11831; SEQ ID NO:8602 and 11830; SEQ ID NO:8392 and 6304; SEQ ID NO:8391 and 6303; SEQ ID NO:8204 and 13555; SEQ ID NO:8203 and 13554; SEQ ID NO:13893 and 6230; SEQ ID NO:13892 and 6229; SEQ ID NO: 14682 and 13278; SEQ ID NO:14681 and 13277; SEQ ID NO:14349 and 4896; SEQ ID NO:14348 and 4895; SEQ ID NO:14274 and 5423; SEQ ID NO:14273 and 5422; SEQ ID NO: 13877 and 7945; SEQ ID NO: 13876 and 7944; SEQ ID NO: 15390 and 6409; SEQ ID NO: 15389 and 6408; SEQ ID NO: 14499 and 13168; SEQ ID NO: 14498 and 13167; SEQ ID NO:6300 and 4969; SEQ ID NO:6299 and 4968; SEQ ID NO:12293 and 10611; SEQ ID NO: 12292 and 10610; SEQ ID NO: 15398 and 6427; SEQ ID NO: 15397 and 6426; SEQ ID NO: 14643 and 6445; SEQ ID NO: 14642 and 6444; SEQ ID NO: 14491 and 13459; SEQ ID NO: 14490 and 13458; SEQ ID NO:14065 and 7316; SEQ ID NO:14064 and 7315; SEQ ID NO:14359 and 13160; SEQ ID NO: 14358 and 13159; SEQ ID NO:8446 and 6353; SEQ ID NO:8445 and 6352; SEQ ID NO:8016 and 5105; SEQ ID NO:8015 and 5104; SEQ ID NO:6708 and 13549; SEQ ID NO:6707 and 13548; SEQ ID NO:12287 and 5011; SEQ ID NO:12286 and 5010; SEQ ID NO:8188 and 10642; SEQ ID NO:8187 and 10641; SEQ ID NO:13865 and 4756; SEQ ID NO:13864 and 4755; SEQ ID NO:9171 and 5841; SEQ ID NO:9170 and 5840; SEQ ID NO:13688 and 17525; SEQ ID NO:13687 and 17524; SEQ ID NO:8018 and 5487; SEQ ID NO:8017 and 5486; SEQ ID NO:14063 and 17515; SEQ ID NO:14062 and 17514; SEQ ID NO:15542 and 4850; SEQ ID NO:15541 and 4849; SEQ ID NO:8816 and 13266; SEQ ID NO:8815 and 13265; SEQ ID NO: 14409 and 5243; SEQ ID NO: 14408 and 5242; SEQ ID NO:6664 and 13214; SEQ ID NO:6663 and 13213; SEQ ID NO: 14399 and 5705; SEQ ID NO: 14398 and 5704; SEQ ID NO: 14712 and 4718; SEQ ID NO: 14705 and 4716; SEQ ID NO:14381 and 13492; SEQ ID NO:14380 and 13491; SEQ ID NO:14367 and 17523; SEQ ID NO:14366 and 17522; SEQ ID NO: 14511 and 5695; SEQ ID NO:14510 and 5694; SEQ ID NO:14375 and 5379; SEQ ID NO:14374 and 5378; SEQ ID NO: 14357 and 13210; SEQ ID NO: 14356 and 13209; SEQ ID NO: 15024 and 5625; SEQ ID NO: 15023 and 5624; SEQ ID NO:13680 and 7508; SEQ ID NO:13679 and 7507; SEQ ID NO:14371 and 13467; SEQ ID NO:14370 and 13466; SEQ ID NO:13861 and 7374; SEQ ID NO:13860 and 7373; SEQ ID NO:14457 and 17521; SEQ ID NO:14456 and 17520; SEQ ID NO:15147 and 5129; SEQ ID NO:15146 and 5128; SEQ ID NO:15386 and 13316; SEQ ID NO:15385 and 13315; SEQ ID NO:8266 and 10642; SEQ ID NO:8265 and 10641; SEQ ID NO: 14493 and 7408; SEQ ID NO: 14492 and 7407; SEQ ID NO: 15321 and 6363; SEQ ID NO: 15320 and 6362; SEQ ID NO:12213 and 17336; SEQ ID NO:12212 and 17335; SEQ ID NO:8358 and 17519; SEQ ID NO:8357 and 17518; SEQ ID NO:13863 and 7014; SEQ ID NO:13862 and 7013; SEQ ID NO:14278 and 6347; SEQ ID NO:14277 and 6346; SEQ ID NO:8314 and 13188; SEQ ID NO:8313 and 13187; SEQ ID NO:14373 and 17507; SEQ ID NO:14372 and 17506; SEQ ID NO:14507 and 5769; SEQ ID NO: 14506 and 5768; SEQ ID NO:8356 and 13349; SEQ ID NO:8355 and 13348; SEQ ID NO: 12035 and 17301; SEQ ID NO:12034 and 17300; SEQ ID NO:12084 and 10650; SEQ ID NO:12083 and 10649; SEQ ID NO:8734 and 5063; SEQ ID NO:8726 and 5060; SEQ ID NO:8042 and 10634; SEQ ID NO:8041 and 10633; SEQ ID NO:14995 and 13520; SEQ ID NO:14985 and 13515; SEQ ID NO:14583 and 11397; SEQ ID NO: 14582 and 11396; SEQ ID NO: 14409 and 5373; SEQ ID NO: 14408 and 5372; SEQ ID NO:8766 and 4704; SEQ ID NO:8765 and 4703; SEQ ID NO:14061 and 7364; SEQ ID NO: 14060 and 7363; SEQ ID NO:8112 and 5581; SEQ ID NO:8111 and 5580; SEQ ID NO:8919 and 7678; SEQ ID NO:8918 and 7677; SEQ ID NO:8354 and 7766; SEQ ID NO:8353 and 7765; SEQ ID NO:8073 and 13234; SEQ ID NO:8072 and 13233; SEQ ID NO:13851 and 9315; SEQ ID NO:13850 and 9314; SEQ ID NO:13678 and 7506; SEQ ID NO:13677 and 7505; SEQ ID NO:8180 and 7482; SEQ ID NO:8179 and 7481; SEQ ID NO:17686 and 11823; SEQ ID NO:17685 and 11822; SEQ ID NO:14379 and 13465; SEQ ID NO: 14378 and 13464; SEQ ID NO: 14121 and 7668; SEQ ID NO: 14120 and 7667; SEQ ID NO:14343 and 9321; SEQ ID NO:14342 and 9320; SEQ ID NO:14481 and 5163; SEQ ID NO:14480 and 5162; SEQ ID NO: 12184 and 7627; SEQ ID NO: 12183 and 7615; SEQ ID NO: 14515 and 6345; SEQ ID NO: 14514 and 6344; SEQ ID NO:9004 and 10648; SEQ ID NO:9003 and 10647; SEQ ID NO:8336 and 13385; SEQ ID NO:8335 and 13384; SEQ ID NO: 14477 and 13184; SEQ ID NO: 14476 and 13183; SEQ ID NO: 13686 and 17098; SEQ ID NO: 13685 and 17097; SEQ ID NO:8470 and 6898; SEQ ID NO:8469 and 6897; SEQ ID NO:14487 and 13469; SEQ ID NO:14486 and 13468; SEQ ID NO:8824 and 6411; SEQ ID NO:8823 and 6410; SEQ ID NO:15382 and 13286; SEQ ID NO:15381 and 13285; SEQ ID NO:14391 and 17509; SEQ ID NO:14390 and 17508; SEQ ID NO:8416 and 4446; SEQ ID NO:8415 and 4445; SEQ ID NO:14345 and 9319; SEQ ID NO:14344 and 9318; SEQ ID NO:14641 and 6383; SEQ ID NO:14640 and 6382; SEQ ID NO:9022 and 6387; SEQ ID NO:9021 and 6386; SEQ ID NO:15307 and 18236; SEQ ID NO:15306 and 18235; SEQ ID NO:13660 and 6980; SEQ ID NO:13659 and 6979; SEQ ID NO: 15578 and 4450; SEQ ID NO:15577 and 4449; SEQ ID NO:13684 and 13166; SEQ ID NO:13683 and 13165; SEQ ID NO:15384 and 13312; SEQ ID NO:15383 and 13311; SEQ ID NO:18186 and 13198; SEQ ID NO: 18185 and 13197; SEQ ID NO:15133 and 7066; SEQ ID NO:15132 and 7065; SEQ ID NO:14684 and 5877; SEQ ID NO: 14683 and 5876; SEQ ID NO:8888 and 10632; SEQ ID NO:8887 and 10631; SEQ ID NO: 14740 and 6377; SEQ ID NO: 14739 and 6376; SEQ ID NO:8941 and 13373; SEQ ID NO:8940 and 13372; SEQ ID NO: 14276 and 5099; SEQ ID NO: 14275 and 5098; SEQ ID NO:15101 and 13541; SEQ ID NO:15100 and 13540; SEQ ID NO: 14059 and 5723; SEQ ID NO:14058 and 5722; SEQ ID NO: 13932 and 4884; SEQ ID NO:13931 and 4883; SEQ ID NO: 14877 and 7949; SEQ ID NO: 14876 and 7948; SEQ ID NO:8310 and 6763; SEQ ID NO:8309 and 6762; SEQ ID NO: 14589 and 4788; SEQ ID NO: 14588 and 4787; SEQ ID NO:13913 and 4754; SEQ ID NO:13912 and 4753; SEQ ID NO:8587 and 13178; SEQ ID NO:8586 and 13177; SEQ ID NO:8802 and 4515; SEQ ID NO:8801 and 4514; SEQ ID NO:14296 and 13537; SEQ ID NO: 14295 and 13536; SEQ ID NO:6599 and 6395; SEQ ID NO:6598 and 6394; SEQ ID NO:6652 and 7744; SEQ ID NO:6651 and 7743; SEQ ID NO:18180 and 17398; SEQ ID NO:18179 and 17397; SEQ ID NO:15193 and 10598; SEQ ID NO:15192 and 10597; SEQ ID NO:8641 and 7494; SEQ ID NO:8640 and 7493; SEQ ID NO:14010 and 4882; SEQ ID NO:14009 and 4881; SEQ ID NO:8902 and 4955; SEQ ID NO:8901 and 4954; SEQ ID NO:14037 and 5001; SEQ ID NO:14036 and 5000; SEQ ID NO:8256 and 13514; SEQ ID NO:8255 and 13513; SEQ ID NO: 14233 and 9287; SEQ ID NO: 14232 and 9286; SEQ ID NO:15018 and 7784; SEQ ID NO:15017 and 7783; SEQ ID NO:15225 and 12274; SEQ ID NO:15224 and 12273; SEQ ID NO:9193 and 11742; SEQ ID NO:9192 and 11741; SEQ ID NO:6656 and 7740; SEQ ID NO:6655 and 7739; SEQ ID NO: 12189 and 4898; SEQ ID NO:12188 and 4897; SEQ ID NO:8110 and 6888; SEQ ID NO:8109 and 6887; SEQ ID NO:14254 and 13539; SEQ ID NO:14250 and 13538; SEQ ID NO:8262 and 7516; SEQ ID NO:8261 and 7515; SEQ ID NO:8601 and 4760; SEQ ID NO:8600 and 4759; SEQ ID NO: 15249 and 7732; SEQ ID NO: 15248 and 7731; SEQ ID NO:8186 and 7772; SEQ ID NO:8185 and 7771; SEQ ID NO:8202 and 5627; SEQ ID NO:8201 and 5626; SEQ ID NO:8609 and 6401; SEQ ID NO:8608 and 6400; SEQ ID NO:15468 and 4824; SEQ ID NO:15467 and 4823; SEQ ID NO:8098 and 5786; SEQ ID NO:8097 and 5785; SEQ ID NO: 14517 and 4864; SEQ ID NO: 14516 and 4863; SEQ ID NO: 12174 and 17387; SEQ ID NO: 12170 and 17377; SEQ ID NO: 14805 and 5771; SEQ ID NO: 14804 and 5770; SEQ ID NO:8030 and 13343; SEQ ID NO:8029 and 13342; SEQ ID NO:14313 and 4981; SEQ ID NO:14312 and 4977; SEQ ID NO:6591 and 6393; SEQ ID NO:6590 and 6392; SEQ ID NO:8108 and 7400; SEQ ID NO:8107 and 7399; SEQ ID NO:13891 and 4886; SEQ ID NO:13890 and 4885; SEQ ID NO:14657 and 13512; SEQ ID NO:14656 and 13511; SEQ ID NO:13929 and 17394; SEQ ID NO:13921 and 17393; SEQ ID NO:8124 and 7512; SEQ ID NO:8123 and 7511; SEQ ID NO:8710 and 4110; SEQ ID NO:8709 and 4109; SEQ ID NO:9026 and 5733; SEQ ID NO:9025 and 5732; SEQ ID NO:9040 and 13407; SEQ ID NO:9039 and 13406; SEQ ID NO:6654 and 7718; SEQ ID NO:6653 and 7717; SEQ ID NO:13752 and 6435; SEQ ID NO:13751 and 6434; SEQ ID NO: 15470 and 4810; SEQ ID NO: 15469 and 4809; SEQ ID NO:8388 and 6228; SEQ ID NO:8387 and 6227; SEQ ID NO:8138 and 7636; SEQ ID NO:8137 and 7635; SEQ ID NO:6662 and 9335; SEQ ID NO:6661 and 9334; SEQ ID NO: 14969 and 7144; SEQ ID NO:14968 and 7143; SEQ ID NO:8232 and 6860; SEQ ID NO:8231 and 6859; SEQ ID NO:9157 and 4902; SEQ ID NO:9156 and 4901; SEQ ID NO:8927 and 7627; SEQ ID NO:8926 and 7614; SEQ ID NO:8896 and 6928; SEQ ID NO:8895 and 6927; SEQ ID NO:8330 and 7530; SEQ ID NO:8329 and 7529; SEQ ID NO: 14720 and 17412; SEQ ID NO: 14719 and 17411; SEQ ID NO:9145 and 5537; SEQ ID NO:9144 and 5536; SEQ ID NO:15187 and 13365; SEQ ID NO:15186 and 13364; SEQ ID NO: 14973 and 7350; SEQ ID NO: 14972 and 7349; SEQ ID NO:8619 and 13531; SEQ ID NO:8618 and 13530; SEQ ID NO:8174 and 11837; SEQ ID NO:8173 and 11836; SEQ ID NO:14793 and 7652; SEQ ID NO:14792 and 7651; SEQ ID NO:11768 and 5139; SEQ ID NO:11767 and 5138; SEQ ID NO:8226 and 5043; SEQ ID NO:8225 and 5042; SEQ ID NO: 14853 and 10622; SEQ ID NO: 14852 and 10621; SEQ ID NO:9028 and 17424; SEQ ID NO:9027 and 17423; SEQ ID NO:8116 and 4987; SEQ ID NO:8115 and 4986; SEQ ID NO:12119 and 7108; SEQ ID NO:12118 and 7107; SEQ ID NO:8243 and 17331; SEQ ID NO:8238 and 17330; SEQ ID NO:6587 and 7128; SEQ ID NO:6586 and 7127; SEQ ID NO:8617 and 13529; SEQ ID NO:8616 and 13528; SEQ ID NO: 18174 and 7868; SEQ ID NO:18173 and 7867; SEQ ID NO:8516 and 7308; SEQ ID NO:8515 and 7307; SEQ ID NO:8288 and 6864; SEQ ID NO:8287 and 6863; SEQ ID NO:8754 and 17342; SEQ ID NO:8753 and 17341; SEQ ID NO:15127 and 7402; SEQ ID NO:15126 and 7401; SEQ ID NO:8615 and 13535; SEQ ID NO:8614 and 13534; SEQ ID NO: 14541 and 7148; SEQ ID NO: 14540 and 7147; SEQ ID NO: 12086 and 13320; SEQ ID NO: 12085 and 13319; SEQ ID NO: 10972 and 5661; SEQ ID NO: 10971 and 5660; SEQ ID NO:14089 and 5079; SEQ ID NO:14088 and 5078; SEQ ID NO:14361 and 6916; SEQ ID NO: 14360 and 6915; SEQ ID NO: 14715 and 7738; SEQ ID NO: 14704 and 7737; SEQ ID NO: 14921 and 4848; SEQ ID NO:14920 and 4847; SEQ ID NO:8508 and 7556; SEQ ID NO:8507 and 7555; SEQ ID NO:14132 and 5085; SEQ ID NO:14131 and 5084; SEQ ID NO:9113 and 17426; SEQ ID NO:9112 and 17425; SEQ ID NO:15515 and 7484; SEQ ID NO:15514 and 7483; SEQ ID NO:18150 and 7593; SEQ ID NO:18149 and 7592; SEQ ID NO:8575 and 5981; SEQ ID NO:8574 and 5980; SEQ ID NO:13980 and 11744; SEQ ID NO:13979 and 11743; SEQ ID NO: 15165 and 4694; SEQ ID NO:15164 and 4693; SEQ ID NO:8158 and 5659; SEQ ID NO:8157 and 5658; SEQ ID NO: 14907 and 7609; SEQ ID NO: 14906 and 7608; SEQ ID NO:15089 and 6862; SEQ ID NO:15088 and 6861; SEQ ID NO:15185 and 13403; SEQ ID NO:15184 and 13402; SEQ ID NO:12071 and 13517; SEQ ID NO:12070 and 13515; SEQ ID NO:14099 and 13431; SEQ ID NO:14098 and 13430; SEQ ID NO:14718 and 6419; SEQ ID NO:14717 and 6418; SEQ ID NO: 14449 and 9285; SEQ ID NO: 14448 and 9284; SEQ ID NO:8196 and 7648; SEQ ID NO:8195 and 7647; SEQ ID NO:14091 and 5045; SEQ ID NO: 14090 and 5044; SEQ ID NO: 14885 and 7192; SEQ ID NO:14884 and 7191; SEQ ID NO:18162 and 5221; SEQ ID NO:18161 and 5220; SEQ ID NO:8100 and 7682; SEQ ID NO:8099 and 7681; SEQ ID NO:8378 and 5315; SEQ ID NO:8377 and 5314; SEQ ID NO: 14445 and 6926; SEQ ID NO: 14444 and 6925; SEQ ID NO: 15173 and 7002; SEQ ID NO: 15172 and 7001; SEQ ID NO: 14829 and 7538; SEQ ID NO: 14828 and 7537; SEQ ID NO: 14661 and 13490; SEQ ID NO:14660 and 13489; SEQ ID NO:15111 and 5249; SEQ ID NO:15110 and 5248; SEQ ID NO:18182 and 13393; SEQ ID NO:18181 and 13392; SEQ ID NO:14651 and 6381; SEQ ID NO:14650 and 6380; SEQ ID NO: 14765 and 13322; SEQ ID NO: 14764 and 13321; SEQ ID NO: 13795 and 5375; SEQ ID NO:13794 and 5374; SEQ ID NO:14611 and 4822; SEQ ID NO:14610 and 4821; SEQ ID NO: 15245 and 13300; SEQ ID NO:15244 and 13299; SEQ ID NO:8114 and 13500; SEQ ID NO:8113 and 13499; SEQ ID NO:14905 and 6914; SEQ ID NO:14904 and 6913; SEQ ID NO: 13990 and 5073; SEQ ID NO: 13989 and 5072; SEQ ID NO:14268 and 5553; SEQ ID NO:14267 and 5552; SEQ ID NO:15253 and 16579; SEQ ID NO:15252 and 16578; SEQ ID NO:12205 and 13258; SEQ ID NO:12204 and 13257; SEQ ID NO:8350 and 5171; SEQ ID NO:8349 and 5170; SEQ ID NO:7834 and 6976; SEQ ID NO:7833 and 6975; SEQ ID NO:8194 and 7390; SEQ ID NO:8193 and 7389; SEQ ID NO:8800 and 13533; SEQ ID NO:8799 and 13532; SEQ ID NO:15161 and 7118; SEQ ID NO:15160 and 7117; SEQ ID NO:8850 and 7416; SEQ ID NO:8849 and 7415; SEQ ID NO:15213 and 13089; SEQ ID NO:15212 and 13088; SEQ ID NO:12080 and 13284; SEQ ID NO: 12079 and 13283; SEQ ID NO:6260 and 5597; SEQ ID NO:6259 and 5596; SEQ ID NO:8236 and 13061; SEQ ID NO:8235 and 13060; SEQ ID NO: 15199 and 7126; SEQ ID NO: 15198 and 7125; SEQ ID NO:15460 and 5669; SEQ ID NO:15459 and 5668; SEQ ID NO:8051 and 7778; SEQ ID NO:8050 and 7777; SEQ ID NO:8633 and 7575; SEQ ID NO:8632 and 7574; SEQ ID NO:9111 and 17428; SEQ ID NO:9110 and 17427; SEQ ID NO:8322 and 13401; SEQ ID NO:8321 and 13400; SEQ ID NO:6593 and 13077; SEQ ID NO:6592 and 13076; SEQ ID NO:8026 and 10674; SEQ ID NO:8025 and 10673; SEQ ID NO:8768 and 11846; SEQ ID NO:8767 and 11845; SEQ ID NO:8748 and 5983; SEQ ID NO:8747 and 5982; SEQ ID NO:8649 and 5593; SEQ ID NO:8648 and 5592; SEQ ID NO:9221 and 5887; SEQ ID NO:9220 and 5886; SEQ ID NO:8653 and 5591; SEQ ID NO:8652 and 5590; SEQ ID NO: 14215 and 13250; SEQ ID NO: 14214 and 13249; SEQ ID NO:9103 and 5545; SEQ ID NO:9102 and 5544; SEQ ID NO:13674 and 4826; SEQ ID NO:13673 and 4825; SEQ ID NO:6288 and 4772; SEQ ID NO:6287 and 4771; SEQ ID NO: 14280 and 17364; SEQ ID NO: 14279 and 17363; SEQ ID NO: 14272 and 7156; SEQ ID NO:14271 and 7155; SEQ ID NO:6641 and 5677; SEQ ID NO:6640 and 5676; SEQ ID NO:14134 and 7388; SEQ ID NO:14133 and 7387; SEQ ID NO:9185 and 12254; SEQ ID NO:9184 and 12253; SEQ ID NO: 12238 and 11825; SEQ ID NO:12237 and 11824; SEQ ID NO:11045 and 7448; SEQ ID NO:11044 and 7447; SEQ ID NO:8681 and 4780; SEQ ID NO:8680 and 4779; SEQ ID NO:15263 and 7758; SEQ ID NO: 15262 and 7757; SEQ ID NO:8045 and 6222; SEQ ID NO:8044 and 6221; SEQ ID NO:9036 and 4527; SEQ ID NO:9035 and 4526; SEQ ID NO:9211 and 18122; SEQ ID NO:9210 and 18121; SEQ ID NO:15305 and 7104; SEQ ID NO:15304 and 7103; SEQ ID NO:14931 and 7935; SEQ ID NO:14930 and 7934; SEQ ID NO:8812 and 4692; SEQ ID NO:8811 and 4691; SEQ ID NO:6675 and 13599; SEQ ID NO:6674 and 13598; SEQ ID NO:8386 and 11801; SEQ ID NO:8385 and 11800; SEQ ID NO:9034 and 7754; SEQ ID NO:9033 and 7753; SEQ ID NO: 14763 and 5857; SEQ ID NO:14762 and 5856; SEQ ID NO:8708 and 6397; SEQ ID NO:8707 and 6396; SEQ ID NO:8738 and 17323; SEQ ID NO:8737 and 17322; SEQ ID NO:14260 and 7096; SEQ ID NO: 14259 and 7095; SEQ ID NO:8665 and 5523; SEQ ID NO:8664 and 5522; SEQ ID NO: 14529 and 13194; SEQ ID NO: 14528 and 13193; SEQ ID NO:8629 and 4645; SEQ ID NO:8628 and 4644; SEQ ID NO: 14752 and 7762; SEQ ID NO:14751 and 7761; SEQ ID NO:8712 and 4909; SEQ ID NO:8711 and 4908; SEQ ID NO: 14637 and 9293; SEQ ID NO: 14636 and 9292; SEQ ID NO:14403 and 5721; SEQ ID NO: 14402 and 5720; SEQ ID NO:9030 and 5701; SEQ ID NO:9029 and 5700; SEQ ID NO:8120 and 13405; SEQ ID NO:8119 and 13404; SEQ ID NO:13797 and 7760; SEQ ID NO:13796 and 7759; SEQ ID NO:15167 and 5267; SEQ ID NO:15166 and 5266; SEQ ID NO:15271 and 5055; SEQ ID NO: 15270 and 5054; SEQ ID NO:6597 and 5759; SEQ ID NO:6596 and 5758; SEQ ID NO:8400 and 6351; SEQ ID NO:8399 and 6350; SEQ ID NO:14451 and 7623; SEQ ID NO:14450 and 7618; SEQ ID NO: 14455 and 5505; SEQ ID NO: 14454 and 5504; SEQ ID NO:15211 and 6988; SEQ ID NO:15210 and 6987; SEQ ID NO: 12285 and 13486; SEQ ID NO: 12284 and 13485; SEQ ID NO: 12054 and 13274; SEQ ID NO: 12053 and 13273; SEQ ID NO:6700 and 13399; SEQ ID NO:6699 and 13398; SEQ ID NO:15361 and 5419; SEQ ID NO:15360 and 5418; SEQ ID NO:13897 and 4770; SEQ ID NO:13896 and 4769; SEQ ID NO: 13704 and 5515; SEQ ID NO: 13703 and 5514; SEQ ID NO: 13708 and 13109; SEQ ID NO: 13707 and 13108; SEQ ID NO:15239 and 5091; SEQ ID NO:15238 and 5090; SEQ ID NO: 14170 and 4768; SEQ ID NO: 14169 and 4767; SEQ ID NO: 14714 and 17380; SEQ ID NO: 14704 and 17378; SEQ ID NO:13871 and 5427; SEQ ID NO:13870 and 5426; SEQ ID NO:12111 and 5499; SEQ ID NO: 12110 and 5498; SEQ ID NO:14519 and 5017; SEQ ID NO: 14518 and 5016; SEQ ID NO: 12145 and 6220; SEQ ID NO:12144 and 6219; SEQ ID NO: 15287 and 7340; SEQ ID NO: 15286 and 7339; SEQ ID NO:13809 and 13451; SEQ ID NO:13808 and 13450; SEQ ID NO:15580 and 5229; SEQ ID NO:15579 and 5228; SEQ ID NO:13974 and 5151; SEQ ID NO: 13973 and 5150; SEQ ID NO:9038 and 13498; SEQ ID NO:9037 and 13497; SEQ ID NO: 12221 and 12328; SEQ ID NO: 12220 and 12327; SEQ ID NO: 12223 and 12326; SEQ ID NO: 12222 and 12325; SEQ ID NO: 14769 and 6369; SEQ ID NO: 14768 and 6368; SEQ ID NO:13970 and 4744; SEQ ID NO: 13969 and 4743; SEQ ID NO:7986 and 13367; SEQ ID NO:7985 and 13366; SEQ ID NO:12137 and 4794; SEQ ID NO:12136 and 4793; SEQ ID NO:6250 and 13347; SEQ ID NO:6249 and 13346; SEQ ID NO: 12060 and 5589; SEQ ID NO: 12059 and 5588; SEQ ID NO:14288 and 4523; SEQ ID NO:14287 and 4522; SEQ ID NO: 14347 and 7384; SEQ ID NO: 14346 and 7383; SEQ ID NO:11140 and 5081; SEQ ID NO:11139 and 5080; SEQ ID NO: 14047 and 4740; SEQ ID NO: 14046 and 4739; SEQ ID NO:8645 and 5429; SEQ ID NO:8644 and 5428; SEQ ID NO: 14629 and 17410; SEQ ID NO:14628 and 17409; SEQ ID NO:15241 and 5089; SEQ ID NO:15240 and 5088; SEQ ID NO:13738 and 5363; SEQ ID NO:13737 and 5362; SEQ ID NO:8510 and 15552; SEQ ID NO:8509 and 15551; SEQ ID NO: 12074 and 5577; SEQ ID NO: 12073 and 5576; SEQ ID NO: 13934 and 17513; SEQ ID NO: 13933 and 17512; SEQ ID NO:12242 and 5019; SEQ ID NO:12241 and 5018; SEQ ID NO:14290 and 6431; SEQ ID NO:14289 and 6430; SEQ ID NO:8903 and 7621; SEQ ID NO:8901 and 7618; SEQ ID NO:18160 and 11752; SEQ ID NO:18159 and 11751; SEQ ID NO:6256 and 4965; SEQ ID NO:6255 and 4964; SEQ ID NO: 12159 and 5541; SEQ ID NO: 12158 and 5540; SEQ ID NO:8756 and 7696; SEQ ID NO:8755 and 7695; SEQ ID NO:8925 and 7420; SEQ ID NO:8924 and 7419; SEQ ID NO:15016 and 17493; SEQ ID NO:15015 and 17492; SEQ ID NO: 18164 and 7674; SEQ ID NO:18163 and 7673; SEQ ID NO:8476 and 13204; SEQ ID NO:8475 and 13203; SEQ ID NO: 12244 and 13043; SEQ ID NO: 12243 and 13042; SEQ ID NO:15014 and 17491; SEQ ID NO: 15013 and 17490; SEQ ID NO:15030 and 5767; SEQ ID NO:15029 and 5766; SEQ ID NO:14148 and 5053; SEQ ID NO: 14147 and 5052; SEQ ID NO:8410 and 10608; SEQ ID NO:8409 and 10607; SEQ ID NO:8406 and 5663; SEQ ID NO:8405 and 5662; SEQ ID NO:8476 and 13561; SEQ ID NO:8475 and 13560; SEQ ID NO:8476 and 13041; SEQ ID NO:8475 and 13040; SEQ ID NO: 14979 and 5839; SEQ ID NO: 14978 and 5838; SEQ ID NO: 15179 and 6292; SEQ ID NO: 15178 and 6291; SEQ ID NO:8569 and 13563; SEQ ID NO:8568 and 13562; SEQ ID NO:8561 and 17288; SEQ ID NO:8560 and 17287; SEQ ID NO: 15526 and 5115; SEQ ID NO:15525 and 5114; SEQ ID NO:8852 and 5460; SEQ ID NO:8851 and 5459; SEQ ID NO:8476 and 13341; SEQ ID NO:8475 and 13340; SEQ ID NO: 15006 and 5223; SEQ ID NO: 15005 and 5222; SEQ ID NO: 14615 and 7042; SEQ ID NO: 14614 and 7041; SEQ ID NO:8798 and 13461; SEQ ID NO:8797 and 13460; SEQ ID NO:8476 and 4690; SEQ ID NO:8475 and 4689; SEQ ID NO:8476 and 4710; SEQ ID NO:8475 and 4709; SEQ ID NO: 14605 and 13242; SEQ ID NO: 14604 and 13241; SEQ ID NO:15448 and 4448; SEQ ID NO:15447 and 4447; SEQ ID NO:14738 and 13262; SEQ ID NO:14737 and 13261; SEQ ID NO:8008 and 7746; SEQ ID NO:8007 and 7745; SEQ ID NO:8061 and 13441; SEQ ID NO:8060 and 13440; SEQ ID NO:7972 and 4100; SEQ ID NO:7971 and 4099; SEQ ID NO:8559 and 13443; SEQ ID NO:8558 and 13442; SEQ ID NO:8476 and 7670; SEQ ID NO:8475 and 7669; SEQ ID NO:9127 and 5709; SEQ ID NO:9126 and 5708; SEQ ID NO:15528 and 5119; SEQ ID NO:15527 and 5118; SEQ ID NO:15012 and 5851; SEQ ID NO:15011 and 5850; SEQ ID NO:18166 and 13222; SEQ ID NO:18165 and 13221; SEQ ID NO:8055 and 12250; SEQ ID NO:8054 and 12249; SEQ ID NO:13714 and 4076; SEQ ID NO:13713 and 4075; SEQ ID NO:13799 and 6924; SEQ ID NO:13798 and 6923; SEQ ID NO:12133 and 5879; SEQ ID NO: 12132 and 5878; SEQ ID NO:9159 and 5601; SEQ ID NO:9158 and 5600; SEQ ID NO:15347 and 13290; SEQ ID NO:15346 and 13289; SEQ ID NO: 14722 and 7326; SEQ ID NO: 14721 and 7325; SEQ ID NO:6284 and 7200; SEQ ID NO:6283 and 7199; SEQ ID NO:7817 and 5529; SEQ ID NO:7816 and 5528; SEQ ID NO:13656 and 5649; SEQ ID NO:13655 and 5648; SEQ ID NO: 14401 and 7583; SEQ ID NO: 14400 and 7582; SEQ ID NO:13859 and 5729; SEQ ID NO:13858 and 5728; SEQ ID NO:14211 and 17100; SEQ ID NO:14210 and 17099; SEQ ID NO:9199 and 6429; SEQ ID NO:9198 and 6428; SEQ ID NO:8096 and 5535; SEQ ID NO:8095 and 5534; SEQ ID NO:13676 and 7764; SEQ ID NO:13675 and 7763; SEQ ID NO: 14389 and 4892; SEQ ID NO:14388 and 4891; SEQ ID NO: 14025 and 13379; SEQ ID NO: 14018 and 13378; SEQ ID NO: 14990 and 13379; SEQ ID NO: 14985 and 13378; SEQ ID NO: 14825 and 5271; SEQ ID NO:14824 and 5270; SEQ ID NO:8038 and 11827; SEQ ID NO:8037 and 11826; SEQ ID NO:13764 and 5667; SEQ ID NO:13763 and 5666; SEQ ID NO:8420 and 17290; SEQ ID NO:8419 and 17289; SEQ ID NO:18188 and 5573; SEQ ID NO:18187 and 5572; SEQ ID NO:8792 and 13427; SEQ ID NO:8791 and 13426; SEQ ID NO:15494 and 5021; SEQ ID NO:15493 and 5020; SEQ ID NO:8476 and 10602; SEQ ID NO:8475 and 10601; SEQ ID NO:15081 and 7848; SEQ ID NO:15080 and 7847; SEQ ID NO:15073 and 13220; SEQ ID NO: 15072 and 13219; SEQ ID NO:7996 and 4734; SEQ ID NO:7995 and 4733; SEQ ID NO:8034 and 10638; SEQ ID NO:8033 and 10637; SEQ ID NO:8476 and 6734; SEQ ID NO:8475 and 6733; SEQ ID NO:11754 and 7106; SEQ ID NO:11753 and 7105; SEQ ID NO:9226 and 6749; SEQ ID NO:9222 and 6748; SEQ ID NO:7984 and 13455; SEQ ID NO:7983 and 13454; SEQ ID NO: 13942 and 7434; SEQ ID NO: 13941 and 7433; SEQ ID NO:8476 and 6379; SEQ ID NO:8475 and 6378; SEQ ID NO:8476 and 7692; SEQ ID NO:8475 and 7691; SEQ ID NO: 18207 and 4517; SEQ ID NO: 18206 and 4516; SEQ ID NO:8442 and 5377; SEQ ID NO:8441 and 5376; SEQ ID NO:15388 and 6389; SEQ ID NO:15387 and 6388; SEQ ID NO: 14607 and 4778; SEQ ID NO: 14606 and 4777; SEQ ID NO: 14419 and 17334; SEQ ID NO: 14418 and 17333; SEQ ID NO:9227 and 6866; SEQ ID NO:9223 and 6865; SEQ ID NO:15251 and 7850; SEQ ID NO:15250 and 7849; SEQ ID NO:13652 and 5651; SEQ ID NO:13651 and 5650; SEQ ID NO:8496 and 13063; SEQ ID NO:8495 and 13062; SEQ ID NO:8069 and 13419; SEQ ID NO:8068 and 13418; SEQ ID NO:8752 and 5579; SEQ ID NO: 8751 and 5578; SEQ ID NO: 15494 and 4856; SEQ ID NO: 15493 and 4855; SEQ ID NO: 14815 and 5849; SEQ ID NO: 14814 and 5848; SEQ ID NO:15289 and 4708; SEQ ID NO:15288 and 4707; SEQ ID NO:8607 and 13351; SEQ ID NO:8606 and 13350; SEQ ID NO:8756 and 11809; SEQ ID NO:8755 and 11808; SEQ ID NO:9228 and 6918; SEQ ID NO:9224 and 6917; SEQ ID NO: 14623 and 17286; SEQ ID NO: 14622 and 17285; SEQ ID NO:15139 and 6456; SEQ ID NO:15138 and 6455; SEQ ID NO:12215 and 17104; SEQ ID NO: 12214 and 17103; SEQ ID NO:8476 and 6798; SEQ ID NO:8475 and 6797; SEQ ID NO:8756 and 5533; SEQ ID NO:8755 and 5532; SEQ ID NO: 14443 and 7450; SEQ ID NO:14442 and 7449; SEQ ID NO:9165 and 11817; SEQ ID NO:9164 and 11816; SEQ ID NO:8756 and 5567; SEQ ID NO:8755 and 5566; SEQ ID NO:14002 and 5031; SEQ ID NO: 14001 and 5030; SEQ ID NO: 15083 and 6290; SEQ ID NO: 15082 and 6289; SEQ ID NO: 14897 and 7452; SEQ ID NO: 14896 and 7451; SEQ ID NO:9235 and 5575; SEQ ID NO:9234 and 5574; SEQ ID NO: 14591 and 5595; SEQ ID NO:14590 and 5594; SEQ ID NO: 8476 and 7510; SEQ ID NO:8475 and 7509; SEQ ID NO:12109 and 17108; SEQ ID NO:12108 and 17107; SEQ ID NO:15339 and 6858; SEQ ID NO:15338 and 6857; SEQ ID NO:14421 and 7546; SEQ ID NO:14420 and 7545; SEQ ID NO: 14543 and 7158; SEQ ID NO: 14542 and 7157; SEQ ID NO:8476 and 13557; SEQ ID NO:8475 and 13556; SEQ ID NO:13646 and 5683; SEQ ID NO:13645 and 5682; SEQ ID NO:13720 and 5189; SEQ ID NO:13719 and 5188; SEQ ID NO:15291 and 5361; SEQ ID NO:15290 and 5360; SEQ ID NO:9247 and 5547; SEQ ID NO:9246 and 5546; SEQ ID NO:14166 and 17102; SEQ ID NO:14165 and 17101; SEQ ID NO:14043 and 13520; SEQ ID NO:14042 and 13515; SEQ ID NO:9251 and 5719; SEQ ID NO:9250 and 5718; SEQ ID NO:8075 and 11811; SEQ ID NO:8074 and 11810; SEQ ID NO: 14943 and 4679; SEQ ID NO:14942 and 4678; SEQ ID NO:8063 and 13545; SEQ ID NO:8062 and 13544; SEQ ID NO:15093 and 4868; SEQ ID NO:15092 and 4867; SEQ ID NO: 13706 and 7752; SEQ ID NO:13705 and 7751; SEQ ID NO:8472 and 5273; SEQ ID NO:8471 and 5272; SEQ ID NO:8476 and 6854; SEQ ID NO:8475 and 6853; SEQ ID NO:8476 and 7522; SEQ ID NO:8475 and 7521; SEQ ID NO:9205 and 4543; SEQ ID NO:9204 and 4542; SEQ ID NO:9233 and 5575; SEQ ID NO:9232 and 5574; SEQ ID NO:8154 and 10636; SEQ ID NO:8153 and 10635; SEQ ID NO:8476 and 13345; SEQ ID NO:8475 and 13344; SEQ ID NO: 13648 and 5647; SEQ ID NO:13647 and 5646; SEQ ID NO:9229 and 6844; SEQ ID NO:9225 and 6843; SEQ ID NO:18166 and 13196; SEQ ID NO:18165 and 13195; SEQ ID NO:13895 and 4659; SEQ ID NO:13894 and 4658; SEQ ID NO:8476 and 5527; SEQ ID NO:8475 and 5526; SEQ ID NO:14975 and 5339; SEQ ID NO:14974 and 5338; SEQ ID NO:14748 and 13192; SEQ ID NO:14747 and 13191; SEQ ID NO:13650 and 5655; SEQ ID NO:13649 and 5654; SEQ ID NO:14983 and 7690; SEQ ID NO:14982 and 7689; SEQ ID NO: 14489 and 17346; SEQ ID NO: 14488 and 17345; SEQ ID NO:15181 and 5843; SEQ ID NO:15180 and 5842; SEQ ID NO:8476 and 13543; SEQ ID NO:8475 and 13542; SEQ ID NO:15410 and 5806; SEQ ID NO:15409 and 5805; SEQ ID NO: 14939 and 13597; SEQ ID NO:14938 and 13596; SEQ ID NO:8476 and 13496; SEQ ID NO:8475 and 13495; SEQ ID NO:8092 and 7476; SEQ ID NO:8091 and 7475; SEQ ID NO: 14162 and 17489; SEQ ID NO: 14161 and 17488; SEQ ID NO:8476 and 7548; SEQ ID NO: 8475 and 7547; SEQ ID NO:8476 and 13379; SEQ ID NO:8475 and 13378; SEQ ID NO:8476 and 5261; SEQ ID NO:8475 and 5260; SEQ ID NO:15079 and 7660; SEQ ID NO:15078 and 7659; SEQ ID NO:9249 and 5571; SEQ ID NO:9248 and 5570; SEQ ID NO:8172 and 11797; SEQ ID NO:8171 and 11796; SEQ ID NO:14387 and 4894; SEQ ID NO: 14386 and 4893; SEQ ID NO:8476 and 13551; SEQ ID NO:8475 and 13550; SEQ ID NO:18190 and 5691; SEQ ID NO: 18189 and 5690; SEQ ID NO:8476 and 5237; SEQ ID NO:8475 and 5236; SEQ ID NO:9243 and 5497; SEQ ID NO:9242 and 5496; SEQ ID NO:14385 and 4890; SEQ ID NO:14384 and 4889; SEQ ID NO:15201 and 4792; SEQ ID NO:15200 and 4791; SEQ ID NO:8563 and 13559; SEQ ID NO:8562 and 13558; SEQ ID NO:8476 and 5507; SEQ ID NO:8475 and 5506; SEQ ID NO: 14881 and 5313; SEQ ID NO: 14880 and 5312; SEQ ID NO: 13793 and 4888; SEQ ID NO:13792 and 4887; SEQ ID NO:12157 and 5829; SEQ ID NO:12156 and 5828; SEQ ID NO:12155 and 5727; SEQ ID NO:12154 and 5726; SEQ ID NO:9241 and 6870; SEQ ID NO:9240 and 6869; SEQ ID NO: 14257 and 13379; SEQ ID NO: 14256 and 13378; SEQ ID NO:8476 and 4862; SEQ ID NO:8475 and 4861; SEQ ID NO:9237 and 6417; SEQ ID NO:9236 and 6416; SEQ ID NO:8828 and 5525; SEQ ID NO:8827 and 5524; SEQ ID NO:7994 and 4732; SEQ ID NO:7993 and 4731; SEQ ID NO: 14881 and 13379; SEQ ID NO:14880 and 13378; SEQ ID NO:8476 and 5023; SEQ ID NO:8475 and 5022; SEQ ID NO:8854 and 5461; SEQ ID NO:8853 and 5458; SEQ ID NO:17691 and 18120; SEQ ID NO: 17690 and 18119; SEQ ID NO: 11142 and 13270; SEQ ID NO:11141 and 13269; SEQ ID NO:15540 and 5117; SEQ ID NO:15539 and 5116; SEQ ID NO:8476 and 7712; SEQ ID NO:8475 and 7711; SEQ ID NO:8476 and 11805; SEQ ID NO:8475 and 11804; SEQ ID NO: 13654 and 5653; SEQ ID NO: 13653 and 5652; SEQ ID NO: 13992 and 4917; SEQ ID NO:13991 and 4916; SEQ ID NO:9010 and 7170; SEQ ID NO:9009 and 7169; SEQ ID NO:8318 and 7573; SEQ ID NO:8317 and 7572; SEQ ID NO:14913 and 5303; SEQ ID NO:14912 and 5302; SEQ ID NO:8878 and 5491; SEQ ID NO:8877 and 5490; SEQ ID NO:12199 and 13240; SEQ ID NO:12198 and 13239; SEQ ID NO:13698 and 17592; SEQ ID NO:13697 and 17591; SEQ ID NO:6644 and 4939; SEQ ID NO:6643 and 4938; SEQ ID NO:14041 and 5561; SEQ ID NO:14040 and 5560; SEQ ID NO:15509 and 5285; SEQ ID NO:15508 and 5284; SEQ ID NO:8538 and 12262; SEQ ID NO:8526 and 12259; SEQ ID NO:8884 and 5037; SEQ ID NO:8883 and 5036; SEQ ID NO:9070 and 13228; SEQ ID NO:9069 and 13227; SEQ ID NO:14533 and 4995; SEQ ID NO:14532 and 4994; SEQ ID NO:11766 and 5671; SEQ ID NO:11765 and 5670; SEQ ID NO:15051 and 7404; SEQ ID NO: 15050 and 7403; SEQ ID NO: 15043 and 5245; SEQ ID NO: 15042 and 5244; SEQ ID NO:8959 and 5205; SEQ ID NO:8958 and 5204; SEQ ID NO: 14889 and 7650; SEQ ID NO: 14888 and 7649; SEQ ID NO: 14128 and 5748; SEQ ID NO: 14122 and 5736; SEQ ID NO:18192 and 17422; SEQ ID NO:18191 and 17421; SEQ ID NO:8706 and 13099; SEQ ID NO:8705 and 13098; SEQ ID NO:8216 and 7714; SEQ ID NO:8215 and 7713; SEQ ID NO: 14282 and 17360; SEQ ID NO:14281 and 17359; SEQ ID NO:15277 and 7829; SEQ ID NO:15276 and 7828; SEQ ID NO:8826 and 7442; SEQ ID NO:8825 and 7441; SEQ ID NO:8953 and 17319; SEQ ID NO:8952 and 17318; SEQ ID NO:15157 and 5788; SEQ ID NO:15156 and 5787; SEQ ID NO:14801 and 5782; SEQ ID NO:14800 and 5781; SEQ ID NO:8643 and 7528; SEQ ID NO:8642 and 7527; SEQ ID NO: 14397 and 4820; SEQ ID NO: 14396 and 4819; SEQ ID NO:15452 and 6868; SEQ ID NO:15451 and 6867; SEQ ID NO:8770 and 4688; SEQ ID NO:8769 and 4687; SEQ ID NO:12182 and 13519; SEQ ID NO:12166 and 13518; SEQ ID NO: 15010 and 7585; SEQ ID NO:15009 and 7584; SEQ ID NO: 13791 and 7796; SEQ ID NO: 13790 and 7795; SEQ ID NO:8796 and 7627; SEQ ID NO:8795 and 7627; SEQ ID NO:8220 and 7464; SEQ ID NO:8219 and 7463; SEQ ID NO:8772 and 4686; SEQ ID NO:8771 and 4685; SEQ ID NO:13774 and 5753; SEQ ID NO: 13773 and 5752; SEQ ID NO:8913 and 5493; SEQ ID NO:8912 and 5492; SEQ ID NO: 14501 and 13093; SEQ ID NO:14500 and 13092; SEQ ID NO:9149 and 10654; SEQ ID NO:9148 and 10653; SEQ ID NO:14947 and 6906; SEQ ID NO:14946 and 6905; SEQ ID NO:15053 and 7404; SEQ ID NO: 15052 and 7403; SEQ ID NO:6302 and 10606; SEQ ID NO:6301 and 10605; SEQ ID NO: 14750 and 6896; SEQ ID NO: 14749 and 6895; SEQ ID NO: 14599 and 6945; SEQ ID NO: 14598 and 6944; SEQ ID NO:8294 and 10646; SEQ ID NO:8293 and 10645; SEQ ID NO:14879 and 7254; SEQ ID NO: 14878 and 7253; SEQ ID NO:8170 and 4953; SEQ ID NO:8169 and 4952; SEQ ID NO: 14949 and 5365; SEQ ID NO: 14948 and 5364; SEQ ID NO:8251 and 13307; SEQ ID NO:8238 and 13306; SEQ ID NO: 14963 and 4804; SEQ ID NO: 14962 and 4803; SEQ ID NO:8836 and 7544; SEQ ID NO:8835 and 7543; SEQ ID NO:14835 and 4985; SEQ ID NO:14834 and 4984; SEQ ID NO:14417 and 7611; SEQ ID NO:14416 and 7610; SEQ ID NO: 15351 and 5301; SEQ ID NO: 15350 and 5300; SEQ ID NO: 14221 and 5808; SEQ ID NO: 14220 and 5807; SEQ ID NO: 15065 and 7792; SEQ ID NO:15064 and 7791; SEQ ID NO:8702 and 7265; SEQ ID NO:8701 and 7263; SEQ ID NO:9119 and 5697; SEQ ID NO:9118 and 5696; SEQ ID NO:14903 and 5798; SEQ ID NO:14902 and 5797; SEQ ID NO:14965 and 5231; SEQ ID NO:14964 and 5230; SEQ ID NO:9068 and 6880; SEQ ID NO:9067 and 6879; SEQ ID NO:15008 and 5707; SEQ ID NO: 15007 and 5706; SEQ ID NO: 17693 and 13180; SEQ ID NO:17692 and 13179; SEQ ID NO:8486 and 5489; SEQ ID NO:8485 and 5488; SEQ ID NO:8523 and 5143; SEQ ID NO:8522 and 5142; SEQ ID NO:15095 and 5820; SEQ ID NO:15094 and 5819; SEQ ID NO:14021 and 5621; SEQ ID NO:14019 and 5620; SEQ ID NO: 14033 and 13437; SEQ ID NO: 14032 and 13436; SEQ ID NO: 14565 and 4991; SEQ ID NO:14564 and 4990; SEQ ID NO:13855 and 13453; SEQ ID NO: 13854 and 13452; SEQ ID NO:14213 and 5509; SEQ ID NO:14212 and 5508; SEQ ID NO:7964 and 9303; SEQ ID NO:7963 and 9302; SEQ ID NO:14823 and 4951; SEQ ID NO:14822 and 4950; SEQ ID NO:18199 and 13067; SEQ ID NO:18198 and 13066; SEQ ID NO: 14673 and 4980; SEQ ID NO: 14665 and 4978; SEQ ID NO: 14775 and 12318; SEQ ID NO: 14774 and 12317; SEQ ID NO:13873 and 5818; SEQ ID NO:13872 and 5817; SEQ ID NO:9060 and 6846; SEQ ID NO:9059 and 6845; SEQ ID NO:15067 and 5784; SEQ ID NO:15066 and 5783; SEQ ID NO:13784 and 10660; SEQ ID NO:13783 and 10659; SEQ ID NO:8071 and 5699; SEQ ID NO:8070 and 5698; SEQ ID NO:8814 and 7698; SEQ ID NO:8813 and 7697; SEQ ID NO:8876 and 7688; SEQ ID NO:8875 and 7687; SEQ ID NO:15077 and 7722; SEQ ID NO:15076 and 7721; SEQ ID NO:13766 and 5711; SEQ ID NO:13765 and 5710; SEQ ID NO:13664 and 5035; SEQ ID NO:13663 and 5034; SEQ ID NO: 14909 and 6371; SEQ ID NO: 14908 and 6370; SEQ ID NO: 14833 and 7322; SEQ ID NO: 14832 and 7321; SEQ ID NO:8102 and 7514; SEQ ID NO:8101 and 7513; SEQ ID NO:8090 and 7472; SEQ ID NO:8089 and 7471; SEQ ID NO:17727 and 6856; SEQ ID NO:17726 and 6855; SEQ ID NO:14977 and 7300; SEQ ID NO: 14976 and 7299; SEQ ID NO:8184 and 6884; SEQ ID NO:8183 and 6883; SEQ ID NO:14781 and 7188; SEQ ID NO:14780 and 7187; SEQ ID NO:14933 and 7933; SEQ ID NO:14932 and 7932; SEQ ID NO:8067 and 7498; SEQ ID NO:8066 and 7497; SEQ ID NO:9093 and 6912; SEQ ID NO:9092 and 6911; SEQ ID NO:13756 and 9418; SEQ ID NO:13755 and 9417; SEQ ID NO:8352 and 4818; SEQ ID NO:8351 and 4817; SEQ ID NO:8939 and 9311; SEQ ID NO:8938 and 9310; SEQ ID NO:9050 and 6836; SEQ ID NO:9049 and 6835; SEQ ID NO:13857 and 4943; SEQ ID NO:13856 and 4942; SEQ ID NO:8539 and 11840; SEQ ID NO:8526 and 11839; SEQ ID NO:8344 and 4941; SEQ ID NO:8343 and 4940; SEQ ID NO:15317 and 13282; SEQ ID NO:15316 and 13281; SEQ ID NO:8272 and 7622; SEQ ID NO:8271 and 7618; SEQ ID NO: 14955 and 7354; SEQ ID NO: 14954 and 7353; SEQ ID NO:14993 and 7266; SEQ ID NO:14985 and 7263; SEQ ID NO:12181 and 7624; SEQ ID NO:12170 and 7618; SEQ ID NO:15149 and 13256; SEQ ID NO:15148 and 13255; SEQ ID NO:13666 and 5033; SEQ ID NO: 13665 and 5032; SEQ ID NO: 14473 and 13085; SEQ ID NO: 14472 and 13084; SEQ ID NO:9231 and 5191; SEQ ID NO:9230 and 5190; SEQ ID NO:14883 and 5835; SEQ ID NO:14882 and 5834; SEQ ID NO:8750 and 7623; SEQ ID NO:8749 and 7618; SEQ ID NO: 14513 and 13218; SEQ ID NO: 14512 and 13217; SEQ ID NO:13736 and 5477; SEQ ID NO:13735 and 5476; SEQ ID NO: 12123 and 17372; SEQ ID NO:12122 and 17371; SEQ ID NO:8298 and 7724; SEQ ID NO:8297 and 7723; SEQ ID NO:14087 and 4989; SEQ ID NO:14086 and 4988; SEQ ID NO:14158 and 5047; SEQ ID NO:14157 and 5046; SEQ ID NO:8856 and 5457; SEQ ID NO:8855 and 5456; SEQ ID NO:13909 and 10616; SEQ ID NO:13908 and 10615; SEQ ID NO:12291 and 13103; SEQ ID NO:12290 and 13102; SEQ ID NO: 8898 and 5493; SEQ ID NO:8897 and 5492; SEQ ID NO:8786 and 7726; SEQ ID NO:8785 and 7725; SEQ ID NO: 15061 and 7222; SEQ ID NO:15060 and 7221; SEQ ID NO:8729 and 7623; SEQ ID NO:8726 and 7618; SEQ ID NO:14867 and 18130; SEQ ID NO:14866 and 18129; SEQ ID NO:15414 and 5847; SEQ ID NO:15413 and 5846; SEQ ID NO:14633 and 12316; SEQ ID NO:14632 and 12315; SEQ ID NO:18178 and 5371; SEQ ID NO:18177 and 5370; SEQ ID NO: 14700 and 17383; SEQ ID NO: 14699 and 17377; SEQ ID NO: 14797 and 7414; SEQ ID NO: 14796 and 7413; SEQ ID NO:8683 and 17311; SEQ ID NO:8682 and 17310; SEQ ID NO: 15026 and 5289; SEQ ID NO: 15025 and 5288; SEQ ID NO: 14535 and 4997; SEQ ID NO: 14534 and 4996; SEQ ID NO: 13710 and 7579; SEQ ID NO: 13709 and 7578; SEQ ID NO:8968 and 4973; SEQ ID NO:8966 and 4972; SEQ ID NO:6631 and 9329; SEQ ID NO:6630 and 9328; SEQ ID NO:14639 and 4758; SEQ ID NO:14638 and 4757; SEQ ID NO: 15315 and 7394; SEQ ID NO: 15314 and 7393; SEQ ID NO: 14773 and 12310; SEQ ID NO: 14772 and 12309; SEQ ID NO:8218 and 12330; SEQ ID NO:8217 and 12329; SEQ ID NO:8957 and 10630; SEQ ID NO:8956 and 10629; SEQ ID NO:9048 and 13224; SEQ ID NO:9047 and 13223; SEQ ID NO: 13700 and 4997; SEQ ID NO: 13699 and 4996; SEQ ID NO:11961 and 5125; SEQ ID NO:11960 and 5124; SEQ ID NO:8880 and 7599; SEQ ID NO:8879 and 7598; SEQ ID NO:8247 and 17387; SEQ ID NO:8237 and 17377; SEQ ID NO:15131 and 7058; SEQ ID NO: 15130 and 7057; SEQ ID NO: 15209 and 10628; SEQ ID NO:15208 and 10627; SEQ ID NO:8466 and 13389; SEQ ID NO:8465 and 13388; SEQ ID NO:14759 and 13162; SEQ ID NO:14758 and 13161; SEQ ID NO:8414 and 7238; SEQ ID NO: 8413 and 7237; SEQ ID NO: 15125 and 5473; SEQ ID NO: 15124 and 5472; SEQ ID NO:8274 and 11829; SEQ ID NO:8273 and 11828; SEQ ID NO:14761 and 13164; SEQ ID NO: 14760 and 13163; SEQ ID NO:8374 and 7591; SEQ ID NO:8373 and 7590; SEQ ID NO: 12191 and 13302; SEQ ID NO: 12190 and 13301; SEQ ID NO:8268 and 6959; SEQ ID NO:8267 and 6952; SEQ ID NO:15281 and 17106; SEQ ID NO:15280 and 17105; SEQ ID NO: 14837 and 7130; SEQ ID NO: 14836 and 7129; SEQ ID NO:8976 and 7642; SEQ ID NO:8975 and 7641; SEQ ID NO: 14957 and 4933; SEQ ID NO:14956 and 4932; SEQ ID NO:14249 and 7432; SEQ ID NO: 14248 and 7431; SEQ ID NO:14845 and 7212; SEQ ID NO: 14844 and 7211; SEQ ID NO:11963 and 5123; SEQ ID NO:11962 and 5122; SEQ ID NO: 15103 and 7134; SEQ ID NO: 15102 and 7133; SEQ ID NO:15231 and 13238; SEQ ID NO:15230 and 13237; SEQ ID NO: 8663 and 4949; SEQ ID NO:8662 and 4948; SEQ ID NO:7966 and 5369; SEQ ID NO:7965 and 5368; SEQ ID NO:8168 and 4657; SEQ ID NO:8167 and 4656; SEQ ID NO:8937 and 7734; SEQ ID NO:8936 and 7733; SEQ ID NO: 14911 and 7684; SEQ ID NO:14910 and 7683; SEQ ID NO:9115 and 7542; SEQ ID NO:9114 and 7541; SEQ ID NO:13946 and 4082; SEQ ID NO:13945 and 4081; SEQ ID NO:9219 and 5211; SEQ ID NO:9218 and 5210; SEQ ID NO: 13950 and 5467; SEQ ID NO: 13949 and 5466; SEQ ID NO:14767 and 11856; SEQ ID NO:14766 and 11855; SEQ ID NO:8892 and 5145; SEQ ID NO:8891 and 5144; SEQ ID NO:15107 and 7852; SEQ ID NO:15106 and 7851; SEQ ID NO:12129 and 13107; SEQ ID NO: 12128 and 13106; SEQ ID NO:9002 and 7518; SEQ ID NO:9001 and 7517; SEQ ID NO: 14821 and 5479; SEQ ID NO: 14820 and 5478; SEQ ID NO: 11770 and 5665; SEQ ID NO: 11769 and 5664; SEQ ID NO: 14670 and 11840; SEQ ID NO: 14665 and 11839; SEQ ID NO: 14831 and 7194; SEQ ID NO:14830 and 7193; SEQ ID NO:14559 and 7680; SEQ ID NO:14558 and 7679; SEQ ID NO:13952 and 13510; SEQ ID NO:13951 and 13509; SEQ ID NO:8292 and 7564; SEQ ID NO:8291 and 7563; SEQ ID NO: 15022 and 4947; SEQ ID NO:15021 and 4946; SEQ ID NO:8258 and 7623; SEQ ID NO:8257 and 7618; SEQ ID NO:9046 and 13226; SEQ ID NO:9045 and 13225; SEQ ID NO:8198 and 6828; SEQ ID NO:8197 and 6827; SEQ ID NO:14411 and 13200; SEQ ID NO:14410 and 13199; SEQ ID NO:14545 and 5822; SEQ ID NO: 14544 and 5821; SEQ ID NO:8627 and 12252; SEQ ID NO:8626 and 12251; SEQ ID NO:8498 and 7640; SEQ ID NO:8497 and 7639; SEQ ID NO:14413 and 7873; SEQ ID NO: 14412 and 7872; SEQ ID NO: 14865 and 7526; SEQ ID NO: 14864 and 7525; SEQ ID NO: 11772 and 4999; SEQ ID NO:11771 and 4998; SEQ ID NO: 13742 and 7314; SEQ ID NO:13741 and 7313; SEQ ID NO:8290 and 5629; SEQ ID NO:8289 and 5628; SEQ ID NO:8364 and 5411; SEQ ID NO:8363 and 5410; SEQ ID NO:8923 and 6337; SEQ ID NO:8922 and 6336; SEQ ID NO:8140 and 7558; SEQ ID NO:8139 and 7557; SEQ ID NO: 14601 and 13037; SEQ ID NO:14600 and 13036; SEQ ID NO:8832 and 7638; SEQ ID NO:8831 and 7637; SEQ ID NO:13730 and 6226; SEQ ID NO:13729 and 6225; SEQ ID NO: 14467 and 11841; SEQ ID NO:14466 and 11838; SEQ ID NO:13962 and 17532; SEQ ID NO:13961 and 17531; SEQ ID NO:18204 and 4529; SEQ ID NO:18203 and 4528; SEQ ID NO:14101 and 17408; SEQ ID NO:14100 and 17407; SEQ ID NO:14915 and 6771; SEQ ID NO:14914 and 6770; SEQ ID NO: 14855 and 7172; SEQ ID NO: 14854 and 7171; SEQ ID NO:14571 and 5307; SEQ ID NO: 14570 and 5306; SEQ ID NO:15105 and 7581; SEQ ID NO:15104 and 7580; SEQ ID NO:8890 and 7470; SEQ ID NO:8889 and 7469; SEQ ID NO: 14365 and 13105; SEQ ID NO: 14364 and 13104; SEQ ID NO:9095 and 6892; SEQ ID NO:9094 and 6891; SEQ ID NO:14435 and 13520; SEQ ID NO:14425 and 13515; SEQ ID NO:8921 and 6339; SEQ ID NO:8920 and 6338; SEQ ID NO:7962 and 7571; SEQ ID NO:7961 and 7570; SEQ ID NO:15269 and 5837; SEQ ID NO:15268 and 5836; SEQ ID NO:8635 and 5347; SEQ ID NO:8634 and 5346; SEQ ID NO:8106 and 7418; SEQ ID NO:8105 and 7417; SEQ ID NO:8320 and 9309; SEQ ID NO:8319 and 9308; SEQ ID NO: 14701 and 13424; SEQ ID NO: 14698 and 13422; SEQ ID NO: 14663 and 5679; SEQ ID NO: 14662 and 5678; SEQ ID NO:8316 and 6824; SEQ ID NO:8315 and 6823; SEQ ID NO:8210 and 7720; SEQ ID NO:8209 and 7719; SEQ ID NO:13869 and 5051; SEQ ID NO:13868 and 5050; SEQ ID NO: 14863 and 7917; SEQ ID NO: 14862 and 7916; SEQ ID NO:8372 and 6769; SEQ ID NO:8371 and 6768; SEQ ID NO:7960 and 7488; SEQ ID NO:7959 and 7487; SEQ ID NO:15171 and 13411; SEQ ID NO:15170 and 13410; SEQ ID NO:18158 and 17354; SEQ ID NO:18157 and 17353; SEQ ID NO:8736 and 12268; SEQ ID NO:8735 and 12267; SEQ ID NO:14140 and 4104; SEQ ID NO:14139 and 4103; SEQ ID NO:8150 and 7567; SEQ ID NO:8149 and 7565; SEQ ID NO:15063 and 7790; SEQ ID NO:15062 and 7789; SEQ ID NO: 14083 and 17374; SEQ ID NO: 14082 and 17373; SEQ ID NO: 14150 and 4870; SEQ ID NO:14149 and 4869; SEQ ID NO:15203 and 5353; SEQ ID NO: 15202 and 5352; SEQ ID NO: 13734 and 4957; SEQ ID NO:13733 and 4956; SEQ ID NO:13722 and 17321; SEQ ID NO:13721 and 17320; SEQ ID NO:14531 and 4997; SEQ ID NO:14530 and 4996; SEQ ID NO: 14567 and 4993; SEQ ID NO: 14566 and 4992; SEQ ID NO: 14081 and 5131; SEQ ID NO: 14080 and 5130; SEQ ID NO: 14849 and 5824; SEQ ID NO: 14848 and 5823; SEQ ID NO:8376 and 5861; SEQ ID NO:8375 and 5860; SEQ ID NO: 14597 and 13592; SEQ ID NO:14596 and 13591; SEQ ID NO:8276 and 5551; SEQ ID NO:8275 and 5550; SEQ ID NO:9014 and 4116; SEQ ID NO:9013 and 4115; SEQ ID NO: 13867 and 5049; SEQ ID NO:13866 and 5048; SEQ ID NO:8270 and 7706; SEQ ID NO:8269 and 7705; SEQ ID NO:8382 and 5003; SEQ ID NO:8381 and 5002; SEQ ID NO:7988 and 4854; SEQ ID NO:7987 and 4853; SEQ ID NO:12045 and 5389; SEQ ID NO:12044 and 5388; SEQ ID NO:9024 and 7562; SEQ ID NO:9023 and 7561; SEQ ID NO:14819 and 5867; SEQ ID NO: 14818 and 5866; SEQ ID NO: 14351 and 9305; SEQ ID NO: 14350 and 9304; SEQ ID NO:8362 and 5367; SEQ ID NO:8361 and 5366; SEQ ID NO:8543 and 6938; SEQ ID NO:8542 and 6937; SEQ ID NO: 14549 and 10604; SEQ ID NO: 14548 and 10603; SEQ ID NO:8390 and 7672; SEQ ID NO:8389 and 7671; SEQ ID NO:14471 and 5355; SEQ ID NO:14470 and 5354; SEQ ID NO:12153 and 13115; SEQ ID NO: 12152 and 13114; SEQ ID NO: 14595 and 17075; SEQ ID NO:14594 and 17074; SEQ ID NO:8565 and 17078; SEQ ID NO:8564 and 17077; SEQ ID NO:12107 and 13371; SEQ ID NO:12106 and 13370; SEQ ID NO:13978 and 4696; SEQ ID NO:13977 and 4695; SEQ ID NO: 14160 and 17297; SEQ ID NO:14159 and 17296; SEQ ID NO:9052 and 5717; SEQ ID NO:9051 and 5716; SEQ ID NO: 14186 and 4730; SEQ ID NO: 14185 and 4729; SEQ ID NO: 14207 and 13190; SEQ ID NO: 14206 and 13189; SEQ ID NO:8282 and 9307; SEQ ID NO:8281 and 9306; SEQ ID NO: 14649 and 13359; SEQ ID NO: 14648 and 13358; SEQ ID NO:8820 and 5197; SEQ ID NO:8819 and 5196; SEQ ID NO: 14653 and 4700; SEQ ID NO: 14652 and 4699; SEQ ID NO: 14353 and 5265; SEQ ID NO: 14352 and 5264; SEQ ID NO:15377 and 4784; SEQ ID NO:15374 and 4783; SEQ ID NO:8990 and 6986; SEQ ID NO:8989 and 6985; SEQ ID NO:12121 and 5569; SEQ ID NO: 12120 and 5568; SEQ ID NO:14229 and 6990; SEQ ID NO:14228 and 6989; SEQ ID NO: 14227 and 7000; SEQ ID NO: 14226 and 6999; SEQ ID NO:8882 and 7468; SEQ ID NO:8881 and 7467; SEQ ID NO:14235 and 5281; SEQ ID NO: 14234 and 5280; SEQ ID NO: 14686 and 7631; SEQ ID NO: 14685 and 7627; SEQ ID NO: 14205 and 7273; SEQ ID NO: 14197 and 7268; SEQ ID NO:18110 and 6814; SEQ ID NO:18109 and 6813; SEQ ID NO:15121 and 5141; SEQ ID NO:15120 and 5140; SEQ ID NO: 13638 and 7827; SEQ ID NO: 13637 and 7826; SEQ ID NO: 15456 and 10600; SEQ ID NO: 15455 and 10599; SEQ ID NO: 13782 and 5685; SEQ ID NO:13781 and 5684; SEQ ID NO: 15505 and 17313; SEQ ID NO:15504 and 17312; SEQ ID NO:9139 and 4935; SEQ ID NO:9138 and 4934; SEQ ID NO:14355 and 5263; SEQ ID NO: 14354 and 5262; SEQ ID NO: 15273 and 4714; SEQ ID NO:15272 and 4713; SEQ ID NO:9129 and 5287; SEQ ID NO:9128 and 5286; SEQ ID NO:11918 and 4702; SEQ ID NO: 11917 and 4701; SEQ ID NO: 14789 and 7262; SEQ ID NO: 14788 and 7261; SEQ ID NO: 15097 and 6405; SEQ ID NO:15096 and 6404; SEQ ID NO:8334 and 7748; SEQ ID NO:8333 and 7747; SEQ ID NO:15331 and 4651; SEQ ID NO:15330 and 4650; SEQ ID NO:8872 and 7480; SEQ ID NO:8871 and 7479; SEQ ID NO:9215 and 13136; SEQ ID NO:9214 and 13135; SEQ ID NO:9054 and 6820; SEQ ID NO:9053 and 6819; SEQ ID NO:14029 and 9325; SEQ ID NO: 14028 and 9324; SEQ ID NO:13670 and 16615; SEQ ID NO:13669 and 16614; SEQ ID NO:8551 and 17309; SEQ ID NO:8550 and 17308; SEQ ID NO:14182 and 5359; SEQ ID NO:14181 and 5358; SEQ ID NO:18184 and 13318; SEQ ID NO:18183 and 13317; SEQ ID NO:11914 and 17350; SEQ ID NO:11913 and 17349; SEQ ID NO:15075 and 7382; SEQ ID NO:15074 and 7381; SEQ ID NO:11910 and 5687; SEQ ID NO:11909 and 5686; SEQ ID NO: 14697 and 4706; SEQ ID NO: 14696 and 4705; SEQ ID NO: 14405 and 5859; SEQ ID NO: 14404 and 5858; SEQ ID NO: 12115 and 17086; SEQ ID NO: 12114 and 17085; SEQ ID NO:15115 and 5871; SEQ ID NO:15114 and 5870; SEQ ID NO:14635 and 13369; SEQ ID NO: 14634 and 13368; SEQ ID NO:8553 and 7656; SEQ ID NO:8552 and 7655; SEQ ID NO:13658 and 6978; SEQ ID NO:13657 and 6977; SEQ ID NO: 15408 and 12258; SEQ ID NO: 15407 and 12257; SEQ ID NO: 14555 and 4655; SEQ ID NO:14554 and 4654; SEQ ID NO:12217 and 5796; SEQ ID NO:12216 and 5795; SEQ ID NO:14395 and 7478; SEQ ID NO: 14394 and 7477; SEQ ID NO:9008 and 5247; SEQ ID NO:9007 and 5246; SEQ ID NO: 11965 and 7024; SEQ ID NO:11964 and 7023; SEQ ID NO: 13718 and 5193; SEQ ID NO:13717 and 5192; SEQ ID NO:13801 and 7012; SEQ ID NO:13800 and 7011; SEQ ID NO:14225 and 7870; SEQ ID NO:14224 and 7869; SEQ ID NO: 13976 and 4698; SEQ ID NO: 13975 and 4697; SEQ ID NO: 14331 and 13254; SEQ ID NO:14330 and 13253; SEQ ID NO: 14475 and 7786; SEQ ID NO:14474 and 7785; SEQ ID NO:8591 and 6403; SEQ ID NO:8590 and 6402; SEQ ID NO:15045 and 5715; SEQ ID NO:15044 and 5714; SEQ ID NO:13780 and 5599; SEQ ID NO:13779 and 5598; SEQ ID NO:18172 and 9313; SEQ ID NO:18171 and 9312; SEQ ID NO: 12233 and 6349; SEQ ID NO: 12232 and 6348; SEQ ID NO:13879 and 5295; SEQ ID NO:13878 and 5294; SEQ ID NO:18170 and 5633; SEQ ID NO:18169 and 5632; SEQ ID NO:13938 and 4673; SEQ ID NO: 13937 and 4672; SEQ ID NO: 14407 and 5279; SEQ ID NO: 14406 and 5278; SEQ ID NO: 14194 and 5583; SEQ ID NO:14193 and 5582; SEQ ID NO: 14959 and 4728; SEQ ID NO:14958 and 4727; SEQ ID NO: 14302 and 17352; SEQ ID NO: 14301 and 17351; SEQ ID NO: 12283 and 5511; SEQ ID NO: 12282 and 5510; SEQ ID NO:8541 and 13264; SEQ ID NO:8540 and 13263; SEQ ID NO:8577 and 7196; SEQ ID NO:8576 and 7195; SEQ ID NO: 14521 and 13338; SEQ ID NO: 14520 and 13337; SEQ ID NO:11916 and 4653; SEQ ID NO:11915 and 4652; SEQ ID NO:14106 and 5623; SEQ ID NO:14104 and 5622; SEQ ID NO:8974 and 5241; SEQ ID NO:8973 and 5240; SEQ ID NO:8804 and 5643; SEQ ID NO:8803 and 5642; SEQ ID NO:14184 and 13186; SEQ ID NO:14183 and 13185; SEQ ID NO:9179 and 17414; SEQ ID NO:9178 and 17413; SEQ ID NO:8480 and 9333; SEQ ID NO:8479 and 9332; SEQ ID NO:8915 and 10640; SEQ ID NO:8914 and 10639; SEQ ID NO:14142 and 5165; SEQ ID NO:14141 and 5164; SEQ ID NO:17169 and 17495; SEQ ID NO:17168 and 17494; SEQ ID NO:14961 and 11852; SEQ ID NO:14960 and 11851; SEQ ID NO:15323 and 4649; SEQ ID NO: 15322 and 4648; SEQ ID NO:12229 and 5855; SEQ ID NO:12228 and 5854; SEQ ID NO:8156 and 7742; SEQ ID NO:8155 and 7741; SEQ ID NO:14393 and 7076; SEQ ID NO: 14392 and 7075; SEQ ID NO:8136 and 6882; or SEQ ID NO:8135 and 6881.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of”.

“Individual, “subject,” and “patient” are used interchangeably and can refer to a human or non-human.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-G: The anchor epitope is a common target of stalk-binding antibodies. a, Negative stain EM of representative 2D class averages and 3D reconstructions of Fabs binding to A/California/July 2009 E376K HA. b, Juxtaposed 3D reconstructions of Fabs binding to A/California/July 2009 E376K HA. c, Binding footprints of anchor-binding Fabs relative to mAbs targeting the CS epitope (CR9114 and FI6v3). d, Competition of stalk-binding mAbs withCR9114 or 047-09 4F04. e, Neutralization potency of anchor-(n=50) and CS-binding (n=37) mAbs against A/California/July 2009. f, Proportion of anchor-(n=50) and CS-targeting (n=37)mAbs binding to other group 1 influenza virus A subtypes. g, Representative 2D class averages,3D reconstructions, and footprints of 222-1C06 binding to H2 and the relative footprint on H1. Data in e are represented as mean±S.D and were analyzed by two-tailed unpaired non-parametric Mann-Whitney test. Data in f were analyzed by Fisher's Exact tests. See also FIGS. 5-7.

FIG. 2A-K: Anchor-targeting mAbs bind the HA fusion peptide via public binding motifs. a andb, VH (a) and VK (b) gene usage by anchor-binding mAbs. c, Cryo-EM structure of anchor-targeting 222-1C06 (blue) and lateral patch-targeting 045-09 2B05 (dark gray; see methods) binding to A/California/July 2009 (E376K) HA (light gray). d, Heavy and light chain footprint of 222-1C06 binding to HA based on cryo-EM structure. e, HA epitope contact residues (maroon) and heavy chain (green) and light chain (yellow) antibody contact residues of the 222-1C06 paratope. Peach highlighted amino acids represent the fusion peptide of HA2. f, K-CDR3 NWP and H-CDR2 Y58 motifs of 222-1C06. g, Major contacts of 222-1C06 K-CDR1 and H-CDR3 (normal typeface) binding to HA (bold residues). h and i, Weblogo plot and germline sequenceof Y58 following the H-CDR2 (h) and K-CDR3 NWP motif (i). j and k, Amino acid conservationof s contact residues across human, swine, and avian H1 viruses (j) and group 1 influenza A viruses (k). Strain information used for conservation models in j and k are in Supplementary Table 4 and 5, respectively. See also Extended Data FIG. 4. Shown in FIG. 2H are SEQ ID NOS:18650, 18651, and 18652, respectively. Shown in FIG. 2I are SEQ ID NOS:10711, 12433, 12775, and 12772, respectively.

FIG. 3A-D: MBCs and serum antibodies commonly target the anchor epitope. a, Proportion of all cH5/1⁺ B cells with repertoire features of anchor-binding mAbs, VH1-69/kappa (CS epitope), or with other repertoire features. b, Proportion of B cells with anchor-binding mAb features or that use VH1-69/kappa chain by participant (n=20 donors). Lines connect the same participant. c, EMPEM summary of pAbs in serum collected at day 7 and 14 following 2014 QIV inparticipants 236 and 241 binding to A/Michigan/45/2015 H A. Fabs shown as graphics withdotted lines represent predicted placements due to limited particle representation. d, Overlap of 241 IgA 2F04 Fab and pAb from participant 241 binding the anchor epitope. Data in b were analyzed using a two-tailed paired non-parametric Wilcoxon matched-pairs signed rank test. See also FIG. 9.

FIG. 4A-E: cHA vaccination in humans robustly recalls MBCs targeting the anchor epitope. a, cHA vaccine trial design including vaccine groups (right; group 1 n=10 participants; group 2 n=7 participants; group 4 n=7 participants; group 3&5 n=6). i.n., intranasal; i.m., intramuscular. b-c, Fold change by participant of serum antibodies competing for binding against the anchor and CS epitopes after the prime (d29/d1; b) and boost (d113/85; c). d-e, Proportion of participants that seroconverted (d) and half maximal effective concentration titers (EC₅₀; e) against the anchor and CS epitopes. Individuals in the cHA (IIV+AS03) cohort were those that received the cHA vaccine with adjuvant (cH8/1 IIV+AS03 Prime and cH5/1 IIV+AS03 Boost; n=17 donors) and the IIV cohort were those that received licensed IIV vaccines (2009 MIV, 2010 TIV, and 2014 QIV; n=1 1 donors). Data in b, c, and e are mean±S.D. Dashed line represents limit of detection. Data in b and c were analyzed by two-tailed non-parametric Kruskal-Wallis Tests, data in d were analyzed by Fisher's Exact test, and data in e were analyzed by two-tailed unpaired non-parametric Mann-Whitney test. See also FIG. 10.

FIG. 5A-L: Binding and neutralization features of anchor epitope-bindingmAbs. Related to FIG. 1. a, Proportion of HA⁺ mAbs binding to distinct HA domains (left) and proportion of stalk-binding mAbs binding the CS domain (right). Number in the center of the pie graphs represent the number of mAbs tested. b, Proportion of mAbs per cohort that bind the HAstalk domain. c, Negative stain 2D class averages of 047-09 4F04, 241 IgA 2F04, and 222-1C06 binding to H1 (A/California/July 2009 E376K HA). d, Overlay of 047-09 4F04, 241 IgA 2F04, 222-1C06, and FISW84 (PDB:6HJQ) Fabs binding the anchor epitope of A/California/April 2009 HA. e, Overlay of CR8020 (PDB:3SDY), CR8043 (PDB:4NM8), and FISW84 (PDB:6HJQ) modeled on A/California/July 2009 E376G (PDB:4M4Y). f, Footprints of anchor mAb 222-1C06 on H1 (top; PDB: 4M4Y) and CR8020 and CR8043 on H3 (bottom; PDB:4WE4). g, Heatmap of apparent affinity (K_{d}; M) of anchor-targeting mAbs binding to historical and recent H1N1 viruses.h, Neutralization potency of anchor-binding mAbs (n=15) against H1-expressing viruses. i, Representative microneutralization curves of anchor-(n=42) and CS-binding (n=29) mAbs against A/California/July 2009. j, IC₅₀ of anchor- and CS-binding mAbs against A/California/July 2009.k, ADCC activity of mAbs targeting the CS and anchor epitopes. Dashed line represents the limit of detection (L.O.D). 1, ADCC potency of mAbs targeting the anchor (n=18 mAbs) and CS (n=8 mAbs) epitopes. Data in h, j, and 1 are represented as mean±S.D. Data in j and 1 were analyzed using a two-tailed unpaired non-parametric Mann-Whitney test.

FIG. 6A-G: Anchor-targeting mAb binding to influenza subtype, viral mutants, and polyreactivity antigen panel. Related to FIG. 1. a, Proportion of anchor- (n=50 mAbs) andCS-targeting mAbs (n=37) binding influenza B viruses and H3N2 viruses. b, Negative stain 2D class averages of 222-1C06 binding to H2 (A/Singapore/January 1957), and H5 (A/Indonesia/May 2005). c, H2N2 neutralizing data of anchor- (n=11 mAbs) and CS-binding mAbs (n=4) represented as minimum neutralizing concentration. The limit of detection (L.O.D.) is 30 mg/ml. d, Proportion of mAbs targeting the anchor (n=50 mAbs) or CS (n=50 mAbs) epitope that are polyreactive. e, LPS binding strength, represented as area under the curve (AUC), of polyreactive mAbs targeting the anchor (n=30 mAbs) and central stalk (n=43 mAbs) epitopes. Data are mean±S.D. f and g, Anchor- and CS epitope-binding mAbs were tested for binding to A/California/July 2009 HA with naturally occurring and experimentally determined mutations induced by 045-09 2B06, a CS-binding mAb. f, Location of mutations modeled onA/California/April 2009 HA (PDB: 4JTV). Residues in blue are located on HA1 and residues in red are located on HA2. Outlines represent binding footprints of 047-09 4F04 (sky blue) and CR9114 (green). g, Heatmap of mAb binding to WT and mutant HAs shown as the proportion of signal relative to mAb binding to the WT HA. Data in a and d were analyzed using Fisher's Exact tests. Data in e were analyzed using a two-tailed unpaired non-parametric Mann-Whitney test.

FIG. 7A-D: Anchor epitope-targeting mAbs are potently protective in vivo and lack ADCC activity. Related to FIG. 1. a-b, Mice were prophylactically (2 hours prior to infection; a) or therapeutically (48 hours after infection; b) administered i.p. a cocktail of mAbs (n=5 mAbs/cocktail) against the anchor- or CS-, or an anthrax-specific antibody. Mice were infected with 10 LD₅₀ of A/Netherlands/602/2009 H1N1. Weight loss (top) and survival (bottom) of mice in each treatment group. c, Lung viral titers of mice in each prophylactic treatment group infected with 1 LD₅₀ of A/Netherlands/602/2009. dpi, days post infection. d, Mice were prophylactically (2 hours prior to infection) administered i.p. a cocktail of mAbs (n=5 mAbs/cocktail) against the anchor- or CS-, or an anthrax-specific antibody. Mice were infected with 10 LD₅₀ of A/Fort Monmouth/January 1947 H1N1. Weight loss (top) and survival (bottom) of mice in each treatment group. For a, b, and d, 10 mice per treatment group were used and data are pooled from two independent experiments. For c, 5 mice per treatment group and timepointwere used except for anchor cocktail group at dpi 3 only 4 mice were used. Data in a, b, and d are represented as mean±S.D and data in c are represented by mean S.E.M. Kaplan-Meier curves in a, b, d were analyzed using a Log-rank Mantel-Cox test, and data in c were analyzed using multiple two-tailed unpaired non-parametric Kruskal-Wallis tests.

FIG. 8A-Q: Additional repertoire and structural features of mAbs binding the anchor epitope. Related to FIG. 2. a, VH locus usage by anchor- (n=52 mAbs) and CS-binding mAbs (n=37 mAbs). b, VH1 gene usage of mAbs targeting the CS epitope. c, VK locus usage by anchor- (n=52 mAbs) and CS-binding mAbs (n=37 mAbs). d, JK gene usage by anchor epitope-binding mAbs. e, Clonal expansions of anchor epitope-targeting mAbs. Numbers indicate heavy and light chain parings, which are described in Extended Data Table 2. f, Heavy and light chain sequences of the public clone. g and h, Mutations (g) and CDR3 amino acid (AA) lengths (h) of heavy and light chains of mAbs binding the anchor (n=52 mabs) or CS (n=37mAbs) epitopes. Data are mean±S.D. i, Cryo-EM map of 222-1C06 binding to A/California/July 2009 E376K HA. j and k, Local resolution (j) and Fourier Shell Correlation (k) of 222-1C06 binding to HA. 1, Aromatic pockets of 222-1C06 binding A/California/July 2009 E376K and FISW84 binding to A/duck/Alberta/35/1976 (PDB:6HJQ; top) and overlay ofepitope:paratope interaction (bottom). m, MD simulations demonstrating the K-CDR3 NWP and H-CDR2 Y58 motifs of 222-1C06, FISW84, 241 IgA 2F04, and SFV009 3G01 binding to HA A/California/July 2009 H A. For left-hand panels in 1 and all panels in m, HA epitope contact residues (maroon) and heavy chain (green) and light chain (yellow) antibody contact residues of anchor mAb paratopes. Peach highlighted amino acids represent the fusion peptide of HA2. n, Fab-Fab interactions of the aromatic pocket of 222-1C06. o, MD simulation of the paratope flexibility of 222-1C06, highlighting the p-stacking of H-CDR2 and K-CDR3. p, Conservation of side-chain contacts of 222-1C06 across seasonal human H1N1 viruses circulating between 1918-2019. q, Deep mutational scanning of the side-chain contacts of 222-1C06. Data in a andc were analyzed using a Chi-square test, and data in g and h were analyzed by two-tailed unpaired non-parametric Mann-Whitney tests. Shown in FIG. 8F are SEQ ID NOS: 18653, 18654, 18655, 7623, 7678 and 7627, respectively.

FIG. 9A-E: Features of anchor-targeting MBCs and EMPEM 2D classes. Related to FIG. 3. a, 33 mAbs with anchor epitope-binding mAb repertoire features weregenerated and tested for competing for binding with 047-09 4F04. b-c, Number of heavy chain mutations (b) and isotype usage (c) of B cells with repertoire features of anchor-binding mAbs (n=119 cells) or utilize VH1-69/kappa (n=365 cells). d-e, 2D class averages of pAbs from donors236 (d) and 241 (e) at days 7 and 14 post immunization binding to A/Michigan/45/2015 H A. The last row of 2D classes in d is HA monomer complexes processed independently from trimer complexes. Data in b are represented as mean±S.D. Data in b were analyzed using a two-tailed paired non-parametric Wilcoxon matched-pairs signed rank test.

FIG. 10A-I: Serum antibody kinetics of anchor- and CS-epitope binding antibodies after cHA vaccination and mAb binding to recombinant HAs. Related to FIG. 4. a-b, EC50s of serum antibodies competing for binding with 047-09 4F04 for binding to the anchor epitope (a) and CR9114 for binding to the CS epitope (b). a-b, Kinetics of serumantibody responses against the anchor (a) and CS (b) epitopes. Data are mean±S.D. c-d, Proportion of stalk⁺ mAbs per donor (c) or proportion of donors with an isolated anchor mAb (d) upon first exposure to the pH1N1 virus (2009 MIV cohort) relative to donors who have repeatedly been exposed to pH1N1 (2010 TIV and 2014 QIV). Data in c are mean±S.D. Data in c includes only donors with an isolated anti-stalk mAb, whereas d includes all donors. e-f, Antibody titers (EC₅₀) of serum antibodies collected on day 113 and day 420 against the anchor epitope (e) and the CS epitope (f). Lines connect titers from the same donor and each pair of symbols represents one donor. g, Proportion of anchor epitope-binding mAbs binding to cHA (cH6/1) or mini-HA (n=50). h, Representative flow cytometry plots of mAbs binding to A/California/July 2009 Cal09 HA and mini-HA (left) and geometric mean fluorescence intensity (gMFI) of mAbs binding to Cal09 and mini-HA (right). Data represent the mean±S.D. and each symbol represents an individual mAb. i, Proportion of anchor epitope-targeting mAbs binding to A/California/July 2009 recombinant HA with a GCN4 or fibritin trimerization domain (n=50). Fordata in a, b, e, and f, Group 1 n=10 participants, group 2 n=7 participants, group 4 n=7 participants, group 3&5 n=6. For data in c, first exposure n=7 donors and repeated exposure n=4 donors. For data in d, first exposure n=10 donors and repeated exposure n=13 donors. Data in a and b were analyzed using a two-tailed two-way ANOVA testing for simple effects within rows, data in c and h were analyzed using a two-tailed unpaired non-parametric Mann-Whitney test, data in d, g, and i were analyzed using Fisher's Exact test, and data in c and d were analyzed using a two-tailed paired non-parametric Wilcoxon matched-pairs signed rank test. See also Supplementary FIG. 1 for gating strategy for panel h.

FIG. 11. Gating Strategy for FIG. 10h. Examples of gating for total cells and mAb+ cells in samples that were transfected to express membrane-bound A/California/July 2009 HA relating gMFI presented in FIG. 10h.

FIG. 12A-F. The anchor epitope is a common target of HA stalk binding antibodies. (A-B), Negative stain EM 2D class averages and 3D reconstructions of negative stain EM of 047-09 4F04 Fab (A) and 241 IgA 2F04 Fab (B) binding to A/California/April 2009 HA. (C) Overlay of 047-09 4F04, 241 IgA 2F04, and FISW84 (PDB: 6HJQ) Fabs binding the anchor epitope of A/California/04/2009 HA. (D) Binding footprints of 047-09 4F04, 241 IgA 2F04, CR9114, and FI6v3 on A/California/04/2009 HA. (E) Competition of stalk binding mAbs with CR9114 or 047-09 4F04. (F) Proportion of mAbs binding to the anchor epitope, the BN stalk epitope, or an unknown stalk epitope based on competition with 047-09 4F04 or CR9114. Number in the center of the pie graph represents the number of mAbs tested. See also FIG. 19 and Table S2.

FIG. 13A-D. Anchor epitope targeting antibodies are broadly reactive amongst H1 expressing viruses. (A) Proportion of anchor epitope and BN stalk epitope targeting mAbs binding to other group 1 subtypes. (B) Apparent affinity of anchor and BN stalk binding mAbs to A/California/July 2009 virus. Data are represented as mean±S.D. (C-D) Anchor and BN stalk binding mAbs were tested for binding to A/California/July 2009 HA with naturally occurring mutations and experimentally determined mutations induced by 045-09 2B06, a BN stalk epitope binding mAb. (C) Location of mutations modeled on A/California/04/2009 HA (PDB: 4JTV). Residues in blue are located on HA1 and residues in red are located on HA2. Outlines represent binding footprints of 047-09 4F04 (sky blue) and CR9114 (green). (D) Heatmap of mAb binding to WT and mutant HAs shown as the proportion of signal relative to mAb binding to the WT HA. Data in A were analyzed by Fisher's Exact tests and B were analyzed by unpaired non-parametric Mann-Whitney test. See also FIG. 20, FIG. 21, Table S3.

FIG. 14A-E. Anchor epitope targeting mAbs are broadly neutralizing amongst H1 viruses and potently protective in vivo. (A) Neutralization potency of mAbs binding the anchor or BN stalk epitope against A/California/July 2009 H1N1. (B) Neutralization potency of anchor epitope binding mAbs against H1-expressing viruses. (C-E) Mice were prophylactically administered i.p. a cocktail of mAbs (n=5 mAbs/cocktail) against the anchor epitope or BN stalk epitope, or an anthrax specific antibody. Mice were infected 2 hours later with 10 LD50 ofA/Netherlands/602/2009 HIN1. (C) Experiment design. Weight loss (D) and survival (E) of mice in each treatment group. N=10 mice per treatment group and are pooled from two independent experiments. Data in A, B, and D are represented as mean±S.D. Data in A were analyzed by unpaired non-parametric Mann-Whitney test. See also Table S4.

FIG. 15A-H. Anchor epitope targeting B cells are induced by licensed influenza virus vaccines. (A-C) MAbs were generated from plasmablasts isolated 7 days after influenza virus vaccination with the 2009 MIV and the 2014 QIV. (A) Domain binding of HA+ mAbs. (B-C) Epitope specificity of stalk domain binding mAbs by vaccine cohort (B) and by subjects (C). (D-F) EMPEM of serum collected at day 7 and 14 following 2014 QIV in subjects 236 and 241 binding to A/Michigan/45/2015 HA. (D) Summary ofpAbs at day 7 and d14. (E) Overlap of 241 IgA 2F04 fab and pAb binding anchor epitope from subject 241. (F) Binding footprint of 241 IgA 2F04 (sky blue) and pAb from subject 241 (purple). (G) Overlap of anchor epitope binding pAbs from subjects 236 (lavender) and 241 (purple). (H) Binding footprint of pAbs from subjects 236 (lavender) and 241 (purple) relative to 241 IgA 2F04 and 047-09 4F04. Data in A and B were analyzed using Chi-square tests. See also FIG. 22.

FIG. 16A-D. cHA vaccination in humans recalls MBCs targeting the anchor epitope. (A-B) Subjects enrolled in a phase 1 clinical trial received a prime-boost of cHA vaccine, where the prime used cH8/1 and the boost used cH5/1. On the prime, subjects either received a LAIV or IIV with adjuvant (AS03). On the boost, subjects received the IIV with or without adjuvant (AS03). Serum was collected before and after vaccination and monitored for competing serum antibodies against the anchor epitope (047-09 4F04) and BN stalk epitope (CR9114). LAIV/IIV+AS03 (n=10); LAIV/IIV (n=7); IIV+AS03/IIV+AS03 (n=7); Placebo (n=6). (A) Trial design. (B) EC50s of serum antibodies competing for binding with 047-09 4F04 for binding to the anchor epitope (left) and CR9114 for binding to the BN stalk epitope (right). Data are mean±S.D. (C) Proportion of anchor epitope binding mAbs binding to cHA or mini-HA. (D) MAb binding to HEK293T cells expressing full length A/California/July 2009 HA (Cal09) or mini-HA with a transmembrane domain. Representative flow cytometry plots of mAbs binding to Cal09 HA and mini-HA (left) and geometric mean fluorescence intensity (gMFI) of mAbs binding to Cal09 and mini-HA (right). Data represent the median and each symbol represents a distinct mAb. Data in B were analyzed using a two-way ANOVA testing for simple effects within rows, data in C were analyzed by Fisher's Exact test and data in D were analyzed by unpaired non-parametric Mann-Whitney test. See also FIG. 23.

FIG. 17A-J. Anchor targeting mAbs use a highly restricted repertoire and possess a conserved binding motif within the K-CDR3. (A-C) Heavy chain VH classes (A) and gene usage of mAbs binding the anchor epitope (B) and the BN stalk epitope (C). Only VH1 gene usage of BN stalk epitope binding mAbs is shown in C. (D) Heavy chain clonality of mAbs binding the anchor epitope. (E) Light chain VK/VL classes usage of mAbs binding the anchor epitope or BN stalk epitope. (F-G) VK (F) and JK (G) gene usage of mAbs binding the anchor epitope. (H) Light chain clonality of mAbs binding the anchor epitope. (I) Private and public clones that share heavy and light chains. (J) Alignment of VDJ of the VH3-48 public clone. (K) Sequence logo of the K-CDR3 and the germline sequence of the K-CDR3 of VK3-11/VK3-15 combined with JK4/JK5. NWP motif is highlighted. (L) Sequence logo of the H-CDR2 with the tyrosines directly following the H-CDR2 and the germline sequence of the H-CDR2 of VH3-23, VH3-30, and VH3-48. Data in A and E were analyzed using Chi-square tests. See also FIG. 24. Shown in FIG. 17J are SEQ ID NOS:18653, 18654, and 18655, respectively. Shown in FIG. 17K are SEQ ID NOS:10711, 12433, 12775, and 12772, respectively. Shown in FIG. 17L are SEQ ID NOS:18650, 18651, and 18652, respectively.

FIG. 18A-F. Humans possess MBCs targeting the anchor epitope. cH5/1+ B cells from PBMCs were sorted from subjects 28 days following a booster with the cH5/1 and were subjected to single-cell RNA-sequencing. (A) Proportion of all B cells with features of anchor antibodies, VH1-69/kappa (BN stalk epitope), or with other repertoire features. (B) Proportion of B cells with anchor binding antibody features or that use VH1-69/kappa chain by subject. Lines connect the same subject. (C) VH/VK pairing of B cells with features of anchor epitope binding antibodies. (D) 34 mAbs with anchor epitope binding mAb repertoire features and tested for competing for binding with 047-09 4F04. (E-F) number of heavy chain mutations (E) and isotype usage (F) of B cells with repertoire features of anchor binding antibodies or VH1-69/kappa. Data in E are represented as mean±S.D. Data in D were analyzed using a paired non-parametric Wilcoxon matched-pairs signed rank test and data in E were analyzed by unpaired non-parametric Mann-Whitney test.

FIG. 19A-E. Binding features of isolated mAbs and 2D class average of anchor epitope binding mAbs. Related to FIG. 12. (A) Proportion of HA+ mAbs binding to distinct HA domains. (B) Proportion of stalk-binding mAbs binding the BN stalk domain. (C-D) Negative stain 2D class averages of 047-09 4F04 (C) and 241 IgA 2F04 (D) binding A/California/April 2009 HA. (E) Binding footprint of mAbs binding the anchor epitope (047-09 4F04, 241 IgA 2F04, and FISW84) and the BN stalk epitope (CR9114 and FI6v3) on A/California/July 2009.

FIG. 20A-D. MAbs targeted the anchor epitope binding to distinct HIN1 viruses, H3N2, influenza B viruses and comparison of mAbs binding virus versus HA. Related to FIG. 13. (A) Heatmap of apparent affinity (Kd; M) of anchor mAb binding to historical and recent HIN1 viruses. (B) Proportion of anchor binding and BN stalk binding mAbs binding to influenza B viruses and H3N2 viruses. (C) Conservation of group 1 influenza virus HAs, as shown on A/California/04/2009 (PDB: 4JTV), with the binding footprint of CR9114 (maroon) and 047-09 4F04 (orange) outlined. (D) Apparent affinity (Kd; M) of mAbs binding anchor, BN stalk, or head epitope binding to recombinant HA from A/California/July 2009 HIN1 and A/California/July 2009 virus. Lines connect the same mAb. Data in B were analyzed by Fisher's Exact tests and data in D were analyzed using a paired non-parametric Wilcoxon matched-pairs signed rank test.

FIG. 21A-E. Deep mutational scanning of 045-09 2B06 and mAb affinity for A/Michigan/45/2015 H1N1. Related to FIG. 13. (A-B) 045-09 2B06 was subjected to deep mutational scanning with a mutant library of A/WSN/1933 (H1N1). (A) Differential selection at each site in HA (H3 numbering). (B) Logo plot of key sites of escape, with the height of each letter proportional to the antibody selection for mutations to that amino acid at that site. (C) Differences in mutations found on the stalk domain of A/Michigan/45/2015 relative to the binding footprints of 047-09 4F04 and 241 IgA 2F04. (D-E) Apparent Affinity (Kd; M) of anchor binding mAbs (D) and BN stalk binding mAbs (E) to A/California/July 2009 and A/Michigan/45/2009. Lines connect the same mAb. Data in D and E were analyzed using a paired non-parametric Wilcoxon matched-pairs signed rank test.

FIG. 22A-E. Characterization of stalk binding mAbs induced by influenza virus vaccines. Related to FIG. 15. (A) Proportion of subjects with stalk+ mAbs from the 2009 MIV or 2014 QIV cohorts. (B) Proportion of mAbs targeting the stalk per person in the 2009 MIV and 2014 QIV cohorts. Red line represents the median. Each symbol in B represents one subject. Only subjects with stalk+ mAbs were included in the analysis. (C-D) 2D class averages of pAbs from subject 236 (C) and 241 (D) at days 7 and 14 post immunization binding to A/Michigan/45/2015 HA. (E) 3D reconstruction of the overlapping of 047-09 4F04, 236 pAb, and FISW84. Data in A were analyzed by Fisher's Exact tests and data in B were analyzed by unpaired non-parametric Mann-Whitney test.

FIG. 23A-F. Seroconversion against anchor and BN stalk epitopes after cHA vaccination and mAb binding to various recombinant HAs. Related to FIG. 16. (A-B) Seroconversion (d28/d0) of antibodies competing for binding against the anchor epitope (A) and the BN stalk epitope (B). Bar represents the median and each symbol represents one subject. (C-D) Antibody titers (EC50) of serum antibodies collected on day 112 and day 420 against the anchor epitope (C) and the BN stalk epitope (D). Lines connect titers from the same subject and each pair of symbols represents one subject. (E) Anchor and BN stalk targeting mAbs binding to mini-HA and cH6/1. (F) Proportion of anchor epitope targeting mAbs binding to A/California/July 2009 recombinant HA with a GCN4 or fibritin trimerization domain. Data in A and B were analyzed by non-parametric Kruskal-Wallis Tests, data in C and D were analyzed using a paired non-parametric Wilcoxon matched-pairs signed rank test, and data in F were analyzed by a Fisher's Exact test.

FIG. 24A-G. Additional repertoire features of mAs binding the anchor epitope. Related to FIG. 17. (A) JH gene usage by anchor and BN stalk binding antibodies. (B) JK gene usage of mAbs binding the BN stalk epitope. (C) VJ sequences of kappa chains of the public clone paired VH3-48. (D-E) Mutations (D) and CDR3 amino acid (AA) lengths (E) of heavy and light chains of mAbs binding the anchor or BN stalk epitopes. Red bar represents median. (F) NWP motif of K-CDR3 and Y59 following H-CDR2 (H) of FISW84 (PDB: 6HJQ). (G) H-CDR2 sequences of germline VH3 genes. H-CDR2 is highlighted in blue and the first amino acid following H-CDR2 is highlighted in red. Data in A were analyzed using a Chi-square test, and data in D and E were analyzed by unpaired non-parametric Mann-Whitney tests. Shown in FIG. 24C are SEQ ID NOS:7623, 7678, and 7627, respectively. Shown in FIG. 24G are SEQ ID NOS: 18656-18668, respectively.

### DETAILED DESCRIPTION OF THE INVENTION

Broadly neutralizing antibodies (bnAbs) targeting epitopes of the influenza virus hemagglutinin (HA) have the potential to provide near universal protection against influenza virus infection¹. However, viral mutants that escape bnAbs have been reported^{2,3}. The identification of bnAb classes that can neutralize viral escape mutants is critical for universal influenza virus vaccine design. Here, we report a distinct class of bnAbs targeting a discrete membrane-proximal anchor epitope of the HA stalk domain. Anchor epitope-targeting antibodies are broadly neutralizing across H1 viruses and can cross-react with pandemic-threat H2 and H5 viruses. Antibodies targeting this anchor epitope utilize a highly restricted repertoire, which encodes for two public binding motifs that make extensive contacts with conserved residues in the fusion peptide. Moreover, anchor epitope-targeting B cells are common in the human memory B cell (MBC) repertoire and were recalled in humans by an oil-in-water adjuvanted chimeric HA (cHA) vaccine^{4,5}, a potential universal influenza virus vaccine. To maximize protection against seasonal and pandemic influenza viruses, vaccines should aim to boost this previously untapped source of bnAbs that are widespread in the human MBC pool.

### I. Antibodies

Described herein are antibodies, antigen binding fragments thereof, or polypeptides capable of specifically binding to an influenza antigen

The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

### A. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-L1, CDR-L2, and CDR-L3. The L3 (CDR-L3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as CDR-H1, CDR-H2 and CDR-H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include:
1) Computational predictions of the tertiary structure of the antibody/epitope binding interactions based on the chemical nature of the amino acid sequence of the antibody variable region and composition of the epitope.
2) Hydrogen-deuterium exchange and mass spectroscopy
3) Polypeptide fragmentation and peptide mapping approaches in which one generates multiple overlapping peptide fragments from the full length of the polypeptide and evaluates the binding affinity of these peptides for the epitope.
4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### B. Functional Antibody Fragments and Antigen-Binding Fragments

### 1. Antigen-Binding Fragments

Certain aspects relate to antibody fragments, such as antibody fragments that bind to flu antigens. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment (also “Fab”) means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

### 2. Fragment Antigen Binding Region, Fab

Fab polypeptides of the disclosure include the Fab antigen binding fragment of an antibody. Unless specifically stated otherwise, the term “Fab” relates to a polypeptide excluding the Fc portion of the antibody. The Fab may be conjugated to a polypeptide comprising other components, such as further antigen binding domains, costimulatory domains, linkers, peptide spacers, transmembrane domains, endodomains, and accessory proteins. Fab polypeptides can be generated using conventional techniques known in the art and are well-described in the literature.

### 3. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

### C. Polypeptides with Antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### D. Antibody Binding

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

### 1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain embodiments may have an affinity constant (Ka) of about, at least about, or at most about 10⁶, 10⁷, 10⁸, 10⁹, or 10¹⁰ M or any range derivable therein. Similarly, in some embodiments, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the invention is said to “specifically bind” its target antigen when the dissociation constant (KD) is ≤10⁻⁸ M. The antibody specifically binds antigen with “high affinity” when the KD is ≤5×10⁻⁹ M, and with “very high affinity” when the KD is ≤5×10⁻¹⁰ M.

### 2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

### 3. Modification of Antibody Antigen-Binding Domains

It is understood that the antibodies of the present invention may be modified, such that they are substantially identical to the antibody polypeptide sequences, or fragments thereof, and still bind the epitopes of the present invention. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present invention, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with VH and VL region pairs that bind either the same antigen (multi-valent) or a different antigen (multi-specific).

### E. Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the invention to obtain PEGylated derivatives of antibodies. See, e.g., EP 0 154 316 and EP 0 401 384. In some aspects, the antibody is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

### 1. Conjugation

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses).

Optionally, an antibody or an immunological portion of an antibody can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0 486 525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

### a. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody (Ab) is conjugated to one or more drug moieties (D) through a linker (L). The ADC may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Antibody drug conjugates may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate. In yet another aspect, the antibody may be conjugated to a “receptor” (such as streptavidin) for utilization in tumor or cancer cell pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Examples of an antibody-drug conjugates known to a person skilled in the art are pro-drugs useful for the local delivery of cytotoxic or cytostatic agents, i.e. drugs to kill or inhibit tumor cells in the treatment of cancer (Syrigos and Epenetos, Anticancer Res. 19:605-614 (1999); Niculescu-Duvaz and Springer, Adv. Drg. Del. Rev. 26:151-172 (1997); U.S. Pat. No. 4,975,278). In contrast, systematic administration of these unconjugated drug agents may result in unacceptable levels of toxicity to normal cells as well as the target tumor cells (Baldwin et al., Lancet 1:603-5 (1986); Thorpe, (1985) “Antibody Carriers of Cytotoxic Agents in Cancer Therapy: A Review,” In: Monoclonal Antibodies '84: Biological and Clinical Applications, A. Pincera et al., (eds.) pp. 475-506). Both polyclonal antibodies and monoclonal antibodies have been reported as useful in these strategies (Rowland et al., Cancer Immunol. Immunother. 21:183-87 (1986)).

In certain aspects, ADC include covalent or aggregative conjugates of antibodies, or antigen-binding fragments thereof, with other proteins or polypeptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912. Oligomers that contain one or more antibody polypeptides may be employed as antagonists. Oligomers may be in the form of covalently linked or non-covalently linked dimers, trimers, or higher oligomers. Oligomers comprising two or more antibody polypeptides are contemplated for use. Other oligomers include heterodimers, homotrimers, heterotrimers, homotetramers, heterotetramers, etc. In certain aspects, oligomers comprise multiple antibody polypeptides joined via covalent or non-covalent interactions between peptide moieties fused to the antibody polypeptides. Such peptides may be peptide linkers (spacers), or peptides that have the property of promoting oligomerization. Leucine zippers and certain polypeptides derived from antibodies are among the peptides that can promote oligomerization of antibody polypeptides attached thereto, as described in more detail below.

### b. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., 1987).

### II. Antibody Production

### A. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques. See, for example, Stewart and Young, (1984); Tam et al, (1983); Merrifield, (1986); and Barany and Merrifield (1979), each incorporated herein by reference.

Briefly, a polyclonal antibody is prepared by immunizing an animal with an antigen or a portion thereof and collecting antisera from that immunized animal. The antigen may be altered compared to an antigen sequence found in nature. In some embodiments, a variant or altered antigenic peptide or polypeptide is employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography (Harlow and Lane, Antibodies: A Laboratory Manual 1988).

Methods of making monoclonal antibodies are also well known in the art (Kohler and Milstein, 1975; Harlow and Lane, 1988, U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after two to three weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001).

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m2) (Johnson/Mead, NJ), cytokines such as β-interferon, IL-2, or IL-12, or genes encoding proteins involved in immune helper functions, such as B-7.A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

### B. Fully Human Antibody Production

Methods are available for making fully human antibodies. Using fully human antibodies can minimize the immunogenic and allergic responses that may be caused by administering non-human monoclonal antibodies to humans as therapeutic agents. In one embodiment, human antibodies may be produced in a non-human transgenic animal, e.g., a transgenic mouse capable of producing multiple isotypes of human antibodies to protein (e.g., IgG, IgA, and/or IgE) by undergoing V-D-J recombination and isotype switching. Accordingly, this aspect applies to antibodies, antibody fragments, and pharmaceutical compositions thereof, but also non-human transgenic animals, B-cells, host cells, and hybridomas that produce monoclonal antibodies. Applications of human antibodies include, but are not limited to, detect a cell expressing an anticipated protein, either in vivo or in vitro, pharmaceutical preparations containing the antibodies of the present invention, and methods of treating disorders by administering the antibodies.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90:2551-2555 (1993); Jakobovits et al., Nature 362:255-258 (1993); Bruggermann et al., Year in Immunol. 7:33 (1993). In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

The transgenic mice described above, referred to herein as “HuMAb” mice, contain a human immunoglobulin gene minilocus that encodes unrearranged human heavy (μ and γ) and κ light chain immunoglobulin sequences, together with targeted mutations that inactivate the endogenous μ and κ chain loci (Lonberg et al., Nature 368:856-859 (1994)). Accordingly, the mice exhibit reduced expression of mouse IgM or κ chains and in response to immunization, the introduced human heavy and light chain transgenes undergo class switching and somatic mutation to generate high affinity human IgG κ monoclonal antibodies (Lonberg et al., supra; Lonberg and Huszar, Intern. Ref. Immunol. 13:65-93 (1995); Harding and Lonberg, Ann. N.Y. Acad. Sci. 764:536-546 (1995)). The preparation of HuMAb mice is described in detail in Taylor et al., Nucl. Acids Res. 20:6287-6295 (1992); Chen et al., Int. Immunol. 5:647-656 (1993); Tuaillon et al., J. Immunol. 152:2912-2920 (1994); Lonberg et al., supra; Lonberg, Handbook of Exp. Pharmacol. 113:49-101 (1994); Taylor et al., Int. Immunol. 6:579-591 (1994); Lonberg and Huszar, Intern. Ref. Immunol. 13:65-93 (1995); Harding and Lonberg, Ann. N.Y. Acad. Sci. 764:536-546 (1995); Fishwild et al., Nat. Biotechnol. 14:845-851 (1996); the foregoing references are herein incorporated by reference in their entirety for all purposes. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807; as well as International Patent Application Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., Nat. Genetics 15:146-156 (1997), which are herein incorporated by reference. For example, the HCo7 and HCo12 transgenic mice strains can be used to generate human antibodies.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in Hoogenboom et al., J. Mol. Biol. 227:381 (1991); and Marks et al., J. Mol. Biol. 222:581 (1991)). One such technique is described in International Patent Application Publication No. WO 99/10494 (herein incorporated by reference), which describes the isolation of high affinity and functional agonistic antibodies for MPL- and msk-receptors using such an approach.

### C. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (VL and VH). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different VL- and VH-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, VL and VH, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by amino-terminal and/or carboxy-terminal deletions, where the remaining amino acid sequence is substantially identical to the corresponding positions in the naturally occurring sequence deduced, for example, from a full-length cDNA sequence.

Antibodies may also be generated using peptide analogs of the epitopic determinants disclosed herein, which may consist of non-peptide compounds having properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987). Liu et al. (2003) also describe “antibody like binding peptidomimetics” (ABiPs), which are peptides that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the invention are proteins that are structurally similar to an antibody displaying a desired biological activity, such as the ability to bind a protein, but have one or more peptide linkages optionally replaced by a linkage selected from: —CH2NH—, —CH2S—, —CH2—CH2—, —CH═CH—(cis and trans), —COCH2—, —CH(OH)CH2—, and —CH2SO— by methods well known in the art. Systematic substitution of one or more amino acids of a consensus sequence with a D-amino acid of the same type (e.g., D-lysine in place of L-lysine) may be used in certain embodiments of the invention to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of the Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used.

### III. Obtaining Encoded Antibodies

In some aspects, there are nucleic acid molecule encoding antibody polypeptides (e.g., heavy or light chain, variable domain only, or full-length). These may be generated by methods known in the art, e.g., isolated from B cells of mice that have been immunized and isolated, phage display, expressed in any suitable recombinant expression system and allowed to assemble to form antibody molecules.

### A. Expression

The nucleic acid molecules may be used to express large quantities of recombinant antibodies or to produce chimeric antibodies, single chain antibodies, immunoadhesins, diabodies, mutated antibodies, and other antibody derivatives. If the nucleic acid molecules are derived from a non-human, non-transgenic animal, the nucleic acid molecules may be used for antibody humanization.

### 1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the antibodies, or antigen-binding fragments thereof, DNAs encoding partial or full-length light and heavy chains are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### 2. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### 3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624,5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 4. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

The nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### IV. Antibodys, Antigen Binding Fragments, and Polypeptides

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity. The term polypeptide also includes and antibody fragment described herein as well as antibody domains, such as HCDR1, HCDR2, HCDR3, LCDR1, LCDR2, LCDR3, HFRW1, HFRW2, HFRW3, HFRW4, LFRW1, LFRW2, LFRW3, LFRW4, VH, VL, CH, or CL.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of an antibody, antigen binding fragment, protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The antibody, antigen binding fragment, polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of a polypeptide of table 1, a nucleic acid of table 2, a polypeptide encoded by a nucleic acid of table 2, or the polypeptide or nucleic acid of any one of SEQ ID NOS: 1-18668.

In some embodiments, the antibody, antigen binding fragment, protein, or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of a polypeptide of table 1, a nucleic acid of table 2, a polypeptide encoded by a nucleic acid of table 2, or the polypeptide or nucleic acid of any one of SEQ ID NOS:1-18668.

In some embodiments, the antibody, antigen binding fragment, or polypeptide may comprise 1,2, 3,4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407,408,409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702,703, 704, 705, 706,707, 708, 709, 710, 711, 712, 713, 714, 715,716, 717, 718, 719,720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912,913, 914, 915, 916,917, 918, 919, 920, 921, 922, 923, 924, 925,926, 927, 928, 929,930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids or nucleic acids of a polypeptide of table 1, a nucleic acid of table 2, a polypeptide encoded by a nucleic acid of table 2, or the polypeptide or nucleic acid of any one of SEQ ID NOS: 1-18668.

In some embodiments, the antibody, antigen binding fragment, protein, or polypeptide may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of a polypeptide of table 1, a polypeptide encoded by a nucleic acid of table 2, a nucleic acids of table 2, or the polypeptide or nucleic acid of any one of SEQ ID NOS: 1-18668 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of the polypeptides of table 1, nucleic acids of table 2, polypeptides encoded by the nucleic acids of table 2, or the polypeptide or nucleic acid of any one of SEQ ID NOS: 1-18668.

In some aspects there is a nucleic acid molecule, antibody, antigen binding fragment, protein, or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of the polypeptides of table 1, polypeptides encoded by the nucleic acids of table 2, nucleic acid of table 2, or the polypeptide or nucleic acid of any one of SEQ ID NOS:1-18668 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of a polypeptide of table 1, nucleic acid of table 2, a polypeptide encoded by a nucleic acid of table 2, or the polypeptide or nucleic acid of any one of SEQ ID NOS: 1-18668.

In some embodiments, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of the heavy chain, light chain, VH, VL, HCDR1, HCDR2, HCDR3, LCDR1, LCDR2, LCDR3, HFRW1, HFRW2, HFRW3, HFRW4, LFRW1, LFRW2, LFRW3, or LFRW4 of a polypeptide of table 1, a polypeptide encoded by a nucleic acid of table 2, or a or the polypeptide of any one of SEQ ID NOS:1-18668, is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some embodiments, a polypeptide (e.g., antibody, antibody fragment, Fab, etc.) of the disclosure comprises a CDR that is at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any range derivable therein) in sequence to one of a polypeptide of table 1, a polypeptide encoded by a nucleic acid of table 2, or the polypeptide of any one of SEQ ID NOS: 1-18668. In some embodiments, a polypeptide comprises 1, 2, and/or 3 CDRs from an antibody clone of table 1 or the antibody clones encoded by the nucleic acids of table 2. The CDR may be one that has been determined by Kabat, IMGT, or Chothia. In further embodiments, a polypeptide may have CDRs that have 1, 2, and/or 3 amino acid changes (e.g., addition of 1 or 2 amino acids, deletions of 1 or 2 amino acids, substitution) with respect to these 1, 2, or 3 CDRs. In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

From amino to carboxy terminus the CDRs are CDR1, CDR2, and CDR3. In some embodiments, a polypeptide may have CDRs that have 1, 2, and/or 3 amino acid changes (e.g., addition of 1 or 2 amino acids, deletions of 1 or 2 amino acids, substitution) with respect to CDR1, CDR2, or CDR3. In some embodiments, the CDRs of the heavy and light chains of Table 1 or Table 2 (or those in SEQ ID NOS:1-18668) may further comprise 1, 2, 3, 4, 5, or 6 additional amino acids at the amino or carboxy terminus of the CDR, The additional amino acids may be from the heavy and/or light chain framework regions of the HCs and LCs of Table 1, that are shown as immediately adjacent to the CDRs. Accordingly, embodiments relate to polypeptides comprising an HCDR1 (i.e., CDR-H1), HCDR2(i.e., CDR-H2), HCDR3(i.e., CDR-H3), LCDR1(i.e., CDR-L1), LCDR2(i.e., CDR-L2), and/or LCDR3(i.e., CDR-L3) with at least or at most or exactly 1, 2, 3, 4, 5, 6 or 7 amino acids at the amino end of the CDR or at the carboxy end of the CDR, wherein the additional amino acids are the 1, 2, 3, 4, 5, 6, or 7 amino acids of Table 1 that are shown as immediately adjacent to the CDRs. Other embodiments relate to antibodies comprising one or more CDRs, wherein the CDR is a fragment of Table 1 and wherein the fragment lacks 1, 2, 3, 4, or 5 amino acids from the amino or carboxy end of the CDR. In some embodiments, the CDR may lack one, 2, 3, 4, 5, 6, or 7 amino acids from the carboxy end and may further comprise 1, 2, 3, 4, 5, 6, 7, or 8 amino acids from the framework region of the amino end of the CDR. In some embodiments, the CDR may lack one, 2, 3, 4, 5, 6, or 7 amino acids from the amino end and may further comprise 1, 2, 3, 4, 5, 6, 7, or 8 amino acids from the framework region of the carboxy end of the CDR. In further embodiments, an antibody may be alternatively or additionally humanized in regions outside the CDR(s) and/or variable region(s). In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

In other embodiments, a polypeptide or protein comprises 1, 2, 3, 4, 5, or 6 CDRs from either or both of the light and heavy variable regions of the antibodies of Table 1, and 1, 2, 3, 4, 5, or 6 CDRs may have 1, 2, and/or 3 amino acid changes with respect to these CDRs. In some embodiments, parts or all of the antibody sequence outside the variable region have been humanized. A protein may comprise one or more polypeptides. In some aspects, a protein may contain one or two polypeptides similar to a heavy chain polypeptide and/or 1 or 2 polypeptides similar to a light chain polypeptide.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### V. Sequences

Polypeptide, antibody, and antigen binding fragment embodiments are shown below in the following tables.

Lengthy table referenced here
US20250059260A1-20250220-T00001
Please refer to the end of the specification for access instructions.

Lengthy table referenced here
US20250059260A1-20250220-T00002
Please refer to the end of the specification for access instructions.

**The sequences associated with the GenBank accession numbers listed in Table 1 are incorporated by reference for all purposes.

### A. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### B. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within 2 is included. In some aspects of the invention, those that are within +1 are included, and in other aspects of the invention, those within +0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### VI. Nucleic Acids

In certain embodiments, nucleic acid sequences can exist in a variety of instances such as: isolated segments and recombinant vectors of incorporated sequences or recombinant polynucleotides encoding peptides and polypeptides of the disclosure, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 550° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antigenic peptide or polypeptide) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific nucleic acid sequences. For instance, a nucleic acid molecule probe may be used in diagnostic methods or a nucleic acid molecule PCR primer may be used to amplify regions of DNA that could be used, inter alia, to isolate nucleic acid sequences for use in producing the engineered cells of the disclosure. In a preferred embodiment, the nucleic acid molecules are oligonucleotides.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### VII. Polypeptide Expression

In some aspects, there are nucleic acid molecule encoding polypeptides, antibodies, or antigen binding fragments of the disclosure. The nucleic acid molecules may be used to express large quantities of polypeptides. If the nucleic acid molecules are derived from a non-human, non-transgenic animal, the nucleic acid molecules may be used for humanization of the antibody or TCR genes.

### A. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody heavy and/or light chain, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### B. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### C. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624,5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### VIII. Pharmaceutical Compositions

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection. In some embodiments, compositions of the present disclosure (e.g., compositions comprising flu antigen-binding polypeptides) are administered to a subject intravenously.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most or at least 3, 4, 5, 6, 7, 8, 9, 10 or more. The administrations may range from 2-day to 12-week intervals, more usually from one to two week intervals.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

Pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (e.g., polypeptides of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IX. Detectable Labels

In some aspects of this disclosure, it will be useful to detectably or therapeutically label a Fab polypeptide or protein G Fab-binding domain. Methods for conjugating polypeptides to these agents are known in the art. For the purpose of illustration only, polypeptides can be labeled with a detectable moiety such as a radioactive atom, a chromophore, a fluorophore, or the like. Such labeled polypeptides can be used for diagnostic techniques, either in vivo, or in an isolated test sample or in methods described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue.™., and Texas Red. Other suitable optical dyes are described in the Haugland, Richard P. (1996) Handbook of Fluorescent Probes and Research Chemicals (6.sup.th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/polypeptides, e.g., rhodamine/anti-rhodamine, biotin/avidin and biotin/strepavidin.

The coupling of polypeptides to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten polypeptides. See, Harlow and Lane (1988) supra.

### X. Sample Preparation

In certain aspects, methods involve obtaining or evaluating a sample from a subject. The sample may include a sample obtained from any source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport ofthe biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### XI. Host Cells

As used herein, the terms “cell,” “cell line,” and “cell culture” may be used interchangeably. All of these terms also include both freshly isolated cells and ex vivo cultured, activated or expanded cells. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

In certain embodiments transfection can be carried out on any prokaryotic or eukaryotic cell. In some aspects electroporation involves transfection of a human cell. In other aspects electroporation involves transfection of an animal cell. In certain aspects transfection involves transfection of a cell line or a hybrid cell type. In some aspects the cell or cells being transfected are cancer cells, tumor cells or immortalized cells. In some instances tumor, cancer, immortalized cells or cell lines are induced and in other instances tumor, cancer, immortalized cells or cell lines enter their respective state or condition naturally. In certain aspects the cells or cell lines can be A549, B-cells, B16, BHK-21, C2C12, C6, CaCo-2, CAP/, CAP-T, CHO, CHO2, CHO-DG44, CHO-K1, COS-1, Cos-7, CV-1, Dendritic cells, DLD-1, Embryonic Stem (ES) Cell or derivative, H1299, HEK, 293, 293T, 293FT, Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH 3T3, NIH3T3L1, K562, NK-cells, NS0, Panc-1, PC12, PC-3, Peripheral blood cells, Plasma cells, Primary Fibroblasts, RBL, Renca, RLE, SF21, SF9, SH-SY5Y, SK-MES-1, SK—N—SH, SL3, SW403, Stimulus-triggered Acquisition of Pluripotency (STAP) cell or derivate SW403, T-cells, THP-1, Tumor cells, U2OS, U937, peripheral blood lymphocytes, expanded T cells, hematopoietic stem cells, or Vero cells.

### XII. Kits

Certain aspects of the present invention also concern kits containing compositions of the disclosure or compositions to implement methods of the disclosure. In some embodiments, kits can be used to detect the presence of an influenza virus or antigen in a sample. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, a kit contains one or more polypeptides capable of binding to an influenza antigen. For example, a kit may comprise at least 1, 2, 3, 4, 5, 6, 7, 8, 9, 10 or more Fabs disclosed herein for detecting an influenza antigen. In some embodiments, a kit comprises a detection pair. In some embodiments, a kit comprises an enzyme. In some embodiments, a kit comprises a substrate for an enzyme.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. In certain aspects, negative and/or positive control nucleic acids, probes, and inhibitors are included in some kit embodiments.

Kits may further comprise instructions for use. For example, in some embodiments, a kit comprises instructions for detecting an influenza antigen in a sample.

It is contemplated that any method or composition described herein can be implemented with respect to any other method or composition described herein and that different embodiments may be combined. The claims originally filed are contemplated to cover claims that are multiply dependent on any filed claim or combination of filed claims.

### XIII. EXAMPLES

The following examples are included to demonstrate certain embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1—Broadly Neutralizing Antibodies Target a Hemagglutinin Anchor Epitope

Antibodies against the major surface glycoprotein hemagglutinin (HA) are critical for providing protection against influenza virus infection^{6,7}. However, most HA-binding antibodies target variable epitopes of the HA head domain, which provide limited protection against antigenically similar influenza virus strains³. Vaccine formulations that preferentially induce antibodies against conserved epitopes of the HA head and stalk domains could provide broad and potent protectionagainst a wide array of influenza viruses. Several leading universal influenza virus candidates are designed to induce antibodies specifically against the stalk domain, but the spectrum of distinct epitopes on the stalk targeted by the human B cell repertoire remains to be determined. By analyzing the specificities of B cells targeting the H1 stalk through the generation of monoclonal antibodies (mAbs), our study reveals a new class of bnAbs against an underappreciated epitope where HA anchors itself into the viral membrane. Next-generation vaccine platforms should take advantage of this finding to elicit antibodies against the conservedanchor epitope.

### A. Results

### 1. Discovery of Anchor Epitope Binding mAbs

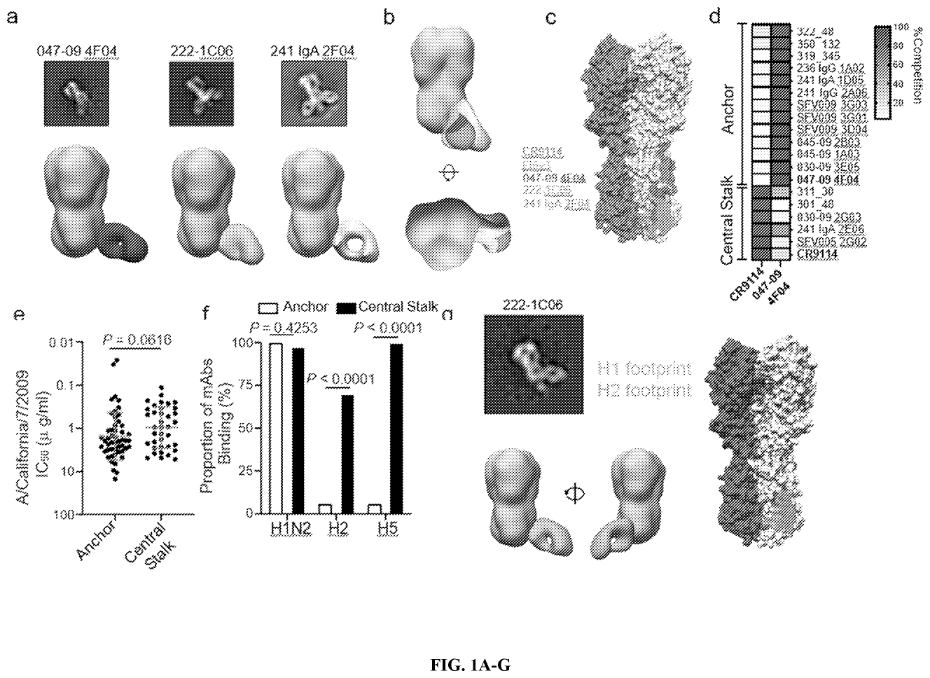

To investigate the specificities of HA-specific antibodies, we generated 358 mAbs from plasmablasts and HA⁺ memory B cells (MBCs) isolated from volunteers that were vaccinated against or naturally infected with seasonal influenza viruses or were participants in a phase 1 clinical trial of cHA vaccine^{4,5}. Of all mAbs tested, nearly half targeted the HA stalk domain, 21% of which targeted the well-characterized central stalk (CS) epitope (FIG. 5a). Notably, stalk-binding mAbs were disproportionally isolated from the infected, 2009 pandemic HIN1 (pH1N1) monovalent inactivated influenza vaccine (MIV), and cHA vaccine cohorts (FIG. 5b), as these exposure routes have previously been shown to induce antibody responses against the HA stalk^{5,8,9}. To investigate which epitopes the remaining 79% of stalk-binding mAbs were targeting, we performed negative stain electron microscopy with three non-CR9114¹⁰ competing stalk domain-binding mAbs: 047-09 4F04, 241 IgA 2F04, and 222-1C06. All three mAbs bound an epitope near the anchor of the HA stalk and were oriented at an upward angle towards the epitope (FIG. 1a-b and FIG. 5c), suggesting this epitope may be partially obstructed by the lipid membrane and may only be exposed forantibody binding as the HA trimers flex on the membrane. FISW84, a recently identified stalk-binding mAb”, targets an epitope that overlaps with the three identified anchor-binding mAbs (FIG. 5d), suggesting the anchor epitope is a common stalk epitope. Moreover, a proximal epitope was previously identified on group 2 viruses that is targeted by mAbs CR8020¹² and CR8043¹³. Despite some overlap, the epitope targeted by the group 2 mAbs was considerably farther up and to the right on the HA stalk relative to the anchor epitope, and mAbsCR8020 and CR8043 targeted the stalk from above (FIG. 5e-f), at an angle and positioning more akin to antibodies targeting the CS epitope. MAbs binding the CS epitope (CR9114¹⁰ and FI6v3¹⁴) did not have overlapping footprints or compete for HA binding with the anchor-binding 047-09 4F04 mAb in an HA competition assay (FIG. 1c-d). In total, we identified 50 distinct mAbs that competed for binding to the anchor epitope from a total of 21 individuals (FIG. 1d and Extended Data Table 1 and 2). Of these, 34 anchor-binding mAbs from 15 donors were isolated from the cHA vaccine trial (Extended Data Table 1 and 2).

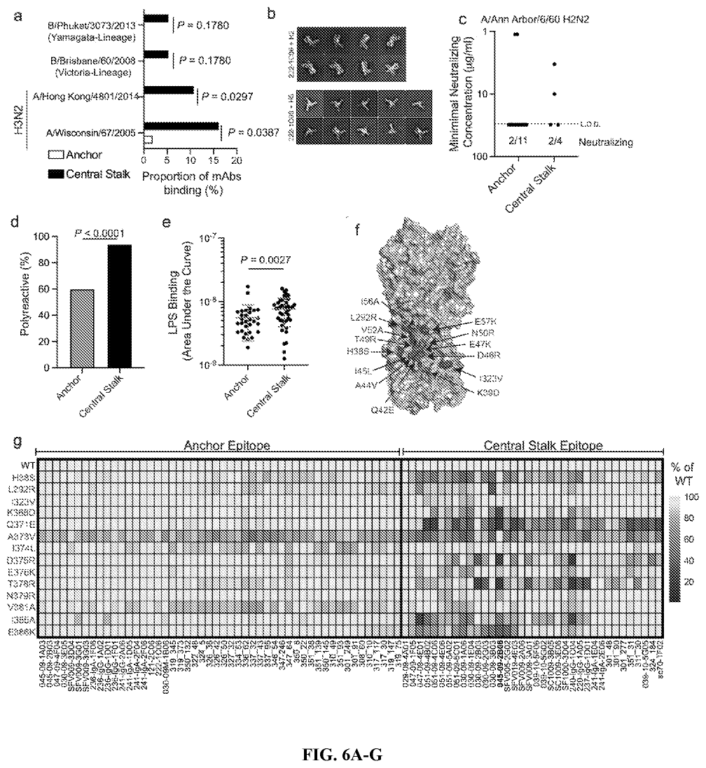

Anchor-binding mAbs were broadly reactive and broadly neutralizing against pre- and post-pandemic H1N1 viruses and a swine-origin H1N2 virus (FIG. 1e and FIG. 5g-h). Notably, anchor-binding mAbs had similar neutralizing potency against pH1N1 relative to mAbs against the central stalk epitope (FIG. 1e and FIG. 5i-j). Many stalk-targeting antibodies mediate protection via Fc-mediated functions, including antibody-dependentcellular cytotoxicity (ADCC)^{15,16}. Anchor epitope-binding mAbs largely did not possess ADCC activity (15/18; FIG. 5k-1), potentially due to the upward angle of approach of anchor-binding antibodies which may position the Fc distantly from effector cells. Despite pan-H1 binding, anchor-targeting mAbs rarely cross-reacted with other HA subtypes tested, including H3, a group 2 subtype, other group 1 subtypes, including H2 and H5, and influenza B viruses (FIG. 1f and FIG. 6a). Despite this, 222-1C06 mAb cross-reacted with H2 and H5 HA (FIG. 1g and FIG. 6b) and several anchor-binding antibodies could neutralize an H2N2 virus (FIG. 6c), suggesting antibodies targeting the anchor epitope can cross-neutralize other group 1 influenza A viruses. We recently demonstrated that bnAbs against the HA stalk are often polyreactive¹⁷, which may limit the activation of B cells expressing bnAbs. Relative to mAbs targeting the central stalk epitope, we identified thatanchor-binding mAbs were proportionally less likely to be polyreactive and those that were polyreactive had weaker relative affinity for LPS (FIG. 6d-e). These data suggest that although polyreactivity is selected for within the anti-anchor epitope B cell pool, it is not to the same level as B cells against the CS epitope.

H1N1 viruses have acquired several mutations within the HA stalk domain, likely due to antibody selective pressures or to increase stability. To understand whether these mutations affect antibody binding to the anchor epitope, we screened mAbs against naturally occurringand experimentally identified viral escape mutants of mAbs binding to the CS epitope (FIG. 6f-g and Extended Data Table 3). Anchor epitope-binding mAbs were mostly unaffected by these mutants whereas most of the CS-binding mAbs showed reduced binding to at least one mutant (FIG. 6g). Notably, most mAbs had reduced binding to A388V of HA2, which was recently shown to preferentially grow in the presence of mAbs againstthe CS epitope². While A373 is distant from the anchor epitope, the A373V mutation was shown to affect the conformation of the HA stalk², explaining the broad reduction of HA binding by antibodies targeting either stalk epitope. Anchor-binding mAbs only demonstrated a 10-30% reduction in binding (FIG. 6g), indicating they are still likely to neutralize viruses carrying the A373V mutation.

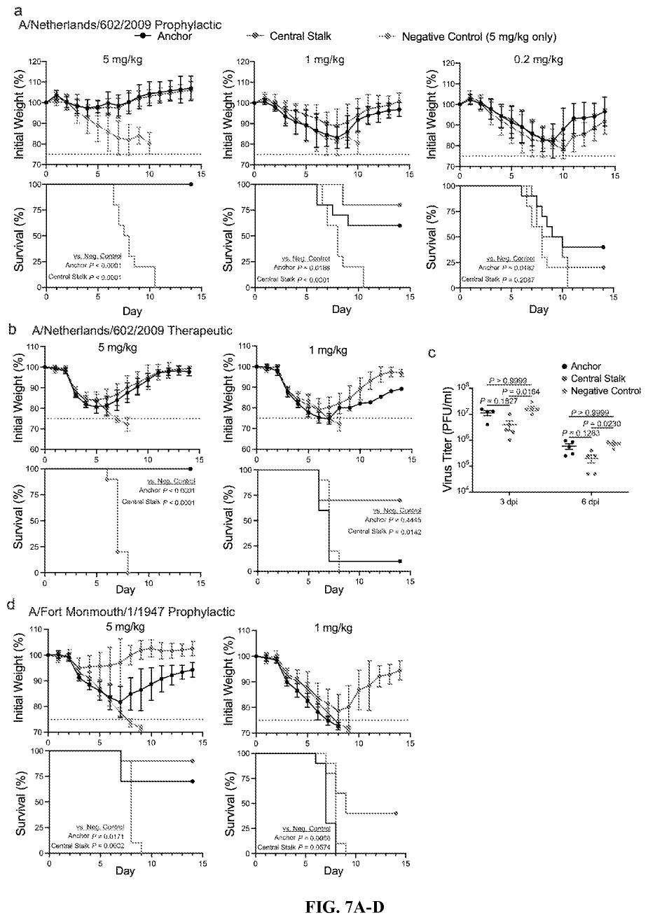

To test whether mAbs targeting the anchor epitope were protective in vivo, we prophylactically and therapeutically (48 hours after infection) administered a cocktail of 5 mAbs targeting the anchor epitope or the CS epitope to mimic a polyclonal response against these epitopes and infected mice with a lethal dose of a mouse-adapted pH1N1 virus (A/Netherlands/602/2009; Supplementary Table 1). Mice that received a prophylactic or therapeutic anti-anchor cocktail experienced similar weight loss and mortality, with 5 mg/kg doseproviding 100% protection (FIG. 7a-b). No differences in lung viral titers were detected in mice that received the anti-anchor cocktail prophylactically relative to the negative control mAb administered mice (FIG. 7c). Anti-stalk antibodies do not provide sterilizing immunity but are known to neutralize subsequent rounds and limit disease progression¹⁸. As a result, lung viral titers do not necessarily reflect protection from morbidity and mortality. Lastly, anti-anchor cocktail given prophylactically provided 70% protection againstlethal A/Fort Monmouth/January 1947 infection (FIG. 7d), a virus that circulated prior to the birth of any of the donors in our study (Extended Data Table 1). Since anchor-binding mAbs do not engage in ADCC for the most part, antibodies targeting the anchor epitope likely provide protection in vivo through direct neutralization of virus. Together, these data indicate that antibodies against the anchor epitope are pan-H1 neutralizing and protective in vivo.

### 2. Structure of an Anchor-Binding Antibody

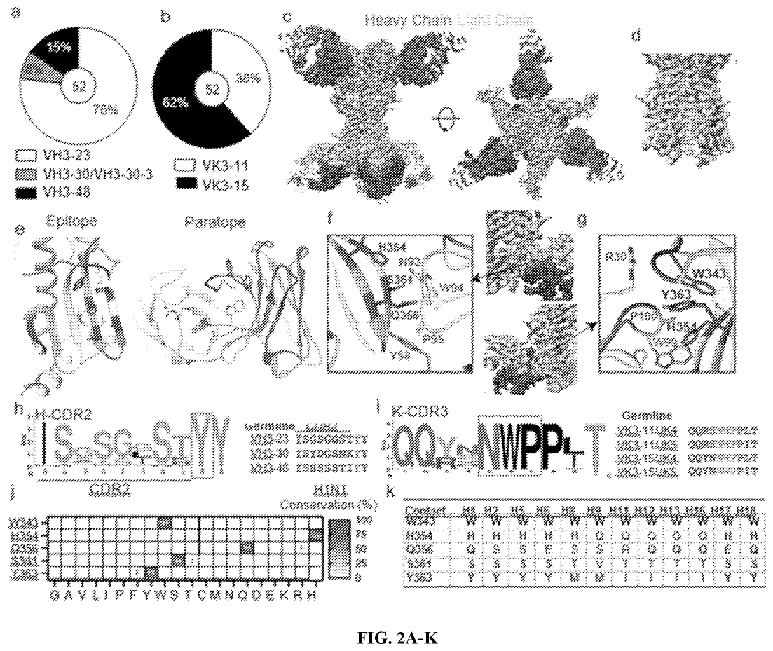

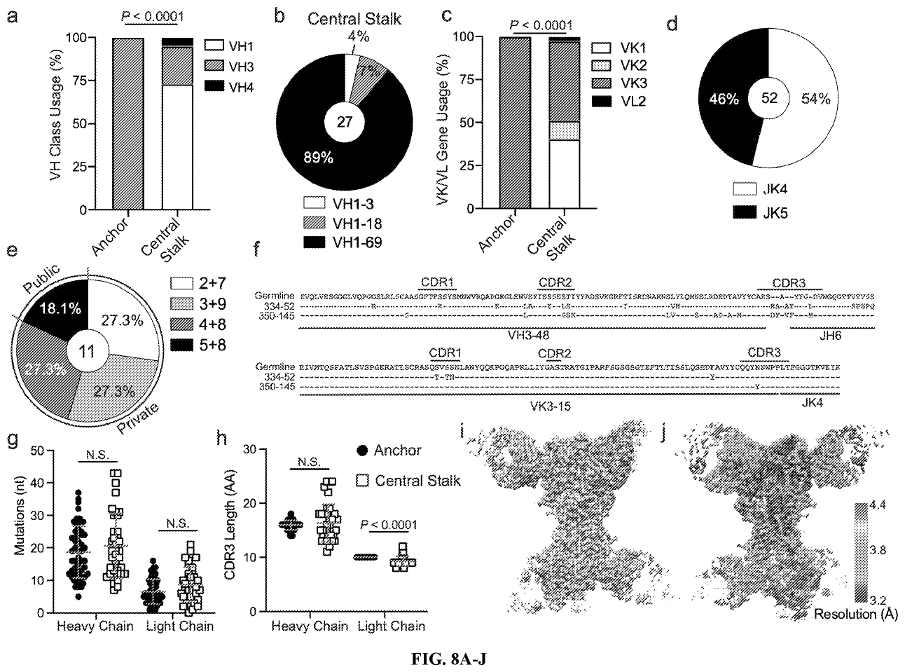

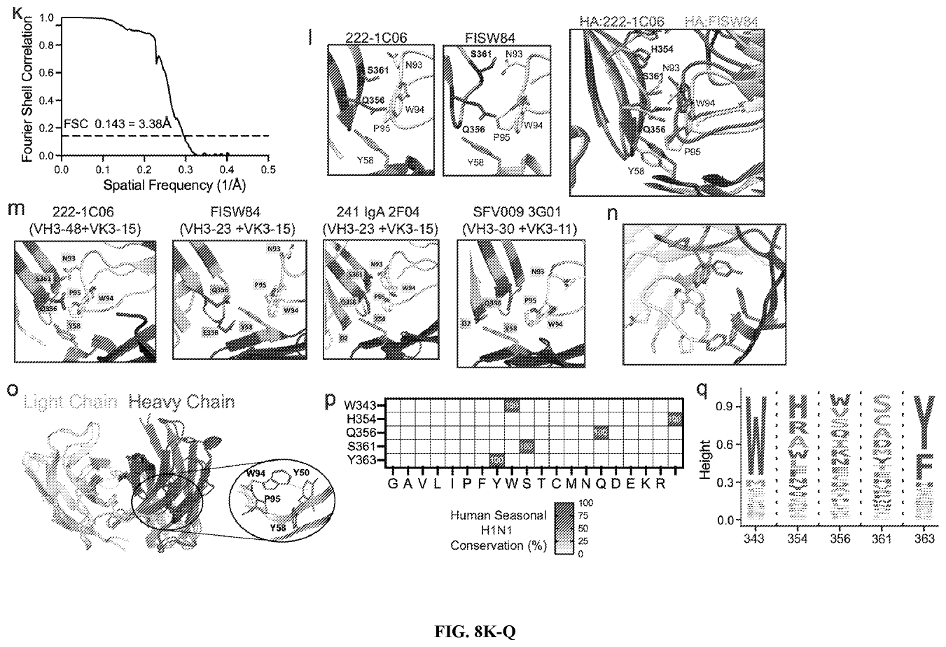

All anchor epitope-binding mAbs utilized one of four VH3 genes: VH3-23, VH3-30/VH3-30-3, and VH3-48, in contrast to mAbs targeting the CS epitope which commonly use VH1-69 (FIG. 2a and FIG. 8a-b). Anchor epitope-binding mAbs also utilized a highly restricted lightchain repertoire, with all mAbs utilizing a combination of VK3-11 or VK3-15 combined with JK4or JK5 (FIG. 2b and FIG. 8c-d). Furthermore, all but one light chain of the anchor-targeting mAbs were clonally related (Extended Data Table 2), indicating the light chains were very similar across mAbs and study participants. We identified four distinct clonal expansions, with one public clone found across two donors (FIG. 8e-f and Extended Data Table 2). Anchor epitope- and CS-targeting mAbs exhibited similar levels of somatic hypermutations (FIG. 8g). The K-CDR3 length of anchor epitope-binding mAbs was highly restricted, with all K-CDR3s being ten amino acids long (FIG. 8h).

To investigate the binding motif of anchor-targeting mAbs, we generated a high-resolution (3.38 Å) cryo-EM structure of 222-1C06 bound to A/California/July 2009 HA (FIG. 2c and FIG. 8i-k). The broad paratope of 222-1C06 made extensive contacts across the HA fusion peptide (FIG. 2d-e)¹⁹, which mediates viral membrane fusion with the host membrane. Fusion peptide binding was largely mediated by an NWP motif within the K-CDR3, a Y58 directly following the H-CDR2, and a W99 in the H-CDR3, with these HA-binding motifs acting independently and in combination via an aromatic pocket (FIG. 2f-g and Supplementary Table2). Moreover, both the K-CDR3 NWP and H-CDR2 Y58 were found in all the anchor-binding mAbs and were germline encoded (FIG. 2h-i), which could have led to the selection of B cells utilizing these variable genes. Notably, FISW84 utilizes VH3-23 and VK3-15 and comparison of the paratopes showed the NWP and Y58 motifs of FISW84 similarly form an aromatic pocketbut orient towards the fusion peptide slightly differently than 222-1C06 (FIG. 8l). Molecular dynamic (MD) simulations additionally showed that VH3-23 and VH3-30 utilizing mAbs from our study and FISW84 targeted the HA fusion peptide similarly to the cryo-EM structure of 222-1C06 via the aromatic pocket created by the K-CDR3 NWP and H-CDR2 Y58 motifs albeit at different orientations (FIG. 8m). Crucially, MD and cryo-EM analyses revealed numerous intra-Fab interactions of hydrophobic and aromatic amino acids, including p-stacking of K-CDR3-P95 with K-CDR3-W94 and H-CDR2-Y58 that rigidified the paratope (FIG. 8n-o and Supplementary Table 3).

Broad analysis of human, swine, and avian H1 viruses revealed the side-chain contacts of 222-1C06 were highly conserved (94-100% conserved; FIG. 2j). In addition, the side-chain contacts were 100% conserved across 100 years of H1N1 virus evolution in humans (FIG. 8p). Deep mutational scanning of potential H1 viruses at these contact residues indicated substantial permissibility (FIG. 8q), although these mutations appear to not have been selected for in nature (FIG. 2j). The five side-chain contacts of this broad epitope were also highly conserved across group 1 viruses, with the W343 contact being 100% conserved across all group 1 viruses (FIG. 2k). Together, these data reveal B cells targeting the anchor epitope utilized a highly restricted V(D)J gene repertoire, and the specific features within this repertoire made critical and extensive contacts with the conserved anchor epitope.

### 3. The Anchor Epitope is a Common Target

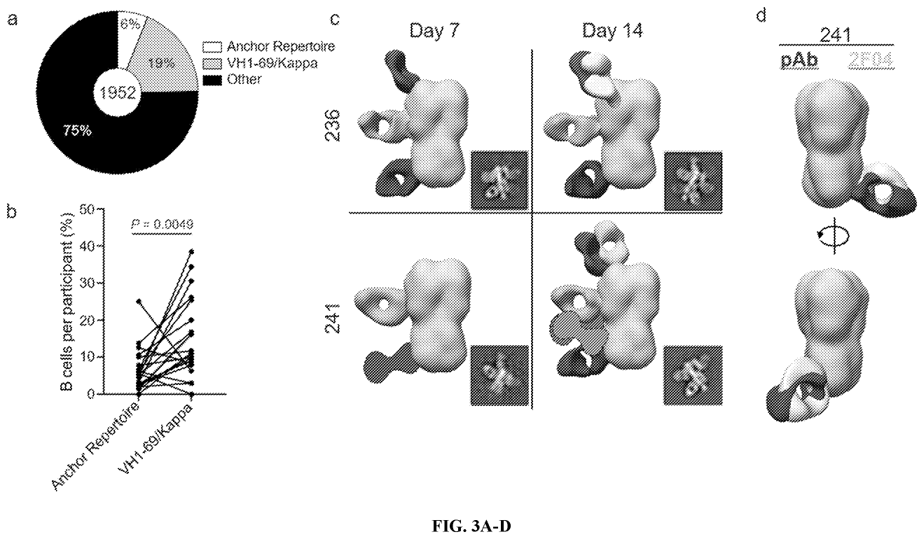

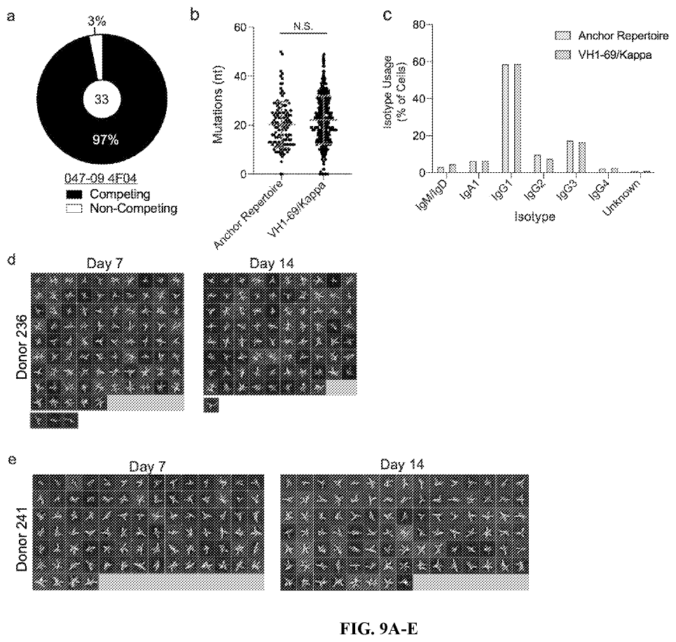

Due to the restricted repertoire features of anchor-targeting mAbs, we next determined the relative proportion of B cells with these features by interrogating single-cell repertoire sequencing of HA-specific B cells isolated from 20 human participants following cHA vaccination^{4,5}. The cHA vaccine platform is intended to specifically induce antibodies against thestalk domain by retaining the stalk domain of H1 and replacing the head domain of HA with that of an avian subtype, either H8 (prime) or H5 (boost) for this trial^{4,5}. To investigate the proportion of B cells with anchor epitope-binding repertoire features, we selected B cells that used VH3-23/VH3-30/VH3-30-3/VH3-48, VK3-11/VK3-15, JK4/JK5, a 10 amino acid length K-CDR3, and possessed an NWP motif within the K-CDR3. We also segregated B cells expressing VH1-69 and a kappa chain, which are commonly used by B cells targeting the CS epitope. We identified that B cells with features of antibodies binding the anchor epitope were abundant within the human B cell repertoire, with 6% of all B cells identified fitting within this defined repertoire (FIG. 3a). Moreover, all but one participant had at least one B cell with anchor-binding repertoire features (FIG. 3b). Additionally, 32 out of 33 mAbs generated from the selected anchor-targeting B cell list competed for HA binding with 047-09 4F04 (FIG. 9a), indicating this population was greatly enriched for anchor-targeting B cells. Moreover, the anchor-binding B cells were highly mutated and were largely class-switched to IgG1 (ig. 9b-c), suggesting these B cells were MBCs that had undergone affinity maturation and class switch recombination. Together, these data indicate that the anchor epitope is a common target of the human MBC repertoire following cHA vaccination.

To confirm that anchor epitope-targeting mAbs were representative of the serum antibody response, we performed electron microscopy polyclonal epitope mapping (EMPEM²⁰) with serum antibodies from participants 236 and 241 from the 2014 quadrivalent inactivated influenza vaccine (QIV) cohort (Extended Data Table 1). Both participants had detectableantibodies targeting the anchor epitope at days 7 and 14 post vaccination (FIG. 3c; FIG. 9d-e). Comparison of anchor epitope-binding polyclonal antibodies (pAbs) identified in participant 241 revealed that 241 IgA 2F04 mAb strongly overlapped with the 241 pAb (FIG. 3d), suggesting the pAb derived from this clonal expansion. Together, these data indicate humoral immunity against the anchor epitope is common within the MBC pool and polyclonal serum antibody response after vaccination.

### 4. cHA Induces Anchor-Binding Antibodies

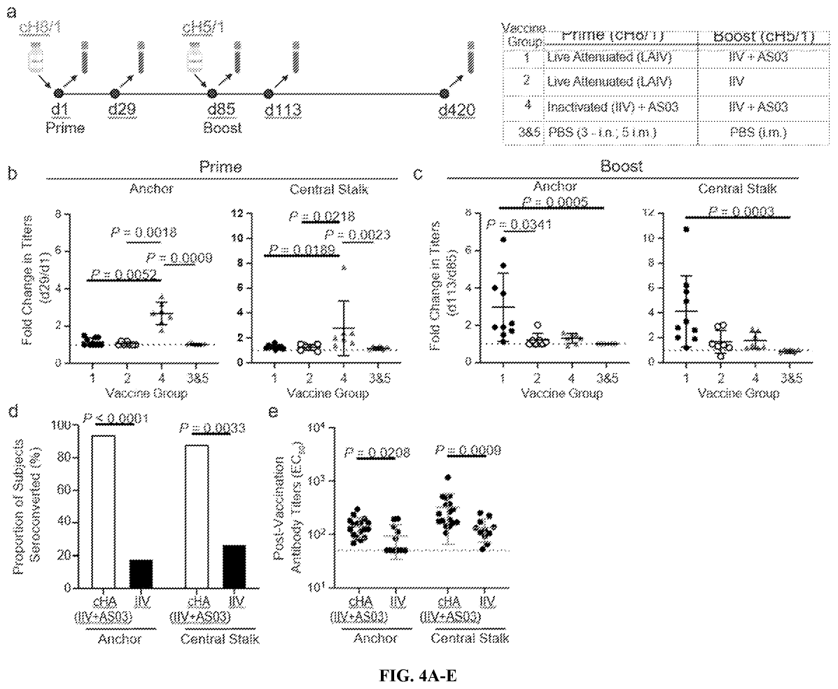

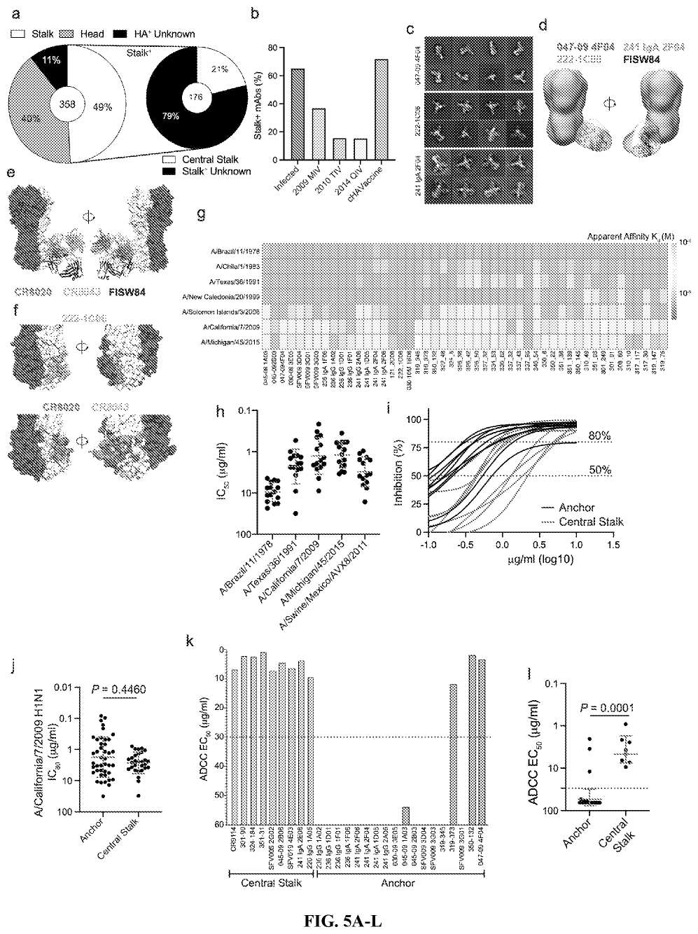

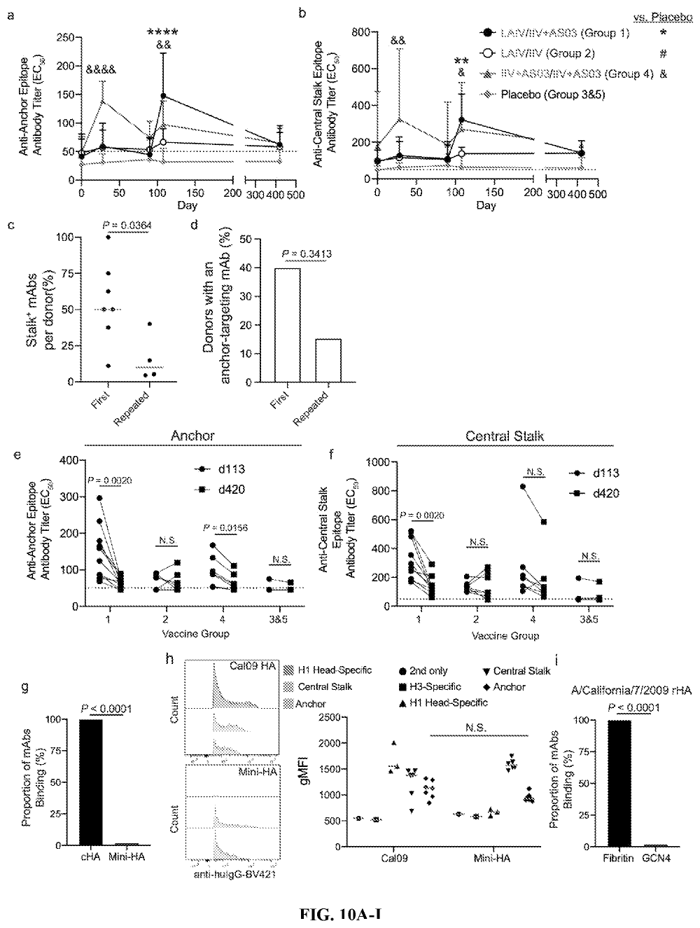

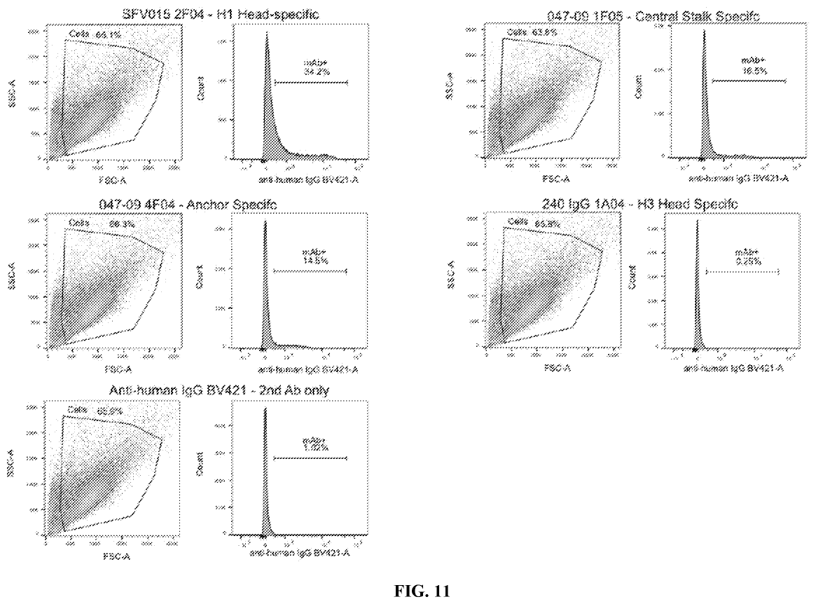

To investigate whether participants enrolled in a phase I clinical trial of the cHA vaccine (FIG. 4a)mounted an antibody response against the anchor and the CS epitope, we adapted the competition ELISA to detect serum antibody responses that could compete for binding with 047-09 4F04 and CR9114, respectively. 3 different vaccine formulations were used in this trial, with participants being primed with either a cH8/1 inactivated influenza vaccine (IIV) with an oil-in-water adjuvant (AS03) or cH8/1 live-attenuated influenza vaccine (LAIV) followed by a boostwith a cH5/1 IIV with or without AS03 (FIG. 4a). Only participants that received the IIV+AS03 on either the prime or boost were capable of seroconverting against both the anchor and CS epitopes (FIG. 4b-c and FIG. 10a-b). Participants that received the cH8/1 IIV+AS03prime did not further boost serum antibodies against either stalk epitope after the cH5/1+AS03 boost (FIG. 4c), suggesting these B cells were refractory to continued activation. Serum titers against the anchor and CS epitopes (FIG. 10a-b) closely matched that of serum neutralizing titers against a cH6/1N8 virus and an avian-swine HIN1 virus⁵, suggesting the anchor- and CS-targeting serum antibodies were responsible for neutralization.

All but one participant in the IIV+AS03 groups seroconverted against the anchor epitope (FIG. 4d) and had higher titers against the anchor epitope relative to participants that received the 2009 MIV or seasonal influenza virus vaccines (FIG. 4e). However, the precise role of the cHA immunogen versus the AS03 adjuvant in inducing anti-stalk antibody responses could not be resolved in our study. Notably, those individuals that received the IIV alone had weak plasmablast responses relative to those individuals that received the IIV+AS03⁴, suggesting the oil-in-water adjuvant, not the cHA immunogen, was essential for robust B cell activation and anti-anchor antibody responses. Moreover, considerable pre-existing antibodies may hinder recall of B cells against the stalk. Individuals first exposed to the 2009 pH1N1, a virus for which individuals had low pre-existing humoral immunity, had proportionally more isolated mAbsagainst the stalk and were more likely to have an anti-anchor mAb than individuals that had repeatedly been exposed to the pH1N1 virus in subsequent influenza seasons (FIG. 10c-d), therefore suggesting pre-existing antibodies may limit antibody responses against theHA stalk. Despite robust recall of antibodies against the anchor and CS epitopes by theadjuvanted vaccines, titers decreased one year after vaccination (day 420; FIG. 10e-f). Together, these data indicate that the adjuvanted inactivated cHA vaccine can robustly induce antibodies against multiple stalk epitopes, including the anchor.

Headless HA antigens, including mini-HA²¹, are attractive universal influenza virus vaccine antigens, as these antigens lack the immunodominant epitopes of the HA head^{21,22}. However, only 1/50 anchor-binding mAbs bound the mini-HA antigen²¹, whereas all anchor epitope-binding mAbs bound cH6/1 (FIG. 10g). Compared to full-length HA, the membrane-proximal region of the mini-HA splays by approximately 14.5 Å²¹, which may disrupt the antigenicity of the anchor epitope. To understand whether anchor epitope-targeting antibodies could bind the mini-HA in a more native setting, we generated a membrane-bound mini-HA and observed that mAbs targeting the anchor and CS epitopes readily bind both the full-length membrane-bound A/California/July 2009 HA (Cal09) and the membrane-bound mini-HA (FIG. 10h), indicating the mini-HA is antigenic when natively presented. Further, we demonstrated that anchor epitope-targeting antibodies bound HA with a fibritin but not a GCN4 trimerization domain (FIG. 10i), highlighting the trimerization domain selection as an important factor for vaccine design. Together, these data demonstrate native-like HA antigens, such as the cHA vaccine, can recall MBCs targeting the anchor epitope.

### B. Discussion

In this study, we identified a public class of bnAbs targeting an epitope at the anchor of the HA stalk domain near the membrane. The anchor-targeting mAbs were public clonotypes across participants, with all antibodies possessing two conserved, germline-encoded binding motifs: a Y58 directly following the H-CDR2 and an NWP motif within the K-CDR3. The neutralizing activity of anchor epitope-targeting mAbs against pre- and post-pH1N1 viruses anda swine-origin H1-expressing virus indicates the anchor epitope is an important target for pan-subtype neutralizing antibodies. As two of the last four influenza virus pandemics were caused by H1N1 viruses and a recent report shows antigenically novel H1-expressing viruses commonly spilling over from swine into humans²³, it is critically important to generate pan-H1 vaccines to prevent the next influenza pandemic. Moreover, the ability of anti-anchor antibodies to neutralize an H2 virus and the general conservation of contact residues suggests that anchor-targeting antibodies have the potential to acquire cross-neutralization against group 1 viruses.

Our study highlights an additional broadly protective epitope of the HA stalk and provides guidance on how vaccines can be designed to drive broadly protective antibodies against multiple distinct epitopes, which can work cooperatively to provide optimal protection while avoiding the generation of antibody escape mutants. Notably, studies in the HIV-1 field have shown that bnAb monotherapy can lead to the development of antibody resistant viral variants²⁴⁻²⁶. whereas combination bnAb therapy does not²⁷. Additionally, immune focusing toward a singleepitope can lead to the generation and selection of viral escape mutants at these highlyconserved epitopes^{2,3,28,29}. Therefore, it is critical that future universal influenza virus vaccines elicit antibodies against multiple conserved epitopes to prevent the generation of bnAb escape viruses.

The angle of approach of anchor-binding mAbs and the proximity of the epitope to the viral membrane suggest this epitope is typically obstructed, limiting antibody recognition and B cell activation. However, membrane-bound HA typically flexes between 0° to 25° and up to 52° on its threefold axis¹¹, suggesting antibodies and B cells can easily access the anchor epitope during this flexing process. Moreover, H1 viruses possess a highly conserved glycosylation site on the HA stalk that lies above the anchor epitope^{11,30}, which may obstruct antibodies from targeting this epitope from above. Similarly, a neutralizing mAb against the Middle East respiratory syndrome coronavirus targets an epitope on S2 from an upward angle to avoid glycans³¹. Therefore, the upward angle of approach may be a common feature of antibodies thatrecognize epitopes below glycans.

Our study shows that humans have pre-existing immunity against the anchor epitopeand influenza virus vaccination can recall MBCs to secrete antibodies against this epitope. However, vaccine HA antigens must have a native-like conformation near the transmembrane domain, as trimer splaying potentially due to the GCN4 trimerization domain ablates antibody binding to the anchor epitope. Moreover, our study reveals that the cHA vaccine strategyrecalled MBCs against the anchor and CS epitopes, as these MBCs do not need to compete against MBCs targeting the immunodominant variable HA head epitopes^{3,9,17,32}. Similarly, the mini-HA/headless HA vaccine strategy has the potential to also recall MBCs against multiple epitopes of the HA stalk domain, if displayed natively^{22,33}. The addition of an oil-in-water AS03 adjuvant to the cHA was critical for recalling MBCs against the anchor epitope. Oil-in-water adjuvants largely function to emulsify antigen, which may prevent sequestration of antigen by circulating antibodies, increase delivery of free antigen to lymph nodes, and help stimulateinnate immune receptors^{34,35}. Notably, an oil-in-water adjuvanted inactivated H5N1 vaccine induced higher neutralizing antibody titers, antibodies against more HA epitopes, and higher avidity antibodies³⁶. Therefore, the inclusion of oil-in-water adjuvants may play an important role in generating bnAbs and may improve vaccine effectiveness of seasonal influenza vaccines. Together, our study shows that novel influenza vaccination strategies, such as the cHA vaccine with the AS03 adjuvant, have the capability to robustly induce antibodies against the previously unappreciated anchor epitope and can provide broad protection against H1 viruses.

### C. Materials and Methods

### 1. Study Approvals, Cohorts, and Human Materials

Human peripheral blood mononuclear cells (PBMCs) and serum were obtained from multiple donors from multiple cohorts, which is outlined in Extended Data Table 1. Informed consent wasobtained from all participants. All studies were performed with the approval of the University of Chicago Institutional Review Board (IRB; ID #09-043-A). The 2009 pH1N1 infection and 2009 MIV cohorts were also approved by the IRB at Emory University. The chimeric HA vaccine studycohort is identified as clinical trial NCT03300050 and further details on trial design are outlined elsewhere^{4,5}. The study was approved by IRBs at local clinical sites, including Icahn School of Medicine at Mount Sinai, Duke University, and Cincinnati Children's Hospital Medical Center. Allexperiments performed with mice were done in accordance with the University of Chicago and Icahn School of Medicine at Mount Sinai Institutional Animal Care and Use Committees.

### 2. Cell Lines

Human Embryonic Kidney HEK293T (female, #CRL-11268), Madin Darby canine kidney (MDCK; female, #CCL-34, NBL-2), human A549 (#CCL-185) cells were purchased andauthenticated by the American Type Culture Collection (ATCC). MDCK-SIAT1 cells were generated previously³⁷ All cells were maintained in a humidified atmosphere of 5% CO₂ at 37° C. HEK293T cells were maintained in Advanced-DMEM supplemented with 2% ultra-low IgG fetal bovine serum (FBS; Invitrogen), 1% L-glutamine (Invitrogen) and 1% antibiotic-antimycotic(Invitrogen). MDCK, MDCK-SIAT1, and A549 cells were maintained in DMEM supplemented with 10% FBS (Invitrogen), 1% L-glutamine (Invitrogen) and 1% penicillin-streptomycin (Invitrogen). Jurkat cells expressing FcgRIIIa and FcgRI with NFAT-driven luciferase reporter gene (#G7010) were acquired and validated by Promega and were directly used for ADCC assays. Cell lines were not authenticated after receiving from suppliers and were not tested for mycoplasma.

### 3. Monoclonal Antibody Production

Monoclonal antibodies were generated as previously described³⁸⁻⁴⁰. Peripheral blood was obtained from each donor approximately 7 days after vaccination or infection or obtained 28+ days post-vaccination. Lymphocytes were isolated and enriched for B cells using RosetteSep. Total PBs (CD3⁻CD19⁺CD27^{hi}CD38^{hi}; all cohorts except 2014 QIV), IgG⁺PBs (CD3⁻CD19⁺IgM⁻ CD27^{hi}CD38^{hi}IgG⁺IgA⁻; 2014 QIV), IgA⁺PBs (CD3⁻CD19⁺IgM⁻CD27^{hi}CD38^{hi}IgG⁻IgA⁺; 2014 QIV cohort), or HA⁺ bait-sorted MBCs (CD3⁻CD19⁺CD27⁺CD38^{lo/+}HA⁺, for 030-09M 1B06) weresingle-cell sorted into 96-well plates. Immunoglobulin heavy and light chain genes were amplified by reverse transcriptase polymerase chain reaction (RT-PCR), sequenced, cloned intohuman IgG1, human kappa chain, or human lambda expression vectors, and co-transfected intoHEK293T cells. Secreted mAbs were purified from the supernatant using protein A agarose beads. For mAbs generated from the 2014 QIV cohort, mAb names include the original isotype of the sorted PB, and all mAbs were expressed as human IgG1. cH5/1-binding B cells (CD19+CD27+cH5/1+) were sorted from donors 28 days after cH5/1 vaccination(NCT03300050). Cells were sorted with A/Califomia/04/2009 HA probe (for 030-09M 1B06) or cH5/1 probe, each with a Y98F mutation to ablate non-specific binding to sialic acids on B cells. MAb heavy chain and light chain sequences were synthesized from single-cell RNA-sequencingdata of cH5/1-baited B cells (IDT), and cloned into the human IgG1, human kappa chain, or human lambda expression vectors. B cell clones were determined by aligning all the V(D)J sequences sharing identical progenitor sequences, as predicted by IgBLAST using our in-house software, VGenes. Consensus sequence analysis was performed using WebLogo⁴¹ and sequence alignments were determined using Clustal Omega.

### 4. Viruses and Recombinant Proteins

Influenza viruses used in all assays were grown in-house in specific pathogen free (SPF) eggs, harvested, purified, and titered. Recombinant HA, cHA, and mini-HA were obtained from BEI Resources or generated in-house. Recombinant HA mutant proteins used in Extended Data FIG. 2 were generated with identified mutations from the deep mutational scanning experiments (see below) or with known mutations that have arisen naturally or were identified in other studies^{2,10,42-52} (Extended Data Table 3). All mutations were made on HA from A/California/July 2009 and were expressed in HEK293T cells and purified using Ni-NTA agarose beads (Qiagen).

### 5. Antigen-Specific ELISA

High protein-binding microtiter plates (Costar) were coated with 8 hemagglutination units (HAU) of virus in carbonate buffer or with recombinant HA, including HA mutants described below, at 2 μg/ml in phosphate-buffered saline (PBS) overnight at 4° C. Plates were washed the nextmorning with PBS 0.05% Tween and blocked with PBS containing 20% fetal bovine serum(FBS) for 1 hour at 37° C. Antibodies were then serially diluted 1:3 starting at 10 μg/ml and incubated for 1.5 hour at 37° C. Horseradish peroxidase (HRP)-conjugated goat anti-human IgG antibody diluted 1:1000 (Jackson Immuno Research) was used to detect binding of mAbs, and plates were subsequently developed with Super Aquablue ELISA substrate (eBiosciences). Absorbance was measured at 405 nm on a microplate spectrophotometer (BioRad). To standardize the assays, control antibodies with known binding characteristics were included on each plate, and the plates were developed when the absorbance of the control reached 3.0 OD units. All ELISAs were performed in duplicate twice. To define antibodies as targeting the H1 stalk, mAbs were tested for binding to cH5/1, which utilizes the head domain from H5-expressing viruses and the stalk domain from the pH1N1 virus⁵³, and for hemagglutination inhibition (HAI) activity against pH1N1 (A/California/July 2009). MAbs that bound the cHA and lacked HAI activity were classified as those binding the HA stalk domain. To classify antigen-specificity, mAbs that did not definitively bind the HA head or stalk are listed as binding unknownHA⁺ epitopes. Affinity measurements, as represented as K_{d} at a molar concentration (M), were calculated using Prism 9 (Graphpad) by performing a non-linear regression. All experiments were performed in duplicate and technically replicated twice.

### 6. Competition ELISAs

Plates were coated with 50 μl of A/California/July 2009 HA at a concentration of 1 μg/ml and incubated overnight at 4° C. To biotinylate the antibodies with known epitope specificities, CR9114 (CS epitope) and 047-09-4F04 (anchor epitope) were incubated at 4° C. with EZ-Link™ Sulfo-NHS-Biotin (Thermo Scientific) for 24h or 48 h prior to use, respectively. After blocking the plates with PBS 20% FBS for 1 h at 37° C., serum samples were incubated (starting dilution of 1:50 for human serum or 20 □g/ml for mAbs) in the coated wells for 2 h at room temperature. Either biotinylated CR9114 or 047-09-4F04 was then added at a concentration equal to twice its K_{d} and incubated in the wells with the serum or mAbs for 2 h at room temperature. The biotinylated antibodies were desalted before addition to remove free biotin using Zeba™ spin desalting columns, 7 k MWCO (Thermo Scientific). After washing the plates, wells were incubated with HRP-conjugated streptavidin (Southern Biotech) at 37° C. for 1 h for detection of the biotinylated antibody. Super Aquablue ELISA substrate (eBiosciences) was then added, andabsorbance was measured at 405 nm on a microplate spectrophotometer (Bio-Rad). To standardize the assays, biotinylated CR9114 or 047-09 4F04 was incubated in designated wells on each plate without any competing serum or mAb, and data were recorded when the absorbance of these wells reached an optical density (OD) of 1 to 1.5 units. After subtracting background, percent competition by serum samples was then determined by dividing a sample'sobserved OD by the OD reached by the positive control, subtracting this value from 1, and multiplying by 100. For the serum data, ODs were log transformed and analyzed by non-linear regression to determine EC₅₀ values using Prism software (Graphpad). For FIG. 4 and Extended Data FIG. 5, only donors with serum for all timepoints were included. All experiments were performed in duplicate and technically replicated twice. 7. Polyreactive ELISAs

High-protein binding microtiter plates (Costar) were coated with 10 □g/ml calf thymus dsDNA (Thermo Fisher Scientific), 2 □g/ml Salmonella enterica serovar Typhimurium flagellin(Invitrogen), 5 □g/ml human insulin (Sigma-Aldrich), 10 □g/ml KLH (Invitrogen), and 10 □g/ml Escherichia coli LPS (Sigma-Aldrich) in PBS. Plates were coated with 10 □g/ml cardiolipin in 100% ethanol and allowed to dry overnight. Plates were washed with water and blocked with PBS/0.05% Tween/1 mM EDTA. MAbs were diluted 1 □g/ml in PBS and serially diluted 4-fold andadded to plates for 1.5 hours. Plates were washed and goat anti-human IgG-HRP (Jackson Immunoresearch) was diluted 1:2000 in PBS/0.05% Tween/1 mM EDTA. Plates were washed with water and were blocked with PBS/0.05% Tween/1 mM EDTA for 5 minutes. Plates were washed again with water and were developed with Super Aquablue ELISA substrate (eBioscience) until the positive control mAb, 3H9⁵⁴, reached an A₄₅₀ of 3. All experiments were performed in duplicate and technically replicated twice. 8. Deep mutational scanning for stalk domain mutants

The mutant libraries used herein were previously described⁵⁵. The libraries consist of all single amino-acid mutations to A/WSN/1933 (H1N1). The experiments were performed by using biological triplicate libraries. The mutational antigenic profiling of the 045-09 2B06, a CS epitope-binding mAb, was performed as previously outlined⁵⁶. In brief, 10⁶ TCID₅₀ of two of the virus library biological replicates was diluted in 1 ml in IGM (Opti-MEM supplemented with 0.01% FBS, 0.3% BSA, and 100 mg/ml calcium chloride) and incubated with an equal volume of 045-09 2B06 antibody at a final concentration of 50 or 25 □g/mL for 1.5 hours at 37° C. MDCK-SIAT1cells were infected with the virus antibody mixtures. 2 hours post-infection, the media was removed, the cells washed with 1 ml PBS, and 2 ml of fresh IGM was added. 15 hours post-infection, viral RNA was extracted, reverse-transcribed using primers WSNHA-For (5′-AGCAAAAGCAGGGGAAAATAAAAACAAC-3′) and WSNHA-Rev (5′-AGTAGAAACAAGGGTGTTTTTCCTTATATTTCTG-3′), and PCR amplified according to the barcoded-subamplicon library preparation as previously described⁵. The overall fraction of virions that survive antibody neutralization was estimated using qRT-PCR targeting the viral nucleoprotein (NP) and cellular GAPDH as previously described⁵⁶. Using 10-fold serial dilutions of the virus libraries, we infected cells with no antibody selection to serve as a standard curve of infectivity. qPCR Ct values from the standard curve samples compared to the virus-antibody mixsamples are determined for NP and GAPDH. We then generate a linear regression to fit the difference between the NP and GAPDH Ct values for the standard curve samples, and then usethis curve to interpolate the fraction surviving for the antibody-virus selection samples. Across the three library replicates the fraction of virus surviving antibody selection was 0.17, 0.1, and 0.14. Illumina(R) deep sequencing data was analyzed using dms_tools2 version 2.4.12 software package⁵⁷ which can be found at https://github.com/jbloomlab/dms_tools2. All of the computer code used is at https://github.com/jbloomlab/2B06_DMS, and the Jupyter notebook that performs most of the analysis is at https://github.com/jbloomlab/2B06_DMS/blob/master/analysis_notebook.ipynb. The sequencing counts were processed to estimate the differential selection for each mutation, which is the log enrichment of that mutation in the antibody-selected condition versus the control56. The numerical measurements of the differential selection that 2B06 imposes on each mutation can be found here: https://github.com/jbloomlab/2B06_DMS/blob/master/results/diffsel/tidy_diffsel.csv.

### 9. Deep Mutational Scanning for H1 Variants

Amino-acid preferences for the hemagglutinin (HA) ofA/WSN/1933 (H1N1) were previously determined⁵⁵. Briefly, deep mutational scanning was performed by passaging virus libraries at a low MOI in MDCK-SIAT1 cells. Following deep sequencing of the resulting virus, amino-acid preferences were determined using the Python package dms_tools (http://jbloomlab.github.io/dms_tools/), version 1.1.12. This program aligns subamplicon reads to the reference HA sequence, counts the number of mutations at each amino-acid site, and determines amino-acid preferences based on the mutation counts pre- and post-selection.

### 10. Microneutralization Assays

Microneutralization assays for mAb characterization were carried out as previously described^{58,59}. MDCK cells were maintained in DMEM supplemented with 10% FBS, 1% penicillin-streptomycin, and 1% L-glutamine at 37° C. with 5% CO₂. The day before the experiment, 25,000MDCK cells were added to each well of a 96-well plate. Serial two-fold dilutions of mAb were mixed with an equal volume of 100 TCID₅₀ of virus for 1 hour and added to MDCK cells for 1 hour at 37° C. The mixture was removed, and cells were cultured for 20 hours at 37° C. with 1× MEM supplemented with 1 μg/ml tosyl phenylalanyl chloromethyl ketone (TPCK)-treated trypsin and appropriate mAb concentration. Cells were washed twice with PBS, fixed with 80% ice cold acetone at −20° C. for at least 1 hour, washed 3 times with PBS, blocked for 30 min with 3% BSA-PBS, and then treated for 30 min with 2% H₂O₂. Cells were incubated with a mouse anti-nucleoprotein antibody (1:1000; Millipore) in 3% BSA-PBS for 1 hour at room temperature (RT), followed by goat anti-mouse IgG HRP (1:1000; Southern Biotech) in 3% BSA-PBS for 1 hour at RT. The plates were developed with Super Aquablue ELISA substrate at 405 nm until virus only controls reached an OD of 1. The signal from uninfected wells was averaged to represent 100% inhibition. The signal from infected wells without mAb was averaged to represent 0% inhibition. Duplication wells were used to calculate the mean and SD of neutralization, and inhibitory concentration 50 (IC₅₀) was determined by a sigmoidal dose response curve. The inhibition ratio (%) was calculated as: ((OD Pos. Control−OD Sample)/(OD Pos. Control−OD Neg. Control))*100. The final IC₅₀ was determined using Prism software (GraphPad). All experiments were performed in duplicate and technically replicated twice.

### 11. H2N2 Neutralization Assays

Twenty thousand MDCK cells were seeded per well in a 96-well cell culture plate (Corning) and the cells were used the next morning to perform the neutralization assay. Antibody dilutionswere prepared starting at 30 □g/ml with 3-fold subsequent dilutions in 1× MEM. Each respective dilution was mixed with 10,000 PFUs of cold adapted A/Ann Arbor/June 1960 (H2N2) virus for 1 hour at room temperature. After an hour, cells were washed with PBS and 100 □l of antibody-virus mixture was added onto the cells for 1 hour at 37° C. Next, antibody-virus mixture was removed and 60 μl of 1× MEM containing TPCK was added to each well. 60 □l of each corresponding antibody dilution was also added to each well and the cells were incubated at 33° C. for 3 days. On the 3rd day, a hemagglutination assay was performed using turkey red blood cells to assess the HAU at each antibody dilution.

### 12. In Vivo Challenge Infections

MAb cocktails (FIG. 6b) were passively transferred into 6- to 8-week-old female BALB/c mice (Jackson Laboratories) by intraperitoneal injection of 0.2, 1, and 5 mg/kg mAb cocktail, which are further detailed in Supplementary Table 1. Negative control mice received 5 mg/kg of the anthrax-specific mAb 003-15D03 as an isotype control. MAbs were administered two hours before infection for prophylactic treatment and 48 hours post infection for therapeutic treatment. For prophylactic mAb studies with A/Netherlands/602/2009 (FIG. 7a), mice were anesthetized with isoflurane and intranasally challenged with 10 LD₅₀ of mouse-adapted A/Netherlands/602/2009 H1N1 virus, with 10 μl of virus administered into each nostril (20 μl total). For therapeutic treatment of A/Netherlands/602/2009 and prophylactic treatment of A/Fort Monmouth/January 1947, mice were anesthetized with a ketamine-xylazine-water cocktail (0.15 mg ketamine/kg and 0.03 mg/kg xylazine; 100 ml intraperitoneally) and infected with 10 LD₅₀ of A/Netherlands/602/2009 or A/Fort Monmouth/January 1947. As a read out, survival and weight loss were monitored 1-2 times daily for two weeks. Mice were euthanized upon 25% weight loss or at the end of the experiment (14 days post challenge). 5 mice/condition/experiment with 2 biological replicates were utilized based on a previously performed power analysis. Data were pooled for analysis.

To determine differences in lung viral load, 5 mg/kg of the antibody cocktails were administered prophylactically as described above. Two hours after mAb administration, mice (n=5 mice/group) were anesthetized and intranasally challenged with 1 LD₅₀ of A/Netherlands/602/2009. Lungs were collected at day 3 and day 6 post infection, homogenized and viral load determined via plaque assay. All experiments were done in accordance with the University of Chicago and Icahn School of Medicine at Mount Sinai Institutional Animal Care andUse Committees. Animals studies were not blinded.

### 13. Plaque Assay

For determination of viral load in murine lung tissues a standard plaque assay was performed. Confluent monolayers of MDCK cells were infected with serial dilutions of homogenized lung tissue ranging from 1:10 to 1:1000000 diluted in 1× MEM (1% penicillin/streptomycin antibiotics mix, 1% HEPES, 1% L-Glutamine and 1% sodium-bicarboante (Gibco)) for 1 hour at 33° C., with shaking every 15 minutes. Afterwards, an overlay containing 2% Oxoid agar (ThermoFisher), H₂O, 2×MEM, DEAE, and TPCK-treated trypsin was added to the cells. The plates wereincubated at 33° C. for 3 days and then fixed with 3.7% paraformaldehyde overnight at 4° C. Plaques were visualized by immunostaining. Here, the agar overlay was removed and the platesblocked with 3% milk/PBS. The blocking solution was removed and primary antibody ((H1N1 guinea pig anti-sera (generated in house)) diluted 1:3000 in 1% milk/PBS was added for 1 hour. The plates were washed 3× with PBS and secondary antibody (anti-mouse IgG H&L peroxidase-conjugated (Rockland) diluted 1:3000 in 1% milk/PBS was added for 1 hour. The plates were washed 3× with PBS and developed by using KPL TrueBlue Peroxidase Substrate (SeraCare).

Antibody-dependent cellular cytotoxicity reporter assay A549 cells were maintained in DMEM supplemented with 10% FBS, 10 U/ml Penicillin, and 10 mg/ml Streptomycin) and were plated in 96-well, white-walled plates (Costar) at 2.5×10⁵ cells/ml overnight at 37° C. with 5% CO₂. The following day, cells were washed with PBS and infected with A/Netherlands/602/2009 at a multiplicity of infection of 5 in UltraMDCK media (Lonza) for24 hours in the absence of TPCK-treated trypsin. MAbs were serially diluted in assay buffer (RPMI 1640 supplemented with 4% ultra-low IgG FBS; Gibco), starting at 60 μg/ml and diluted 3-fold. Aspirate cell medium and add 25 μl of assay buffer and 25 μl of diluted antibody to each well. Jurkat cells expressing human FcgRIIIa with NFAT-driven luciferase reporter gene (Promega) were diluted to 3×10⁶ cells/ml, 25 μl of cells was added to each well and incubated at 37° C. with 5% CO₂ for 6 hours. Plates were removed from incubator and placed at roomtemperature for 15 minutes. 75 μl BioGlo luciferase substrate (Promega) was added to eachwell and luminescence was read immediately using a Syngery H1 hybrid multimode microplate reader (Biotek). EC₅₀ values were determined using Prism 8 (GraphPad).

### 14. HA:Antibody Binding Footprint Mapping

The footprints of three mAbs (FISW84 (PDB: 6HJQ), CR9114 (PDB: 4FQI), and FI6v3 (PDB: 3ZTN)) were mapped onto one HA protomer (A/California/April 2009, PDB: 4M4Y) using UCSF Chimera⁶⁰ and Adobe Photoshop. Negative stain EM maps of HA:Fab complexes were aligned in UCSF Chimera and estimated footprints were mapped onto one HA protomer. Individual protomers of the HA trimer are indicated in different shades of gray.

### 15. Negative Stain EM

Immune complexes were prepared by incubating Fab with HA (A/California/April 2009 with E376K or E376G stabilizing mutations) at greater than 3:1 molar ratio for 2 hours at room temperature (RT). Samples were deposited at −1011 g/mL on glow-discharged, carbon-coated 400 mesh copper grids (Electron Microscopy Sciences, EMS) and stained with 2% w/v uranyl formate. Samples were imaged at 52,000× magnification, 120 kV, on a Tecnai Spirit T12 microscope equipped with an Eagle CCD 4k camera (FEI) or 62,000 magnification, 200 kV, on a Tecnai T20 microscope equipped with a CMOS 4k camera (TVIPS). Micrographs were collected with Leginon, single particles were processed with Appion, Relion, and XQuartz, and footprints were mapped with UCSF Chimera, and figures were made with UCSF Chimera⁶⁰⁻⁶³.

### 16. Cryo-EM

222-1C06 and 045-09 2B05 Fabs were incubated at greater than 3:1 molar ratio with HA (A/California/July 2009, E376K) for 1 hour at room temperature. 045-09 2B05 Fab, targeting the lateral patch, was added to the immune complex to induce particle tumbling and increase angular sampling³. Using a ThermoFisher Vitrobot, the immune complex (0.5 mg/ml) incubated with lauryl maltose neopentyl glycol (5 μM, Anatrace) was deposited onto glow-discharged Au 1.2/1.3 300 mesh grids (Electron Microscopy Sciences), blotted for 7 seconds, and plunge-frozen in liquid ethane. Samples were imaged at 36,000× nominal magnification on a 200 kV Talos Arctica electron microscope (FEI) with a CETA 4K CMOS camera (FEI, total dose 49.92 e/A²) and Gatan K2 Summit detector in counting mode. 2243 micrographs were collected, aligned, and CTF-corrected using Leginon, MotionCor2 in Appion, and Patch-CTF in CryoSPARC2, respectively^{61,62,64,65}. In CryoSPARC2, particles were picked using apo HA templates, selected through reference-free 2D classification, and reconstructed through 3Dclassification and refinement. The final map resolved to a global 3.75 Å resolution with C3 symmetry and 44,224 particles. Figures were made in Prism 8 (GraphPad) and UCSF Chimera⁶⁰.

### 17. Model Building and Refinement

A predicted model of 222-1C06 Fab was generated using abYsis (http://www.abysis.org/abysis/)and docked into EM density along with an initial model of CA09 H1 HA+045-09 2B05 (PDB: 7MEM). The initial model was iteratively refined using COOT and Rosetta⁶⁶′6⁷. The final model was numbered using the H3 and Kabat numbering schemes. The final model and map were evaluated using MolProbity, EMRinger^{68,69}, Phenix, and the PDB validation server. After modeling the immune complex, we segmented the Fab density from HA in the cryo-EM map andmapped the footprint of the 222-1C06 model in the HA density. Cryo-EM data collection and refinement statistics are included in Extended Data Table 4.

### 18. EMPEM

Human serum samples were heat-inactivated at 55° C. for 30 min before incubating on Capture Select IgG-Fc (ms) Affinity Matrix (Fisher) to bind IgG at 4° C. for 72 hours on a rotator. Samples with IgG bound to resin were centrifuged at 4,000 rpm and supernatant was collected. IgG samples were washed 3 times with PBS followed by centrifugation to remove supernatant. Samples were buffer exchanged into buffer containing 100 mM Tris, 2 mM EDTA, and 10 mM L-cysteine through centrifugation with Amicon filters, then incubated with papain for 4 hours at 37° C. shaking at 80 rpm. The digestion reactions were quenched with 50 mM iodoacetamide, buffer exchanged to TBS, and separated by size-exclusion chromatography (SEC) with aSuperdex 200 increase 10/300 column (GE Healthcare). Fab and undigested IgG were collectedand concentrated and 500 μg Fab was complexed with 10 □g HA for 18 hours at room temperature. Reactions were purified by SEC and immune complexes were collected andconcentrated. Negative stain EM grids were prepared as described above.

### 19. Membrane-Bound HA and mAb Staining

HEK293T cells were plated into a 6-well plate and transfected overnight with 0.2 μg of plasmid and 10 μg/ml PEI. After 12-16 hours, media was replaced with PFHM-II (Gibco) and cells were rested for 3 days. Transfected cells were trypsinized, washed, and aliquoted. Cells were stainedwith 10 μg/ml of individual mAbs for 30 minutes. Cells were washed and stained with anti-human IgG Fc-BV421 for 30 minutes. Cells were washed 2 times and run on a BD LSRFortessaand collected with BD FACSDiva software. Data were analyzed using FlowJo v10.

### 20. Single-Cell RNA-Seq and Repertoire Analysis

cH5/1+ memory B cells (CD19+CD27+HA+) were bulk sorted and partitioned into nanoliter-scale Gel Bead-In-Emulsions (GEMs) to achieve single cell resolution using the 10× Genomics Chromium Controller and according to the manufacturer's instruction (10× Genomics). The sorted single cells were processed according to 5′ gene expression and B cell Immunoglobulin (Ig) enrichment instruction to prepare the libraries for sequencing. Libraries were sequenced using an Illumina HiSeq 4000 at Northwestern University or an Illumina NextSeq 500 at the University of Chicago. Cellranger Single-Cell Software Suite (version 3.0) was used to perform sample de-multiplexing, barcode processing, and single-cell 5′ and V(D)J counting, and Cellranger mkfastq was used to de-multiplex raw base call (BCL) files into sample-specific fastq files. Subsequently, GRCh38-1.2.0 and cellranger-vdj-GRCh38-alta-ensembl-2.0.0 were used as references for the transcriptome and V(D)J assembly, respectively. Cellranger counts and Cellranger vdj package were used to identify gene expression and assemble V(D)J pairs of antibodies.

Single cell datasets were analyzed using Seurat 3 toolkit (Version 3.2.0). We performed conventional pre-process steps for all 20 donors including cell quality control (QC),normalization, identification of highly variable features, data scaling, and linear dimensional reduction. More specifically, we only kept cells with more than 200 and less then 2500 detected genes for QC step. We also filtered out cells with high mitochondrial gene expression using a “softThreshold” function in the R package LinQ-View (Version 0.99)⁷⁰. We normalized the RNA data using conventional log normalization. We identified 2000 highly variable genes for each dataset and performed principle component analysis (PCA) in linear dimensional reduction step. We then integrated all 20 single cell datasets from vaccinated participants to remove batch effects using the Anchor method in Seurat 3. In this analysis, we filtered our dataset and only kept cells with both transcriptome and full length and paired heavy and light chain V(D)Jsequences (n=1952). From these cells, we identified a group of “VH1-69/Kappa” B cells that used the VH1-69 gene and kappa light chain, which is enriched for B cells targeting the BN stalkepitope. We also identified a group of “anchor epitope” specific B cells by the following rules: 1) VH locus: VH3-23, VH3-30, VH3-30-3, or VH-3-48; 2) VK locus: VK3-11 or VK3-15; 3) JK locus: JK4 or JK5; 4) K-CDR3 length equal to 10; 5) a “NWP” pattern in K-CDR3 peptide.HA conservation modeling Pan-H1 conservation models are based on consensus strains (listed in Supplementary Table 4) of distinct H1 clades isolated from humans, swine, and avian sources, as described in Zhuanget al.⁷¹ and inclusion of the Eurasian swine-like A/swine/Jiangsu/J004/2018²³. To generate the group 1 HA conservation model, we selected one representative sequence for each group 1 HA subtype from FluDB (https://www.fludb.org/; Supplementary Table 5) according to a prior study⁷². A multiple sequence alignment from these HA protein sequences was generated using MUSCLE⁷³ and the conservation of each residue was quantified using an entropy model⁴¹. Seasonal H1 conservation models are based on consensus strains ofHIN1 viruses (59 strains total) circulating between 1918-1957 and 1976-2019, which was previously described³. Amino acid alignments and H3 numbering were performed using Librator⁷⁴ and Burke and Smith HA numbering⁷².

### 21. Structure Prediction

To predict the structures of the investigated Fv fragments (222-1C06, FISW84, 241 IgA 2F04 and SFV009 3G01) with A/California/April 2009 E47G HA (PDB:7MEM) we applied the program Rosettaantibody^{67,75,76}. The Fvs were protonated using the Protonate 3D tool^{77,78}. Charge neutrality was ensured by utilizing the uniform background plasma approach in AMBER^{79,80}. Using the tleap tool of the AmberTools20⁸¹ package, the structure models were soaked in cubic water boxes of TIP3P water molecules with a minimum wall distance of 10 Å to the proteins². Parameters for all antibody models derive from the AMBER force field 14SB⁸³. The Fvs were carefully equilibrated using a multistep equilibration protocol⁸⁴.

### 22. Metadynamics Simulations

To enhance the sampling of the conformational space, well-tempered bias-exchange metadynamics⁸⁵⁻⁸⁷ simulations were performed in GROMACS^{88,89} with the PLUMED 2 implementation⁹⁰. We chose metadynamics as it enhances sampling on predefined collective variables (CV). The sampling is accelerated by a history-dependent bias potential, which is constructed in the space of the CVs⁸⁵⁻⁵⁶⁻⁹¹. As collective variables, we used a well-established protocol, boosting a linear combination of sine and cosine of the ψ torsion angles of all six CDR loops calculated with functions MATHEVAL and COMBINE implemented in PLUMED 2⁹⁰. As discussed previously, the ψ torsion angle captures conformational transitions comprehensively⁹². The underlying method presented in this paper has been validated in various studies against a large number of experimental results⁹³. The simulations were performed at 300 K in an NpT ensemble using the GPU implementation of the pmemd module⁹⁴ to be as close to the experimental conditions as possible and to obtain the correct density distributions of both protein and water. We used a Gaussian height of 10.0 kJ/mol and a width of 0.3 rad. Gaussian deposition occurred every 1000 steps and a biasfactor of 10 was used. 500 ns of bias-exchange metadynamics simulations were performed for the prepared Fv structures. The resulting trajectories were aligned to the whole Fv and clustered with the program cpptraj^{80,95} using the average linkage hierarchical clustering algorithm with a RMSD cut-off criterion of 1.2 Å resulting in a large number of clusters. The cluster representatives for the antibody fragments were equilibrated and simulated for 100 ns using the AMBER 20⁸¹ simulation package.

### 23. Molecular Dynamics Simulations

Molecular dynamics simulations were performed in an NpT ensemble using the pmemd.cuda module of AMBER 20⁸⁰. Bonds involving hydrogen atoms were restrained with the SHAKEalgorithm⁹⁶, allowing a time step of 2.0 fs. Atmospheric pressure (1 bar) of the system was set by weak coupling to an external bath using the Berendsen algorithm⁹⁷. The Langevin thermostat⁹⁸ used to maintain the temperature during simulations at 300 K. With the obtained trajectories we performed a time-lagged independent component analysis (tICA) using the python library PyEMMA 2 employing a lag time of 10 ns. tICA was applied to identify the slowest movements of the investigated Fab fragments and consequentlyto obtain a kinetic discretization of the sampled conformational space”⁹. Based on the tICA conformational spaces, thermodynamics and kinetics were calculated with a Markov-state modello¹⁰⁰ of all 6 CDR loops by using PyEMMA 2. The resulting kinetically dominant ensemble insolution was further used to predict the interactions of H1 with the Fvs. To model the complex and to predict interactions in the binding interface, we used the crystal structure of the full-lengthinfluenza hemagglutinin (PDB:7MEM) as template structure. In addition, the obtained complex structure was further minimized and equilibrated.

### 24. Statistical Analysis

All statistical analyses were performed using Prism software (Graphpad Version 8 and 9) or R. Sample sizes (n) for the number of mAbs tested are indicated in corresponding figures or in the center of pie graphs. Number of biological repeats for experiments and specific tests for statistical significance used are indicated in the corresponding figure legends. P values less than or equal to 0.05 were considered significant. * P≤0.05, ** P≤0.01, *** P≤0.001, **** P≤0.0001.

### 25. Data Availability

Repertoire data generated from single cell RNA-sequencing data is deposited at Mendeley Data(https://data.mendeley.com/datasets/jzsx489pmk/1). Accession numbers for all other anchor-targeting mAbs are included in Supplementary Table 6. Electron microscopy maps were deposited
to the Electron Microscopy DataBank under accession IDs: D_100025433, D_1000254374, D_1000254375, D_1000254376, D 1000254377, D_1000254378, D 1000254383, D_1000254384, D_1000254385, D 1000254386, D_1000254388, D_1000254379, D 1000254391, and D_1000254382. All next generation sequencing data for 045-09 2B06 deep mutational scanning and for the H1N1 mutational scanning can be found on the Sequence Read Archive under BioProject accession number PRJNA309339. The following Protein Database accession numbers were downloaded and included in the manuscript-6HJQ, 3SDY, 4NM8, 6HJQ, 4M4Y, 4WE4, 4JTV, 4FQI, 3ZTN, and 7MEM. All source data are includedwith the manuscript.

### D. Tables

TABLE 1
Extended Data:
Donor information and demographics. Related to FIG. 1.

| Donor | Vaccine/Infection | Age | Sex |
|---|---|---|---|
| 029-09 | 2009 MIV | 24 | M |
| 030-09 | 2009 MIV | 31 | F |
| 045-09 | 2009 MIV | 24 | M |
| 047-09 | 2009 MIV | 22 | M |
| 051-09 | 2009 MIV | 42 | M |
| SFV005 | 2009 MIV | 60 | F |
| SFV009 | 2009 MIV | 31 | F |
| SFV015 | 2009 MIV | 48 | F |
| SFV018 | 2009 MIV | 58 | F |
| SFV019 | 2009 MIV | 48 | F |
| SFV020 | 2009 MIV | 64 | F |
| sc70 | 2009 pH1N1 Infection | 38 | F |
| sc1009 | 2009 pH1N1 Infection | 21 | M |
| SF1000 | 2009 pH1N1 Infection | 37 | M |
| 008-10 | 2010 TIV | 26 | F |
| 011-10 | 2010 TIV | 30 | M |
| 014-10 | 2010 TIV | 27 | M |
| 017-10 | 2010 TIV | 24 | M |
| 019-10 | 2010 TIV | 22 | F |
| 028-10 | 2010 TIV | 38 | M |
| 034-10 | 2010 TIV | 39 | F |
| 039-10 | 2010 TIV | 25 | M |
| 051-10 | 2010 TIV | 43 | M |
| 220-14 | 2014 QIV | 24 | F |
| 221-14 | 2014 QIV | 34 | F |
| 236-14 | 2014 QIV | 32 | F |
| 237-14 | 2014 QIV | 32 | F |
| 240-14 | 2014 QIV | 28 | M |
| 241-14 | 2014 QIV | 29 | F |

| | | | | |
|---|---|---|---|---|
| 121/324 | (d8/d113) | cHA - Group 4 | 24 | F |
| 222/310 | (d92/d113) | cHA - Group 1 | 25 | F |

| | | | |
|---|---|---|---|
| 301 | cHA - Group 4 | 34 | F |
| 308 | cHA - Group 1 | 29 | F |
| 311 | cHA - Group 4 | 28 | M |
| 317 | cHA - Group 1 | 37 | M |
| 319 | cHA - Group 1 | 31 | F |
| 322 | cHA - Group 4 | 20 | F |
| 326 | cHA - Group 2 | 27 | M |
| 327 | cHA - Group 1 | 22 | F |
| 328 | cHA - Group 2 | 40 | M |
| 334 | cHA - Group 1 | 33 | M |
| 336 | cHA - Group 1 | 36 | F |
| 337 | cHA - Group 1 | 27 | F |
| 342 | cHA - Group 2 | 22 | M |
| 343 | cHA - Group 4 | 27 | M |
| 346 | cHA - Group 2 | 20 | F |
| 347 | cHA - Group 1 | 31 | F |
| 350 | cHA - Group 1 | 35 | F |
| 351 | cHA - Group 4 | 27 | F |

Footnote:
2009 MIV—2009 pandemic H1N1 monovalent inactivated influenza vaccine; pH1N1—2009 pandemic H1N1 virus; 2010 TIV—2010-2011 trivalent inactivated influenza vaccine; 2014 QIV—2014-2015 quadrivant inactivated influenza vaccine; cHA—Group 1, primed with cH8/1 LAIV and boosted with cH5/1 IIV + AS03; cHA—Group 2, primed with cH8/1 LAIV and boosted with cH5/1 IIV; cHA—Group 4, primed with cH8/1 IIV + AS03 and boosted with cH5/1 IIV + AS03. LAIV—live-attenuated influenza vaccine; IIV—inactivated influenza vaccine; AS03—Adjuvant System 03.

TABLE 2
Extended Data:
Anchor epitope-binding mAb information. Related to FIG. 1 and 2.

| | Cellular | | | | HC Clone | | | KC Clone |
|---|---|---|---|---|---|---|---|---|
| mAb | Source | VH | DH | JH | # | VK | JK | # |
| 030-09 3E05 | Plasmablast | 3-23 | 3-22 | 4 | Non-clonal | 3-15 | 5 | 9 |
| 030-09M 1B06 | MBC | 3-23 | 3-10 | 4 | Non-clonal | 3-11 | 5 | 7 |
| 045-09 1A03 | Plasmablast | 3-23 | 6-13 | 4 | Non-clonal | 3-11 | 4 | 6 |
| 045-09 2B03 | Plasmablast | 3-23 | 5-12 | 4 | Non-clonal | 3-11 | 5 | 7 |
| 047-09 4F04 | Plasmablast | 3-48 | 6-19 | 4 | Non-clonal | 3-15 | 4 | 8 |
| SFV009 3D04 | Plasmablast | 3-23 | 2-2 | 4 | Non-clonal | 3-15 | 4 | 8 |
| SFV009 3G01 | Plasmablast | 3-30 | 3-16 | 4 | Non-clonal | 3-11 | 5 | 7 |
| SFV009 3G03 | Plasmablast | 3-23 | 3-15-3 | 5 | Non-clonal | 3-11 | 5 | 7 |
| 236 IgG 1A02 | Plasmablast | 3-23 | 2-2 | 4 | 2 | 3-11 | 5 | 7 |
| 236 IgG 1D01 | Plasmablast | 3-23 | 6-19 | 4 | Non-clonal | 3-11 | 5 | 7 |
| 236 IgG 1F01 | Plasmablast | 3-23 | 2-2 | 4 | 2 | 3-11 | 5 | 7 |
| 236 IgA 1F06 | Plasmablast | 3-23 | 2-2 | 4 | 2 | 3-11 | 5 | 7 |
| 241 IgG 2A06 | Plasmablast | 3-23 | 6-19 | 5 | Non-clonal | 3-15 | 4 | 8 |
| 241 IgA 1D05 | Plasmablast | 3-23 | 5-18 | 4 | 3 | 3-15 | 5 | 9 |
| 241 IgA 2F04 | Plasmablast | 3-23 | 2-21 | 4 | 3 | 3-15 | 5 | 9 |
| 241 IgA 2F06 | Plasmablast | 3-23 | 3-9 | 4 | 3 | 3-15 | 5 | 9 |
| 121 2C06 | Plasmablast | 3-23 | 2-15 | 4 | Non-clonal | 3-15 | 4 | 8 |
| 222 1C06 | Plasmablast | 3-48 | 2-15-2 | 4 | Non-clonal | 3-15 | 4 | 8 |
| 301_91 | MBC | 3-23 | 3-9 | 5 | Non-clonal | 3-11 | 5 | 7 |
| 301_249 | MBC | 3-48 | 5-24 | 3 | Non-clonal | 3-11 | 4 | Non-clonal |
| 301_275 | MBC | 3-23 | 2-21 | 4 | 1 | 3-15 | 4 | 8 |
| 308_60 | MBC | 3-23 | 1-26 | 4 | Non-clonal | 3-11 | 4 | 8 |
| 310_10 | MBC | 3-23 | 1-14 | 4 | Non-clonal | 3-15 | 4 | 8 |
| 310_49 | MBC | 3-23 | 3-22 | 4 | Non-clonal | 3-15 | 5 | 9 |
| 317_30 | MBC | 3-23 | 6-13 | 4 | Non-clonal | 3-15 | 4 | 8 |
| 317_117 | MBC | 3-23 | 2-2 | 4 | Non-clonal | 3-15 | 5 | 9 |
| 319_75 | MBC | 3-23 | 3-22 | 3 | Non-clonal | 3-11 | 5 | 7 |
| 319_147 | MBC | 3-48 | 3-15-3 | 6 | Non-clonal | 3-15 | 4 | 8 |
| 319_345 | MBC | 3-23 | 3-9 | 4 | 4 | 3-15 | 4 | 8 |
| 319_373 | MBC | 3-23 | 6-19 | 4 | 4 | 3-15 | 4 | 8 |
| 322_48 | MBC | 3-23 | 1-26 | 4 | 1 | 3-15 | 5 | 9 |
| 324_5 | MBC | 3-23 | 2-21 | 3 | Non-clonal | 3-15 | 5 | 9 |
| 326_38 | MBC | 3-23 | 1-26 | 4 | Non-clonal | 3-11 | 4 | 6 |
| 326_42 | MBC | 3-23 | 3-16 | 5 | Non-clonal | 3-15 | 4 | 8 |
| 326_50 | MBC | 3-30 | 5-12 | 4 | Non-clonal | 3-15 | 5 | 9 |
| 327_32 | MBC | 3-23 | 3-10 | 4 | Non-clonal | 3-15 | 4 | 8 |
| 334_52 | MBC | 3-48 | 6-13 | 6 | 5 | 3-15 | 4 | 8 |
| 334_53 | MBC | 3-23 | 2-2 | 4 | Non-clonal | 3-15 | 5 | 9 |
| 334_62 | MBC | 3-23 | 2-2 | 1 | Non-clonal | 3-11 | 4 | 6 |
| 337_32 | MBC | 3-30 | 5-24 | 4 | Non-clonal | 3-11 | 5 | 7 |
| 337_43 | MBC | 3-23 | 5-24 | 4 | Non-clonal | 3-15 | 4 | 8 |
| 337_95 | MBC | 3-48 | 3-16 | 6 | Non-clonal | 3-15 | 5 | 9 |
| 346_54 | MBC | 3-30-3 | 3-16 | 5 | Non-clonal | 3-11 | 4 | 6 |
| 347_64 | MBC | 3-23 | 1-7 | 2 | Non-clonal | 3-11 | 5 | 7 |
| 347_246 | MBC | 3-23 | 1-1 | 5 | Non-clonal | 3-15 | 5 | 9 |
| 350_8 | MBC | 3-23 | 3-16 | 4 | Non-clonal | 3-15 | 4 | 8 |
| 350_22 | MBC | 3-23 | 6-19 | 4 | Non-clonal | 3-11 | 4 | 6 |
| 350_132 | MBC | 3-48 | 1-26 | 4 | Non-clonal | 3-15 | 4 | 8 |
| 350_145 | MBC | 3-48 | 4-17 | 6 | 5 | 3-15 | 4 | 8 |
| 351_38 | MBC | 3-23 | 6-13 | 4 | Non-clonal | 3-15 | 4 | 8 |
| 351_93 | MBC | 3-23 | 1-1 | 4 | Non-clonal | 3-15 | 4 | 8 |
| 351_139 | MBC | 3-23 | 5-24 | 4 | Non-clonal | 3-11 | 4 | 6 |

TABLE 3
Extended Data:
Mutation information for FIG. 6f-g.

| | Naturally | 045-09 2B06 Deep | Other Experimentally | | |
|---|---|---|---|---|---|
| Mutation | Occurring? | Mutational Scan? | Determined? | Note | Source |
| H38S (HA1) | | No | Yes | Affects C179 binding | ⁴² |
| L292R (HA1) | | Yes | | In footprint of many VH1-69 utilizing mAbs | ⁴³ |
| I323V (HA1) | Yes | No | | Present in pH1N1 viruses | ⁴⁴ |
| K368D (HA2) | | Yes | Yes | Contacts with VH3-30 mAb | ⁴⁵ |
| Q371E (HA2) | | Yes | Yes | Interact with CR9114 and FI6v3 | ⁴⁶ |
| A373V (HA2) | | No | Yes | Expands in presence of stalk binding mAbs, | ^{2, 47} |
| | | | | escape mutant of 6F12 | |
| I374L (HA2) | | Yes | Yes | Mutation associated with resistance to group 1 | ^{46, 48, 49} |
| | | | | neutralizing stalk mAbs | |
| D375R (HA2) | | Yes | | Pre-pH1N1 viruses express N375 | |
| E376K (HA2) | Yes | Yes | | Associated with HA protein stability | ⁵⁰ |
| T378R (HA2) | | Yes | | Mutation associated with resistance to group 1 | ^{48, 49} |
| | | | | neutralizing stalk mAbs | |
| N379R (HA2) | | Yes | | Interacts with CR9114 | ¹⁰ |
| V381A (HA2) | | No | Yes | Affects FI6v3 mAb binding | ⁴⁶ |
| I385A (HA2) | | No | Yes | Affects F10 mAb binding | ⁵¹ |
| E386K (HA2) | | Yes | | Critical residue for membrane fusion | ⁵² |

TABLE 4
Extended Data:
Cryo-EM data collection and refinement statistics for
222-1C06 binding H1. Related to FIG. 2.

| | (EMDB-25655) |
|---|---|
| | (PDB 7T3D) |
| Data collection and processing | |
| Magnification | 36,000 |
| Voltage (kV) | 200 |
| Electron exposure (e−/Å²) | 49.9 |
| Defocus range (μm) | −0.8 to −1.8 |
| Pixel size (Å) | 1.15 |
| Symmetry imposed | C3 |
| Initial particle images (no.) | 92,514 (post 2D |
| | classification) |
| Final particle images (no.) | 48,846 |
| Map resolution (Å) | 3.38 |
| FSC threshold | 0.143 |
| Map resolution range (Å) | 3.2-4.4 |
| Refinement | |
| Initial model used (PDB code) | 7MEM |
| Model resolution (Å) | 3.5 |
| FSC threshold | 0.5 |
| Map sharpening B factor (Å²) | −88.1 |
| Model composition | |
| Non-hydrogen atoms | 22122 |
| Protein residues | 2796 |
| Ligands | NAG:21 |
| R.m.s. deviations | |
| Bond lengths (Å) | 0.022 |
| Bond angles (°) | 1.732 |
| Validation | |
| MolProbity score | 0.9 |
| Clashscore | 1.54 |
| Poor rotamers (%) | 0 |
| Ramachandran plot | |
| Favored (%) | 98.40 |
| Allowed (%) | 1.96 |
| Disallowed (%) | 0.00 |

SUPPLEMENTARY TABLE 1
mAbs used in cocktails for in vivo studies and neutralization
potency against A/Netherlands/602/2009.

| | | A/Netherlands/602/2009 IC₅₀ |
|---|---|---|
| mAb name | Epitope Specificity | (μg/ml) |
| 241 IgG 2A06 | Anchor | 22.99 |
| 047-09 4F04 | Anchor | 66.63 |
| 030-09 3E05 | Anchor | 39.71 |
| SFV009 3G01 | Anchor | 7.588 |
| 236 IgG 1A02 | Anchor | 66.88 |
| 045-09 2B06 | Central Stalk | 55.03 |
| SFV005 2G02 | Central Stalk | 9.342 |
| SFV019 4E03 | Central Stalk | 7.221 |
| 220 IgG 1A05 | Central Stalk | 9.536 |
| 241 IgA 2E06 | Central Stalk | 10.40 |

SUPPLEMENTARY TABLE 2
HA and 222-1C06 Fab interactions.

| HA Residues | Fab |
|---|---|
| S361 | K-CDR3 N93 |
| Q356 | K-CDR3-W94 |
| | H-CDR3 W99 |
| | H-CDR2 Y58 |
| E358 | H-CDR2 Y58 |
| backbone | |
| Aromatic Pocket: | Aromatic Pocket: |
| W343 | H-CDR3 W99 |
| H354 | H-CDR3 P100 |
| Y363 | K-CDR3 W94 |
| backbone | K-CDR1 R30 |
| positions 344-345 | |

SUPPLEMENTARY TABLE 3
Fab:Fab interactions of 222-1C06.
Fab:Fab interactions
Intra-fab aromatic pocket

K-CDR3 W94, P95, P96 -
Heavy Chain W47, Y50, Y59, W99, Y100c
K-CDR3 W94 - K-CDR3 P95
K-CDR3 P95 - HCDR2 Y59
K-CDR3 W94 - H-CDR3 Y100c
K-CDR3 P96 - H-FR2 W47

SUPPLEMENTARY TABLE 4
Reference H1 strains used in FIG. 2j.

| Subtype | Strain Name |
|---|---|
| H1N1 | A/Quebec/25/2013 |
| H1N1 | A/Czech Republic/4/2016 |
| H1N1 | A/Helsinki/77/2013 |
| H1N1 | A/New York/18/2009 |
| H1N1 | A/swine/Mexico/AVX27/2012 |
| H1N2 | A/swine/Korea/PZ4/2006 |
| H1N2 | A/swine/Ohio/8910/2001 |
| H1N1 | A/swine/North Carolina/SG1279/2007 |
| H1N1 | A/swine/Minnesota/00991/2006 |
| H1N1 | A/swine/Hong Kong/1937/1994 |
| H1N1 | A/swine/Wisconsin/1915/1988 |
| H1N1 | A/swine/Wisconsin/11/1976 |
| H1N2 | A/swine/Miyagi/5/2003 |
| H1N1 | A/swine/Manitoba/D0270/2013 |
| H1N1 | A/swine/Ohio/23/1935 |
| H1N1 | A/swine/USA/1976/1931 |
| H1N1 | A/South Carolina/1/1918 |
| H1N1 | A/Iran/1417/2016 |
| H1N1 | A/Henry/1936 |
| H1N1 | A/Albany/1618/1951 |
| H1N1 | A/Roma/1949 |
| H1N2 | A/swine/England/463180/1994 |
| H1N2 | A/swine/Italy/233139/2005 |
| H1N2 | A/swine/England/448813/1994 |
| H1N2 | A/swine/England/063782/2013 |
| H1N1 | A/Singapore/6/1986 |
| H1N1 | A/Ostrova/801/1998 |
| H1N2 | A/swine/Minnesota/A01201102/2011 |
| H1N2 | A/swine/Minnesota/A01567011/2014 |
| H1N2 | A/swine/North Carolina/2858/2010 |
| H1N1 | A/swine/IL/00685/2005 |
| H1N1 | A/Auckland/585/2000 |
| H1N1 | A/California/UR060479/2007 |
| H1N3 | A/pintail/Aomori/1130/2008 |
| H1N3 | A/duck/Fujian/FJ1239/2014 |
| H1N1 | A/duck/Bavaria/1/1977 |
| H1N9 | A/thickbilled murre/Alaska/390/1976 |
| H1N9 | A/northern shoveler/California/K168/2005 |
| H1N1 | A/yellow-billed pintail/Chile/1/2012 |
| H1N1 | A/mallard duck/Chile/C4079/2015 |
| H1N1 | A/swine/Arnsberg/6554/1979 |
| H1N1 | A/swine/Germany/8533/1991 |
| H1N2 | A/swine/Italy/195369/2010 |
| H1N1 | A/swine/Hong Kong/1559/2008 |
| H1N1 | A/swine/Cotes d'Armor/1624/2002 |
| H1N1 | A/swine/Jiangsu/J004/2018 |

### E. REFERENCES

The following references and the references cited throughout the specification, to the extent that they provide exemplary procedural or other details supplementary to those set forth herein, are specifically incorporated herein by reference.
1. Paules, C. I., Marston, H. D., Eisinger, R. W., Baltimore, D. & Fauci, A. S. The Pathway to a Universal Influenza Vaccine. Immunity 47, 599-603, doi:10.1016/j.immuni.2017.09.007 (2017).
2. Park, J. K. et al. Pre-existing immunity to influenza virus hemagglutinin stalk might drive selection for antibody-escape mutant viruses in a human challenge model. Nat Med 26, 1240-1246, doi:10.1038/s41591-020-0937-x (2020).
3. Guthmiller, J. J. et al. First exposure to the pandemic H1N1 virus induced broadly neutralizing antibodies targeting hemagglutinin head epitopes. Sci Transl Med 13, doi: 10.1 126/scitranslmed.abg4535 (2021).
4. Bernstein, D. I. et al. Immunogenicity of chimeric haemagglutinin-based, universal influenza virus vaccine candidates: interim results of a randomised, placebo-controlled, phase 1 clinical trial. Lancet Infect Dis 20, 80-91, doi:10.1016/S1473-3099(19)30393-7 (2020).
5. Nachbagauer, R. et al. A chimeric hemagglutinin-based universal influenza virus vaccine approach induces broad and long-lasting immunity in a randomized, placebo-controlled phase I trial. Nat Med, doi:10.1038/s41591-020-1118-7 (2020).
6. Ng, S. et al. Novel correlates of protection against pandemic H1N1 influenza A virus infection. Nat Med 25, 962-967, doi:10.1038/s41591-019-0463-x (2019).
7. Aydillo, T. et al. Pre-existing Hemagglutinin Stalk Antibodies Correlate with Protection of Lower Respiratory Symptoms in Flu-Infected Transplant Patients. Cell Rep Med 1, 100130, doi:10.1016/j.xcrm.2020.100130 (2020).
8. Wrammert, J. et al. Broadly cross-reactive antibodies dominate the human B cell response against 2009 pandemic H1N1 influenza virus infection. J Exp Med 208, 181-193, doi:10.1084/jem.20101352 (2011).
9. Andrews, S. F. et al. Immune history profoundly affects broadly protective B cell responses to influenza. Sci Transl Med 7, 316ra192, doi:10.1126/scitranslmed.aad0522 (2015).
10. Dreyfus, C. et al. Highly conserved protective epitopes on influenza B viruses. Science 337, 1343-1348, doi:10.1126/science.1222908 (2012).
11. Benton, D. J. et al. Influenza hemagglutinin membrane anchor. Proc Natl Acad Sci USA 115, 10112-10117, doi:10.1073/pnas.1810927115 (2018).
12. Ekiert, D. C. et al. A highly conserved neutralizing epitope on group 2 influenza A viruses. Science 333, 843-850, doi: 10.1126/science.1204839 (2011).
13. Friesen, R. H. et al. A common solution to group 2 influenza virus neutralization. Proc Natl Acad Sci USA 111, 445-450, doi:10.1073/pnas.1319058110 (2014). 14. Corti, D. et al. A neutralizing antibody selected from plasma cells that binds to group 1 and group 2 influenza A hemagglutinins. Science 333, 850-856, doi: 10.1126/science.1205669 (2011).
15. DiLillo, D. J., Palese, P., Wilson, P. C. & Ravetch, J. V. Broadly neutralizing anti-influenza antibodies require Fc receptor engagement for in vivo protection. J Clin Invest 126, 605-610, doi: 10.1 172/JC184428 (2016).
16. He, W. et al. Epitope specificity plays a critical role in regulating antibody-dependent cell-mediated cytotoxicity against influenza A virus. Proc Natl Acad Sci USA 113, 11931-11936, doi:10.1073/pnas.1609316113 (2016).
17. Guthmiller, J. J. et al. Polyreactive Broadly Neutralizing B cells Are Selected to Provide Defense against Pandemic Threat Influenza Viruses. Immunity, doi: 10.1016/j.immuni.2020.10.005 (2020).
18. Sutton, T. C. et al. In Vitro Neutralization Is Not Predictive of Prophylactic Efficacy of Broadly Neutralizing Monoclonal Antibodies CR6261 and CR9114 against Lethal H2 Influenza Virus Challenge in Mice. J Virol 91, doi:10.1128/JVI.01603-17 (2017).
19. Benton, D. J., Gamblin, S. J., Rosenthal, P. B. & Skehel, J. J. Structural transitions in influenza haemagglutinin at membrane fusion pH. Nature 583, 150-153, doi:10.1038/s41586-020-2333-6 (2020).
20. Han, J. et al. Polyclonal epitope mapping reveals temporal dynamics and diversity of human antibody responses to H5N1 vaccination. Cell Rep 34, 108682, doi:10.1016/j.celrep.2020.108682 (2021).
21. Impagliazzo, A. et al. A stable trimeric influenza hemagglutinin stem as a broadly protective immunogen. Science 349, 1301-1306, doi:10.1126/science.aac7263 (2015).
22. Yassine, H. M. et al. Hemagglutinin-stem nanoparticles generate heterosubtypic influenza protection. Nat Med 21, 1065-1070, doi:10.1038/nm.3927 (2015).
23. Sun, H. et al. Prevalent Eurasian avian-like HiN1 swine influenza virus with 2009 pandemic viral genes facilitating human infection. Proc Natl Acad Sci USA 117, 17204-17210, doi:10.1073/pnas.1921186117 (2020).
24. Caskey, M. et al. Viraemia suppressed in HIV-1-infected humans by broadly neutralizing antibody 3BNC117. Nature 522, 487-491, doi:10.1038/naturel4411 (2015).
25. Caskey, M. et al. Antibody 10-1074 suppresses viremia in HIV-1-infected individuals. Nat Med 23, 185-191, doi:10.1038/nm.4268 (2017).
26. Bar, K. J. et al. Effect of HIV Antibody VRCO1 on Viral Rebound after Treatment Interruption. N Engl J Med 375, 2037-2050, doi:10.1056/NEJMoa1608243 (2016).
27. Mendoza, P. et al. Combination therapy with anti-HIV-1 antibodies maintains viral suppression. Nature 561, 479-484, doi:10.1038/s41586-018-0531-2 (2018).
28. Raymond, D. D. et al. Conserved epitope on influenza-virus hemagglutinin head defined by a vaccine-induced antibody. Proc Natl Acad Sci USA 115, 168-173, doi:10.1073/pnas.1715471115 (2018).
29. Linderman, S. L. et al. Potential antigenic explanation for atypical HIN1 infections among middle-aged adults during the 2013-2014 influenza season. Proc Natl Acad Sci USA 111, 15798-15803, doi:10.1073/pnas.1409171111 (2014).
30. Tate, M. D. et al. Playing hide and seek: how glycosylation of the influenza virus hemagglutinin can modulate the immune response to infection. Viruses 6, 1294-1316, doi: 10.3390/v6031294 (2014).
31. Pallesen, J. et al. Immunogenicity and structures of a rationally designed prefusion MERS-CoV spike antigen. Proc Natl Acad Sci USA 114, E7348-E7357, doi: 10.1073/pnas.1707304114 (2017).
32. Dugan, H. L. et al. Preexisting immunity shapes distinct antibody landscapes after influenza virus infection and vaccination in humans. Sci Transl Med 12, doi: 10.1 126/scitranslmed.abd3601 (2020).
33. van der Lubbe, J. E. M. et al. Mini-hemagglutinin vaccination induces cross-reactive antibodies in pre-exposed NHP that protect mice against lethal influenza challenge. NPJ Vaccines 3, 25, doi:10.1038/s41541-018-0063-7 (2018).
34. Cantisani, R. et al. Vaccine adjuvant MF59 promotes retention of unprocessed antigen in lymph node macrophage compartments and follicular dendritic cells. J Immunol 194, 1717-1725, doi: 10.4049/jimmunol.1400623 (2015).
35. Liang, F. et al. Vaccine priming is restricted to draining lymph nodes and controlled by adjuvant-mediated antigen uptake. Sci Transl Med 9, doi:10.1126/scitranslmed.aal2094 (2017).
36. Khurana, S. et al. Vaccines with MF59 adjuvant expand the antibody repertoire to target protective sites of pandemic avian H5N1 influenza virus. Sci Transl Med 2, 15ra15, doi: 10.1 126/scitranslmed.3000624 (2010).
37. Bloom, J. D., Gong, L. I. & Baltimore, D. Permissive secondary mutations enable the evolution of influenza oseltamivir resistance. Science 328, 1272-1275, doi:10.1126/science.1187816 (2010).
38. Guthmiller, J. J., Dugan, H. L., Neu, K. E., Lan, L. Y. & Wilson, P. C. An Efficient Method to Generate Monoclonal Antibodies from Human B Cells. Methods Mol Biol 1904, 109-145, doi: 10.1007/978-1-4939-8958-4_5 (2019).
39. Wrammert, J. et al. Rapid cloning of high-affinity human monoclonal antibodies against influenza virus. Nature 453, 667-671, doi:10.1038/nature06890 (2008).
40. Smith, K. et al. Rapid generation of fully human monoclonal antibodies specific to a vaccinating antigen. Nat Protoc 4, 372-384, doi:10.1038/nprot.2009.3 (2009).
41. Crooks, G. E., Hon, G., Chandonia, J. M. & Brenner, S. E. WebLogo: a sequence logo generator. Genome Res 14, 1188-1190, doi:10.1101/gr.849004 (2004).
42. Doud, M. B., Lee, J. M. & Bloom, J. D. How single mutations affect viral escape from broad and narrow antibodies to H1 influenza hemagglutinin. Nat Commun 9, 1386, doi:10.1038/s41467-018-03665-3 (2018).
43. Lang, S. et al. Antibody 27F3 Broadly Targets Influenza A Group 1 and 2 Hemagglutinins through a Further Variation in VH1-69 Antibody Orientation on the H A Stem. Cell Rep 20, 2935-2943, doi:10.1016/j.celrep.2017.08.084 (2017).
44. Xu, R. et al. Functional balance of the hemagglutinin and neuraminidase activities accompanies the emergence of the 2009 H1N1 influenza pandemic. J Virol 86, 9221-9232, doi:10.1128/JVI.00697-12 (2012).
45. Fu, Y. et al. A broadly neutralizing anti-influenza antibody reveals ongoing capacity of haemagglutinin-specific memory B cells to evolve. Nat Commun 7, 12780, doi:10.1038/ncomms12780 (2016).
46. Wu, N. C. et al. Different genetic barriers for resistance to H A stem antibodies in influenza H3 and H1 viruses. Science 368, 1335-1340, doi:10.1126/science.aaz5143 (2020).
47. Tan, G. S. et al. A pan-H1 anti-hemagglutinin monoclonal antibody with potent broad-spectrum efficacy in vivo. J Virol 86, 6179-6188, doi: 10.1128/JVI.00469-12 (2012).
48. Yassine, H. M. et al. Use of Hemagglutinin Stem Probes Demonstrate Prevalence of Broadly Reactive Group 1 Influenza Antibodies in Human Sera. Sci Rep 8, 8628, doi:10.1038/s41598-018-26538-7 (2018).
49. Lingwood, D. et al. Structural and genetic basis for development of broadly neutralizing influenza antibodies. Nature 489, 566-570, doi:10.1038/nature11371 (2012).
50. Cotter, C. R., Jin, H. & Chen, Z. A single amino acid in the stalk region of the HIN1pdm influenza virus H A protein affects viral fusion, stability and infectivity. PLoS Pathog 10, e1003831, doi:10.1371/journal.ppat.1003831 (2014).
51. Sui, J. et al. Structural and functional bases for broad-spectrum neutralization of avian and human influenza A viruses. Nat Struct Mol Biol 16, 265-273, doi:10.1038/nsmb.1566 (2009).
52. Vanderlinden, E. et al. Novel inhibitors of influenza virus fusion: structure-activity relationship and interaction with the viral hemagglutinin. J Virol 84, 4277-4288, doi:10.1128/JVI.02325-09 (2010).
53. Hai, R. et al. Influenza viruses expressing chimeric hemagglutinins: globular head and stalk domains derived from different subtypes. J Virol 86, 5774-5781, doi:10.1128/JVI.00137-12 (2012).
54. Shlomchik, M. J., Aucoin, A. H., Pisetsky, D. S. & Weigert, M. G. Structure and function of anti-DNA autoantibodies derived from a single autoimmune mouse. Proc Natl Acad Sci USA 84, 9150-9154 (1987).
55. Doud, M. B. & Bloom, J. D. Accurate Measurement of the Effects of All Amino-Acid Mutations on Influenza Hemagglutinin. Viruses 8, doi:10.3390/v8060155 (2016).
56. Doud, M. B., Hensley, S. E. & Bloom, J. D. Complete mapping of viral escape from neutralizing antibodies. PLoS Pathog 13, e1006271, doi:10.1371/journal.ppat.1006271 (2017).
57. Bloom, J. D. Software for the analysis and visualization of deep mutational scanning data. BMC Bioinformatics 16, 168, doi:10.1186/s12859-015-0590-4 (2015).
58. Henry Dunand, C. J. et al. Preexisting human antibodies neutralize recently emerged H7N9 influenza strains. J Clin Invest 125, 1255-1268, doi:10.1172/JCI74374 (2015).
59. Chen, Y. Q. et al. Influenza Infection in Humans Induces Broadly Cross-Reactive and Protective Neuraminidase-Reactive Antibodies. Cell 173, 417-429 e410, doi:10.1016/j.cell.2018.03.030 (2018).
60. Pettersen, E. F. et al. UCSF Chimera—a visualization system for exploratory research and analysis. J Comput Chem 25, 1605-1612, doi:10.1002/jcc.20084 (2004).
61. Suloway, C. et al. Automated molecular microscopy: the new Leginon system. J Struct Biol 151, 41-60, doi:10.1016/j.jsb.2005.03.010 (2005).
62. Lander, G. C. et al. Appion: an integrated, database-driven pipeline to facilitate E M image processing. J Struct Biol 166, 95-102, doi:10.1016/j.jsb.2009.01.002 (2009).
63. Scheres, S. H. RELION: implementation of a Bayesian approach to cryo-E M structure determination. J Struct Biol 180, 519-530, doi:10.1016/j.jsb.2012.09.006 (2012).
64. Zheng, S. Q. et al. MotionCor2: anisotropic correction of beam-induced motion for improved cryo-electron microscopy. Nat Methods 14, 331-332, doi: 10.1038/nmeth.4193 (2017).
65. Punjani, A., Rubinstein, J. L., Fleet, D. J. & Brubaker, M. A. cryoSPARC: algorithms for rapid unsupervised cryo-E M structure determination. Nat Methods 14, 290-296, doi: 10.1038/nmeth.4169 (2017).
66. Emsley, P. & Cowtan, K. Coot: model-building tools for molecular graphics. Acta Crystallogr D Biol Crystallogr 60, 2126-2132, doi:10.1107/S0907444904019158 (2004).
67. Weitzner, B. D. et al. Modeling and docking of antibody structures with Rosetta. Nat Protoc 12, 401-416, doi:10.1038/nprot.2016.180 (2017).
68. Chen, V. B. et al. MolProbity: all-atom structure validation for macromolecular crystallography. Acta Crystallogr D Biol Crystallogr 66, 12-21, doi:10.1107/S0907444909042073 (2010).
69. Barad, B. A. et al. EMRinger: side chain-directed model and map validation for 3D cryo-electron microscopy. Nat Methods 12, 943-946, doi: 10.1038/nmeth.3541 (2015).
70. Li, L. et al. Improved integration of single-cell transcriptome and surface protein expression by LinQ-View. Cell Reports Methods, 100056, doi:https://doi.org/10.1016/j.crmeth.2021.100056 (2021).
71. Zhuang, Q. et al. Diversity and distribution of type A influenza viruses: an updated panorama analysis based on protein sequences. Virol J 16, 85, doi:10.1186/s12985-019-1188-7 (2019).
72. Burke, D. F. & Smith, D. J. A recommended numbering scheme for influenza A H A subtypes. PLoS One 9, e112302, doi:10.1371/journal.pone.0112302 (2014).
73. Edgar, R. C. MUSCLE: multiple sequence alignment with high accuracy and high throughput. Nucleic Acids Res 32, 1792-1797, doi:10.1093/nar/gkh340 (2004).
74. Li, L. et al. Librator, a platform for optimized sequence editing, design, and expression of influenza virus proteins. bioRxiv, 2021.2004.2029.441999, doi:10.1101/2021.04.29.441999 (2021).
75. Weitzner, B. D., Kuroda, D., Marze, N., Xu, J. & Gray, J. J. Blind prediction performance of RosettaAntibody 3.0: grafting, relaxation, kinematic loop modeling, and full CDR optimization. Proteins 82, 1611-1623, doi:10.1002/prot.24534 (2014).
76. Stein, A. & Kortemme, T. Improvements to robotics-inspired conformational sampling in rosetta. PLoS One 8, e63090, doi:10.1371/journal.pone.0063090 (2013).
77. Labute, P. Protonate3D: assignment of ionization states and hydrogen coordinates to macromolecular structures. Proteins 75, 187-205, doi:10.1002/prot.22234 (2009).
78. Molecular Operating Environment (MOE). 1010 Sherbrooke St. West, S., Montreal, Q C, Canada,H3A 2R7: 2020..
79. Hub, J. S., de Groot, B. L., Grubmuller, H. & Groenhof, G. Quantifying Artifacts in Ewald Simulations of Inhomogeneous Systems with a Net Charge. J Chem Theory Comput 10, 381-390, doi: 10.1021/ct400626b (2014).
80. Roe, D. R. & Cheatham, T. E., 3rd. PTRAJ and CPPTRAJ: Software for Processing and Analysis of Molecular Dynamics Trajectory Data. J Chem Theory Comput 9, 3084-3095, doi:10.1021/ct400341p (2013).
81. D. A. Case, K. B., I. Y. Ben-Shalom, S. R. Brozell, D. S. Cerutti, T. E. Cheatham, III, V. W. D. Cruzeiro, T. A. Darden, R. E. Duke, G. Giambasu, M. K. Gilson, H. Gohlke, A. W. Goetz,R Harris, S. Izadi, S. A. Izmailov, K. Kasavajhala, A. Kovalenko, R. Krasny, T. Kurtzman, T. S. Lee, S. LeGrand, P. Li, C. Lin, J. Liu, T. Luchko, R. Luo, V. Man, K. M. Merz, Y. Miao, O. Mikhailovskii, G. Monard, H. Nguyen, A. Onufriev, F., Pan, S. Pantano, R. Qi, D. R. Roe, A. Roitberg, C. Sagui, S. Schott-Verdugo, J. Shen, C. L. Simmerling, N. R., Skrynnikov, J. Smith, J. Swails, R. C. Walker, J. Wang, L. Wilson, R. M. Wolf, X. Wu, Y. Xiong, Y. Xue,, D. M. York and P. A. Kollman (2020), AMBER 2020, University of California, San Francisco. 2020;.
82. Jorgensen, W. L., Chandrasekhar, J., Madura, J. D., Impey, R. W. & Klein, M. L. Comparison of simple potential functions for simulating liquid water. The Journal of Chemical Physics 79, 926-935, doi:10.1063/1.445869 (1983).
83. Maier, J. A. et al. ffl4S B: Improving the Accuracy of Protein Side Chain and Backbone Parameters from ff99S B. J Chem Theory Comput 11, 3696-3713, doi:10.1021/acs.jctc.5b00255 (2015).
84. Wallnoefer, H. G., Liedl, K. R. & Fox, T. A challenging system: free energy prediction for factor Xa. J Comput Chem 32, 1743-1752, doi:10.1002/jcc.21758 (2011).
85. Barducci, A., Bonomi, M. & Parrinello, M. Metadynamics. WIREs Computational Molecular Science 1, 826-843, doi:https://doi.org/10.1002/wcms.31 (2011).
86. Barducci, A., Bussi, G. & Parrinello, M. Well-tempered metadynamics: a smoothly converging and tunable free-energy method. Phys Rev Lett 100, 020603, doi: 10.1 103/PhysRevLett.100.020603 (2008).
87. Biswas, M., Lickert, B. & Stock, G. Metadynamics Enhanced Markov Modeling of Protein Dynamics. J Phys Chem B 122, 5508-5514, doi:10.1021/acs.jpcb.7b11800 (2018).
88. Abraham, M. J. et al. GROMACS: High performance molecular simulations through multi-level parallelism from laptops to supercomputers. SoftwareX 1-2, 19-25, doi:10.1016/j.softx.2015.06.001 (2015).
89. Pronk, S. et al. GROMACS 4.5: a high-throughput and highly parallel open source molecular simulation toolkit. Bioinformatics 29, 845-854, doi:10.1093/bioinformatics/btt055 (2013).
90. Tribello, G. A., Bonomi, M., Branduardi, D., Camilloni, C., Bussi, G. PLUMED 2: New feathers for an old bird. Computer Physics Communications Volume 185, Issue 2,, 604-613, doi:https://doi.org/10.1016/j.cpc.2013.09.018 (2014).
91. Ilott, A. J., Palucha, S., Hodgkinson, P. & Wilson, M. R. Well-tempered metadynamics as a tool for characterizing multi-component, crystalline molecular machines. J Phys Chem B 117, 12286-12295, doi:10.1021/jp4045995 (2013).
92. Ramachandran, G. N., Ramakrishnan, C. & Sasisekharan, V. Stereochemistry ofpolypeptide chain configurations. J Mol Biol 7, 95-99, doi:10.1016/s0022-2836(63)80023-6 (1963).
93. Fernandez-Quintero, M. L. et al. Antibodies exhibit multiple paratope states influencing V H-V L domain orientations. Commun Biol 3, 589, doi:10.1038/s42003-020-01319-z (2020).
94. Salomon-Ferrer, R., Gotz, A. W., Poole, D., Le Grand, S. & Walker, R. C. Routine Microsecond Molecular Dynamics Simulations with AMBER on GPUs. 2. Explicit Solvent Particle Mesh Ewald. J Chem Theory Comput 9, 3878-3888, doi:10.1021/ct400314y (2013).
95. Shao, J., Tanner, S. W., Thompson, N. & Cheatham, T. E. Clustering Molecular Dynamics Trajectories: 1. Characterizing the Performance of Different Clustering Algorithms. J Chem Theory Comput 3, 2312-2334, doi:10.1021/ct700119m (2007).
96. Miyamoto S, K. P. Settle: An analytical version of the SHAKE and RATTLE algorithm for rigid water models. J Comput Chem, 952-962 (1992).
97. Berendsen H, P. J., van Gunsteren W, DiNola A, Haak J R. Molecular-Dynamics with Coupling to An External Bath. J Chem Phys, 3684 (1984).
98. Adelman, S. A. & Doll, J. D. Generalized Langevin equation approach for atom/solid-surface scattering: General formulation for classical scattering off harmonic solids. The Journal of Chemical Physics 64, 2375-2388, doi:10.1063/1.432526 (1976).
99. Scherer, M. K. et al. PyEMMA 2: A Software Package for Estimation, Validation, and Analysis of Markov Models. J Chem Theory Comput 11, 5525-5542, doi:10.1021/acs.jctc.5b00743 (2015).
100. Chodera, J. D. & Noe, F. Markov state models of biomolecular conformational dynamics. Curr Opin Struct Biol 25, 135-144, doi:10.1016/j.sbi.2014.04.002 (2014).

### Example 2—Broadly Neutralizing Antibodies Target a Hemagglutinin Anchor Epitope

Broadly neutralizing antibodies against influenza virus hemagglutinin (HA) have the potential to provide universal protection against influenza virus infections. Here, we report a distinct class of broadly neutralizing antibodies targeting an epitope toward the bottom of the HA stalk domain where HA is “anchored” to the viral membrane. Antibodies targeting this membrane-proximal anchor epitope utilized a highly restricted repertoire, which encode for two conserved motifs responsible for HA binding. Anchor targeting B cells were common in the human memory B cell repertoire across subjects, indicating pre-existing immunity against this epitope. Antibodies against the anchor epitope at both the serological and monoclonal antibody levels were potently induced in humans by a chimeric HA vaccine, a potential universal influenza virus vaccine. Altogether, this study reveals an underappreciated class of broadly neutralizing antibodies against H1-expressing viruses that can be robustly recalled by a candidate universal influenza virus vaccine.

### A. Results

### 1. Identification of Antibodies Targeting the Anchor Epitope

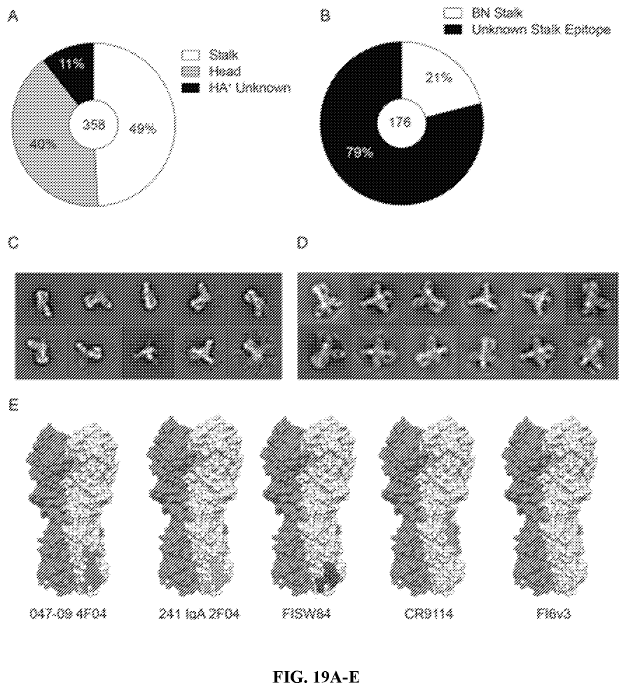

To dissect conserved HA stalk domain epitopes, we generated mAbs from acutely activated plasmablasts isolated from subjects who received licensed or experimental influenza virus vaccines or were naturally infected with pH1N1 during the 2009 pandemic (Table S1). Notably, plasmablasts found in the blood of subjects after infection or vaccination derive from pre-existing MBCs (Andrews et al., 2015), and generation of mAbs from plasmablasts allows for the dissection of how distinct influenza viruses recall pre-existing immunity. We also generated mAbs from sorted HA+ B cells one month following vaccination with an experimental cHA vaccine that utilized the head domain from an avian influenza virus and stalk domain from pH1N1 (Bernstein et al., 2020). We specifically focused our studies on mAbs targeting the stalk domain of H1-expressing viruses, as prior studies have shown first exposure to the 2009 pandemic H1N1 virus induce antibodies against the stalk domain (Li et al., 2012; Wrammert et al., 2011). To define antibodies as targeting the H1 stalk, mAbs were tested for binding to cH5/1, which utilizes the head domain from H5-expressing viruses and the stalk domain from the pH1N1 virus (Hai et al., 2012), and for hemagglutination inhibition (HAI) activity against pH1N1 (A/California/July 2009), a feature of head binding antibodies. MAbs that bound the cHA and that lacked HAI activity were classified as those binding the HA stalk domain. Of all mAbs tested, nearly 49% targeted the HA stalk domain, whereas 40% targeted the HA head domain (FIG. 19A). To investigate what proportion were binding the BN stalk domain epitope, we competed the stalk binding mAbs from our cohorts with CR9114, a well-defined antibody targeting the BN stalk epitope (Dreyfus et al., 2012). We identified that only 21% of mAbs targeting the stalk domain had greater than 80% competition with CR9114 (FIG. 19B), indicating most H1 stalk domain targeting antibodies were binding other epitopes of the HA stalk.

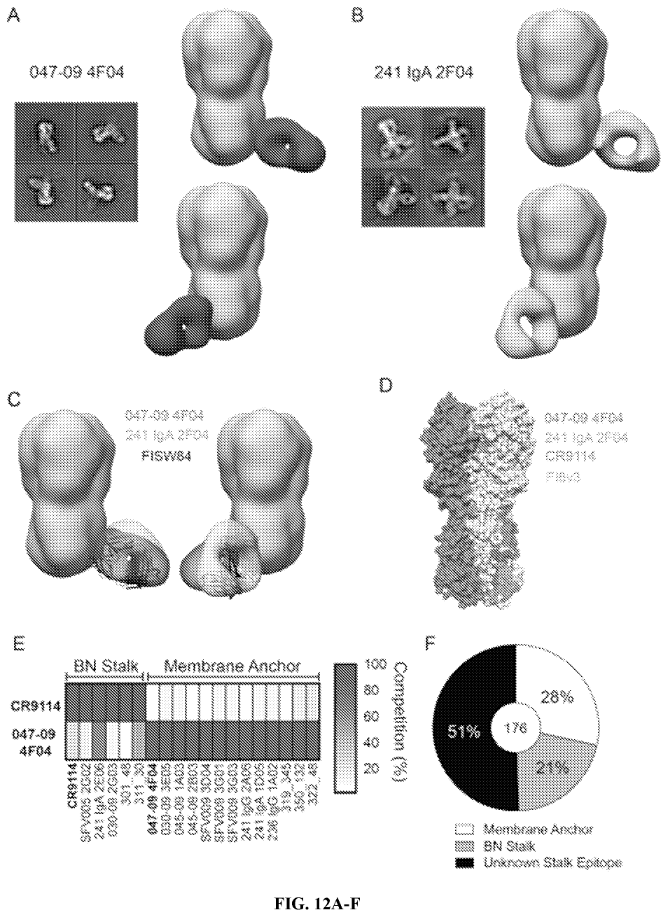

To investigate which epitopes the remaining 79% of mAbs were targeting on the stalk domain, we performed negative stain electron microscopy with two stalk domain binding mAbs. Both mAbs bound an epitope near the anchor of the HA stalk, towards the lower portion of the HA protomer (FIG. 12A-B; FIG. 19C-D). Both mAbs were oriented at an upward angle towards the epitope (FIG. 12A-B), suggesting this epitope may be partially obstructed by the lipid membrane and may only be exposed for antibody binding as the HA trimers flex on the viral membrane (Benton et al., 2018). FISW84, a recently identified anchor binding mAb, (Benton et al., 2018), overlap with both 047-09 4F04 and 241 IgA 2F04 (FIG. 12C), suggesting this epitope is a common target of stalk binding antibodies. The footprint of several BN stalk epitope binding mAbs (CR9114 and FI6v3) did not overlap with those of 047-09 4F04 and 241 IgA 2F04 (FIG. 12D; FIG. 19E), indicating the anchor epitope and the BN stalk epitope are distinct epitopes on the HA stalk. To understand what proportion of stalk binding mAbs were binding to the anchor epitope, we competed 047-09 4F04 mAb with the remaining stalk binding mAbs that did not compete with CR9114. In total, we identified 50 distinct mAbs that competed for binding to the anchor epitope from a total of 21 subjects (Table S2) and accounted for 28% of all stalk mAbs identified (FIG. 12E-F). Together, these data indicate that the anchor epitope is a common target of antibodies binding the H1 stalk domain.

### 2. Antibodies Binding the Anchor are Broadly Reactive Amongst H1 Viruses

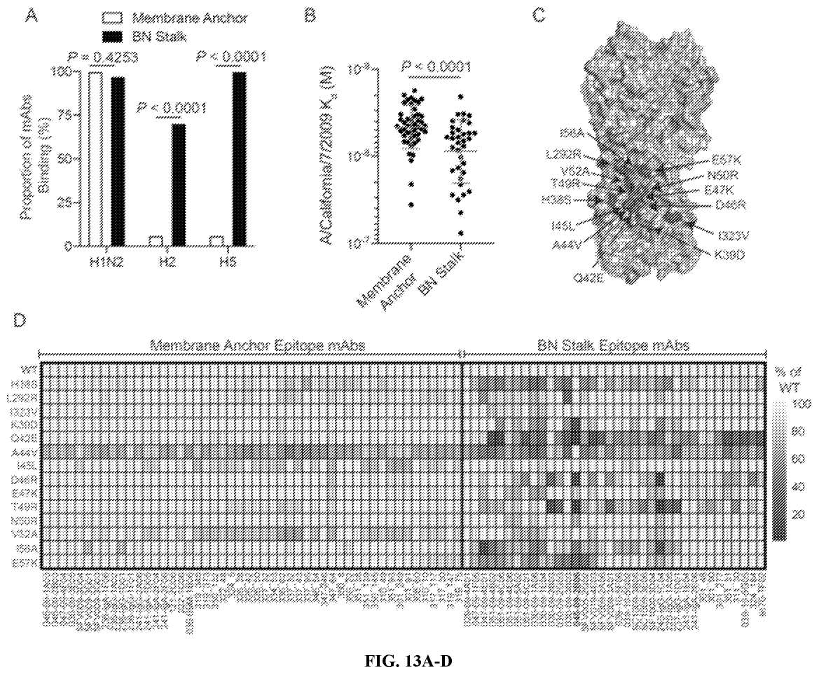

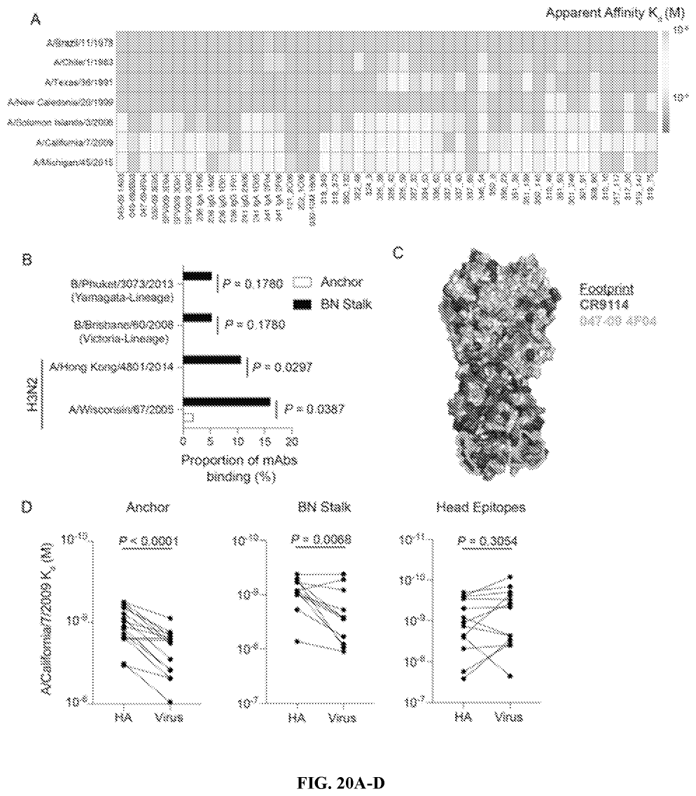

As the stalk domain is conserved amongst influenza viruses, we next determined the viral binding breadth of antibodies targeting the anchor epitope. Anchor mAbs were broadly reactive amongst H1-expressing viruses, including a swine origin H1N2 virus, but rarely cross-reacted with other influenza subtypes (FIG. 13A; FIG. 20A-B), as often occurs for antibodies targeting the BN stalk epitope (FIG. 13A; FIG. 20B). While highly conserved amongst H1 viruses, the anchor epitope was poorly conserved across divergent group 1 viral subtypes (FIG. 20C). Anchor epitope targeting mAbs had nearly a 2-fold higher affinity for pH1N1 virus than mAbs targeting the BN stalk epitope (FIG. 13B). Because the anchor epitope is partially obstructed by the lipid membrane, we next tested whether anchor binding mAbs had reduced affinity for whole virus relative to recombinant HA (rHA). MAbs binding the anchor epitope and the BN stalk epitope both exhibited reduced affinity for the whole virus (A/California/July 2009) relative to rHA from the same virus, whereas mAbs targeting the HA head domain had similar affinity for whole virus and rHA (FIG. 20D). These data indicate that antibodies targeting the anchor epitope are broadly reactive amongst H1-expressing viruses.

### 3. Antibodies Targeting the Anchor Epitope Maintain Binding to HA Mutants in the Stalk Domain

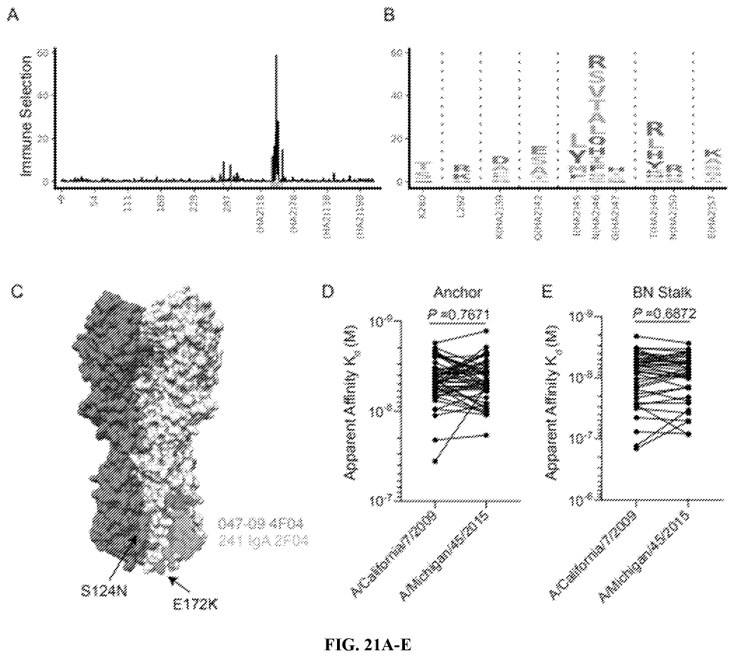

H1N1 viruses have acquired several mutations within the HA stalk domain, likely due to antibody selective pressures or to increase stability (Cotter et al., 2014). To understand whether these mutations have affected antibody binding to the anchor epitope, we screened mAbs against naturally occurring mutants and experimentally identified viral escape mutants of BN stalk epitope binding mAbs (FIG. 13C-D; FIG. 21A-B; Table S3). Anchor epitope binding mAbs were mostly unaffected by the mutants tested, whereas most of the mAbs targeting the BN stalk epitope were affected by at least one mutant, notably Q42E mutation in HA2 (FIG. 13D). Regardless of mAb specificity, most antibodies had reduced binding to A44V of HA2, which was recently shown to preferentially grow in the presence of mAbs against the BN stalk epitope (Park et al., 2020). While A44 is distant from the anchor epitope, the A44V mutation was shown to affect the conformation of the HA stalk (Park et al., 2020) and could explain the broad reduction of HA binding by antibodies targeting either the anchor epitope or the BN stalk epitope. Furthermore, A/Michigan/45/2015 acquired mutations at S124N and E172K of HA2 (Clark et al., 2017), which lay near the binding footprint of 047-09 4F04 and 241 IgA 2F04 (FIG. 21C). Despite this, mAbs targeting the anchor epitope or BN stalk epitope bind A/California/July 2009 (S124, E172) with nearly identical affinity as they do to A/Michigan/45/2015 (N124, K172; FIG. 21D-E), indicating these mutations were likely not driven by selective pressures of mAbs targeting these residues. Together, these data indicate that known mutations within the HA stalk largely do not affect the binding of antibodies to the anchor epitope.

### 4. Antibodies Targeting the Anchor Epitope are Broadly Neutralizing Against H1N1 Viruses and are Potently Protective In Vivo

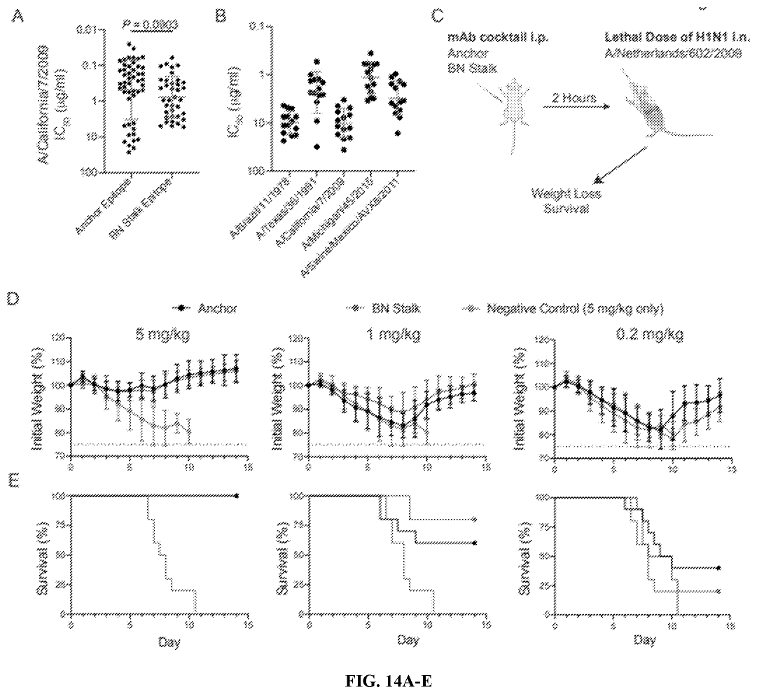

We next determined whether mAbs targeting the anchor epitope were neutralizing. All mAbs targeting the anchor epitope and the BN stalk epitope were neutralizing against the pH1N1 virus (A/California/July 2009) and had similar neutralizing potency relative to mAbs binding the BN stalk epitope (FIG. 14A). Furthermore, anchor targeting mAbs were broadly neutralizing against historical and recent H1N1 viruses, as well as a swine H1N2 (A/swine/Mexico/AVX August 2011) virus (FIG. 14B). Together, these data indicate that antibodies against the anchor epitope and other broadly neutralizing epitopes could work in tandem to be potently neutralizing against antigenically drifted and shifted H1-expressing viruses.

To test whether mAbs targeting the anchor epitope were protective in vivo, we prophylactically administered a cocktail of 5 mAbs targeting the anchor epitope or the BN stalk epitope to mimic a polyclonal response against these epitopes and infected mice with a lethal dose of a mouse-adapted pH1N1 virus (A/Netherlands/602/2009; FIG. 14C). Mice that received either cocktail lost a similar amount of weight and experienced similar mortality (FIG. 14D-E). Notably, mice were completely protected at 5 mg/kg of mAb cocktail, whereas only 60-80% of animals survived at 1 mg/kg (FIG. 14E). Together, these data reveal mAbs targeting the anchor epitope are protective in vivo and could provide broad protection against H1-expressing viruses.

### 5. Anchor Epitope Targeting Antibodies are Induced by Seasonal Influenza Virus Vaccines

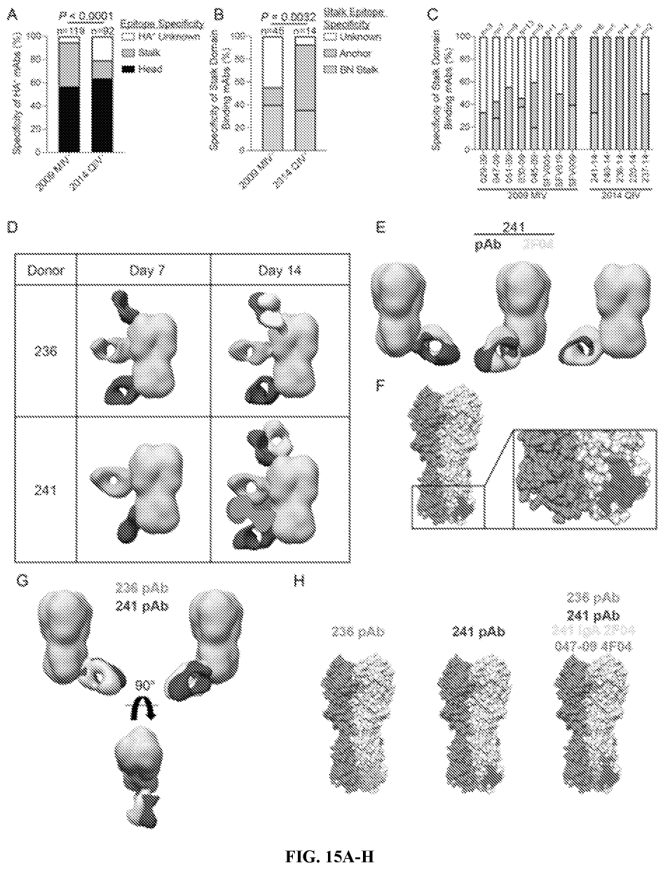

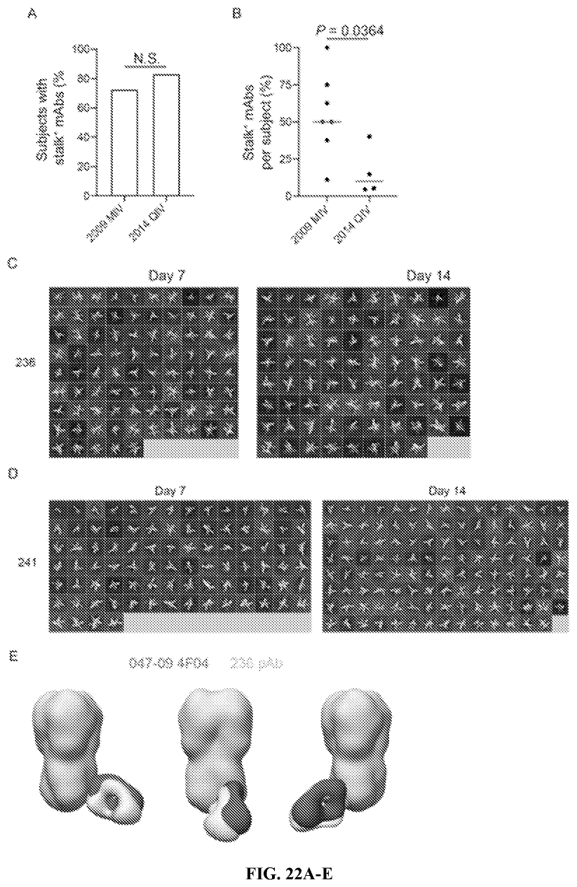

Antibodies induced by influenza vaccination are biased toward variable epitopes of the HA head (FIG. 15A). However, novel exposure to the 2009 pH1N1 virus robustly recalled MBCs targeting the conserved epitopes of the HA head and stalk domains, likely because subjects had low pre-existing antibody titers against the variable epitopes of the HA head (Andrews et al., 2015; Guthmiller et al., 2020; Li et al., 2012; Wrammert et al., 2011). In contrast, subjects that have been repeatedly exposed to the pH1N1 virus tend to recall MBCs targeting the variable epitopes of the HA head (Guthmiller et al., submitted for publication). Consistent with this, subjects that received the 2009 monovalent influenza virus (MIV) vaccine robustly induced a plasmablast response against the stalk domain (38%), as determined by generated mAbs, whereas only 15% of mAbs isolated from subjects that received the seasonal vaccine 2014 quadrivalent influenza virus vaccine (QIV) targeted the stalk domain (FIG. 15A). Most subjects in each cohort had at least one stalk domain-targeting mAb isolated, although the frequency of stalk domain-binding mAbs per subject was higher in the subjects that received the 2009 MIV relative to subjects in the 2014 QIV cohort (FIG. 22A-B). When broken down by the specific stalk epitopes targeted, nearly 40% of stalk binding mAbs isolated targeted the BN stalk epitope (FIG. 15B). A larger proportion of mAbs (57%) targeting the anchor epitope were isolated from subjects that received the 2014 QIV relative to those subjects that received the 2009 MIV (16%; FIG. 15B). Anchor epitope binding mAbs were detected in two out of six subjects in the 2014 QIV cohort (33.3%) and four out of eleven subjects in the 2009 MIV cohort (36%; FIG. 15C), demonstrating that this epitope is commonly targeted after influenza virus vaccinations.

To confirm that anchor epitope targeting mAbs generated from plasmablasts were representative of the serum antibody response, we performed electron microscopy polyclonal epitope mapping (EMPEM) to dissect the targets of the polyclonal serum antibody response mounted by subjects 236 and 241 from the 2014 QIV cohort. Both subjects had detectable antibodies targeting the anchor epitope at days 7 and 14 post vaccination, whereas only subject 241 had detectable antibodies against the BN stalk epitope at day 14 (FIG. 15D-E; FIG. 22C-D). Notably, subject 241 had more complexes with antibodies targeting the anchor than the BN stalk epitope (FIG. 22D), suggesting this subject more readily mounted an antibody response against the anchor epitope. Comparison of anchor epitope binding polyclonal antibodies (pAbs) identified in subjects 236 and 241 revealed the 241 IgA 2F04 mAb strongly overlapped with the 241 pAb from the same donor (FIG. 15E-F) while the 236 pAb sat slightly anterior to the HA trimer, similar to 047-09 4F04 (FIG. 15G-H; FIG. 22E). Together, these data indicate influenza virus vaccination can recall MBCs targeting the anchor epitope.

### 6. The cHA Universal Influenza Virus Vaccine Candidate Robustly Induced Antibodies Against the Anchor Epitope

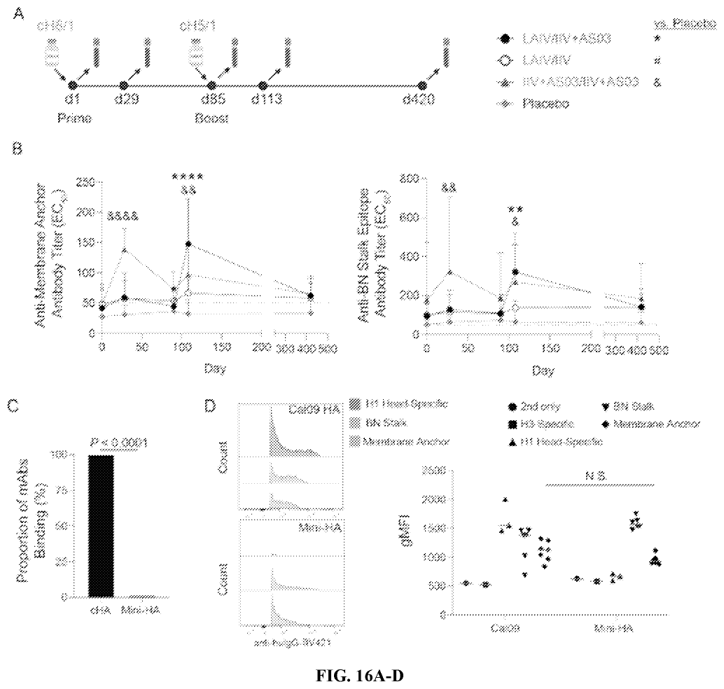

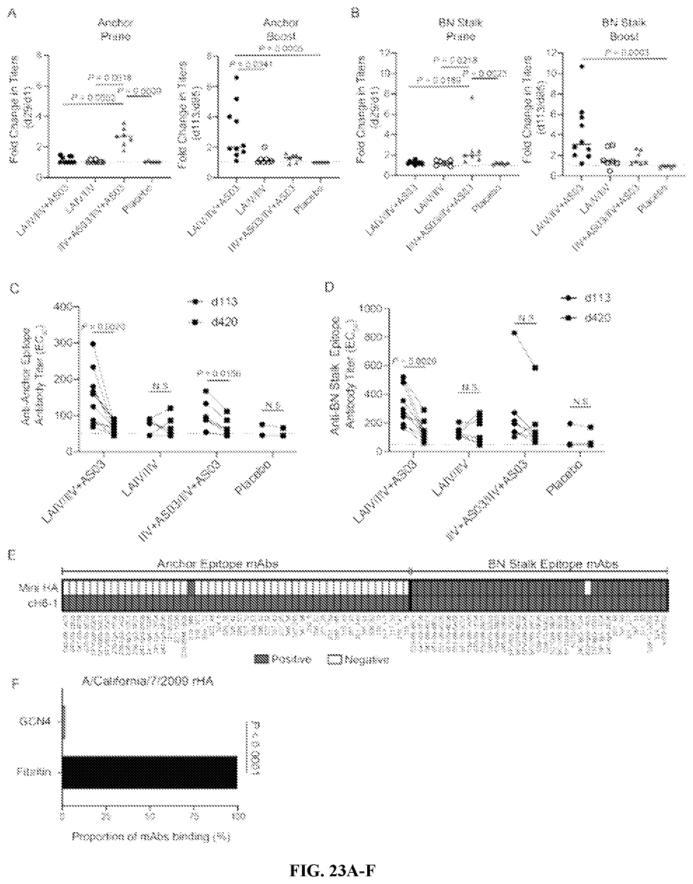

Several experimental universal influenza virus vaccine candidates currently being tested in clinical trials are intended to induce antibodies against the stalk domain. Notably, a cHA vaccine platform was shown to specifically induce antibodies against the stalk domain (Bernstein et al., 2020; Nachbagauer et al., 2020). To investigate whether subjects who received the cHA vaccine mounted an antibody response against the anchor epitope and the BN stalk epitope, we adapted the competition ELISA to detect serum antibody responses that could compete for binding with 047-09 4F04 and CR9114, respectively. Subjects enrolled in the cHA clinical trial received a prime-boost regimen of cHA, with the prime being a cH8/1 inactivated influenza virus vaccine with an adjuvant (IIV+ASO3) or cH8/1 live-attenuated influenza virus (LAIV) followed by a boost 3 months later with a cH5/1 IIV with or without adjuvant (FIG. 16A). On the prime vaccination, subjects that received the cH8/1 IIV+ASO3 had a dramatic increase in serum antibody responses against the anchor epitope and BN stalk epitope relative to the placebo group (FIG. 16B) and had a 3-fold increase in antibodies binding the anchor epitope over the day 0 time point (FIG. 23A-B). However, these titers drastically dropped after 3 months post-prime (FIG. 16B). Subjects that received the cH8/1 LAIV did not have an increase in serum antibody responses against either the anchor epitope or the BN stalk epitope (FIG. 16B; FIG. 23A-B). After the cH5/1 boost, subjects in the LAIV/IIV+ASO3 group dramatically increased antibody titers against both the anchor epitope and BN stalk epitope, whereas subjects in the LAIV/IIV cohort with no adjuvant did not have a substantial increase in serum antibodies compared to the placebo cohort (FIG. 16B). Subjects that received the cH8/1 IIV+ASO3 followed by the cH5/1 IIV+ASO3 also boosted antibody responses against both the anchor and BN stalk epitopes compared to the placebo controls (FIG. 16B), although the fold-change in titers was not statistically greater than the placebo (FIG. 23A-B). Furthermore, only subjects in the LAIV/IIV+ASO3 cohort had a significant fold-increase in antibodies targeting both the anchor and BN stalk epitopes relative to pre-boost titers (FIG. 23A-B). At a 1-year time point, subjects within the LAIV/IIV+ASO3 and the IIV+ASO3/IIV+ASO3 had a significant decrease in serum antibodies against the anchor epitope and subjects within the LAIV/IIV+ASO3 cohort had a significant decrease in serum antibodies against the BN stalk epitopes (FIG. 23C-D). Furthermore, we identified and generated anchor epitope targeting mAbs from acutely activated plasmablasts isolated from one subject that received the cH8/1 IIV+ASO3 prime and one subject that received the cH5/1 IIV+ASO3 boost (Table S1 and Table S2). Together, these data indicate that the cHA vaccine strategy can robustly induce antibodies against the anchor epitope.

Headless HA antigens, or mini-HAs, are attractive universal influenza virus vaccine antigens, as these antigens lack the immunodominant epitopes of the HA head (Impagliazzo et al., 2015; Yassine et al., 2015). We next tested whether the anchor epitope was present on the recombinant mini-HA antigen (Impagliazzo et al., 2015) by performing ELISAs with the anchor epitope targeting antibodies. Only 1 out of 50 anchor antibodies bound the mini-HA antigen, whereas all anchor epitope binding mAbs bound cH6/1 (FIG. 16C; FIG. 23E). In contrast, all but one BN stalk epitope targeting mAbs were capable of binding to both the mini-HA and the cH6/1 (FIG. 23E), suggesting the anchor epitope is specifically disrupted on this antigen. Compared to full-length HA, the membrane proximal region of the mini-HA splays by approximately 14.5 Å (Impagliazzo et al., 2015), suggesting this splaying may disrupt the antigenicity of the anchor epitope. Notably, the mini-HA antigen utilizes a GCN4 trimerization domain, whereas the cH6/1 utilizes a fibritin trimerization domain. Therefore, we next tested whether the loss of antigenicity of the anchor epitope could be due to the utilization of the GCN4 trimerization domain. We identified that anchor epitope targeting antibodies could bind A/California/July 2009 rHA with a fibritin trimerization domain, but not A/California/July 2009 rHA with a GCN4 trimerization domain (FIG. 23F), indicating a GCN4 trimerization domain affected the antigenicity of this epitope. To understand whether anchor epitope targeting antibodies could bind the mini-HA in a more native setting, we modified the mini-HA antigen to remove the trimerization domain and include a transmembrane domain, which would lead to the HA being membrane-bound. Transfected HEK293T cells were stained with mAbs targeting H1 head epitopes, the BN stalk epitope, or the anchor epitope, and flow cytometry was performed. MAbs targeting the anchor and BN stalk epitopes readily bound both the full-length membrane-bound A/California/July 2009 HA (Cal09) and the membrane-bound mini-HA, whereas the H1 head-specific mAbs only bound the full-length Cal09 HA (FIG. 16D). These data indicate that the anchor epitope is antigenic when HA is trimerized more similarly to membrane-bound HA. Additionally, these data indicate native-like HA antigens are likely to recall MBCs targeting the anchor epitope, such as the cHA vaccine candidate.

### 7. Anchor Epitope Targeting mAbs Utilize a Restricted Antibody Repertoire

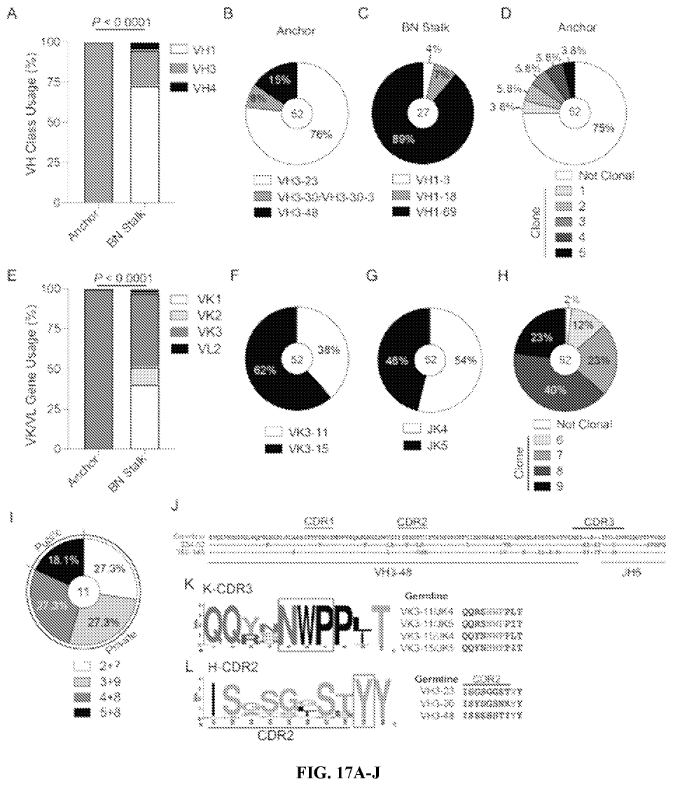

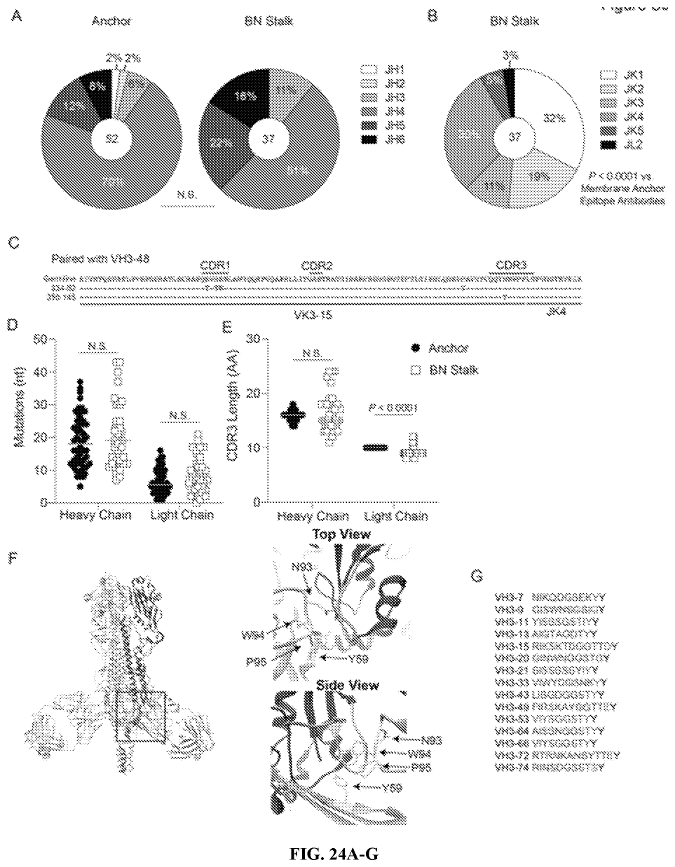

We next investigated the repertoire features of mAbs targeting the anchor epitope. All mAbs that targeted the anchor epitope utilized one of four VH3 genes: VH3-23, VH3-30/VH3-30-3, and VH3-48, with over three-quarters utilizing VH3-23 (FIG. 17A-B). MAbs targeting the BN stalk epitope commonly used VH1 genes, of which the vast majority used VH1-69 (FIG. 17A, C). Anchor epitope targeting mAbs used a variety of DH genes (Table S2) and JH genes, although 70% of anchor targeting mAbs utilized JH4 (FIG. 24A). Amongst the anchor targeting mAb heavy chain sequences, 75% were non-clonal (FIG. 17D), indicating most anchor targeting utilize similar but distinct heavy chain VDJ recombinations. Similar to the heavy chain, anchor epitope binding mAbs utilized a highly restricted light chain repertoire relative to the BN stalk binding mAbs, with all mAbs utilizing a combination of VK3-11 or VK3-15 combined with JK4 or JK5 (FIG. 17E-G; FIG. 24B). In contrast, mAbs targeting the BN stalk epitope used a wide array of VK/VL genes (FIG. 17E) and JK/JL genes (FIG. 24B). Furthermore, all but one light chain of the anchor targeting mAbs were clonal (FIG. 17H), indicating the light chains were very similar across mAbs and subjects. By determining paired heavy and light chain clones, we identified 4 distinct clonal expansions, with one public clonal expansion found across multiple subjects (FIG. 17I-J; FIG. 24C). Anchor epitope targeting mAbs were mutated to a similar extent as mAbs targeting the BN stalk epitope (FIG. 24D). The K-CDR3 length of anchor epitope binding mAbs was highly restricted, with all K-CDR3s being ten amino acids in length (FIG. 24E). Together, these data indicate that anchor epitope targeting mAbs utilize a highly restricted repertoire, particularly for the light chain.

FISW84 (Benton et al., 2018) similarly uses VH3-23/VK3-15 and largely makes interactions with the epitope via an NWP motif within the K-CDR3 loop and a tyrosine (Y) immediately following the H-CDR2 (FIG. 24F). We identified that all anchor targeting mAbs possessed this NWP motif at the exact same location within the K-CDR3, which was present in the germline sequence of the various VK/JK pairings (FIG. 17K). Moreover, all anchor binding mAbs utilized a germline encoded tyrosine at position 59 (FIG. 17L), suggesting this residue could have led to the selection of B cells utilizing these particular VH3 genes. Despite this, nearly 2/3 of VH3 genes utilize a tyrosine at this exact position (FIG. 24G), suggesting other features of the heavy chain may lead to the preferential selection of these particular VH3 genes into the B cell repertoire against the anchor epitope. Together, these data reveal B cells targeting the anchor epitope utilized a highly restricted V(D)J gene repertoire, and these specific features of the repertoire are likely critical for binding the anchor epitope.

### 8. Humans Possess MBCs with Features of Anchor Epitope Targeting Antibodies

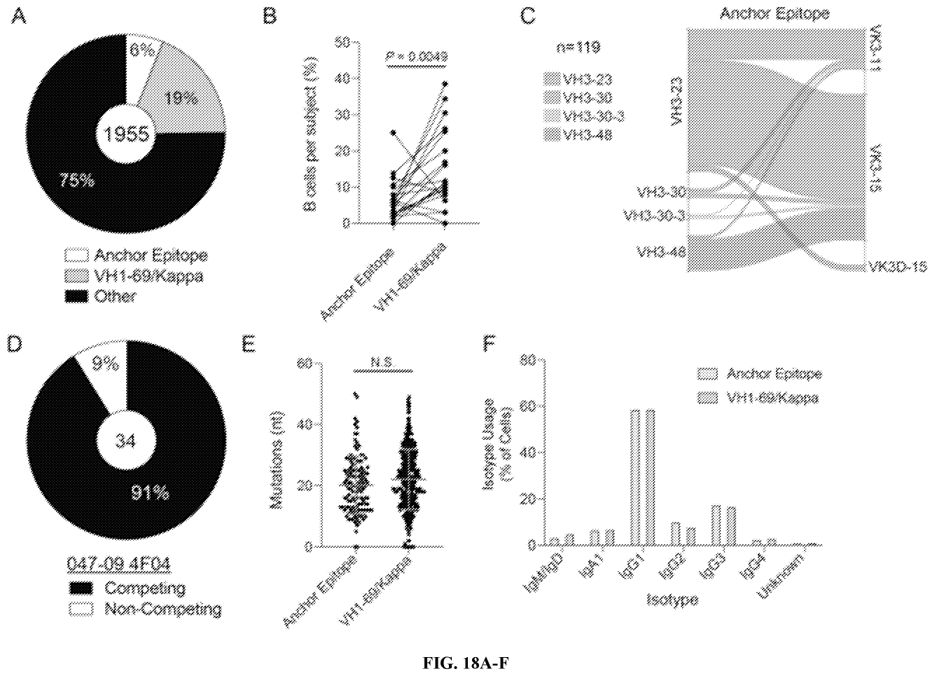

Due to the restricted repertoire features of anchor targeting mAbs, we next determined the relative proportion of B cell subsets with these features by integrating single-cell RNA-sequencing and repertoire sequencing of HA-specific B cells isolated from 22 subjects following cH5/1 vaccination (d112; FIG. 16A). Notably, most B cells isolated likely target the H1 stalk domain as we sequenced sorted cH5/1+ B cells and humans have no measurable pre-existing immunity against the H5 head domain (Han et al., 2020). However, subjects may have recruited naïve B cells against the H5 head component of the cH5/1 vaccine, therefore the isolated B cell pool is likely a heterogenous population of mostly H1 stalk domain-reactive B cells and some H5 head domain-reactive B cells. To investigate the proportion of B cells with repertoire features of B cells targeting the anchor epitope, we selected B cells that used VH3-23/VH3-30/VH3-30-3/VH3-48, VK3-11/VK3-15, JK4/JK5, a 10 amino acid length K-CDR3, and possessed an NWP motif within the K-CDR3. For reference, we additionally segregated out B cells expressing VH1-69 and a kappa chain, as these are the dominate repertoire features of B cells targeting the BN stalk epitope (FIG. 17A, C, E). We identified that B cells with features of antibodies binding the anchor epitope were abundant within the human B cell repertoire, with 6% of all B cells identified fitting within this defined repertoire (FIG. 18A). Of subjects with ten or more VDJ+ B cells (n=20), we identified anchor targeting B cells in all but one subject (FIG. 18B). The anchor targeting B cell pool largely used VH3-23/VK3-15 pairing (FIG. 18C). Additionally, we generated 34 mAbs from the selected anchor targeting B cell list and 31 of these mAbs competed with 047-09 4F04 (FIG. 18D). The anchor epitope B cells had a similar number of mutations as VH1-69/kappa B cells (FIG. 18E) and were largely class-switched to IgG1 and IgG3 (FIG. 18F), indicative of prior class-switch recombination and B cell selection within germinal centers. Together, these data indicate that the anchor epitope is a common target of the human MBC repertoire against HA. Moreover, this study indicates that most adults have pre-existing immunity against this epitope that can be harnessed by a potential universal influenza virus vaccine candidate to provide broad protection against H1-expressing viruses.

### B. Experimental Model and Subject Details

### 1. Human Materials

Human PBMCs were obtained from multiple subjects from multiple cohorts, which is outlined in Table S1. All studies were performed with the approval of the University of Chicago Institutional Review Board (ID #09-043-A). The chimeric HA vaccine study cohort is identified as clinical trial NCT03300050.

### 2. Cell Lines

Human Embryonic Kidney HEK293T (female, #CRL-11268) and Madin Darby canine kidney MDCK (female, #CCL-34, NBL-2) cells were purchased and authenticated by the American Type Culture Collection (ATCC). All cells were maintained in a humidified atmosphere of 5% CO2 at 37° C. HEK293T cells were maintained in Advanced-DMEM supplemented with 2% ultra-low IgG fetal bovine serum (FBS) (Invitrogen), 1% L-glutamine (Invitrogen) and 1% antibiotic-antimycotic (Invitrogen). MDCK cells were maintained in DMEM supplemented with 10% FBS (Invitrogen), 1% L-glutamine (Invitrogen) and 1% penicillin-streptomycin (Invitrogen).

### C. Method Details

### 1. Monoclonal Antibody Production

Monoclonal antibodies were generated as previously described (Guthmiller et al., 2019; Smith et al., 2009; Wrammert et al., 2008). Peripheral blood was obtained from each subject approximately 7 days after vaccination or infection or obtained 28+ days post-vaccination. Lymphocytes were isolated and enriched for B cells using RosetteSep. Total PBs (CD3⁻CD19⁺CD27^{hi}CD38^{hi}; all cohorts except 2014 QIV), IgG⁺PBs (CD3⁻CD19⁺IgM⁻CD27^{hi}CD38^{hi}IgG⁺IgA⁻; 2014 QIV), IgA PBs (CD3⁻CD19⁺IgM⁻CD27^{hi}CD38^{hi}IgG⁻IgA⁺; 2014 QIV cohort), or HA⁺ bait-sorted MBCs (CD3⁻CD19⁺CD27⁺CD381^{lo/+}HA⁺, for 030-09M 1B06) were single-cell sorted into 96-well plates. Immunoglobulin heavy and light chain genes were amplified by reverse transcriptase polymerase chain reaction (RT-PCR), sequenced, cloned into human IgG1, human kappa chain, or human lambda expression vectors, and co-transfected into HEK293T cells. Secreted mAbs were purified from the supernatant using protein A agarose beads. For mAbs generated from the 2014 QIV cohort, mAb names include the original isotype of the sorted PB, and all mAbs were expressed as human IgG1. cH5/1-binding B cells (CD19⁺CD27⁺cH5/1⁺) were sorted from subjects 28 days after cH5/1 vaccination (NCT03300050). Cells were sorted with A/California/04/2009 HA (for 030-09M 1B06) or cH5/1 probe with a Y98F mutation to ablate non-specific binding to sialic acids on B cells. MAb heavy chain and light chain sequences were synthesized from single-cell RNA-sequencing data of cH5/1-baited B cells (IDT), and cloned into the human IgG1, human kappa chain, or human lambda expression vectors. B cell clones were determined by aligning all the V(D)J sequences sharing identical progenitor sequences, as predicted by IgBLAST using our in-house software, VGenes. Consensus sequence analysis was performed using WebLogo (Crooks et al., 2004) and sequence alignments were determined using Clustal Omega.

### 2. Viruses and Recombinant Proteins

Influenza viruses used in all assays were grown in-house in specific pathogen free (SPF) eggs, harvested, purified, and titered. The A/swine/Mexico/AVX August2011 H1N2 virus (Mena et al., 2016) was provided by Ignacio Mena, Adolfo Garcia-Sastre, and Sean Liu at Icahn School of Medicine at Mount Sinai. Recombinant HA, cHA, and mini-HA were obtained from BEI Resources or kindly provided by the Krammer laboratory at Icahn School of Medicine at Mount Sinai, the Coughlan laboratory at The University of Maryland School of Medicine, or the Wilson laboratory at the University of Chicago. Recombinant HA mutant proteins used in FIG. 13 were generated with identified mutations from the deep mutational scanning experiments (see below) or with known mutations that have arisen naturally or were identified in other studies (FIG. 21). All mutations were made on HA from A/California/July 2009. Mutant HAs were expressed in HEK293T cells and purified using Ni-NTA agarose beads (Qiagen).

### 3. Antigen Specific ELISA

High protein-binding microtiter plates (Costar) were coated with 8 hemagglutination units (HAU) of virus in carbonate buffer or with recombinant HA, including HA mutants described below, at 2 g/ml in phosphate-buffered saline (PBS) overnight at 4° C. Plates were washed the next morning with PBS 0.05% Tween and blocked with PBS containing 20% fetal bovine serum (FBS) for 1 hr at 37° C. Antibodies were then serially diluted 1:3 starting at 10 g/ml and incubated for 1.5 hr at 37° C. Horseradish peroxidase (HRP)-conjugated goat anti-human IgG antibody diluted 1:1000 (Jackson Immuno Research) was used to detect binding of mAbs, and plates were subsequently developed with Super Aquablue ELISA substrate (eBiosciences). Absorbance was measured at 405 nm on a microplate spectrophotometer (BioRad). To standardize the assays, control antibodies with known binding characteristics were included on each plate, and the plates were developed when the absorbance of the control reached 3.0 OD units. All ELISAs were performed in duplicate twice. To determine mAb affinity, a non-linear regression was performed on background subtracted ODs and Kd values were reported. To classify antigen-specificity, mAbs that did not definitively bind the HA head or stalk are listed as binding unknown HA⁺ epitopes. Affinity measurements, as represented as Kd at a molar concentration (M), were calculated using Prism 9 (Graphpad) by performing a non-linear regression.

### 4. Deep Mutational Scanning for Stalk Domain Mutants

The mutant libraires used herein were previously described (Doud and Bloom, 2016). The libraries consist of all single amino-acid mutations to A/WSN/1933 (H1N1). The experiments were performed by using biological triplicate libraries. The mutational antigenic profiling of the 045-09 2B06 was performed as previously outlined (Doud et al., 2017). In brief, 10⁶ TCID50 of two of the virus library biological replicates was diluted in 1 mL in IGM (Opti-MEM supplemented with 0.01% FBS, 0.3% BSA, and 100 g/ml calcium chloride) and incubated with an equal volume of 045-09 2B06 antibody at a final concentration of 50 or 25 g/mL for 1.5 hours at 37° C. MDCK-SIAT1 cells were infected with the virus antibody mixtures. 2 hours post-infection, the media was removed, the cells washed with 1 ml PBS, and 2 ml of fresh IGM was added. 15 hours post-infection, viral RNA was extracted, and PCR amplified according to the barcoded-subamplicon library preparation as previously described (Doud and Bloom, 2016). The overall fraction of virions that survive antibody neutralization was estimated using qRT-PCR targeting the viral nucleoprotein (NP) and cellular GAPDH as previously described (Doud et al., 2017). Using 10-fold serial dilutions of the virus libraries, we infected cells with no antibody selection to serve as a standard curve of infectivity. qPCR Ct values from the standard curve samples compared to the virus-antibody mix samples are determined for NP and GAPDH. We then generate a linear regression to fit the difference between the NP and GAPDH Ct values for the standard curve samples, and then use this curve to interpolate the fraction surviving for the antibody-virus selection samples. Across the three library replicates the fraction of virus surviving antibody selection was 0.17, 0.1, and 0.14.

Illumina(R) deep sequencing data was analyzed using dms_tools2 version 2.4.12 software package (Bloom, 2015) which can be found online at github.com/jbloomlab/dms_tools2. All of the computer code used is at https://github.com/jbloomlab/2B06_DMS, and the Jupyter notebook that performs most of the analysis is at github.com/jbloomlab/2B06_DMS/blob/master/analysis_notebook.ipynb. The sequencing counts were processed to estimate the differential selection for each mutation, which is the log enrichment of that mutation in the antibody-selected condition versus the control (Doud et al., 2017). The numerical measurements of the differential selection that 2B06 imposes on each mutation can be found here: github.com/jbloomlab/2B06_DMS/blob/master/results/diffsel/tidy_diffsel.csv.

### 5. Competition ELISAs

Plates were coated with 50 μl of A/California/July 2009 HA at a concentration of 1 g/ml and incubated overnight at 4° C. To biotinylate the antibodies with known epitope specificities, CR9114 and 047-09-4F04, were incubated at 4° C. with EZ-Link™ Sulfo-NHS-Biotin (Thermo Scientific) for 24 h or 48 h prior to use, respectively. After blocking the plates with PBS 20% FBS for 1 h at 37° C., serum samples were incubated (starting dilution of 1:50 for human serum or 20 g/ml for mAbs) in the coated wells for 2 h at room temperature. Either biotinylated CR9114 or 047-09-4F04 was then added at a concentration equal to twice its Kd and incubated in the wells with the serum or mAbs for 2 h at room temperature. The biotinylated antibodies were desalted before addition to remove free biotin using Zeba™ spin desalting columns, 7k MWCO (Thermo Scientific). After washing the plates, wells were incubated with HRP-conjugated streptavidin (Southern Biotech) at 37° C. for 1 h for detection of the biotinylated antibody. Super Aquablue ELISA substrate (eBiosciences) was then added and absorbance was measured at 405 nm on a microplate spectrophotometer (Bio-Rad). To standardize the assays, biotinylated CR9114 or TS-09-4F04 was incubated in designated wells on each plate without any competing serum or mAb, and data were recorded when the absorbance of these wells reached an optical density (OD) of 1 to 1.5 units. After subtracting background, percent competition by serum samples was then determined by dividing a sample's observed OD by the OD reached by the positive control, subtracting this value from 1, and multiplying by 100. For the serum data, ODs were log transformed and analyzed by non-linear regression to determine EC50 values using Prism software (Graphpad). For FIG. 16 and FIG. 22, only subjects with serum for all timepoints were included.

### 6. Microneutralization Assays

Microneutralization assays for mAb characterization were carried out as previously described (Chen et al., 2018; Henry Dunand et al., 2015). MDCK cells were maintained in DMEM supplemented with 10% FBS, 1% penicillin-streptomycin, and 1% L-glutamine at 37° C. with 5% CO2. The day before the experiment, 25,000 MDCK cells were added to each well of a 96-well plate. Serial two-fold dilutions of mAb were mixed with an equal volume of 100 TCID50 of virus for 1 hr and added to MDCK cells for 1 hr at 37° C. The mixture was removed, and cells were cultured for 20 hrs at 37° C. with 1× MEM supplemented with 1 g/ml tosyl phenylalanyl chloromethyl ketone (TPCK)-treated trypsin and appropriate mAb concentration. Cells were washed twice with PBS, fixed with 80% ice cold acetone at 20° C. for at least 1 hr, washed 3 times with PBS, blocked for 30 min with 3% BSA, and then treated for 30 min with 2% H2O2. Cells were incubated with a mouse anti-nucleoprotein antibody (1:1000; Millipore) in 3% BSA-PBS for 1 hr at room temperature (RT), followed by goat anti-mouse IgG HRP (1:1000; Southern Biotech) in 3% BSA-PBS for 1 hr at RT. The plates were developed with Super Aquablue ELISA substrate at 405 nm until virus only controls reached an OD of 1. The signal from uninfected wells was averaged to represent 100% inhibition. The signal from infected wells without mAb was averaged to represent 0% inhibition. Duplication wells were used to calculate the mean and SD of neutralization, and inhibitory concentration 50 (IC50) was determined by a sigmoidal dose response curve. The inhibition ratio (%) was calculated as below: ((OD Pos. Control−OD Sample)/(OD Pos. Control−OD Neg. Control))*100. The final IC50 was determined using Prism software (GraphPad).

### 7. In Vivo Challenge Infections

MAb cocktails (Table S4) were passively transferred into 6- to 8-week-old female BALB/c mice (Jackson Laboratories) by intraperitoneal injection of 0.2, 1, and 5 mg/kg mAb cocktail, which are further detailed in FIG. 22. Negative control mice received 5 mg/kg of the anthrax-specific mAb 003-15D03 as an isotype control. Two hours post-mAb injection, mice were anesthetized with isoflurane and intranasally challenged with 10 LD50 of mouse-adapted A/Netherlands/602/2009 H1N1 virus, with 10 l of virus administered into each nostril (20 μl total). As a read out, survival and weight loss were monitored 1-2 times daily for two weeks. Mice were euthanized upon 25% weight loss or at the end of the experiment (14 days post challenge). All experiments were done in accordance with the University of Chicago Institutional Animal Care and Use Committee.

### 8. HA Footprint Mapping

The footprints of three mAbs (FISW84 (PDB: 6HJQ), CR9114 (PDB: 4FQI), and FI6v3 (PDB: 3ZTN)) were mapped onto one HA protomer (A/California/April 2009, PDB: 4M4Y) using UCSF Chimera (Pettersen et al., 2004) and Adobe Photoshop. EM maps of HA:fab complexes were aligned in UCSF Chimera and footprints were mapped onto one HA protomer. Individual protomers of the HA trimer are indicated in different shades of gray.

### 9. Negative Stain EM

Immune complexes were prepared by incubating Fab with HA (A/California/04/2009 with E47K or E47G stabilizing mutations) at greater than 3:1 molar ratio for 2 hours at room temperature (RT). Samples were deposited at −10 g/mL on glow-discharged, carbon-coated 400 mesh copper grids (Electron Microscopy Sciences, EMS) and stained with 2% w/v uranyl formate. Samples were imaged at 52,000× magnification, 120 kV, on a Tecnai Spirit T12 microscope equipped with an Eagle CCD 4 k camera (FEI) or 62,000 magnification, 200 kV, on a Tecnai T20 microscope equipped with a CMOS 4k camera (TVIPS). Micrographs were collected with Leginon, single particles were processed with Appion and Relion, footprints were mapped with UCSF Chimera, and figures were made with UCSF Chimera (Lander et al., 2009; Pettersen et al., 2004; Scheres, 2012; Suloway et al., 2005).

### 10. EMPEM

Human serum samples were heat-inactivated at 55° C. for 30 min before incubating on Capture Select IgG-Fc (ms) Affinity Matrix (Fisher) to bind IgG at 4° C. for 72 hours on a rotator. Samples with IgG bound to resin were centrifuged at 4,000 rpm and supernatant was collected. IgG samples were washed 3 times with PBS followed by centrifugation to remove supernatant. Samples were buffer exchanged into buffer containing 100 mM Tris, 2 mM EDTA, and 10 mM L-cysteine through centrifugation with Amicon filters, then incubated with papain for 4 hours at 37° C. shaking at 80 rpm. The digestion reactions were quenched with 50 mM iodoacetamide, buffer exchanged to TBS, and separated by size-exclusion chromatography (SEC) with a Superdex 200 increase 10/300 column (GE Healthcare). Fab and undigested IgG were collected and concentrated and 500 g Fab was complexed with 10 g HA for 18 hours at room temperature. Reactions were purified by SEC and immune complexes were collected and concentrated. Negative stain EM grids were prepared as described above.

### 11. Membrane-Bound HA and mAb Staining

HEK293T cells were plated into a 6-well plate and transfected overnight with 0.2 g of plasmid and 10 g/ml PEI. After 12-16 hours, media was replaced with PFHM-II and cells were rested for 3 days. Transfected cells were trypsinized, washed, and aliquoted. Cells were stained with 10 g/ml of individual mAbs for 30 minutes. Cells were washed and stained with anti-human IgG Fc-BV421 for 30 minutes. Cells were washed 2 times and run on a BD LSRFortessa X-20. Data were analyzed using FlowJo v10.

### 12. Single-Cell RNA-Seq and Repertoire Analysis

cH5/1⁺ memory B cells (CD19⁺CD27⁺HA⁺) were bulk sorted and partitioned into nanoliter-scale Gel Bead-In-Emulsions (GEMs) to achieve single cell resolution using the 10× Genomics Chromium Controller and according to the manufacturer's instruction (10× Genomics). The sorted single cells were processed according to 5′ gene expression and B cell Immunoglobulin (Ig) enrichment instruction to prepare the libraries for sequencing. Libraries were sequenced using an Illumina HiSeq 4000 at Northwestern University or an Illumina NextSeq 500 at the University of Chicago. Cellranger Single-Cell Software Suite (version 3.0) was used to perform sample de-multiplexing, barcode processing, and single-cell 5′ and V(D)J counting, and Cellranger mkfastq was used to de-multiplex raw base call (BCL) files into sample-specific fastq files. Subsequently, reads were aligned to the GRCh38 human genome. Cellranger counts and Cellranger vdj package were used to identify gene expression and assemble V(D)J pairs of antibodies.

Single cell datasets were analyzed using Seurat 3 toolkit. We performed conventional pre-process steps for all 22 subjects including cell quality control (QC), normalization, identification of highly variable features, data scaling, and linear dimensional reduction. More specifically, we only kept cells with more than 200 and less then 2500 detected genes for QC step. We normalized the RNA data using conventional log normalization. We identified 2000 highly variable genes for each dataset and performed principle component analysis (PCA) in linear dimensional reduction step. We then integrated all 22 single cell datasets from vaccinated subjects to remove batch effects. In this analysis, we filtered our dataset and only kept cells with both transcriptome and full length and paired heavy and light chain V(D)J sequences (n=1955). From these cells, we identified a group of “VH1-69/Kappa” B cells that used the VH1-69 gene and kappa light chain. We also identified a group of “anchor epitope” B cells by the following rules: 1) VH locus: VH3-23, VH3-30, VH3-30-3, or VH-3-48; 2) VK locus: VK3-11 or VK3-15; 3) JK locus: JK4 or JK5; 4) K-CDR3 length equal to 10; 5) a “NWP” pattern in K-CDR3 peptide.

### 13. HA Conservation Modeling

To generate the group 1 HA conservation model, we selected one representative sequence for each group 1 HA subtype from FluDB (https://www.fludb.org/; Table S5) according to a prior study (Burke and Smith, 2014). A multiple sequence alignment from these HA protein sequences was generated using MUSCLE (Edgar, 2004) and the conservation of each residue was quantified using an entropy model (Crooks et al., 2004). HA conservation was visualized on a H1 protein (PDB: 4JTV) using PyMOL (Schrodinger).

### 14. Statistical Analysis

All statistical analyses were performed using Prism software (Graphpad Version 7.0) or R. Sample sizes (n) for the number of mAbs tested are indicated in corresponding figures or in the center of pie graphs. Number of biological repeats for experiments and specific tests for statistical significance used are indicated in the corresponding figure legends. P values less than or equal to 0.05 were considered significant. * P≤0.05, ** P≤0.01, *** P≤0.001, **** P≤0.0001.

### D. Tables

TABLE S1
Subject information and demographics.

| Subject | | Vaccine/Infection | Age | Sex |
|---|---|---|---|---|
| 029-09 | | 2009 MIV | 24 | M |
| 030-09 | | 2009 MIV | 31 | F |
| 045-09 | | 2009 MIV | 24 | M |
| 047-09 | | 2009 MIV | 22 | M |
| 051-09 | | 2009 MIV | 42 | M |
| SFV005 | | 2009 MIV | 60 | F |
| SFV009 | | 2009 MIV | 31 | F |
| SFV015 | | 2009 MIV | 48 | F |
| SFV018 | | 2009 MIV | 58 | F |
| SFV019 | | 2009 MIV | 48 | F |
| SFV020 | | 2009 MIV | 64 | F |
| sc70 | | 2009 pH1N1 Infection | 38 | F |
| sc1009 | | 2009 pH1N1 Infection | 21 | M |
| SF1000 | | 2009 pH1N1 Infection | 37 | M |
| 008-10 | | 2010 TIV | 26 | F |
| 011-10 | | 2010 TIV | 30 | M |
| 014-10 | | 2010 TIV | 27 | M |
| 017-10 | | 2010 TIV | 24 | M |
| 019-10 | | 2010 TIV | 22 | F |
| 028-10 | | 2010 TIV | 38 | M |
| 034-10 | | 2010 TIV | 39 | F |
| 039-10 | | 2010 TIV | 25 | M |
| 051-10 | | 2010 TIV | 43 | M |
| 220-14 | | 2014 QIV | 24 | F |
| 221-14 | | 2014 QIV | 34 | F |
| 236-14 | | 2014 QIV | 32 | F |
| 237-14 | | 2014 QIV | 32 | F |
| 240-14 | | 2014 QIV | 28 | M |
| 241-14 | | 2014 QIV | 29 | F |
| 121/324 | (d7/d112) | cHA IIV + AS03/IIV + AS03 | 24 | F |
| 222/310 | (d91/d112) | cHA LAIV/IIV + AS03 | 25 | F |

| | | | |
|---|---|---|---|
| 301 | cHA IIV + AS03/IIV + AS03 | 34 | F |
| 308 | cHA LAIV/IIV + AS03 | 29 | F |
| 311 | cHA IIV +AS03/IIV + AS03 | 28 | M |
| 317 | cHA LAIV/IIV + AS03 | 37 | M |
| 319 | cHA LAIV/IIV + AS03 | 31 | F |
| 322 | cHA IIV + AS03/IIV + AS03 | 20 | F |
| 326 | cHA LAIV/IIV | 27 | M |
| 327 | cHA LAIV/IIV + AS03 | 22 | F |
| 334 | cHA LAIV/IIV + AS03 | 33 | M |
| 336 | cHA LAIV/IIV + AS03 | 36 | F |
| 337 | cHA LAIV/IIV + AS03 | 27 | F |
| 346 | cHA LAIV/IIV | 20 | F |
| 347 | cHA LAIV/IIV + AS03 | 31 | F |
| 350 | cHA LAIV/IIV + AS03 | 35 | F |
| 351 | cHA IIV + AS03/IIV + AS03 | 27 | F |

TABLE S2
Anchor epitope binding mAb information.

| | Cellular | | | | HC Clone | | | KC Clone |
|---|---|---|---|---|---|---|---|---|
| mAb | Source | VH | DH | JH | # | VK | JK | # |
| 030-09 3E05 | Plasmablast | 3-23 | 3-22 | 4 | Non-clonal | 3-15 | 5 | 9 |
| 030-09M 1B06 | MBC | 3-23 | 3-10 | 4 | Non-clonal | 3-11 | 5 | 7 |
| 045-09 1A03 | Plasmablast | 3-23 | 6-13 | 4 | Non-clonal | 3-11 | 4 | 6 |
| 045-09 2B03 | Plasmablast | 3-23 | 5-12 | 4 | Non-clonal | 3-11 | 5 | 7 |
| 047-09 4F04 | Plasmablast | 3-48 | 6-19 | 4 | Non-clonal | 3-15 | 4 | 8 |
| SFV009 3D04 | Plasmablast | 3-23 | 2-2 | 4 | Non-clonal | 3-15 | 4 | 8 |
| SFV009 3G01 | Plasmablast | 3-30 | 3-16 | 4 | Non-clonal | 3-11 | 5 | 7 |
| SFV009 3G03 | Plasmablast | 3-23 | 3-15-3 | 5 | Non-clonal | 3-11 | 5 | 7 |
| 236 IgG 1A02 | Plasmablast | 3-23 | 2-2 | 4 | 2 | 3-11 | 5 | 7 |
| 236 IgG 1D01 | Plasmablast | 3-23 | 6-19 | 4 | Non-clonal | 3-11 | 5 | 7 |
| 236 IgG 1F01 | Plasmablast | 3-23 | 2-2 | 4 | 2 | 3-11 | 5 | 7 |
| 236 IgA 1F06 | Plasmablast | 3-23 | 2-2 | 4 | 2 | 3-11 | 5 | 7 |
| 241 IgG 2A06 | Plasmablast | 3-23 | 6-19 | 5 | Non-clonal | 3-15 | 4 | 8 |
| 241 IgA 1D05 | Plasmablast | 3-23 | 5-18 | 4 | 3 | 3-15 | 5 | 9 |
| 241 IgA 2F04 | Plasmablast | 3-23 | 2-21 | 4 | 3 | 3-15 | 5 | 9 |
| 241 IgA 2F06 | Plasmablast | 3-23 | 3-9 | 4 | 3 | 3-15 | 5 | 9 |
| 121 2C06 | Plasmablast | 3-23 | 2-15 | 4 | Non-clonal | 3-15 | 4 | 8 |
| 222 1C06 | Plasmablast | 3-48 | 2-15-2 | 4 | Non-clonal | 3-15 | 4 | 8 |
| 301_91 | MBC | 3-23 | 3-9 | 5 | Non-clonal | 3-11 | 5 | 7 |
| 301_249 | MBC | 3-48 | 5-24 | 3 | Non-clonal | 3-11 | 4 | Non-clonal |
| 301_275 | MBC | 3-23 | 2-21 | 4 | 1 | 3-15 | 4 | 8 |
| 308_60 | MBC | 3-23 | 1-26 | 4 | Non-clonal | 3-11 | 4 | 8 |
| 310_10 | MBC | 3-23 | 1-14 | 4 | Non-clonal | 3-15 | 4 | 8 |
| 310_49 | MBC | 3-23 | 3-22 | 4 | Non-clonal | 3-15 | 5 | 9 |
| 317_30 | MBC | 3-23 | 6-13 | 4 | Non-clonal | 3-15 | 4 | 8 |
| 317_117 | MBC | 3-23 | 2-2 | 4 | Non-clonal | 3-15 | 5 | 9 |
| 319_75 | MBC | 3-23 | 3-22 | 3 | Non-clonal | 3-11 | 5 | 7 |
| 319_147 | MBC | 3-48 | 3-15-3 | 6 | Non-clonal | 3-15 | 4 | 8 |
| 319_345 | MBC | 3-23 | 3-9 | 4 | 4 | 3-15 | 4 | 8 |
| 319_373 | MBC | 3-23 | 6-19 | 4 | 4 | 3-15 | 4 | 8 |
| 322_48 | MBC | 3-23 | 1-26 | 4 | 1 | 3-15 | 5 | 9 |
| 324_5 | MBC | 3-23 | 2-21 | 3 | Non-clonal | 3-15 | 5 | 9 |
| 326_38 | MBC | 3-23 | 1-26 | 4 | Non-clonal | 3-11 | 4 | 6 |
| 326_42 | MBC | 3-23 | 3-16 | 5 | Non-clonal | 3-15 | 4 | 8 |
| 326_50 | MBC | 3-30 | 5-12 | 4 | Non-clonal | 3-15 | 5 | 9 |
| 327_32 | MBC | 3-23 | 3-10 | 4 | Non-clonal | 3-15 | 4 | 8 |
| 334_52 | MBC | 3-48 | 6-13 | 6 | 5 | 3-15 | 4 | 8 |
| 334_53 | MBC | 3-23 | 2-2 | 4 | Non-clonal | 3-15 | 5 | 9 |
| 334_62 | MBC | 3-23 | 2-2 | 1 | Non-clonal | 3-11 | 4 | 6 |
| 337_32 | MBC | 3-30 | 5-24 | 4 | Non-clonal | 3-11 | 5 | 7 |
| 337_43 | MBC | 3-23 | 5-24 | 4 | Non-clonal | 3-15 | 4 | 8 |
| 337_95 | MBC | 3-48 | 3-16 | 6 | Non-clonal | 3-15 | 5 | 9 |
| 346_54 | MBC | 3-30-3 | 3-16 | 5 | Non-clonal | 3-11 | 4 | 6 |
| 347_64 | MBC | 3-23 | 1-7 | 2 | Non-clonal | 3-11 | 5 | 7 |
| 347_246 | MBC | 3-23 | 1-1 | 5 | Non-clonal | 3-15 | 5 | 9 |
| 350_8 | MBC | 3-23 | 3-16 | 4 | Non-clonal | 3-15 | 4 | 8 |
| 350_22 | MBC | 3-23 | 6-19 | 4 | Non-clonal | 3-11 | 4 | 6 |
| 350_132 | MBC | 3-48 | 1-26 | 4 | Non-clonal | 3-15 | 4 | 8 |
| 350_145 | MBC | 3-48 | 4-17 | 6 | 5 | 3-15 | 4 | 8 |
| 351_38 | MBC | 3-23 | 6-13 | 4 | Non-clonal | 3-15 | 4 | 8 |
| 351_93 | MBC | 3-23 | 1-1 | 4 | Non-clonal | 3-15 | 4 | 8 |
| 351_139 | MBC | 3-23 | 5-24 | 4 | Non-clonal | 3-11 | 4 | 6 |

TABLE S3
Mutation information for FIG. 2C-D.

| | | 045-09 2B06 | | | |
|---|---|---|---|---|---|
| | | Deep | Other | | |
| | Naturally | Mutational | Experimentally | | |
| Mutation | Occurring? | Scan? | Determined? | Note | Source |
| H38S (HA1) | | No | Yes | Affects C179 | (Doud et al., 2018) |
| | | | | binding | |
| L292R (HA1) | | Yes | | In footprint of | (Lang et al., 2017) |
| | | | | many VH1-69 | |
| | | | | utilizing mAbs | |
| I323V (HA1) | Yes | No | | Present in pH1N1 | (Xu et al., 2012) |
| | | | | viruses | |
| K39D (HA2) | | Yes | Yes | Contacts with | (Fu et al., 2016) |
| | | | | VH3-30 mAb | |
| Q42E (HA2) | | Yes | Yes | Interact with | (Wu et al., 2020) |
| | | | | CR9114 and | |
| | | | | FI6v3 | |
| A44V (HA2) | | No | Yes | Expands in | (Park et al., 2020; |
| | | | | presence of stalk | Tan et al., 2012) |
| | | | | binding mAbs, | |
| | | | | escape mutant of | |
| | | | | 6F12 | |
| I45L (HA2) | | Yes | Yes | Mutation | (Lingwood et al., |
| | | | | associated with | 2012; Wu et al., |
| | | | | resistance to | 2020; Yassine et |
| | | | | group 1 | al., 2018) |
| | | | | neutralizing stalk | |
| | | | | mAbs | |
| D46R (HA2) | | Yes | | Pre-pH1N1 | |
| | | | | viruses express | |
| | | | | N46 | |
| E47K (HA2) | Yes | Yes | | Associated with | (Cotter et al., 2014) |
| | | | | HA protein | |
| | | | | stability | |
| T49R (HA2) | | Yes | | Mutation | (Lingwood et al., |
| | | | | associated with | 2012; Yassine et |
| | | | | resistance to | al., 2018) |
| | | | | group 1 | |
| | | | | neutralizing stalk | |
| | | | | mAbs | |
| N50R (HA2) | | Yes | | Interacts with | (Dreyfus et al., |
| | | | | CR9114 | 2012) |
| v52A (HA2) | | No | Yes | Affects FI6v3 | (Wu et al., 2020) |
| | | | | mAb binding | |
| I56A (HA2) | | No | Yes | Affects F10 mAb | (Sui et al., 2009) |
| | | | | binding | |
| E57K (HA2) | | Yes | | Critical residue | (Vanderlinden et |
| | | | | for membrane | al., 2010) |
| | | | | fusion | |

TABLE S4
MAbs used in Figure 3 infection studies.

| mAb name | Epitope Specificity |
|---|---|
| 241 IgG 2A06 | Anchor |
| 047-09 4F04 | Anchor |
| 030-09 3E05 | Anchor |
| SFV009 3G01 | Anchor |
| 236 IgG 1A02 | Anchor |
| 045-09 2B06 | BN Stalk |
| SFV005 2G02 | BN Stalk |
| SFV019 4E03 | BN Stalk |
| 220 IgG 1A05 | BN Stalk |
| 241 IgA 2E06 | BN Stalk |

TABLE S5
Group 1 HA strains used in FIG. S2C.

| HA Subtype | Strain Name |
|---|---|
| H1 | A/California/04/2009 |
| H2 | A/Singapore/1/1957 |
| H5 | A/mallard/Italy/3401/2005 |
| H6 | A/chicken/Taiwan/0705/1999 |
| H8 | A/turkey/Ontario/6118/1968 |
| H9 | A/swine/HongKong/9/1998 |
| H11 | A/duck/England/1/1056 |
| H12 | A/duck/Alberta/60/1976 |
| H13 | A/gull/Maryland/704/1977 |
| H16 | A/black-headed-gull/Turkmenistan/13/1976 |
| H17 | A/little-yellow-shouldered-bat/Guatemala/060/2010 |
| H18 | A/flat-facedbat/Peru/033/2010 |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

LENGTHY TABLES
The patent application contains a lengthy table section. A copy of the table is available in electronic form from the USPTO web site (https://seqdata.uspto.gov/?pageRequest=docDetail&DocID=US20250059260A1). An electronic copy of the table will also be available from the USPTO upon request and payment of the fee set forth in 37 CFR 1.19(b)(3).

1. An antibody or antigen binding fragment comprising a heavy chain variable region and a light chain variable region, wherein the heavy chain variable region comprises a HCDR1, HCDR2, and HCDR3 having at least 80% sequence identity to the HCDR1, HCR2, HCR3 from a heavy chain variable region encoded by the heavy chain nucleic acid of an antibody clone of Table 1 and wherein the light chain variable region comprises a LCDR1, LCDR2, and LCDR3 having at least 80% sequence identity to the LCDR1, LCDR2, and LCDR3 from the light chain variable region encoded by the light chain nucleic acid of the same antibody clone of Table 1.

2. The antibody or antigen binding fragment of claim 1, wherein the heavy chain variable region comprises a HCDR1, HCDR2, and HCDR3 having the amino acid sequence of an of a HCDR1, HCDR2, and HCDR3 of a clone encoded by the heavy chain nucleic acid of Table 1 and wherein the light chain variable region comprises a LCDR1, LCDR2, and LCDR3 comprising the amino acid sequence of the LCDR1, LCDR2, and LCDR3 from the light chain variable region of the same clone encoded by the light chain nucleic acid of Table 1.

3. The antibody or antigen binding fragment of claim 1 or 2, wherein the HCDR1, HCDR2, HCDR2, LCDR1, LCDR2, and LCDR3 each comprise an amino acid sequence that has at least 80% sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone.

4. The antibody or antigen binding fragment of claim 1 or 2, wherein the HCDR1, HCDR2, HCDR2, LCDR1, LCDR2, and LCDR3 each comprise the amino acid sequence of an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone.

5. The antibody or antigen binding fragment of any one of claims 1-4, wherein the heavy chain variable region comprises an amino acid sequence with at least 80% sequence identity to a heavy chain variable region of an antibody clone encoded by the heavy chain nucleic acid of Table 1 and/or the light chain variable region comprises an amino acid sequence with at least 80% sequence identity to the light chain variable region of the same antibody clone encoded by the light chain nucleic acid of Table 1.

6. The antibody or antigen binding fragment of claim 5, wherein the heavy chain variable region comprises the amino acid sequence of a heavy chain variable region of an antibody clone encoded by the heavy chain nucleic acid of Table 1 and/or the light chain variable region comprises the amino acid sequence of the same antibody clone encoded by the light chain nucleic acid of Table 1.

7. The antibody or antigen binding fragment of any one of claims 1-6, wherein the antibody or antigen binding fragment comprises a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 comprises an amino acid sequence with at least 80% sequence identity to an HFR1, HFR2, HFR3, and HFR4, respectively, of an antibody clone encoded by the heavy chain nucleic acid of Table 1, and the LFR1, LFR2, LFR3, and LFR4 comprises an amino acid sequence with at least 80% sequence identity to the LFR1, LFR2, LFR3, and LFR4, respectively, ofthe same antibody clone encoded by the light chain nucleic acid of Table 1.

8. The antibody or antigen binding fragment of any one of claims 1-6, wherein the HFR1, HFR2, HFR3, and HFR4 comprises the amino acid sequence of an HFR1, HFR2, HFR3, and HFR4, respectively, of an antibody clone encoded by the heavy chain nucleic acid of Table 1, and the LFR1, LFR2, LFR3, and LFR4 comprises the amino acid sequence of the LFR1, LFR2, LFR3, and LFR4, respectively, of the same antibody clone encoded by the light chain nucleic acid of Table 1.

9. The antibody or antigen binding fragment of any one of claims 1-8, wherein the antibody comprises a heavy chain and a light chain and wherein the heavy chain comprises an amino acid sequence with at least 70% sequence identity to a heavy chain of an antibody clone encoded by the heavy chain nucleic acid of Table 1 and the light chain comprises an amino acid sequence with at least 70% sequence identity to the light chain of the same antibody clone encoded by the light chain nucleic acid of Table 1.

10. The antibody or antigen binding fragment of claim 9, wherein the antibody comprises a heavy chain and a light chain and wherein the heavy chain comprises the amino acid sequence of an antibody clone encoded by the heavy chain nucleic acid of Table 1 and the light chain comprises the amino acid sequence of the same antibody clone encoded by the light chain nucleic acid of Table 1.

11. The antibody of any one of claims 1-10, wherein the antibody is human, chimeric, or humanized.

12. The antibody or antigen binding fragment of any one of claims 1-11, wherein the antibody is a neutralizing antibody.

13. The antibody or antigen binding fragment of any one of claims 1-12, wherein the antibody is a human antibody, humanized antibody, recombinant antibody, chimeric antibody, an antibody derivative, a veneered antibody, a diabody, a monoclonal antibody, a single domain antibody, or a single chain antibody.

14. The antigen binding fragment of any one of claims 1-12, wherein the antigen binding fragment is a single chain variable fragment (scFv), F(ab′)₂, Fab′, Fab, Fv, or rIgG.

15. A polypeptide comprising the antigen binding fragment of any one of claims 1-14.

16. The polypeptide of claim 15, wherein the polypeptide comprises at least two antigen binding fragments, wherein each antigen binding fragment is independently selected from an antigen binding fragment of any one of claims 1-14.

17. The polypeptide of claim 15 or 16, wherein the polypeptide is multivalent.

18. The polypeptide of any one of claims 15-17, wherein the polypeptide is bispecific.

19. A composition comprising the antibody or antigen binding fragment of any one of claims 1-18.

20. The composition of claim 19, wherein the composition comprises a pharmaceutical excipient.

21. The composition of claim 19 or 20, wherein the composition further comprises an adjuvant.

22. The composition of any one of claims 19-21, wherein the composition is formulated for parenteral, intravenous, subcutaneous, intramuscular, or intranasal administration.

23. The composition of any one of claims 1-22, wherein the composition comprises at least two antibodies or antigen binding fragments.

24. One or more nucleic acids encoding the antibody or antigen binding fragment of any one of claims 1-14 or the polypeptide of claim 18.

25. A nucleic acid encoding an antibody heavy chain, wherein the nucleic acid has at least 70% sequence identity to a heavy chain nucleic acid of Table 1.

26. A nucleic acid encoding an antibody light chain, wherein the nucleic acid has at least 70% sequence identity to a light chain nucleic acid of Table 1.

27. A vector comprising the nucleic acid(s) of any one of claims 24-26.

28. A host cell comprising the nucleic acid of any one of claims 24-26 or the vector of claim 27.

29. The host cell of claim 28, wherein the host cell is a human cell, B cell, T cell, Chinese hamster ovary, NS0 murine myeloma cell, or PER.C6 cell.

30. A method of a making a cell comprising transferring the nucleic acid(s) of any one of claims 24-26 or the vector of claim 27 into a cell.

31. The method of claim 30, wherein the method further comprises culturing the cell under conditions that allow for expression of a polypeptide from the nucleic acid.

32. The method of claim 31, wherein the method further comprising isolating the expressed polypeptide.

33. The method of any one of claims 30-32, wherein the cell is a human cell, B cell, T cell, Chinese hamster ovary, NS0 murine myeloma cell, or PER.C6 cell.

34. A method for producing a polypeptide comprising transferring the nucleic acid(s) of any one of claims 24-26 or the vector of claim 27 into a cell and isolating polypeptides expressed from the nucleic acid.

35. The method of claim 34, wherein the cell is a human cell, B cell, T cell, Chinese hamster ovary, NS0 murine myeloma cell, or PER.C6 cell.

36. A method for treating or preventing an influenza infection in a subject, the method comprising administering to the subject, the antibody or antigen binding fragment of any one of claims 1-14, the polypeptide of claim 18, or the host cell of claim 28.

37. The method of claim 36, wherein the subject is a human subject.

38. The method of claim 36 or 37, wherein the subject has one or more symptoms of an influenza infection.

39. The method of claim 36 or 37, wherein the subject does not have any symptoms of an influenza infection.

40. The method of any one of claims 35-39, wherein the influenza infection comprises an H1N1 or H1N2 infection.

41. The method of any one of claims 36-40, wherein the subject has been diagnosed with an influenza infection.

42. The method of any one of claims 36-40, wherein the subject has not been diagnosed with an influenza infection.

43. The method of any one of claims 36-42, wherein the subject has been previously vaccinated for influenza.

44. The method of any one of claims 36-42, wherein the subject has not been previously vaccinated for influenza.

45. The method of any one of claims 36-44, wherein the antibody, antigen binding fragment, polypeptide, or cell is administered by parenteral, intravenous, subcutaneous, intramuscular, or intranasal administration.

46. The method of any one of claims 36-45, wherein the subject has been previously treated for an influenza infection.

47. The method of any one of claims 36-46, wherein the subject is administered an additional therapeutic.

48. The method of claim 47, wherein the additional therapeutic comprises a steroid or an anti-viral therapeutic.

49. A method for evaluating a sample from a subject, the method comprising contacting a biological sample from the subject, or extract thereof, with at least one antibody, antigen binding fragment, or polypeptide of any one of claims 1-18.

50. The method of claim 49, wherein the at least one antibody, antigen binding fragment, or polypeptide is operatively linked to a detectable label.

51. The method of claim 49 or 50, wherein the method further comprises incubating the antibody, antigen binding fragment, or polypeptide under conditions that allow for the binding of the antibody, antigen binding fragment, or polypeptide to antigens in the biological sample or extract thereof.

52. The method of any one of claims 49-51, wherein the method further comprises detecting the binding of an antigen to the antibody, antigen binding fragment, or polypeptide.

53. The method of any one of claims 49-52, wherein the method further comprises contacting the biological sample with at least one capture antibody, antigen, or polypeptide.

54. The method of claim 53, wherein the at least one capture antibody, antigen binding fragment, or polypeptide comprises at least one antibody of claims 1-18.

55. The method of claim 53 or 54, wherein the capture antibody is linked to a solid support.

56. The method of any one of claims 49-55, wherein the biological sample comprises a blood sample, urine sample, fecal sample, or nasopharyngeal sample.

57. A method for diagnosing an influenza infection in a subject, the method comprising contacting a biological sample from the subject, or extract thereof, with at least one antibody, antigen binding fragment, or polypeptide of any one of claims 1-18.

58. The method of claim 57, wherein the at least one antibody, antigen binding fragment, or polypeptide is operatively linked to a detectable label.

59. The method of claim 57 or 58, wherein the method further comprises incubating the antibody, antigen binding fragment, or polypeptide under conditions that allow for the binding of the antibody, antigen binding fragment, or polypeptide to antigens in the biological sample or extract thereof.

60. The method of any one of claims 57-59, wherein the method further comprises detecting the binding of an antigen to the antibody, antigen binding fragment, or polypeptide.

61. The method of any one of claims 57-60, wherein the method further comprises contacting the biological sample with at least one capture antibody, antigen, or polypeptide.

62. The method of claim 61, wherein the at least one capture antibody, antigen, or polypeptide comprises at least one antibody, antigen, or polypeptide of claims 1-18.

63. The method of claim 61 or 62, wherein the capture antibody is linked to a solid support.

64. The method of any one of claims 57-63, wherein the biological sample comprises a blood sample, urine sample, fecal sample, or nasopharyngeal sample.
